(19) United States
(12) Patent Application Publication
(10) Pub. No.: US 2026/0048125 A1
(43) Pub. Date: Feb. 19, 2026

(54) COMPOSITIONS AND METHODS FOR TREATING BLOOD CANCERS

(71) Applicant: A2 Biotherapeutics, Inc., Agoura Hills, CA (US)

(72) Inventors: Agnes Hamburger, Newbury Park, CA (US); Breanna Diandreth, Agoura Hills, CA (US); Carl Alexander Kamb, Thousand Oaks, CA (US); John Welch, Agoura Hills, CA (US)

(21) Appl. No.: 19/101,813

(22) PCT Filed: Aug. 8, 2023
(86) PCT No.: PCT/US2023/071839

§ 371 Date: Feb. 6, 2025

Related U.S. Application Data
(60) Provisional application No. 63/370,721, filed on Aug. 8, 2022.
(60) Provisional application No. 63/370,728, filed on Aug. 8, 2022.
(60) Provisional application No. 63/370,722, filed on Aug. 8, 2022.
(60) Provisional application No. 63/373,817, filed on Aug. 29, 2022.
(60) Provisional application No. 63/373,812, filed on Aug. 29, 2022.

(51) Int. Cl. A61K 40/31; A61K 40/11; A61K 40/42; C07K 14/725; C07K 16/28

(52) U.S. Cl. CPC A61K 40/31; A61K 40/11; A61K 40/421; A61K 40/4211; A61K 40/4221; C07K 14/7051; C07K 16/2803; C07K 16/283; C07K 16/2887; C07K 16/2896

(57) ABSTRACT

The disclosure relates to immune cells for treatment of blood cancer comprising an activator receptor comprising an extracellular ligand binding domain specific to an activator antigen expressed by blood cancer cells and an inhibitor receptor comprising an extracellular ligand binding domain specific to an inhibitor antigen expressed by non-cancerous blood cells.

### CROSS REFERENCE TO RELATED APPLICATIONS

This application claims priority to and benefit of U.S. Provisional Patent Application No. 63/370,721 filed on Aug. 8, 2022, U.S. Provisional Patent Application No. 63/370,728 filed on Aug. 8, 2022, U.S. Provisional Patent Application No. 63/370,722 filed on Aug. 8, 2022, U.S. Provisional Patent Application No. 63/373,817 filed on Aug. 29, 2022, U.S. Provisional Patent Application No. 63/373,812 filed on Aug. 29, 2022, the contents of each of which is incorporated herein by reference in its entirety.

### INCORPORATION BY REFERENCE OF SEQUENCE LISTING

The present application is being filed with a Sequence Listing in electronic format. The Sequence Listing is provided as a file entitled 061250-546001WO_SeqList_ST26.xml, created on Aug. 3, 2023, and is 714 kilobytes in size. The information in electronic format of the Sequence Listing is incorporated herein by reference in its entirety.

### BACKGROUND

Immune cells that are engineered with receptors to integrate signals from multiple antigens offer a route to achieve therapeutic selectivity in cancer patients.

In conventional adoptive cell therapies, immune cells are engineered to express specific receptors, for example chimeric antigen receptors (CARs) or T cell receptors (TCRs), which direct the activity of the immune cells to cellular targets via interaction of the receptor with a ligand expressed by the target cell. Identification of suitable target molecules remains challenging, as many targets are expressed in normal tissues which can lead to toxicity when the transplanted cells target normal tissues.

Selective toxicity for tumor cells is the fundamental obstacle to development of new oncology medicines. One promising strategy, called a synthetic signal-integration system, involves engineering effector cells that can integrate multiple signals derived from different profiles of antigens expressed on cancerous vs. normal cells.

One example of a synthetic signal-integration system involves an OR gate, by analogy to logical operations performed by machines, where either of two antigens (A-antigens) is sufficient to activate engineered effector cells. Such approaches have been designed for B cell-lineage tumors and involve two specific antigens: CD19 OR CD20 and CD19 OR CD22 (Zha et al., 2016 PMID: 27059623; Dai et al., 2020 PMID: 32245502). Another example of a synthetic signal-integration system involves AND gates in which two antigens, for example A and B, are needed to stimulate responses in engineered effector cells (Morsut et al., 2016 PMID: 26830878). A third example of a synthetic signal-integration system involves mechanisms that pair activating and inhibitory receptors which are termed NOT gates, for example, A not B. The NOT gate involves use of specific antigens to protect normal tissues via inhibitory receptors (Fedorov V D, et al., Sci Transl Med. 2013; 5, 215).

The clinical success of CAR therapy provides optimism for treating hematological malignancies. One challenge to developing CAR therapy for hematological malignancies is the lack of suitable targets.

There is thus a need in the art for compositions and methods useful in the treatment of blood cancers by adoptive cell therapy.

### SUMMARY

The present disclosure provides an immune cell for treatment of blood cancer, comprising an activator receptor comprising an extracellular ligand binding domain specific to a first activator antigen expressed by blood cancer cells, and an inhibitor receptor comprising an extracellular ligand binding domain specific to a first inhibitor antigen expressed by non-cancerous blood cells.

The present disclosure provides a nanocarrier comprising one or more polynucleotides, wherein the one or more polynucleotides encode: an activator receptor comprising an extracellular ligand binding domain specific to a first activator antigen expressed by blood cancer cells, and an inhibitor receptor comprising an extracellular ligand binding domain specific to a first inhibitor antigen expressed by non-cancerous blood cells.

In some embodiments, the nanocarrier is capable of delivering the one or more polynucleotides to an immune cell in vivo or ex vivo.

In some embodiments, the nanocarrier is a lipid nanoparticle (LNP).

In some embodiments, the one or more polynucleotides are one or more messenger ribonucleic acids (mRNAs) or modified mRNAs (mmRNAs).

In some embodiments, the first activator antigen is expressed by hematopoietic cells.

In some embodiments, the first inhibitor antigen is expressed by myeloid cells.

In some embodiments, the myeloid cells are differentiated.

In some embodiments, the first inhibitor antigen is expressed by hematopoietic stem cells.

In some embodiments, the first activator antigen is SPN or a peptide antigen of SPN.

In some embodiments, the first inhibitor antigen is SPN or a peptide antigen of PECAM-1.

In some embodiments, the first activator antigen is SPN and the first inhibitor antigen is PECAM-1.

In some embodiments, the cancer cell expresses SPN.

In some embodiments, the blood cancer cell is a lymphoma cell, a leukemia cell, a myeloma cell, a Reed-Sternberg cell, a myeloproliferative neoplasm cell, or a Waldenstrom macroglobulinemia cell.

In some embodiments, the blood cancer cell is a leukemia cell or a lymphoma cell.

In some embodiments, the PECAM-1 antigen is expressed by non-cancerous blood cells of a subject.

In some embodiments, non-cancerous blood cells of the subject express both the first activator antigen and the first inhibitor antigen.

In some embodiments, the activator receptor and the inhibitor receptor specifically activate the immune cell when in contact with the blood cancer cell.

In some embodiments, the activator receptor specifically activates the immune cell when in contact with the blood cancer cell.

In some embodiments, the inhibitor receptor specifically inhibits the immune cell when in contact with the non-cancerous blood cell.

In some embodiments, the immune cell is a T cell.

In some embodiments, the T cell is a CD8+ CD4− T cell.

In some embodiments, the SPN antigen comprises a sequence or subsequence at least 95% identical to a sequence or subsequence of any one of SEQ ID NOs: 172-173.

In some embodiments, the activator receptor is a T cell receptor (TCR) or a chimeric antigen receptor (CAR).

In some embodiments, the extracellular ligand binding domain of the activator receptor comprises an antibody fragment, a single chain Fv antibody fragment (scFv), a β chain variable domain (Vβ), or a TCR α chain variable domain and a TCR β chain variable domain.

In some embodiments, the extracellular ligand binding domain of the activator receptor comprises a heavy chain variable (VH) region and a light chain variable (VL) region.

In some embodiments, the VH region comprises the sequence of any one of SEQ ID NOs: 249-252, 257, 259, or 261-280; and the VL region comprises the sequence of any one of SEQ ID NOs: 253-256, 258, 260, or 281-303.

In some embodiments, the CAR comprises a hinge sequence isolated or derived from CD8, CD28, IgG1, or IgG4, or a synthetic hinge.

In some embodiments, the CAR comprises a transmembrane domain isolated or derived from CD8 or CD28.

In some embodiments, the CAR comprises an intracellular domain isolated or derived from CD28, 4-1BB or CD3z, or a combination thereof.

In some embodiments, the inhibitor receptor is a TCR or a CAR.

In some embodiments, the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

In some embodiments, the extracellular ligand binding domain of the inhibitor receptor comprises a heavy chain variable (VH) region and a light chain variable (VL) region.

In some embodiments, the inhibitor receptor comprises a LILRB1 intracellular domain or a functional variant thereof.

In some embodiments, the inhibitor receptor comprises LILRB1 hinge and transmembrane domains, or functional variants thereof.

The present disclosure provides a pharmaceutical composition, comprising an effective amount of the immune cells of the present disclosure.

The present disclosure provides a pharmaceutical composition, comprising an effective amount of the nanocarriers of the present disclosure.

In some embodiments, the pharmaceutical composition further comprises a pharmaceutically acceptable carrier, diluent or excipient.

In some embodiments, the pharmaceutical composition is used as a medicament in the treatment of cancer.

The present disclosure provides a polynucleotide system, comprising one or more polynucleotides comprising polynucleotide sequences encoding an activator receptor comprising an extracellular ligand binding domain specific to a SPN antigen, and an inhibitor receptor comprising an extracellular ligand binding domain specific to a PECAM-1 antigen.

The present disclosure provides a method of making an immune cell therapy, comprising transforming immune cells with the polynucleotide system of the present disclosure.

The present disclosure provides a kit comprising the immune cell of the present disclosure or the pharmaceutical composition of the present disclosure.

The present disclosure provides an immune cell comprising:
(a) an activator receptor comprising an extracellular ligand binding domain specific to a SPN antigen, wherein the extracellular ligand binding domain of the activator receptor is an scFv; and
(b) an inhibitor receptor comprising an extracellular ligand binding domain specific to a PECAM-1 antigen that is not expressed by a blood cancer cell, wherein the extracellular ligand binding domain of the inhibitor receptor is an scFv.

The present disclosure provides a method of treating cancer in a subject, comprising administering to the subject an immune cell comprising
(a) an activator receptor comprising an extracellular ligand binding domain specific to a SPN antigen, wherein the extracellular ligand binding domain of the activator receptor is an scFv; and
(b) an inhibitor receptor comprising an extracellular ligand binding domain specific to a PECAM-1 antigen that is not expressed by a blood cancer cell, wherein the extracellular ligand binding domain of the inhibitory receptor is an scFv.

The present disclosure provides a method of generating engineered immune cells in a subject in need thereof, comprising administering to the subject a nanocarrier of the present disclosure or a pharmaceutical composition of the present disclosure.

The present disclosure provides a method of identifying antigens for use in treating blood cancers with chimeric antigen receptor immune cell therapy, comprising selecting an antigen expressed on blood cancer cells and selecting an antigen expressed on non-cancerous blood cells.

The present disclosure provides a method of killing tumor cells in a subject in need thereof, comprising administering to the subject a nanocarrier according to of the present disclosure or a pharmaceutical composition according to of the present disclosure.

The present disclosure provides a method of treating cancer in a subject in need thereof, comprising administering to the subject a nanocarrier according to of the present disclosure or a pharmaceutical composition of the present disclosure.

In some embodiments, the nanocarrier or pharmaceutical composition is administered in an amount effective to specifically kill blood cancer cells in the subject.

In some embodiments, the nanocarrier is administered by intravenous injection.

In some embodiments, the nanocarrier is a lipid nanoparticle (LNP).

In some embodiments, the one or more polynucleotides are delivered ex vivo.

The present disclosure provides a kit comprising a nannocarrier of the present disclosure or a pharmaceutical composition of the present disclosure.

The present disclosure provides a method of treating cancer in a subject, comprising administering to the subject a nanocarrier comprising one or more polynucleotides encoding:
(a) an activator receptor comprising an extracellular ligand binding domain specific to a SPN antigen, wherein the extracellular ligand binding domain of the activator receptor comprises:
(i) a VH region comprising sequences of any one of SEQ ID NOs: 249-252, 257, 259, or 261-280; and
(ii) a VL region comprising sequences of any one of SEQ ID NOs: 253-256, 258, 260, or 281-303; and
(b) an inhibitor receptor comprising an extracellular ligand binding domain specific to a PECAM-1 antigen that is not expressed by a blood cancer cell, wherein the polynucleotide is an mRNA, and wherein the polynucleotide is delivered to an immune cell without any carrier molecules.

In some embodiments, the nanocarrier is administered by intravenous injection.

The present disclosure provides a polynucleotide encoding an activator receptor comprising an extracellular ligand binding domain specific to a first activator antigen expressed by blood cancer cells; and an inhibitor receptor comprising an extracellular ligand binding domain specific to a first inhibitor antigen expressed by non-cancerous blood cells.

In some embodiments, the method comprises generating an immune cell comprising two or more receptors comprising antigen-binding domains, wherein one receptor binds to a first antigen expressed on blood cancer cells and one receptor binds to the second antigen expressed on non-cancerous blood cells.

The present disclosure provides an immune cell for treatment of blood cancer, comprising an activator receptor comprising an extracellular ligand binding domain specific to a first activator antigen, SPN, expressed by blood cancer cells, and an inhibitor receptor comprising an extracellular ligand binding domain specific to a first inhibitor antigen, PECAM-1, expressed by non-cancerous blood cells.

The present disclosure provides an immune cell for treatment of blood cancer, comprising an activator receptor comprising an extracellular ligand binding domain specific to a first activator antigen, CD45, expressed by blood cancer cells, and an inhibitor receptor comprising an extracellular ligand binding domain specific to a first inhibitor antigen, PECAM-1, expressed by non-cancerous blood cells.

The present disclosure provides an immune cell for treatment of blood cancer, comprising an activator receptor comprising an extracellular ligand binding domain specific to a first activator antigen, CD11a, expressed by blood cancer cells, and an inhibitor receptor comprising an extracellular ligand binding domain specific to a first inhibitor antigen, PECAM-1, expressed by non-cancerous blood cells.

The present disclosure provides an immune cell for treatment of blood cancer, comprising an activator receptor comprising an extracellular ligand binding domain specific to a first activator antigen, SPN, and a second extracellular binding domain specific to a second activator antigen, CD33, expressed by blood cancer cells, and an inhibitor receptor comprising an extracellular ligand binding domain specific to a first inhibitor antigen, PECAM-1, expressed by non-cancerous blood cells.

The present disclosure provides an immune cell for treatment of blood cancer, comprising an activator receptor comprising an extracellular ligand binding domain specific to a first activator antigen, SPN, and a second extracellular binding domain specific to a second activator antigen, FLT3, expressed by blood cancer cells, and an inhibitor receptor comprising an extracellular ligand binding domain specific to a first inhibitor antigen, PECAM-1, expressed by non-cancerous blood cells.

The present disclosure provides an immune cell for treatment of blood cancer, comprising an activator receptor comprising an extracellular ligand binding domain specific to a first activator antigen, CD33, and a second extracellular binding domain specific to a second activator antigen, FLT3, expressed by blood cancer cells, and an inhibitor receptor comprising an extracellular ligand binding domain specific to a first inhibitor antigen, PECAM-1, expressed by non-cancerous blood cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a diagram depicting engineering a cell using an mRNA lipid nanoparticle (LNP).

FIG. 1B is a graph showing the expression of CD33 in non-cancerous “normal” HSCs, AML stage cells, and non-cancerous “normal” monocytes.

FIGS. 2A and 2B are illustrative designs of cellular logic systems to integrate multiple-antigen inputs.

FIG. 2A shows venn diagrams depicting classes of multi-antigen receptor systems that have been constructed in immune effector cells to integrate inputs (logic gates). Teal represents the antigen combination on target cells that activates killing by the engineered immune cells.

FIG. 2B depicts Tmod implementation of a NOT-gate. Cells, receptors and antigens involved in tumor-selective killing. The Tmod NOT-gate system incorporates an inhibitory receptor (blocker) which inhibits T cell activation when engaged with the blocker antigen present on normal cells but not when the blocker antigen is absent on cancer cells. Tmod=immune cells expressing both activator and inhibitor receptors.

FIG. 3 shows a schematic depicting an illustrative model for how the Tmod system functions as a proximity detector to integrate antigen inputs locally in real time. Tmod cells are able to spare normal cells based on detection of activator/blocker antigens on the same vs. different surfaces. Only when activator and blocker antigens are in close proximity (i.e. on the same surface) will the CAR be blocked. Therefore even Tmod cells interacting with many normal cells through blocker binding will not be prevented from killing a cancer cell.

FIG. 4 is a diagram depicting Tmod cell properties of therapeutic relevance. Tmod cells can selectively kill tumor cells while in a sea of normal tissue but also spare nearby normal cells while surrounded by tumor; reversibility: Tmod cells can kill tumor cells after encountering normal cells and also spare normal cells after encountering and killing tumor cells (and back-and-forth).

FIG. 5 is a schematic of an illustrative algorithm to identify A and B antigens for activator and inhibitor receptors for blood cancers.

FIG. 6 is a graph depicting A antigen SPN expression (left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 7 is a graph depicting A antigen CD45 expression (left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 8 is a graph depicting A antigen LFA-1 expression (left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 9 is a graph depicting A antigen SPN expression (top left panel) and A antigen CD33 expression (bottom left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 10 is a graph depicting A antigen SPN expression (top left panel) and A antigen CD45 expression (bottom left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 11 is a graph depicting A antigen SPN expression (top left panel) and A antigen FLT3 expression (bottom left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 12 is a graph depicting A antigen CD33 expression (top left panel) and A antigen FLT3 expression (bottom left panel) measured in transcripts per million (TPM) in AML subtype cells, CD3+ cells, CD19+ cells, HSCs, monocytes (Mono), and neutrophils (Neu). The right panel is a graph depicting B antigen (PECAM-1) expression measured in TPM AML subtype cells, CD3+ cells, CD19+ cells, HSCs, monocytes (Mono), and neutrophils (Neu).

FIG. 13 shows a anti-CD19 CAR, anti-CD20 CAR, or tandem anti-CD19/anti-CD20 activator CAR functioning as an OR gate in combination with an HLA-A*02 inhibitor receptor module. The plot depicts HLA*A*02 blocker IC50 measurements when paired with anti-CD19, anti-CD20, or both anti-CD19 and anti-CD20 tandem activators in Jurkat cells, with titration of HLA-A*02 mRNA in ng, showing HLA-A*02-dose-dependent blocking.

FIG. 14 is a plot showing HLA-A*02 inhibitor receptor module IC50 when paired with anti-CD19, anti-CD20, or both anti-CD19 and anti-CD20 activator tandem CAR, as measured by titration of HLA-A*02 mRNA in ng. K562 (CD19+) target cells, Raji (CD20+ CD19 Knock Out [KO]) target cells, and Raji (CD20+ CD19+) target cells were transfected with titrated amounts of HLA-A*02 mRNA.

FIG. 15 is a chart depicting illustrative expression results obtained through the analysis of normal cell, tumor cell, and tumor cell line RNA-Seq datasets to generate B antigen candidates.

FIG. 16 shows a schematic depicting an illustrative single mRNA encoding a cleavable fusion protein comprising an inhibitor (blocker) receptor operably linked to an activator receptor. Illustrative examples of hinge (H), transmembrane (TM), and intracellular domain components of the activator and blocker receptors are shown.

FIG. 17A is a flow cytometry graph depicting an activator CAR and inhibitor receptor expression on engineered T cells. Three days after fresh PBMCs were activated with TransAct™, B2M was knocked-out via CRISPR/CAS9 gene editing. After 7 days of recovery, unedited cells were depleted using anti-activator antigen antibody, W6/32. mRNA encoding empty vector, activator (C2172), or a bicistronic vector encoding the activator and inhibitor (blocker) (C6177) was transfected 2 days after enrichment and used in functional assays.

FIG. 17B is a flow cytometry graph depicting Protein-L and Goat α-mouse IgG expression on engineered T cells. Three days after fresh PBMCs were activated with TransAct™, B2M was knocked-out via CRISPR/CAS9 gene editing. After 7 days of recovery, unedited cells were depleted using anti-activator antigen antibody, W6/32. mRNA encoding empty vector, activator (C2172), or a bicistronic vector encoding the activator and inhibitor (blocker) (C6177) was transfected 9 days after enrichment and used in functional assays.

FIG. 18 is a graph depicting IFNgamma secretion by primary T cells after co-culture with HeLa target cells. After mRNA transfection encoding the activator (C2172) and blocker (C6177), T cells were co-cultured with HeLa target cells transfected with increasing amounts of mRNA encoding blocker receptor mRNA. T cells co-cultured with HeLa targets that received no blocker receptor mRNA (closed squares, 6177 no) produced a consistent amount of IFNgamma whereas T cells co-cultured with blocker receptor mRNA (open squares, 6177 A2) were inhibited in IFNgamma production. The half maximal inhibitory concentration (IC50) is provided in the table along with the interpolated number of surface blocker receptor molecules from QiFi Kit analysis of HeLa target cells.

FIG. 19 is a panel of graphs showing the ratio of SPN/MME mRNA expression in non-cancerous “normal” HSCs, AML cells, non-cancerous “normal” monocytes, neutrophils, B cells, and T cells (left panel) and the ratio of SPN/MME protein expression in AML cells and non-cancerous “normal” HSCs (right panel).

FIG. 20 is a panel of graphs showing SPN and MME mRNA expression level in transcripts per million (TPM) in AML cell lines, AML bulk tumor cells, non-cancerous “normal” HSCs, non-cancerous “normal” monocytes, neutrophils, B cells, and T cells (left panel) and SPN and MME protein expression level in label-free quantification (LFQ) units in AML cells and non-cancerous “normal” HSCs (right panel). “LOD” denotes the estimated limit of detection.

FIG. 21 is a graph depicting A antigen SPN expression (left panel) measured in transcripts per million (TPM) in AML subtype cells, CD3+ cells, CD19+ cells, HSCs, monocytes (Mono), and neutrophils (Neu). The right panel is a graph depicting B antigen MME expression measured in TPM AML subtype cells, CD3+ cells, CD19+ cells, HSCs, monocytes (Mono), and neutrophils (Neu).

FIG. 22 is a graph depicting A antigen SPN expression (left panel) measured in transcripts per million (TPM) in AML subtype cells, CD3+ cells, CD19+ cells, HSCs, monocytes (Mono), and neutrophils (Neu). The right panel is a graph depicting B antigen FCGR3A expression measured in TPM AML subtype cells, CD3+ cells, CD19+ cells, HSCs, monocytes (Mono), and neutrophils (Neu).

FIG. 23 shows a schematic depicting Tmod implementation of a NOT-gate. The Tmod NOT-gate system incorporates an inhibitory receptor (blocker) which inhibits T cell activation when engaged with a HLA class I “B” antigen present on normal HSC cells. The B antigen matches the haplotype of transplanted stem cells.

FIG. 24 is a graph depicting expression measured in transcripts per million (TPM) of two different A antigens (left top and bottom panels) in AML subtype cells, CD3+ cells, CD19+ cells, HSCs, monocytes (Mono), and neutrophils (Neu). The right panel is a schematic depicting an illustrative Tmod engineered cell in contact with a cell comprising an antigen combination on target cells that inhibits killing by the engineered immune cells.

FIG. 25 shows a schematic depicting an illustrative Tmod cell in contact with a tumor cell comprising an antigen combination that activates killing by the engineered immune cells (left panel). The schematic also depicts an illustrative Tmod cell in contact with a transplanted Hemopoietic Stem Cell (HSC) comprising an antigen combination that inhibits killing by the engineered immune cells (right panel).

FIG. 26 is a panel of flow cytometry plots showing the analysis of CD33 expression in K562 cells in which CD33 was knocked out and then a titration of CD33 mRNA was transfected. CD33 antibody clone WM53 used for staining. CD33 molecule count (molecules/cell) was interpolated using the QIFIKIT™ for quantitative analysis from Agilent.

FIG. 27 is a panel of flow cytometry plots showing anti-CD33 CAR expression in Jurkat reporter cells. Anti-CD33 CAR expression was detected via staining with recombinant CD33 protein that was pre-labeled with Phycoerythrin (PE).

FIG. 28A is a graph showing CD33 responsive CAR expressing Jurkat reporter cells co-culture with K562 target cells that were transfected with a titration of CD33 mRNA. CD33 molecules/cell was interpolated using the QIFIKIT™ for quantitative analysis from Agilent (right panel).

FIG. 28B is a chart showing surface CD33 molecule levels in molecules/cell analyzed in blood cancer cell lines.

FIG. 29 is a graph showing Jurkat cells transfected with an anti-CD33 CAR plasmid and an anti-CD16 Blocker 1 plasmid and co-cultured with K562 cells that overexpressed CD33 and were transfected with a titration of CD16b mRNA.

FIG. 30A is a graph showing CD16b mRNA transfected into either wild type K562 cells (“Low Endogenous CD33”), K562 cells transfected with 200 ng CD33 mRNA (“Medium CD33”), or K562 cells transfected with 500 ng CD33 mRNA (“High CD33”).

FIG. 30B is a panel of graphs showing the titration of CD16b mRNA at different levels of CD33 expression.

FIG. 30C is a graph showing Jurkat cells transfected with an anti-CD33 CAR “CAR-7” plasmid and an anti-CD16 Blocker 1 plasmid and co-cultured with K562 cells that overexpressed CD33 and were transfected with a titration of CD16b mRNA.

FIG. 30D is a graph showing Jurkat cells transfected with an anti-CD33 CAR “CAR-8” plasmid and an anti-CD16 Blocker 1 plasmid and co-cultured with K562 cells that overexpressed CD33 and were transfected with a titration of CD16b mRNA.

FIG. 31A is a diagram of an anti-CD33 activator CAR and anti-CD16 Blocker 1 experiment in primary T cells.

FIG. 31B is a chart showing QIFIKIT™ from Agilent analysis to estimate number of CD33 and CD16b molecules on the surface of the K562 cell line. K562 cells were engineered to model “normal” tissue by overexpressing CD33 and CD16b and K562 cells that only overexpressed CD33 were engineered to model “tumor” tissue.

FIG. 31C is a graph showing Jurkat cells transfected with anti-CD33 CAR plasmid and an anti-CD16 Blocker 1 plasmid and co-cultured with engineered K562 target cells with a titration of Effector: Target (E: T) ratios.

FIG. 32 is a panel of flow cytometry plots showing anti-SPN CAR expression in Jurkat reporter cells. Protein L or soluble recombinant human His-tagged protein (sSPN-His) was used to stain the CARs.

FIG. 33 is a panel of histograms showing SPN staining of JNL SPN knock out (KO) and JNL wild type (WT) cells. JNL SPN KO cells were produced using CRISPR and a guide RNA targeting the SPN gene followed by cell sorting to enrich the SPN KO population. Cells were stained using anti-SPN antibody clone 1G10 followed by analysis by flow cytometry.

FIG. 34 is a panel of graphs showing Jurkat nuclear factor of activated T cells (NFAT) luciferase (JNL) cell activation in a cell titration assay. JNL cells were transfected with 2 μg of activator anti-SPN CAR DNA per 1E6 cells using the Neon electroporation system. 18-24 hours post-transfection, 10,000 JNL cells were co-cultured with serially titrated Target cells (Jurkat E6.1, K562, HeLa, THP-1, and MV4-11) for 6 hours.

FIG. 35 is a panel of flow cytometry histogram plots showing Flag-CD16b mRNA titrated in Jurkat WT cells, detected with anti-CD16 antibody 3G8 and quantified by converting flow-cytometry mean fluorescence intensity (MFI) signal to molecules of CD16b per cell using QIFIKIT™ quantitative analysis kit.

FIG. 36 shows a panel of flow cytometry plots of anti-SPN CAR expression “CAR 1” and “CAR 2” with or without anti-CD16 Blocker 1 in JNL SPN KO cells where anti-SPN CAR staining was performed using Protein L and anti-CD16 Blocker 1 staining was performed using an anti-CD16 Blocker 1 hinge-specific antibody. Percentages for gated populations shown on plots.

FIG. 37 is a graph showing activation of Jurkat NFAT luciferase (JNL) cells transfected with an anti-CD16 Blocker 1 scFv fused to CAR domains in an mRNA titration assay. Jurkat WT target cells were transfected with serially diluted Flag-CD16b mRNA and JNL SPN KO cells were transfected with 2 μg of activator anti-SPN CAR DNA per 1E6 cells using the Neon™ electroporation system. 18-24 hours post-transfection, 10,000 JNL anti-SPN CAR cells were co-cultured with 5000 transfected Jurkat WT target cells for 6 hours. The functional response (RLU) was assessed using ONE™ step luciferase assay system.

FIG. 38A is a graph showing Jurkat WT target cells transfected with serially diluted FLAG-CD16b mRNA and JNL SPN KO cells transfected with 1 μg of activator anti-SPN CAR “CAR-1” DNA and 2 μg of anti-CD16 Blocker 1 DNA per 1E6 cells using the Neon™ electroporation system. 18-24 hours post-transfection, 10,000 JNL cells were co-cultured with 5000 transfected Jurkat cells for 6 hours. The activation of JNL cells was assessed by measuring the functional response (RLU) using the ONE™ step luciferase assay system.

FIG. 38B is a graph showing Jurkat WT target cells transfected with serially diluted FLAG-CD16b mRNA and JNL SPN KO cells transfected with 1 μg of activator anti-SPN CAR “CAR-2” DNA and 2 μg of anti-CD16 Blocker 1 DNA per 1E6 cells using the Neon™ electroporation system. 18-24 hours post-transfection, 10,000 JNL cells were co-cultured with 5000 transfected Jurkat cells for 6 hours. The activation of JNL cells was assessed by measuring the functional response (RLU) using the ONE™ step luciferase assay system.

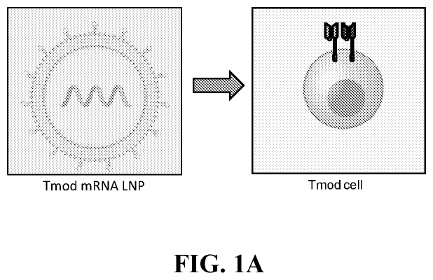

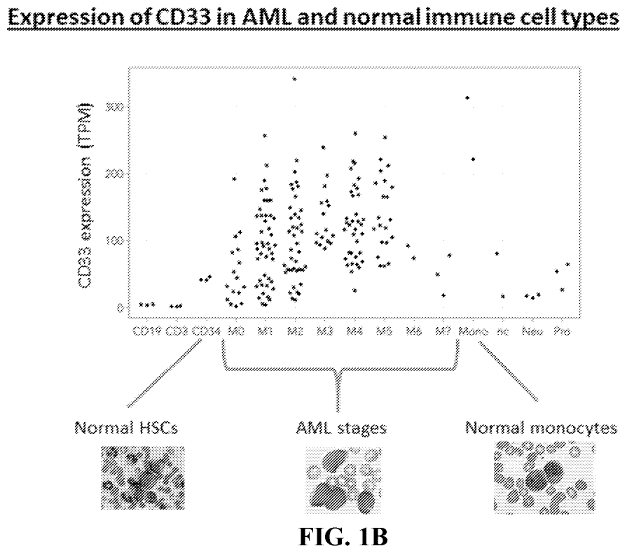

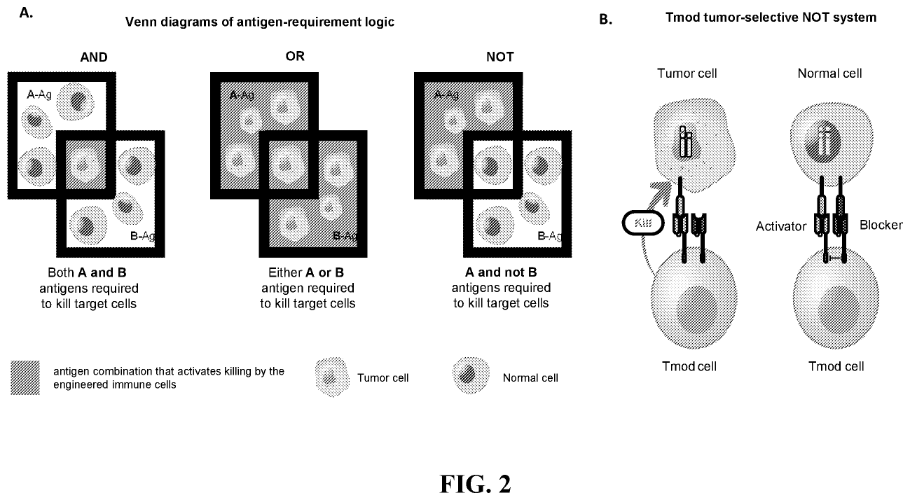

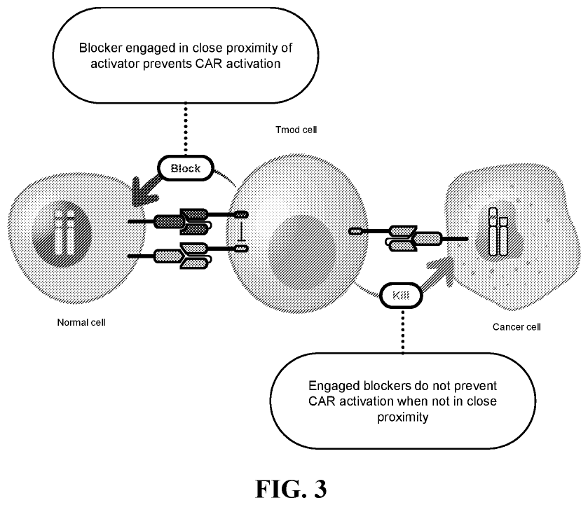

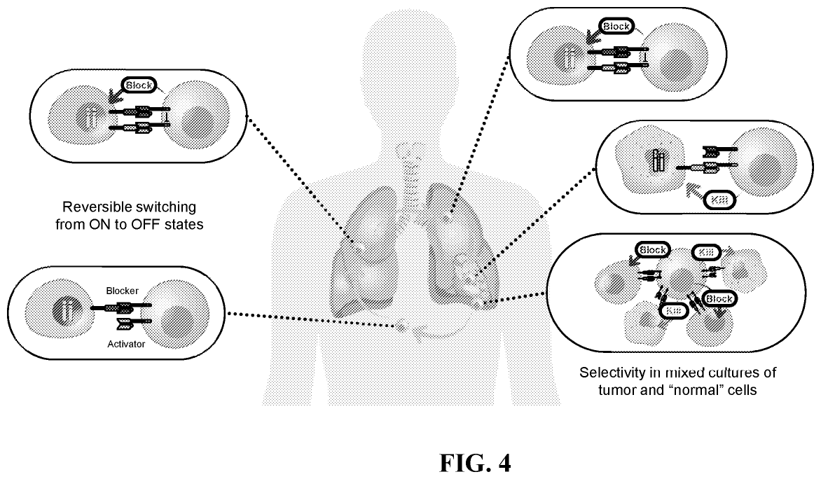

### DETAILED DESCRIPTION

The present disclosure relates, in part, to identification of suitable target molecules for logic-gated cell therapy. Provided herein are methods to identify suitable sets of activator (A) and blocker (B) antigens for use in the treatment of blood cancers. Further provided are immune cells that integrate, through selective binding, signals provided by sets of A and B antigens. The immune cells of the disclosure operate as a “logic gate” wherein signals from A antigens and B antigens together trigger a desired response by the immune cell.

Immune cells of the present disclosure are engineered to specifically activate when contacted with blood cancer cells. The immune cells do not activate when contacted with non-cancerous cells, such as hematopoietic stem cells. Accordingly, adverse side effects caused by damage to non-cancerous cells may be reduced. Sets of A and B antigens, where A antigens activate the engineered immune cells and B antigens inhibit them, may be used to selectively target blood cancer cells using the synthetic signal-integration systems of the present disclosure.

The set of A antigens may comprise a single member A, or the set of A antigens may comprise n antigens denoted A₁, A₂, . . . , Aₙ. The set of B antigens may comprise a single member B, or the set of B antigens may comprise n antigens denoted B₁, B₂, . . . , Bₙ.

Logic gates may include OR-NOT, AND-NOT, and other variations. For example, the disclosure contemplates the following, non-limiting, list of logic gates:
A NOT B
(A₁ OR A₂) NOT B
(A₁ AND A₂) NOT B
(A₁ OR A₂) NOT (B₁ OR B₂)
(A₁ OR A₂) NOT (B₁ AND B₂)
A NOT (B₁ OR B₂)
A NOT (B₁ AND B₂)

NOT logic may be provided by, for example and without limitation, use of an inhibitory receptor that specifically binds a B antigen; when activated, the inhibitory receptor provides a signal that blocks the activator receptor.

OR logic may be provided by pairs of activator receptors or pairs of inhibitor receptors. For example, an (A₁ OR A₂) signal may be provided by a first activator receptor that specifically binds A₁ and a second activator receptor that specifically binds A₂. Likewise, a NOT (B₁ OR B₂) signal may be provided by a first inhibitor receptor that specifically binds B₁ and a second inhibitor receptor that specifically binds B₂.

In a variation, OR logic may be provided by a single receptor that specifically binds two antigens. For example, an (A₁ OR A₂) signal may be provided by an activator receptor that specifically binds A₁ and specifically binds A₂. Likewise, a NOT (B₁ OR B₂) signal may be provided by an inhibitor receptor that specifically binds B₁ and specifically binds B₂.

AND logic may be provided by two receptors that each individually activate in response to an antigen, but provide an activator (or inhibitor) signal that is below a threshold for activation (or inhibition) of the immune cell. For example, an (A₁ AND A₂) signal may be provided by a first activator receptor that specifically binds A₁ and a second activator receptor that specifically binds A₂, where either activator signal alone is too weak to activate the immune cell. Likewise, a NOT (B₁ AND B₂) signal may be provided by a first inhibitor receptor that specifically binds B₁ and a second inhibitor receptor that specifically binds B₂, where either inhibitor signal alone is too weak to block the activation of the immune cell.

In a variation, a single activator (or inhibitor) receptor may be used, where the single receptor is activated only when contacted by both antigens. For example, an (A₁ AND A₂) signal may be provided by an activator receptor that specifically binds A₁ and that specifically binds A₂, where the activator receptor only activates when it binds both A₁ and A₂. Likewise, a NOT (B₁ AND B₂) signal may be provided by an inhibitor receptor that specifically binds B₁ and that specifically binds B₂, where the inhibitor receptor only activates, to block an activator signal, when it binds both B₁ and B₂.

Illustrative, non-limiting, examples of A and B antigens in logic gates contemplated by the present disclosure are shown in Table 1A. Illustrative, non-limiting, examples of A and B antigen combinations in logic gates contemplated by the present disclosure are shown in Table 1B.

TABLE 1A
Illustrative A and B antigens

| A antigen | B antigen |
|---|---|
| SPN (CD43) | PECAM1 |
| CD45 (PTPRC) | HLA-G |
| ITGAL (LFA-1/CD11a) | ABCG2 |
| CD33 | SIGLEC5 |
| FLT3 | PDZK1IP1 |
| CD123 | MME |
| | FCGR3A |

TABLE 1B
Illustrative A and B antigen combinations

| Logic Gate | A antigen(s) | B antigen(s) |
|---|---|---|
| A NOT B | SPN | PECAM-1 |
| A NOT B | SPN | HLA-G |
| A NOT B | SPN | ABCG2 |
| A NOT B | SPN | SIGLEC5 |
| A NOT B | SPN | PDZK1IP1 |
| A NOT B | SPN | MME |
| A NOT B | SPN | FCGR3A |
| (A₁ OR A₂) NOT B | SPN OR CD45 | PECAM-1 |
| (A₁ OR A₂) NOT B | SPN OR ITGAL | PECAM-1 |
| (A₁ OR A₂) NOT B | SPN OR CD33 | PECAM-1 |
| (A₁ OR A₂) NOT B | SPN OR FLT3 | PECAM-1 |
| (A₁ OR A₂) NOT B | SPN OR CD123 | PECAM-1 |
| (A₁ OR A₂) NOT B | SPN OR CD45 | HLA-G |
| (A₁ OR A₂) NOT B | SPN OR ITGAL | HLA-G |
| (A₁ OR A₂) NOT B | SPN OR CD33 | HLA-G |
| (A₁ OR A₂) NOT B | SPN OR FLT3 | HLA-G |
| (A₁ OR A₂) NOT B | SPN OR CD123 | HLA-G |
| (A₁ OR A₂) NOT B | SPN OR CD45 | ABCG2 |
| (A₁ OR A₂) NOT B | SPN OR ITGAL | ABCG2 |
| (A₁ OR A₂) NOT B | SPN OR CD33 | ABCG2 |
| (A₁ OR A₂) NOT B | SPN OR FLT3 | ABCG2 |
| (A₁ OR A₂) NOT B | SPN OR CD123 | ABCG2 |
| (A₁ OR A₂) NOT B | SPN OR CD45 | SIGLEC5 |
| (A₁ OR A₂) NOT B | SPN OR ITGAL | SIGLEC5 |
| (A₁ OR A₂) NOT B | SPN OR CD33 | SIGLEC5 |
| (A₁ OR A₂) NOT B | SPN OR FLT3 | SIGLEC5 |
| (A₁ OR A₂) NOT B | SPN OR CD123 | SIGLEC5 |
| (A₁ OR A₂) NOT B | SPN OR CD45 | PDZK1IP1 |
| (A₁ OR A₂) NOT B | SPN OR ITGAL | PDZK1IP1 |
| (A₁ OR A₂) NOT B | SPN OR CD33 | PDZK1IP1 |
| (A₁ OR A₂) NOT B | SPN OR FLT3 | PDZK1IP1 |
| (A₁ OR A₂) NOT B | SPN OR CD123 | PDZK1IP1 |
| (A₁ OR A₂) NOT B | SPN OR CD45 | MME |
| (A₁ OR A₂) NOT B | SPN OR ITGAL | MME |
| (A₁ OR A₂) NOT B | SPN OR CD33 | MME |
| (A₁ OR A₂) NOT B | SPN OR FLT3 | MME |
| (A₁ OR A₂) NOT B | SPN OR CD123 | MME |
| (A₁ OR A₂) NOT B | SPN OR CD45 | FCGR3A |
| (A₁ OR A₂) NOT B | SPN OR ITGAL | FCGR3A |
| (A₁ OR A₂) NOT B | SPN OR CD33 | FCGR3A |
| (A₁ OR A₂) NOT B | SPN OR FLT3 | FCGR3A |
| (A₁ OR A₂) NOT B | SPN OR CD123 | FCGR3A |
| A NOT B | CD45 | PECAM-1 |
| A NOT B | CD45 | HLA-G |
| A NOT B | CD45 | ABCG2 |
| A NOT B | CD45 | SIGLEC5 |
| A NOT B | CD45 | PDZK1IP1 |
| A NOT B | CD45 | MME |
| A NOT B | CD45 | FCGR3A |
| (A₁ OR A₂) NOT B | CD45 OR ITGAL | PECAM-1 |
| (A₁ OR A₂) NOT B | CD45 OR CD33 | PECAM-1 |
| (A₁ OR A₂) NOT B | CD45 OR FLT3 | PECAM-1 |
| (A₁ OR A₂) NOT B | CD45 OR CD123 | PECAM-1 |
| (A₁ OR A₂) NOT B | CD45 OR ITGAL | HLA-G |
| (A₁ OR A₂) NOT B | CD45 OR CD33 | HLA-G |
| (A₁ OR A₂) NOT B | CD45 OR FLT3 | HLA-G |
| (A₁ OR A₂) NOT B | CD45 OR CD123 | HLA-G |
| (A₁ OR A₂) NOT B | CD45 OR ITGAL | ABCG2 |
| (A₁ OR A₂) NOT B | CD45 OR CD33 | ABCG2 |
| (A₁ OR A₂) NOT B | CD45 OR FLT3 | ABCG2 |
| (A₁ OR A₂) NOT B | CD45 OR CD123 | ABCG2 |
| (A₁ OR A₂) NOT B | CD45 OR ITGAL | SIGLEC5 |
| (A₁ OR A₂) NOT B | CD45 OR CD33 | SIGLEC5 |
| (A₁ OR A₂) NOT B | CD45 OR FLT3 | SIGLEC5 |
| (A₁ OR A₂) NOT B | CD45 OR CD123 | SIGLEC5 |
| (A₁ OR A₂) NOT B | CD45 OR ITGAL | PDZK1IP1 |
| (A₁ OR A₂) NOT B | CD45 OR CD33 | PDZK1IP1 |
| (A₁ OR A₂) NOT B | CD45 OR FLT3 | PDZK1IP1 |
| (A₁ OR A₂) NOT B | CD45 OR CD123 | PDZK1IP1 |
| (A₁ OR A₂) NOT B | CD45 OR ITGAL | MME |
| (A₁ OR A₂) NOT B | CD45 OR CD33 | MME |
| (A₁ OR A₂) NOT B | CD45 OR FLT3 | MME |
| (A₁ OR A₂) NOT B | CD45 OR CD123 | MME |
| (A₁ OR A₂) NOT B | CD45 OR ITGAL | FCGR3A |
| (A₁ OR A₂) NOT B | CD45 OR CD33 | FCGR3A |
| (A₁ OR A₂) NOT B | CD45 OR FLT3 | FCGR3A |
| (A₁ OR A₂) NOT B | CD45 OR CD123 | FCGR3A |
| A NOT B | ITGAL | PECAM-1 |
| A NOT B | ITGAL | HLA-G |
| A NOT B | ITGAL | ABCG2 |
| A NOT B | ITGAL | SIGLEC5 |
| A NOT B | ITGAL | PDZK1IP1 |
| A NOT B | ITGAL | MME |
| A NOT B | ITGAL | FCGR3A |
| (A₁ OR A₂) NOT B | ITGAL OR CD33 | PECAM-1 |
| (A₁ OR A₂) NOT B | ITGAL OR FLT3 | PECAM-1 |
| (A₁ OR A₂) NOT B | ITGAL OR CD123 | PECAM-1 |
| (A₁ OR A₂) NOT B | ITGAL OR CD33 | HLA-G |
| (A₁ OR A₂) NOT B | ITGAL OR FLT3 | HLA-G |
| (A₁ OR A₂) NOT B | ITGAL OR CD123 | HLA-G |
| (A₁ OR A₂) NOT B | ITGAL OR CD33 | ABCG2 |
| (A₁ OR A₂) NOT B | ITGAL OR FLT3 | ABCG2 |
| (A₁ OR A₂) NOT B | ITGAL OR CD123 | ABCG2 |
| (A₁ OR A₂) NOT B | ITGAL OR CD33 | SIGLEC5 |
| (A₁ OR A₂) NOT B | ITGAL OR FLT3 | SIGLEC5 |
| (A₁ OR A₂) NOT B | ITGAL OR CD123 | SIGLEC5 |
| (A₁ OR A₂) NOT B | ITGAL OR CD33 | PDZK1IP1 |
| (A₁ OR A₂) NOT B | ITGAL OR FLT3 | PDZK1IP1 |
| (A₁ OR A₂) NOT B | ITGAL OR CD123 | PDZK1IP1 |
| (A₁ OR A₂) NOT B | ITGAL OR CD33 | MME |
| (A₁ OR A₂) NOT B | ITGAL OR FLT3 | MME |
| (A₁ OR A₂) NOT B | ITGAL OR CD123 | MME |
| (A₁ OR A₂) NOT B | ITGAL OR CD33 | FCGR3A |
| (A₁ OR A₂) NOT B | ITGAL OR FLT3 | FCGR3A |
| (A₁ OR A₂) NOT B | ITGAL OR CD123 | FCGR3A |
| A NOT B | CD33 | PECAM-1 |
| A NOT B | CD33 | HLA-G |
| A NOT B | CD33 | ABCG2 |
| A NOT B | CD33 | SIGLEC5 |
| A NOT B | CD33 | PDZK1IP1 |
| A NOT B | CD33 | MME |
| A NOT B | CD33 | FCGR3A |
| (A₁ OR A₂) NOT B | CD33 OR FLT3 | PECAM-1 |
| (A₁ OR A₂) NOT B | CD33 OR FLT3 | HLA-G |
| (A₁ OR A₂) NOT B | CD33 OR FLT3 | ABCG2 |
| (A₁ OR A₂) NOT B | CD33 OR FLT3 | SIGLEC5 |
| (A₁ OR A₂) NOT B | CD33 OR FLT3 | PDZK1IP1 |
| (A₁ OR A₂) NOT B | CD33 OR FLT3 | MME |
| (A₁ OR A₂) NOT B | CD33 OR FLT3 | FCGR3A |
| (A₁ OR A₂) NOT B | CD33 OR CD123 | PECAM-1 |
| (A₁ OR A₂) NOT B | CD33 OR CD123 | HLA-G |
| (A₁ OR A₂) NOT B | CD33 OR CD123 | ABCG2 |
| (A₁ OR A₂) NOT B | CD33 OR CD123 | SIGLEC5 |
| (A₁ OR A₂) NOT B | CD33 OR CD123 | PDZK1IP1 |
| (A₁ OR A₂) NOT B | CD33 OR CD123 | MME |
| (A₁ OR A₂) NOT B | CD33 OR CD123 | FCGR3A |
| A NOT B | FLT3 | PECAM-1 |
| A NOT B | FLT3 | HLA-G |
| A NOT B | FLT3 | ABCG2 |
| A NOT B | FLT3 | SIGLEC5 |
| A NOT B | FLT3 | PDZK1IP1 |
| A NOT B | FLT3 | MME |
| A NOT B | FLT3 | FCGR3A |
| (A₁ OR A₂) NOT B | FLT3 OR CD123 | PECAM-1 |
| (A₁ OR A₂) NOT B | FLT3 OR CD123 | HLA-G |
| (A₁ OR A₂) NOT B | FLT3 OR CD123 | ABCG2 |
| (A₁ OR A₂) NOT B | FLT3 OR CD123 | SIGLEC5 |
| (A₁ OR A₂) NOT B | FLT3 OR CD123 | PDZK1IP1 |
| (A₁ OR A₂) NOT B | FLT3 OR CD123 | MME |
| (A₁ OR A₂) NOT B | FLT3 OR CD123 | FCGR3A |
| A NOT B | CD123 | PECAM-1 |
| A NOT B | CD123 | HLA-G |
| A NOT B | CD123 | ABCG2 |
| A NOT B | CD123 | SIGLEC5 |
| A NOT B | CD123 | PDZK1IP1 |
| A NOT B | CD123 | MME |
| A NOT B | CD123 | FCGR3A |
| A NOT (B₁ OR B₂) | SPN | MME OR FCGR3A |
| A NOT (B₁ OR B₂) | CD45 | MME OR FCGR3A |
| A NOT (B₁ OR B₂) | ITGAL | MME OR FCGR3A |
| A NOT (B₁ OR B₂) | CD33 | MME OR FCGR3A |
| A NOT (B₁ OR B₂) | FLT3 | MME OR FCGR3A |
| A NOT (B₁ OR B₂) | CD123 | MME OR FCGR3A |

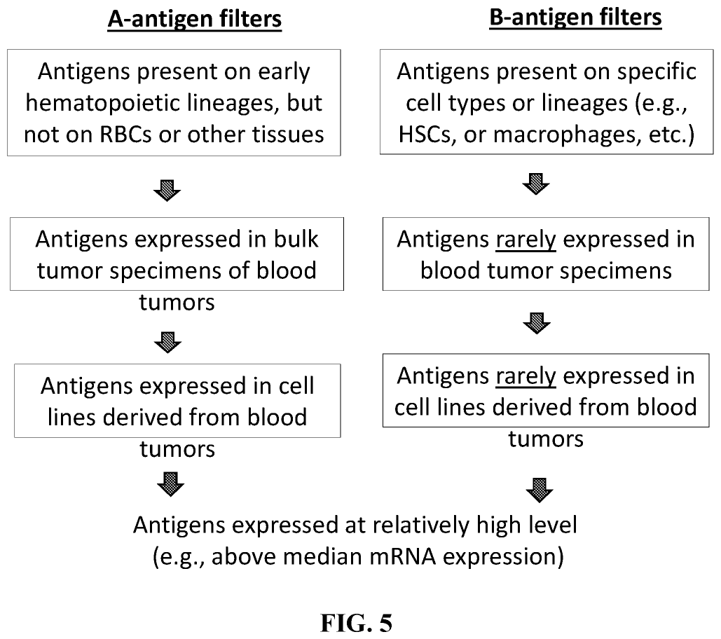

The present disclosure describes an algorithm to identify A and B antigens for use in Tmod™ activator (A) and blocker (B) modules. Unlike other methods that begin with known, specific target antigens, the algorithm described herein (shown in FIG. 5) begins with putative activator antigens that are widely expressed on early hematopoietic lineages, but not on red blood cells (RBCs) and putative inhibitor antigens widely expressed on cell types or lineages that should be protected (e.g., hematopoetic stem cells (HSCs) or macrophages).

In an embodiment, a blood-tumor targeting cell therapy comprises a synthetic signal-integration system that integrates signals from activator and inhibitor antigens to trigger selective tumor cell killing. The synthetic signal-integration system contains receptor(s) that activate an effector response and receptor(s) that inhibit a response.

In some embodiments, A1 and A2 specificities are both expressed on a single activator receptor. In some embodiments, A1 and A2 specificities are expressed on individual activator receptors. In some embodiments, B1 and B2 specificities are both expressed on a single activator receptor. In some embodiments, B1 and B2 specificities are expressed on individual activator receptors.

The present disclosure describes engineered receptors, such as chimeric antigen receptors (CARs) and engineered T cell receptors (TCRs), adoptive cell therapies, and methods of use thereof.

In some embodiments, the present disclosure provides an immune cell that acts as an A NOT B logic gate, where A is SPN and B is PECAM-1. The applicants have identified SPN and PECAM-1 using the algorithm shown in FIG. 5 and described in Example 1. Moreover, testing immune cells that express an activator receptor that specifically binds SPN and an inhibitor receptor that specifically binds PECAM-1 will demonstrate that SPN and PECAM-1 are effective as A and B antigens for an A NOT B logic gate.

Accordingly, in some embodiments, an engineered activator receptor may target the SPN antigen and activate immune cells genetically altered to express the SPN targeting receptor. Immune cells expressing the SPN targeting activator receptors can be used in compositions as part of cell adoptive therapies in the treatment, for example, of blood cancers. The immune cells expressing the SPN targeting activator receptor are also contemplated to further comprise an inhibitory receptor targeting SPN. The inhibitory receptor may prevent or inhibit the activation of an immune cell mediated by the activator receptor. For example, an immune cell engineered to express an activator receptor that specially binds SPN when the activator contacts a cell expressing SPN and an inhibitory receptor that specially binds PECAM-1 will be activated when the inhibitory receptor contacts a cell expressing PECAM-1; collectively, the receptors generate a NOT logic gate such that the immune cell specifically activates when the immune cell contacts a blood cancer cell that expresses SPN but not PECAM-1. The immune cell does not activate, or activates at a reduced level, when the immune cell contacts a non-blood cancer cell (e.g., a healthy blood cell or healthy solid tissue cell), expressing both SPN and PECAM-1.

Selective activation and inhibition of the immune cell is useful for reducing toxicity in adoptive cell therapies. The inventors have found that this selective activation and inhibition strategy can be employed using the SPN-targeting activator receptors and PECAM-1-targeting inhibitor receptors described herein.

### Engineered Receptors

The disclosure provides an activator receptor comprising an extracellular region, the extracellular region comprising a first ligand binding domain capable of specifically binding a first ligand that activates or promotes activation of the receptor, which promotes activation of effector cells expressing the receptor. The disclosure further provides an inhibitor receptor comprising a second ligand binding domain capable of binding a second ligand, wherein binding of the second ligand by the second ligand binding domain inhibits, reduces, or prevents activation of effector cells even when the activator receptor is bound to the first ligand. The first ligand may also be referred to as an activator ligand or antigen. The second ligand may also be referred to as an inhibitor ligand or antigen. The activator and inhibitor receptors that bind to these ligands may also be referred to generally herein as engineered receptors. Engineered receptors can refer to either chimeric antigen receptors (CARs) or T cell receptors (TCRs) described in the disclosure. The term “engineered receptor” may also refer to any receptor designed using the binding domains, hinge regions, transmembrane domains, and/or cytoplasmic domains described herein. Engineered receptors are sometimes referred to herein as fusion proteins.

### Antigens

### CD45

The activator receptors disclosed can specifically bind to a CD45 antigen.

The term “CD45” also known as leukocyte common antigen (LCA), protein tyrosine phosphatase receptor type C (PTPRC), and other aliases refers to human CD45 protein and species, isoforms, and other sequence variants thereof. Thus, CD45 can be the native, full-length protein or can be a truncated fragment or a sequence variant (e.g., a naturally occurring isoform, or recombinant variant) that retains at least one biological activity of the native protein. CD45 is a receptor-linked protein tyrosine phosphatase that is expressed on leukocytes, and which plays an important role in the function of these cells (reviewed in Altin, J G (1997) Immunol Cell Biol. 75 (5): 430-45, incorporated herein by reference). For example, the extracellular domain of CD45 is expressed in several different isoforms on T cells, and the particular isoform(s) expressed depends on the particular subpopulation of cell, their state of maturation, and antigen exposure. Expression of CD45 is important for the activation of T cells via the TCR, and different CD45 isoforms display a different ability to support T cell activation.

In some embodiments, the target antigen is a peptide antigen of CD45. In some embodiments, the CD45 antigen comprises a sequence or subsequence at least 80%, at least 85%, at least 90%, at least 95%, at least 97%, at least 99%, or 100% identical to a sequence or a subsequence of any one of SEQ ID NOs: 1-9 or 327. However, all CD45 isoforms, and sequences and subsequences thereof that act as CD45 antigens are envisaged as within the scope of the disclosure. In some embodiments, A CD45 antigen comprises a protein comprising a sequence any one of SEQ ID NOS: 1-9 or 327, or a peptide fragment of any one of SEQ ID NOS: 1-9 or 327.

### CD11a/LFA-1/ITGAL

The activator receptors disclosed can specifically bind to a CD11a antigen.

The term “CD11a” also known as integrin alpha L, ITGAL, p180, lymphocyte function-associated antigen 1-alpha polypeptide, LFA-1, LFA1A, integrin subunit alpha L, and other aliases refers to the human CD11a protein and species, isoforms, and other sequence variants thereof. Thus, CD11a can be the native, full-length protein or can be a truncated fragment or a sequence variant (e.g., a naturally occurring isoform, or recombinant variant) that retains at least one biological activity of the native protein.

CD11a is expressed on all leukocytes and is involved in leukocyte intercellular adhesion through interactions with its ligands, ICAMs 1-3 (intercellular adhesion molecules 1 through 3), and also functions in lymphocyte costimulatory signaling. CD11a is one of the two components, along with CD18, which form lymphocyte function-associated antigen-1 (LFA-1). LFA-1 plays a role in emigration, which is the process by which leukocytes leave the bloodstream to enter the tissues. Additionally, LFA-1 is involved in the process of cytotoxic T cell mediated killing as well as antibody mediated killing by granulocytes and monocytes.

In some embodiments, the target antigen is a peptide antigen of CD11a. In some embodiments, the CD11a antigen comprises a sequence or subsequence at least 80%, at least 85%, at least 90%, at least 95%, at least 97%, at least 99%, or 100% identical to a sequence or a subsequence of any one of SEQ ID NOs: 169-171. However, all CD11a isoforms, and sequences and subsequences thereof that act as CD11a antigens are envisaged as within the scope of the disclosure. In some embodiments, a CD11a antigen comprises a protein comprising a sequence of any one of SEQ ID NOS: 169-171, or a peptide fragment of any one of SEQ ID NOS: 169-171.

### SPN

The activator receptors disclosed can specifically bind to a sialophorin (SPN) antigen.

The term “sialophorin” also known as SPN, Leukosialin, CD43, GPL115, galactoglycoprotein (GALGP), leukocyte sialoglycoprotein, and other aliases refers to the human SPN protein and species, isoforms, and other sequence variants thereof. Thus, SPN can be the native, full-length protein or can be a truncated fragment or a sequence variant (e.g., a naturally occurring isoform, or recombinant variant) that retains at least one biological activity of the native protein.

SPN is a large, negatively charged type I transmembrane sialoglycoprotein and is expressed on the surface of human T lymphocytes, monocytes, granulocytes, and some B lymphocytes. SPN functions as an anti-adhesion molecule, mediating repulsion among leukocytes. The SPN extracellular domain has a rod-like structure that is predicted to extend from the cell surface and contains more than 80 serine or threonine residues, most of which are modified with heavily sialylated O-linked glycans. SPN's cytoplasmic tail binds ERM-family proteins. Two major SPN glycoforms (115 kD and 130 kD) have been identified in humans and mice. The 130-kD SPN glycoform, modified with sLex-like structures, is reported to bind E-selectin in activated T cells.

In some embodiments, the target antigen is a peptide antigen of SPN. In some embodiments, the SPN antigen comprises a sequence or subsequence at least 80%, at least 85%, at least 90%, at least 95%, at least 97%, at least 99%, or 100% identical to a sequence or a subsequence of any one of SEQ ID NOs: 172-173. However, all SPN isoforms, and sequences and subsequences thereof that act as SPN antigens are envisaged as within the scope of the disclosure. In some embodiments, a SPN antigen comprises a protein comprising a sequence of any one of SEQ ID NOS: 172-173, or a peptide fragment of any one of SEQ ID NOS: 172-173.

### CD33

The activator receptors disclosed can specifically bind to a CD33 antigen.

The term “CD33” also known as siglec-3, sialic acid binding Ig-like lectin 3, gp67, p67, and other aliases refers to the human CD33 protein and species, isoforms, and other sequence variants thereof. Thus, CD33 can be the native, full-length protein or can be a truncated fragment or a sequence variant (e.g., a naturally occurring isoform, or recombinant variant) that retains at least one biological activity of the native protein.

CD33 is a sialoadhesin molecule and a member of the immunoglobulin supergene family. It is expressed by myeloid stem cells, myeloblasts and monoblasts, monocytes/macrophages, granulocyte precursors (with decreasing expression with maturation), and mast cells.

In some embodiments, the target antigen is a peptide antigen of CD33. In some embodiments, the CD33 antigen comprises a sequence or subsequence at least 80%, at least 85%, at least 90%, at least 95%, at least 97%, at least 99%, or 100% identical to a sequence or a subsequence of any one of SEQ ID NOs: 174-176. However, all CD33 isoforms, and sequences and subsequences thereof that act as CD33 antigens are envisaged as within the scope of the disclosure. In some embodiments, a CD33 antigen comprises a protein comprising a sequence of any one of SEQ ID NOS: 174-176, or a peptide fragment of any one of SEQ ID NOS: 174-176.

### FLT3

The activator receptors disclosed can specifically bind to a FLT3 antigen.

The term “FLT3” also known as CD135, fms like tyrosine kinase 3 (FLT-3), receptor-type tyrosine-protein kinase FLT3, fetal liver kinase-2 (Flk2), and other aliases refers to the human FLT3 protein and species, isoforms, and other sequence variants thereof. Thus, FLT3 can be the native, full-length protein or can be a truncated fragment or a sequence variant (e.g., a naturally occurring isoform, or recombinant variant) that retains at least one biological activity of the native protein.

FLT3 is a class III receptor tyrosine kinase that regulates hematopoiesis. FLT3 is activated by binding of the fms-related tyrosine kinase 3 ligand to the extracellular domain, which induces homodimer formation in the plasma membrane leading to auto-phosphorylation of FLT3. The activated receptor kinase subsequently phosphorylates and activates multiple cytoplasmic effector molecules in pathways involved in apoptosis, proliferation, and differentiation of hematopoietic cells in bone marrow.

In some embodiments, the target antigen is a peptide antigen of FLT3. In some embodiments, the FLT3 antigen comprises a sequence or subsequence at least 80%, at least 85%, at least 90%, at least 95%, at least 97%, at least 99%, or 100% identical to a sequence or a subsequence of any one of SEQ ID NOs: 177-178. However, all FLT3 isoforms, and sequences and subsequences thereof that act as FLT3 antigens are envisaged as within the scope of the disclosure. In some embodiments, a FLT3 antigen comprises a protein comprising a sequence of any one of SEQ ID NOS: 177-178, or a peptide fragment of any one of SEQ ID NOS: 177-178.

### CD123

The activator receptors disclosed can specifically bind to a CD123 antigen.

The IL-3R is a heterodimer composed by alpha and beta chains. The term “CD123” also known as the alpha-chain of the interleukin-3 receptor (IL-3RA) and other aliases refers to a glycoprotein composed of an extracellular domain, a predicted Ig-like domain, two FnIII domains, a transmembrane domain, and an intracellular domain. In some embodiments, the target antigen is a peptide antigen of CD123. All CD123 isoforms, and sequences and subsequences thereof that act as CD123 antigens are envisaged as within the scope of the disclosure.

In some embodiments, the anti-CD123 VH region comprises any one of SEQ ID NOs: 408, 410, 412, 414, 416, 418, 424-426, 428, 430, 432, 434, 436, 438, 440, 442, 444, 446, 448, or 450, or a sequence having at least 90%, at least 95%, at least 97%, at least 99% identity, or 100% identical thereto. In some embodiments, the anti-CD123 VL region comprises any one of SEQ ID NOs: 409, 411, 413, 415, 417, 419, 427, 429, 431, 433, 435, 437, 439, 441, 443, 445, 447, 449, or 451 or a sequence having at least 90%, at least 95%, at least 97%, at least 99% identity, or 100% identical thereto. In some embodiments, the binding domain comprises the full length VH region and VL regions on a single polypeptide. In some embodiments, the polypeptide comprises from N-terminal to C-terminal the full length VH regions and the full length VL region. In some embodiments, the polypeptide comprises from N-terminal to C-terminal the full length VL region and the full length VH region.

### Activator Receptors

### Activator Ligands

The disclosure provides an activator ligand and an engineered receptor comprising a ligand binding domain that binds to the activator ligand. In some embodiments, the first engineered receptor is an activator receptor. In some embodiments, the activator receptor is a T cell receptor (TCR) or a chimeric antigen receptor (CAR). In some embodiments, the first activator ligand is a CD45 antigen. In some embodiments, the first activator ligand is a CD11a antigen. In some embodiments, the first activator ligand is a SPN antigen. In some embodiments, the first activator ligand is a CD33 antigen. In some embodiments, the first activator ligand is a FLT3 antigen. In some embodiments, the CD45 antigen comprises a sequence or subsequence that shares at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a sequence or a subsequence of any one of SEQ ID NOs: 1-9. In some embodiments, the CD45 antigen is an isoform produced by alternative splicing. Illustrative CD45 antigen sequences are shown in Table 2.

TABLE 2
Illustrative CD45 sequences

| CD45 | |
|---|---|
| Antigen | Sequence |
| CD45 | MTMYLWLKLL AFGFAFLDTE VFVTGQSPTP SPTGLTTAKM PSVPLSSDPL PTHTTAFSPA |
| Isoform 1 | STFERENDES ETTTSLSPDN TSTQVSPDSL DNASAFNTTG VSSVQTPHLP THADSQTPSA |
| | GTDTQTFSGS AANAKLNPTP GSNAISDVPG ERSTASTEPT DPVSPLTTTL SLAHHSSAAL |
| | PARTSNTTIT ANTSDAYLNA SETTTLSPSG SAVISTTTIA TTPSKPTCDE KYANITVDYL |
| | YNKETKLFTA KLNVNENVEC GNNTCTNNEV HNLTECKNAS VSISHNSCTA PDKTLILDVP |
| | PGVEKFQLHD CTQVEKADTT ICLKWKNIET FTCDTQNITY RFQCGNMIFD NKEIKLENLE |
| | PEHEYKCDSE ILYNNHKFTN ASKIIKTDFG SPGEPQIIFC RSEAAHQGVI TWNPPQRSFH |
| | NFTLCYIKET EKDCLNLDKN LIKYDLQNLK PYTKYVLSLH AYIIAKVQRN GSAAMCHFTT |
| | KSAPPSQVWN MTVSMTSDNS MHVKCRPPRD RNGPHERYHL EVEAGNTLVR NESHKNCDER |
| | VKDLQYSTDY TFKAYFHNGD YPGEPFILHH STSYNSKALI AFLAFLIIVT SIALLVVLYK |
| | IYDLHKKRSC NLDEQQELVE RDDEKQLMNV EPIHADILLE TYKRKIADEG RLFLAEFQSI |
| | PRVFSKFPIK EARKPFNQNK NRYVDILPYD YNRVELSEIN GDAGSNYINA SYIDGFKEPR |
| | KYIAAQGPRD ETVDDFWRMI WEQKATVIVM VTRCEEGNRN KCAEYWPSME EGTRAFGDVV |
| | VKINQHKRCP DYIIQKLNIV NKKEKATGRE VTHIQFTSWP DHGVPEDPHL LLKLRRRVNA |
| | FSNFFSGPIV VHCSAGVGRT GTYIGIDAML EGLEAENKVD VYGYVVKLRR QRCLMVQVEA |
| | QYILIHQALV EYNQFGETEV NLSELHPYLH NMKKRDPPSE PSPLEAEFQR LPSYRSWRTQ |
| | HIGNQEENKS KNRNSNVIPY DYNRVPLKHE LEMSKESEHD SDESSDDDSD SEEPSKYINA |
| | SFIMSYWKPE VMIAAQGPLK ETIGDFWQMI FQRKVKVIVM LTELKHGDQE ICAQYWGEGK |
| | QTYGDIEVDL KDTDKSSTYT LRVFELRHSK RKDSRTVYQY QYTNWSVEQL PAEPKELISM |
| | IQVVKQKLPQ KNSSEGNKHH KSTPLLIHCR DGSQQTGIFC ALLNLLESAE TEEVVDIFQV |
| | VKALRKARPG MVSTFEQYQF LYDVIASTYP AQNGQVKKNN HQEDKIEFDN EVDKVKQDAN |
| | CVNPLGAPEK LPEAKEQAEG SEPTSGTEGP EHSVNGPASP ALNQGS |
| | (SEQ ID NO: 1) |
| CD45 | MTMYLWLKLL AFGFAFLDTE VFVTGQSPTP SPTDAYLNAS ETTTLSPSGS AVISTTTIAT |
| Isoform 2 | TPSKPTCDEK YANITVDYLY NKETKLFTAK LNVNENVECG NNTCTNNEVH NLTECKNASV |
| (AA34-194 | SISHNSCTAP DKTLILDVPP GVEKFQLHDC TQVEKADTTI CLKWKNIETF TCDTQNITYR |
| are | FQCGNMIFDN KEIKLENLEP EHEYKCDSEI LYNNHKFTNA SKIIKTDFGS PGEPQIIFCR |
| missing) | SEAAHQGVIT WNPPQRSFHN FTLCYIKETE KDCLNLDKNL IKYDLQNLKP YTKYVLSLHA |
| | YIIAKVQRNG SAAMCHFTTK SAPPSQVWNM TVSMTSDNSM HVKCRPPRDR NGPHERYHLE |
| | VEAGNTLVRN ESHKNCDFRV KDLQYSTDYT FKAYFHNGDY PGEPFILHHS TSYNSKALIA |
| | FLAFLIIVTS IALLVVLYKI YDLHKKRSCN LDEQQELVER DDEKQLMNVE PIHADILLET |
| | YKRKIADEGR LFLAEFQSIP RVFSKFPIKE ARKPFNQNKN RYVDILPYDY NRVELSEING |
| | DAGSNYINAS YIDGFKEPRK YIAAQGPRDE TVDDFWRMIW EQKATVIVMV TRCEEGNRNK |
| | CAEYWPSMEE GTRAFGDVVV KINQHKRCPD YIIQKLNIVN KKEKATGREV THIQFTSWPD |
| | HGVPEDPHLL LKLRRRVNAF SNFFSGPIVV HCSAGVGRTG TYIGIDAMLE GLEAENKVDV |
| | YGYVVKLRRQ RCLMVQVEAQ YILIHQALVE YNQFGETEVN LSELHPYLHN MKKRDPPSEP |
| | SPLEAEFQRL PSYRSWRTQH IGNQEENKSK NRNSNVIPYD YNRVPLKHEL EMSKESEHDS |
| | DESSDDDSDS EEPSKYINAS FIMSYWKPEV MIAAQGPLKE TIGDFWQMIF QRKVKVIVML |
| | TELKHGDQEI CAQYWGEGKQ TYGDIEVDLK DTDKSSTYTL RVFELRHSKR KDSRTVYQYQ |
| | YTNWSVEQLP AEPKELISMI QVVKQKLPQK NSSEGNKHHK STPLLIHCRD GSQQTGIFCA |
| | LLNLLESAET EEVVDIFQVV KALRKARPGM VSTFEQYQFL YDVIASTYPA QNGQVKKNNH |
| | QEDKIEFDNE VDKVKQDANC VNPLGAPEKL PEAKEQAEGS EPTSGTEGPE HSVNGPASPA |
| | LNQGS |
| | (SEQ ID NO: 2) |
| CD45 | MTMYLWLKLL AFGFAFLDTE VFVTGQSPTP SPTGLTTAKM PSVPLSSDPL PTHTTAFSPA |
| Isoform 3 | STFERENDFS ETTTSLSPDN TSTQVSPDSL DNASAFNTTG VSSVQTPHLP THADSQTPSA |
| (AA147- | GTDTQTFSGS AANAKLNPTP GSNAISDAYL NASETTTLSP SGSAVISTTT IATTPSKPTC |
| 194 are | DEKYANITVD YLYNKETKLF TAKLNVNENV ECGNNTCTNN EVHNLTECKN ASVSISHNSC |
| missing) | TAPDKTLILD VPPGVEKFQL HDCTQVEKAD TTICLKWKNI ETFTCDTQNI TYRFQCGNMI |
| | FDNKEIKLEN LEPEHEYKCD SEILYNNHKF TNASKIIKTD FGSPGEPQII FCRSEAAHQG |
| | VITWNPPQRS FHNFTLCYIK ETEKDCLNLD NSMHVKCRPP LKPYTKYVLS LHAYIIAKVQ |
| | RNGSAAMCHF TTKSAPPSQV WNMTVSMTSD KNLIKYDLQN RDRNGPHERY HLEVEAGNTL |
| | VRNESHKNCD FRVKDLQYST DYTFKAYFHN GDYPGEPFIL HHSTSYNSKA LIAFLAFLII |
| | VTSIALLVVL YKIYDLHKKR SCNLDEQQEL VERDDEKQLM NVEPIHADIL LETYKRKIAD |
| | EGRLFLAEFQ SIPRVESKFP IKEARKPENQ NKNRYVDILP YDYNRVELSE INGDAGSNYI |
| | NASYIDGFKE PRKYIAAQGP RDETVDDFWR MIWEQKATVI VMVTRCEEGN RNKCAEYWPS |
| | MEEGTRAFGD VVVKINQHKR CPDYIIQKLN IVNKKEKATG REVTHIQFTS WPDHGVPEDP |
| | HLLLKLRRRV NAFSNFFSGP IVVHCSAGVG RTGTYIGIDA MLEGLEAENK VDVYGYVVKL |
| | RRQRCLMVQV EAQYILIHQA LVEYNQFGET EVNLSELHPY LHNMKKRDPP SEPSPLEAEF |
| | QRLPSYRSWR TQHIGNQEEN KSKNRNSNVI PYDYNRVPLK HELEMSKESE HDSDESSDDD |
| | SDSEEPSKYI NASFIMSYWK PEVMIAAQGP LKETIGDFWQ MIFQRKVKVI VMLTELKHGD |
| | QEICAQYWGE GKQTYGDIEV DLKDTDKSST YTLRVFELRH SKRKDSRTVY QYQYTNWSVE |
| | QLPAEPKELI SMIQVVKQKL PQKNSSEGNK HHKSTPLLIH CRDGSQQTGI FCALLNLLES |
| | AETEEVVDIF QVVKALRKAR PGMVSTFEQY QFLYDVIAST YPAQNGQVKK NNHQEDKIEF |
| | DNEVDKVKQD ANCVNPLGAP EKLPEAKEQA EGSEPTSGTE GPEHSVNGPA SPALNQGS |
| | (SEQ ID NO: 3) |
| CD45 | MTMYLWLKLL AFGFAFLDTE VFVTGQSPTP SPTGLTTAKM PSVPLSSDPL PTHTTAFSPA |
| Isoform 4 | STFERENDFS ETTTSLSPDN TSTQVSPDSL DNASAFNTTD VPGERSTAST FPTDPVSPLT |
| (AA100- | TTLSLAHHSS AALPARTSNT TITANTSDAY LNASETTTLS PSGSAVISTT TIATTPSKPT |
| 146 are | CDEKYANITV DYLYNKETKL FTAKLNVNEN VECGNNTCTN NEVHNLTECK NASVSISHNS |
| missing) | CTAPDKTLIL DVPPGVEKFQ LHDCTQVEKA DTTICLKWKN IETFTCDTQN ITYRFQCGNM |
| | IFDNKEIKLE NLEPEHEYKC DSEILYNNHK FTNASKIIKT DFGSPGEPQI IFCRSEAAHQ |
| | QRNGSAAMCH FTTKSAPPSQ VWNMTVSMTS DKNLIKYDLQ NLKPYTKYVL SLHAYIIAKV |
| | GVITWNPPQR SFHNFTLCYI KETEKDCLNL DNSMHVKCRP PRDRNGPHER YHLEVEAGNT |
| | LVRNESHKNC DFRVKDLQYS TDYTFKAYFH NGDYPGEPFI LHHSTSYNSK ALIAFLAFLI |
| | IVTSIALLVV LYKIYDLHKK RSCNLDEQQE LVERDDEKQL MNVEPIHADI LLETYKRKIA |
| | DEGRLFLAEF QSIPRVESKF PIKEARKPEN QNKNRYVDIL PYDYNRVELS EINGDAGSNY |
| | INASYIDGFK EPRKYIAAQG PRDETVDDFW RMIWEQKATV IVMVTRCEEG NRNKCAEYWP |
| | SMEEGTRAFG DVVVKINQHK RCPDYIIQKL NIVNKKEKAT GREVTHIQFT SWPDHGVPED |
| | PHLLLKLRRR VNAFSNFFSG PIVVHCSAGV GRTGTYIGID AMLEGLEAEN KVDVYGYVVK |
| | LRRQRCLMVQ VEAQYILIHQ ALVEYNQFGE TEVNLSELHP YLHNMKKRDP PSEPSPLEAE |
| | FQRLPSYRSW RTQHIGNQEE NKSKNRNSNV IPYDYNRVPL KHELEMSKES EHDSDESSDD |
| | DSDSEEPSKY INASFIMSYW KPEVMIAAQG PLKETIGDFW QMIFQRKVKV IVMLTELKHG |
| | DQEICAQYWG EGKQTYGDIE VDLKDTDKSS TYTLRVFELR HSKRKDSRTV YQYQYTNWSV |
| | EQLPAEPKEL ISMIQVVKQK LPQKNSSEGN KHHKSTPLLI HCRDGSQQTG IFCALLNLLE |
| | SAETEEVVDI FQVVKALRKA RPGMVSTFEQ YQFLYDVIAS TYPAQNGQVK KNNHQEDKIE |
| | FDNEVDKVKQ DANCVNPLGA PEKLPEAKEQ AEGSEPTSGT EGPEHSVNGP ASPALNQGS |
| | (SEQ ID NO: 4) |
| CD45 | MTMYLWLKLL AFGFAFLDTE VFVTGQSPTP SPTGVSSVQT PHLPTHADSQ TPSAGTDTQT |
| Isoform 5 | FSGSAANAKL NPTPGSNAIS DVPGERSTAS TFPTDPVSPL TTTLSLAHHS SAALPARTSN |
| (AA34-99 | TTITANTSDA YLNASETTTL SPSGSAVIST TTIATTPSKP TCDEKYANIT VDYLYNKETK |
| are | LFTAKLNVNE NVECGNNTCT NNEVHNLTEC KNASVSISHN SCTAPDKTLI LDVPPGVEKF |
| missing) | QLHDCTQVEK ADTTICLKWK NIETFTCDTQ NITYRFQCGN MIFDNKEIKL ENLEPEHEYK |
| | CDSEILYNNH KFTNASKIIK TDFGSPGEPQ IIFCRSEAAH QGVITWNPPQ RSFHNFTLCY |
| | IKETEKDCLN LDKNLIKYDL QNLKPYTKYV RYHLEVEAGN TLVRNESHKN HFTTKSAPPS |
| | QVWNMTVSMT SDNSMHVKCR PPRDRNGPHE LSLHAYIIAK VQRNGSAAMC CDFRVKDLQY |
| | STDYTFKAYF HNGDYPGEPF ILHHSTSYNS KALIAFLAFL IIVTSIALLV VLYKIYDLHK |
| | KRSCNLDEQQ ELVERDDEKQ LMNVEPIHAD ILLETYKRKI ADEGRLFLAE FQSIPRVFSK |
| | FPIKEARKPF NQNKNRYVDI LPYDYNRVEL SEINGDAGSN YINASYIDGE KEPRKYIAAQ |
| | GPRDETVDDF WRMIWEQKAT VIVMVTRCEE GNRNKCAEYW PSMEEGTRAF GDVVVKINQH |
| | KRCPDYIIQK LNIVNKKEKA TGREVTHIQF TSWPDHGVPE DPHLLLKLRR RVNAFSNFFS |
| | GPIVVHCSAG VGRTGTYIGI DAMLEGLEAE NKVDVYGYVV KLRRQRCLMV QVEAQYILIH |
| | QALVEYNQFG ETEVNLSELH PYLHNMKKRD PPSEPSPLEA EFQRLPSYRS WRTQHIGNQE |
| | ENKSKNRNSN VIPYDYNRVP LKHELEMSKE SEHDSDESSD DDSDSEEPSK YINASFIMSY |
| | WKPEVMIAAQ GPLKETIGDF WQMIFQRKVK VIVMLTELKH GDQEICAQYW GEGKQTYGDI |
| | EVDLKDTDKS STYTLRVFEL RHSKRKDSRT VYQYQYTNWS VEQLPAEPKE LISMIQVVKQ |
| | KLPQKNSSEG NKHHKSTPLL IHCRDGSQQT GIFCALLNLL ESAETEEVVD IFQVVKALRK |
| | ARPGMVSTFE QYQFLYDVIA STYPAQNGQV KKNNHQEDKI EFDNEVDKVK QDANCVNPLG |
| | APEKLPEAKE QAEGSEPTSG TEGPEHSVNG PASPALNQGS |
| | (SEQ ID NO: 5) |
| CD45 | MTMYLWLKLL AFGFAFLDTE VFVTGQSPTP SPTGLTTAKM PSVPLSSDPL PTHTTAFSPA |
| Isoform 6 | STFERENDFS ETTTSLSPDN TSTQVSPDSL DNASAFNTTD AYLNASETTT LSPSGSAVIS |
| (AA100- | TTTIATTPSK PTCDEKYANI TVDYLYNKET KLFTAKLNVN ENVECGNNTC TNNEVHNLTE |
| 194 are | CKNASVSISH NSCTAPDKTL ILDVPPGVEK FQLHDCTQVE KADTTICLKW KNIETFTCDT |
| missing | QNITYRFQCG NMIFDNKEIK LENLEPEHEY KCDSEILYNN HKFTNASKII KTDFGSPGEP |
| | QIIFCRSEAA HQGVITWNPP QRSFHNFTLC YIKETEKDCL NLDKNLIKYD LQNLKPYTKY |
| | VLSLHAYIIA KVQRNGSAAM CHFTTKSAPP SQVWNMTVSM TSDNSMHVKC RPPRDRNGPH |
| | ERYHLEVEAG NTLVRNESHK NCDFRVKDLQ YSTDYTFKAY FHNGDYPGEP FILHHSTSYN |
| | SKALIAFLAF LIIVTSIALL VVLYKIYDLH KKRSCNLDEQ QELVERDDEK QLMNVEPIHA |
| | DILLETYKRK IADEGRLFLA EFQSIPRVES KFPIKEARKP FNQNKNRYVD ILPYDYNRVE |
| | LSEINGDAGS NYINASYIDG FKEPRKYIAA QGPRDETVDD FWRMIWEQKA TVIVMVTRCE |
| | EGNRNKCAEY WPSMEEGTRA FGDVVVKINQ HKRCPDYIIQ KLNIVNKKEK ATGREVTHIQ |
| | FTSWPDHGVP EDPHLLLKLR RRVNAFSNFF SGPIVVHCSA GVGRTGTYIG IDAMLEGLEA |
| | ENKVDVYGYV VKLRRQRCLM VQVEAQYILI HQALVEYNQF GETEVNLSEL HPYLHNMKKR |
| | DPPSEPSPLE AEFQRLPSYR SWRTQHIGNQ EENKSKNRNS NVIPYDYNRV PLKHELEMSK |
| | ESEHDSDESS DDDSDSEEPS KYINASFIMS YWKPEVMIAA QGPLKETIGD FWQMIFQRKV |
| | KVIVMLTELK HGDQEICAQY WGEGKQTYGD IEVDLKDTDK SSTYTLRVFE LRHSKRKDSR |
| | TVYQYQYTNW SVEQLPAEPK ELISMIQVVK QKLPQKNSSE GNKHHKSTPL LIHCRDGSQQ |
| | TGIFCALLNL LESAETEEVV DIFQVVKALR KARPGMVSTF EQYQFLYDVI ASTYPAQNGQ |
| | VKKNNHQEDK IEFDNEVDKV KQDANCVNPL GAPEKLPEAK EQAEGSEPTS GTEGPEHSVN |
| | GPASPALNQG S |
| | (SEQ ID NO: 6) |
| CD45 | FSGSAANAKL NPTPGSNAIS DAYLNASETT TLSPSGSAVI STTTIATTPS TPSAGTDTQT |
| Isoform 7 | MTMYLWLKLL AFGFAFLDTE VFVTGQSPTP SPTGVSSVQT PHLPTHADSQ KPTCDEKYAN |
| (AA34-99 | ITVDYLYNKE TKLFTAKLNV NENVECGNNT CTNNEVHNLT ECKNASVSIS HNSCTAPDKT |
| and | LILDVPPGVE KFQLHDCTQV EKADTTICLK WKNIETFTCD TQNITYRFQC GNMIFDNKEI |
| AA147- | KLENLEPEHE YKCDSEILYN NHKFTNASKI IKTDFGSPGE PQIIFCRSEA AHQGVITWNP |
| 194 are | PQRSFHNFTL CYIKETEKDC LNLDKNLIKY DLQNLKPYTK YVLSLHAYII AKVQRNGSAA |
| missing | MCHFTTKSAP PSQVWNMTVS MTSDNSMHVK CRPPRDRNGP HERYHLEVEA GNTLVRNESH |
| | KNCDFRVKDL QYSTDYTFKA YFHNGDYPGE PFILHHSTSY NSKALIAFLA FLIIVTSIAL |
| | LVVLYKIYDL HKKRSCNLDE QQELVERDDE KQLMNVEPIH ADILLETYKR KIADEGRLFL |
| | AEFQSIPRVF SKFPIKEARK PFNQNKNRYV DILPYDYNRV ELSEINGDAG SNYINASYID |
| | GFKEPRKYIA AQGPRDETVD DFWRMIWEQK ATVIVMVTRC EEGNRNKCAE YWPSMEEGTR |
| | AFGDVVVKIN QHKRCPDYII QKLNIVNKKE KATGREVTHI QFTSWPDHGV PEDPHLLLKL |
| | RRRVNAFSNF FSGPIVVHCS AGVGRTGTYI GIDAMLEGLE AENKVDVYGY VVKLRRQRCL |
| | MVQVEAQYIL IHQALVEYNQ FGETEVNLSE LHPYLHNMKK RDPPSEPSPL EAEFQRLPSY |
| | RSWRTQHIGN QEENKSKNRN SNVIPYDYNR VPLKHELEMS KESEHDSDES SDDDSDSEEP |
| | SKYINASFIM SYWKPEVMIA AQGPLKETIG DFWQMIFQRK VKVIVMLTEL KHGDQEICAQ |
| | YWGEGKQTYG DIEVDLKDTD KSSTYTLRVF ELRHSKRKDS RTVYQYQYTN WSVEQLPAEP |
| | KELISMIQVV KQKLPQKNSS EGNKHHKSTP LLIHCRDGSQ QTGIFCALLN LLESAETEEV |
| | VDIFQVVKAL RKARPGMVST FEQYQFLYDV IASTYPAQNG QVKKNNHQED KIEFDNEVDK |
| | VKQDANCVNP LGAPEKLPEA KEQAEGSEPT SGTEGPEHSV NGPASPALNQ GS |
| | (SEQ ID NO: 7) |
| CD45 | MTMYLWLKLL AFGFAFLDTE VFVTGQSPTP SPTDVPGERS TASTFPTDPV SPLTTTLSLA |
| Isoform 8 | HHSSAALPAR TSNTTITANT SDAYLNASET TTLSPSGSAV ISTTTIATTP SKPTCDEKYA |
| (AA34-146 | NITVDYLYNK ETKLFTAKLN VNENVECGNN TCTNNEVHNL TECKNASVSI SHNSCTAPDK |
| are missing | TLILDVPPGV EKFQLHDCTQ VEKADTTICL KWKNIETFTC DTQNITYRFQ CGNMIFDNKE |
| | IKLENLEPEH EYKCDSEILY NNHKFTNASK IIKTDFGSPG EPQIIFCRSE AAHQGVITWN |
| | PPQRSFHNFT LCYIKETEKD CLNLDKNLIK YDLQNLKPYT KYVLSLHAYI IAKVQRNGSA |
| | AMCHFTTKSA PPSQVWNMTV SMTSDNSMHV KCRPPRDRNG PHERYHLEVE AGNTLVRNES |
| | HKNCDFRVKD LQYSTDYTFK AYFHNGDYPG EPFILHHSTS YNSKALIAFL AFLIIVTSIA |
| | LLVVLYKIYD LHKKRSCNLD EQQELVERDD EKQLMNVEPI HADILLETYK RKIADEGRLF |
| | LAEFQSIPRV FSKFPIKEAR KPFNQNKNRY VDILPYDYNR VELSEINGDA GSNYINASYI |
| | DGFKEPRKYI AAQGPRDETV DDFWRMIWEQ KATVIVMVTR CEEGNRNKCA EYWPSMEEGT |
| | RAFGDVVVKI NQHKRCPDYI IQKLNIVNKK EKATGREVTH IQFTSWPDHG VPEDPHLLLK |
| | LRRRVNAFSN FFSGPIVVHC SAGVGRTGTY IGIDAMLEGL EAENKVDVYG YVVKLRRQRC |
| | LMVQVEAQYI LIHQALVEYN QFGETEVNLS ELHPYLHNMK KRDPPSEPSP LEAEFQRLPS |
| | YRSWRTQHIG NQEENKSKNR NSNVIPYDYN RVPLKHELEM SKESEHDSDE SSDDDSDSEE |
| | PSKYINASFI MSYWKPEVMI AAQGPLKETI GDFWQMIFQR KVKVIVMLTE LKHGDQEICA |
| | QYWGEGKQTY GDIEVDLKDT DKSSTYTLRV FELRHSKRKD SRTVYQYQYT NWSVEQLPAE |
| | PKELISMIQV VKQKLPQKNS SEGNKHHKST PLLIHCRDGS QQTGIFCALL NLLESAETEE |
| | VVDIFQVVKA LRKARPGMVS TFEQYQFLYD VIASTYPAQN GQVKKNNHQE DKIEFDNEVD |
| | KVKQDANCVN PLGAPEKLPE AKEQAEGSEP TSGTEGPEHS VNGPASPALN QGS |
| | (SEQ ID NO: 8) |
| CD45 | QSPTPSPTGL TTAKMPSVPL SSDPLPTHTT AFSPASTFER ENDESETTTS |
| antigen | LSPDNTSTQV SPDSLDNASA FNTTGVSSVQ TPHLPTHADS QTPSAGTDTQ |
| recognition | TFSGSAANAK LNPTPGSNAI SDVPGERSTA STFPTDPVSP LTTTLSLAHH |
| domain | SSAALPARTS NTTITANTSD AYLNASETTT LSPSGSAVIS TTTIATTPSK |
| | PTCDEKYANI TVDYLYNKET KLFTAKLNVN ENVECGNNTC TNNEVHNLTE |
| | CKNASVSISH NSCTAPDKTL ILDVPPGVEK FQLHDCTQVE KADTTICLKW |
| | KNIETFTCDT QNITYRFQCG NMIFDNKEIK LENLEPEHEY KCDSEILYNN |
| | HKFTNASKII KTDFGSPGEP QIIFCRSEAA HQGVITWNPP QRSFHNFTLC |
| | YIKETEKDCL NLDKNLIKYD LQNLKPYTKY VLSLHAYIIA KVQRNGSAAM |
| | CHFTTKSAPP SQVWNMTVSM TSDNSMHVKC |
| | (SEQ ID NO: 9) |

In some embodiments, the activator antigen comprises a peptide of CD45 that lacks amino acids 34-194, as compared with the CD45 peptide of SEQ ID NO: 1. In some embodiments, the activator antigen comprises a peptide of CD45 that lacks amino acids 147-194, as compared with the CD45 peptide of SEQ ID NO: 1. In some embodiments, the activator antigen comprises a peptide of CD45 that lacks amino acids 100-146, as compared with the CD45 peptide of SEQ ID NO: 1. In some embodiments, the activator antigen comprises a peptide of CD45 that lacks amino acids 147-194, as compared with the CD45 peptide of SEQ ID NO: 1. In some embodiments, the activator antigen comprises a peptide of CD45 that lacks amino acids 34-146, as compared with the CD45 peptide of SEQ ID NO: 1.

As used herein, an “activator,” “activator antigen,” or “activator ligand” refers to a first ligand that binds to a first, activator ligand binding domain (LBD) of an engineered receptor of the disclosure, such as a CAR or TCR, thereby mediating activation of a T cell or other immune cell expressing the activator receptor. The activator ligand is expressed by target cells, for example cancer cells, and may also be expressed more broadly than just the cancer cells. For example the activator can be expressed on some, or all types of normal, non-cancerous cells such as hematopoietic stem cells (HSCs) or blood cells.

In some embodiments, the activator ligand is expressed by cancer cells and is not expressed by non-cancerous cells (i.e. normal cells not targeted by the adoptive cell therapy). In some embodiments, the target cells are cancer cells and the normal cells are non-cancerous cells.

In some embodiments, the activator ligand has high cell surface expression on the cancer cells. In some embodiments, the A antigen is expressed by cancer cells and is not expressed by allogenic donor stem cells. In some embodiments, the target cells are a subject's cancer cells and/or the subject's blood cells.

In some embodiments, the A antigen has high cell surface expression on cancerous and non-cancerous bloodcells.

This high cell surface expression confers the ability to deliver large activation signals. Methods of measuring cell surface expression will be known to the person of ordinary skill in the art and include, but are not limited to, immunohistochemistry using an appropriate antibody against the activator ligand, followed by microscopy or fluorescence activated cell sorting (FACS).

The activator ligand is present on all cancer cells. In some embodiments, the target cells are cancer cells, such as blood cancer cells.

In some embodiments, the activator ligand is present on a plurality of cancer cells. In some embodiments, the activator ligand is present on at least 50%, at least 60%, at least 70%, at least 80%, at least 85%, at least 90%, at least 95%, at least 96%, at least 97%, at least 98%, at least 99%, at least 99.5% or at least 99.9% of cancer cells. In some embodiments, the activator ligand is present on at least 95% of cancer cells. In some embodiments, the activator ligand is present on at least 99% of cancer cells.

The A antigen may be present on all blood cells, or on a subset of blood cells. Generally, the subset of blood cells is selected to include cancer cells, although application of the disclosed compositions and methods in a subject not having cancer is also contemplated. For example, the engineered immune cells may be used to condition subjects having aplastic anemia or other non-malignant conditions for stem cell transplant, or to promote engraftment or expansion of stem cells after a subject has received stem cell transplant for cancer or for another non-malignant condition. Accordingly, the A antigen may be an antigen present on blood cells, or a subset of blood cells, but not on cancer cells-particularly in applications in which the subject has no detected cancer.

In some embodiments, the A antigen is present on a plurality of patient blood cells. In some embodiments, the A antigen is present on at least 50%, at least 60%, at least 70%, at least 80%, at least 85%, at least 90%, at least 95%, at least 96%, at least 97%, at least 98%, at least 99%, at least 99.5% or at least 99.9% of patient blood cells. In some embodiments, the A antigen is present on at least 95% of patient blood cells. In some embodiments, the A antigen is present on at least 99% of patient blood cells.

In some embodiments, the activator ligand is expressed by a plurality of cancer cells and a plurality of non-cancerous cells. In some embodiments, the plurality of non-cancerous cells expresses both the activator ligand and the inhibitor ligand.

In some embodiments, and the activator ligand and inhibitor ligand are present on the plurality of non-cancerous cells at a ratio of about 1:100 to about 100:1 of the activator ligand to the inhibitor ligand. In some embodiments, the activator ligand and the inhibitor ligand are present on the plurality of non-cancerous cells at a ratio of about 1:50 to about 50:1 of the activator ligand to the inhibitor ligand. In some embodiments, the activator ligand and inhibitor ligand are present on the plurality of non-cancerous cells at a ratio of about 1:40 to about 40:1 of the activator ligand to the inhibitor ligand. In some embodiments, the activator ligand and inhibitor ligand are present on the plurality of non-cancerous cells at a ratio of about 1:30 to about 30:1 of the activator ligand to the inhibitor ligand. In some embodiments, the activator ligand and inhibitor ligand are present on the plurality of non-cancerous cells at a ratio of about 1:20 to about 2:1 of the activator ligand to the inhibitor ligand. In some embodiments, the activator ligand and inhibitor ligand are present on the plurality of non-cancerous cells at a ratio of about 1:10 to about 10:1 of the activator ligand to the inhibitor ligand. In some embodiments, the activator ligand and inhibitor ligand are present on the plurality of non-cancerous cells at a ratio of about 1:5 to about 5:1 of the activator ligand to the inhibitor ligand. In some embodiments, the activator ligand and inhibitor ligand are present on the plurality of non-cancerous cells at a ratio of about 1:3 to about 3:1 of the activator ligand to the inhibitor ligand. In some embodiments, the activator ligand and inhibitor ligand are present on the plurality of non-cancerous cells at a ratio of about 1:2 to about 2:1 of the activator ligand to the inhibitor ligand. In some embodiments, the activator ligand and inhibitor ligand are present on the plurality of non-cancerous cells at a ratio of about 1:1 of the activator ligand to the inhibitor ligand.

In some embodiments, the A antigen is expressed by a plurality of cancer cells and a plurality of allogeneic donor cells. In some embodiments, the plurality of allogeneic donor cells expresses both the A antigen and the inhibitor ligand.

In some embodiments, and the A antigen and inhibitor ligand are present on the plurality of allogeneic donor cells at a ratio of about 1:100 to about 100:1 of the A antigen to the inhibitor ligand. In some embodiments, the A antigen and the inhibitor ligand are present on the plurality of allogeneic donor cells at a ratio of about 1:50 to about 50:1 of the A antigen to the inhibitor ligand. In some embodiments, the A antigen and inhibitor ligand are present on the plurality of allogeneic donor cells at a ratio of about 1:40 to about 40:1 of the A antigen to the inhibitor ligand. In some embodiments, the A antigen and inhibitor ligand are present on the plurality of allogeneic donor cells at a ratio of about 1:30 to about 30:1 of the A antigen to the inhibitor ligand. In some embodiments, the A antigen and inhibitor ligand are present on the plurality of allogeneic donor cells at a ratio of about 1:20 to about 2:1 of the A antigen to the inhibitor ligand. In some embodiments, the A antigen and inhibitor ligand are present on the plurality of allogeneic donor cells at a ratio of about 1:10 to about 10:1 of the A antigen to the inhibitor ligand. In some embodiments, the A antigen and inhibitor ligand are present on the plurality of allogeneic donor cells at a ratio of about 1:5 to about 5:1 of the A antigen to the inhibitor ligand. In some embodiments, the A antigen and inhibitor ligand are present on the plurality of allogeneic donor cells at a ratio of about 1:3 to about 3:1 of the A antigen to the inhibitor ligand. In some embodiments, the A antigen and inhibitor ligand are present on the plurality of allogeneic donor cells at a ratio of about 1:2 to about 2:1 of the A antigen to the inhibitor ligand. In some embodiments, the A antigen and inhibitor ligand are present on the plurality of allogeneic donor cells at a ratio of about 1:1 of the A antigen to the inhibitor ligand.

### Extracellular Ligand Binding Domains

The disclosure provides chimeric antigen receptors (CARs) comprising a polypeptide. In some embodiments, the polypeptide comprises a ligand binding domain, such as an antigen-binding domain. Suitable antigen-binding domains include, but are not limited to, antigen-binding domains from antibodies, antibody fragments, scFv, antigen-binding domains derived from T cell receptors, and the like. All forms of antigen-binding domains known in the art are envisaged as within the scope of the disclosure.

An “extracellular domain,” as used herein, refers to the extracellular portion of a protein. For example, the TCR alpha and beta chains each comprise an extracellular domain, which comprise a constant and a variable region involved in peptide-MHC recognition. The “extracellular domain” can also comprise a fusion domain, for example of fusions between additional domains capable of binding to and targeting a specific antigen and the endogenous extracellular domain of the TCR subunit.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof that contains the antigen-binding domain, i.e., an antigenic determining variable region of an intact antibody that is sufficient to confer recognition and specific binding of the antibody fragment to a target, such as an antigen and its defined epitope. Examples of antibody fragments include, but are not limited to, Fab, Fab′, F(ab′)2, and Fv fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Light chain variable region” or “VL” with regard to an antibody refers to the fragment of the light chain that contains three CDRs interposed between flanking stretches known as framework regions, these framework regions are generally more highly conserved than the CDRs and form a scaffold to support the CDRs.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The term “antibody light chain,” refers to the smaller of the two types of polypeptide chains present in antibody molecules in their naturally occurring conformations. Kappa (“K”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

In some embodiments, the extracellular ligand binding domain comprises a binding agent for CD45, such as an antibody or antigen-binding fragment thereof. In some embodiments, the anti-CD45 antibody is a human anti-CD45 antibody, a humanized anti-CD45 antibody, or a chimeric anti-CD45 antibody. In some embodiments, the anti-CD45 antibody is an anti-CD45 monoclonal antibody. Illustrative anti-CD45 antibodies include antibodies BC8, 4B2, GAP8.3 or 9.4. In some embodiments, the antibody molecule or antigen-binding fragment that binds to CD45 is specific to one CD45 isoform or binds to more than one CD45 isoform, e.g., is a pan-CD45 antibody or antigen-binding fragment.

In some embodiments, the extracellular ligand binding domain of the activator receptor comprises an antibody fragment, a single chain Fv antibody fragment (scFv), a β chain variable domain (Vβ), or a TCR α chain variable domain and a TCR β chain variable domain. In some embodiments, the extracellular ligand binding domain of the activator receptor comprises a heavy chain variable (VH) region and a light chain variable (VL) region. In some embodiments, the VH and VL regions comprise complement determining regions (CDRs) selected from the group of CDRs disclosed in Table 3.

TABLE 3
CD45 antigen binding domain complement determining regions (CDRs)

| CDR-L1 | CDR-L2 | CDR-L3 | CDR-H1 | CDR-H2 | CDR-H3 |
|---|---|---|---|---|---|
| RASKSVST | LASNLES | QHSRELP | RYWMS | EINPTSSTINFTP | GNYYRYGD |
| SGYSYLH | (SEQ ID | FTFGSGT | (SEQ ID | SLKD | AMDY (SEQ |
| (SEQ ID | NO: 14) | (SEQ ID | NO: 10) | (SEQ ID NO: 11) | ID NO: 12) |
| NO: 13) | | NO: 15) | | | |

In some embodiments, the VH region comprises one or more CDR sequences selected from the group consisting of RYWMS (SEQ ID NO: 10), EINPTSSTINFTPSLKD (SEQ ID NO: 11), and GNYYRYGDAMDY (SEQ ID NO: 12); and the VL region comprises one or more CDR sequences selected from the group consisting of RASKSVSTSGYSYLH (SEQ ID NO: 13), LASNLES (SEQ ID NO: 14), and QHSRELPFTFGSGT (SEQ ID NO: 15).

In some embodiments, the full length VH and VL regions comprise the sequences disclosed in Table 4. In some embodiments, the binding domain comprises the full length VH region and VL regions on a single polypeptide. In some embodiments, the polypeptide comprises from N-terminal to C-terminal the full length VH regions and the full length VL region. In some embodiments, the polypeptide comprises from N-terminal to C-terminal the full length VL region and the full length VH region. In some embodiments, the full length VH and VL comprises the sequence selected from Table 4. In some embodiments, the binding domain comprises SEQ ID NO: 16 and SEQ ID NO: 18. In some embodiments, the binding domain comprises SEQ ID NO: 17 and SEQ ID NO: 18. In some embodiments, the binding domain comprises SEQ ID NO: 20 and SEQ ID NO: 19. In some embodiments, the binding domain comprises SEQ ID NO: 21 and SEQ ID NO: 22. In some embodiments, the binding domain comprises SEQ ID NO: 23 and SEQ ID NO: 24. In some embodiments, the binding domain comprises SEQ ID NO: 25 and SEQ ID NO: 26. In some embodiments, the binding domain comprises SEQ ID NO: 27 and SEQ ID NO: 28.

TABLE 4
VH and VL Domains for anti-CD45 CAR Constructs

| Construct | VH Full Length | VL Full Length |
|---|---|---|
| Anti-CD45 | QVQLVESGGGLVQPGGSLKLS | DIVLTQSPASLAVSLGQRATISCRA |
| scFv heavy | CAASGFDFSRYWMSWVRQAP | SKSVSTSGYSYLHWYQQKPGQPP |
| chain | GKGLEWIGEINPTSSTINFTPSL | KLLIYLASNLESGVPARFSGSGSGT |
| variable | KDKVFISRDNAKNTLYLQMSK | DFTLNIHPVEEEDAATYYCQHSRE |
| region | VRSEDTALYYCARGNYYRYGD | LPFTFGSGTKLEIK |
| | AMDYWGQGTSVTVSKIS | (SEQ ID NO: 18) |
| | (SEQ ID NO: 16) | |
| Anti-CD45 | QVQLVESGGGLVQPGGSLKLS | DIVLTQSPASLAVSLGQRATISCRA |
| scFv heavy | CAASGFDFSRYWMSWVRQAP | SKSVSTSGYSYLHWYQQKPGQPP |
| chain | GKGLEWIGEINPTSSTINFTPSL | KLLIYLASNLESGVPARFSGSGSGT |
| variable | KDKVFISRDNAKNTLYLQMSK | DFTLNIHPVEEEDAATYYCQHSRE |
| region | VRSEDTALYYCARGNYYRYGD | LPFTFGSGTKLEIK |
| | AMDYWGQGTSVTVSK | (SEQ ID NO: 18) |
| | (SEQ ID NO: 17) | |
| Fab BC8 | QVQLVESGGGLVQPGGSLKLS | DIVLTQSPASLAVSLGQRATISCRA |
| anti-CD45 | CAASGFDFSRYWMSWVRQAP | SKSVSTSGYSYLHWYQQKPGQPP |
| antibody | GKGLEWIGEINPTSSTINFTPSL | KLLIYLASNLESGVPARFSGSGSGT |
| | KDKVFISRDNAKNTLYLQMSK | DFTLNIHPVEEEDAATYYCQHSRE |
| | VRSEDTALYYCARGNYYRYGD | LPFTFGSGTKLEIKRTVAAPSVFIFP |
| | AMDYWGQGTSVTVSS | PSDEQLKSGTASVVCLLNNFYPRE |
| | (SEQ ID NO: 20) | AKVQWKVDNALQSGNSQESVTEQ |
| | | DSKDSTYSLSSTLTLSKADYEKHK |
| | | VYACEVTHQGLSSPVTKSFNRGEC |
| | | (SEQ ID NO: 19) |
| Humanized | EVQLVESGGGLVQPGGSLRLSC | EIVLTQSPATLSLSLGERATISCRAS |
| BC8(23) | AASGFDFSRYWMSWVRQAPG | KSVSTSGYSYLHWYQQKPGQAPK |
| anti-CD45 | KGLEWIGEINPTSSTINFADSVK | LLIYLASNRATGVPARFSGSGPGT |
| antibody | GRFTISRDNAKNSLYLQMNSV | DFTLTISSLEPEDFATYYCQHSREL |
| | RDEDTAVYYCARGNYYRYGD | PFTFGQGTKLEIK |
| | AMDYWGQGTLVTVSS | (SEQ ID NO: 22) |
| | (SEQ ID NO: 21) | |
| BC8 anti- | QVQLVESGGGLVQPGGSLKLS | DIVLTQSPASLAVSLGQRATISCRA |
| CD45 | CAASGFDFSRYWMSWVRQA | SKSVSTSGYSYLHWYQQKPGQPP |
| antibody | PGKGLEWIGEINPTSSTINFTPSL | KLLIYLASNLESGVPARFSGSGSGT |
| | KDKVFISRDNAKNTLYLQMSK | DFTLNIHPVEEEDAATYYCQHSRE |
| | VRSEDTALYYCARGNYYRYGD | LPFTFGSGTKLEIK |
| | AMDYWGQGTSVTVSS | (SEQ ID NO: 24) |
| | (SEQ ID NO: 23) | |
| 9.4 anti- | QVQLQQLGAELARPGASVKMS | DIVMTQAAPSVPVTPGESLSISCRS |
| CD45 | CKASGYTFTSYSIQWVKQRPG | SKSLLHSSGITYLYWFLQRPGQSPQ |
| antibody | QGLEWIGYINPSSGYIKYNQHF | LLIYRMSNLASGVPDRFSGSGSGT |
| | RDRATLTADRSSSTAYMQLSSL | AFTLRISR VEAEDVGVYYCMQHLE |
| | TSEDSAVYYCARGNSGSFDYW | YPFTFGGGTKLEIK |
| | GQGTTLTVSS | (SEQ ID NO: 26) |
| | (SEQ ID NO: 25) | |
| 4B2 anti- | QVQLQQSGAELARPGASVKM | DIVITQDELSNPVTSGESVSISCRSS |
| CD45 | SCKASGYTFTSYTMQWVKQR | KSLLYKDGKTYLNWFLQRPGQSP |
| antibody | PGQGLEWIGYINPSSGYIKYNQ | QLLIYLMSTRASGVSDRFSGSGSG |
| | KFKDKVTLTADKSSTTAYMQL | TDFTLEISRVKAEDVGVYYCQQLV |
| | SRLTSEDSAVYYCARRGSYFFD | EYPFTFGGGTKLEVK |
| | FWGQGTSVTVSS | (SEQ ID NO: 28) |
| | (SEQ ID NO: 27) | |

In some embodiments, the binding domain is an anti-CD45 scFv domain. In some embodiments, the anti-CD45 scFv domain is selected from a sequence listed in Table 5. In some embodiments, the anti-CD45 scFv domain comprises a polypeptide comprising SEQ ID NO: 29. In some embodiments, the anti-CD45 scFv domain comprises a polypeptide comprising SEQ ID NO: 30. In some embodiments, the anti-CD45 scFv domain is encoded by a polynucleotide sequence comprising SEQ ID NO: 31. In some embodiments, the anti-CD45 scFv domain comprises a polypeptide comprising SEQ ID NO: 32.

TABLE 5
anti-CD45 ScFv Domains for anti0CD45 CAR Constructs

| Anti-CD45 | | |
|---|---|---|
| CAR | Protein Sequence | DNA Sequence |
| Anti-CD45 scFv | QVQLVESGGGLVQPGGS | |
| | LKLSCAASGFDFSRYW | |
| | MSWVRQAPGKGLEWIG | |
| | EINPTSSTINFTPSLKDKV | |
| | FISRDNAKNTLYLQMSK | |
| | VRSEDTALYYCARGNY | |
| | YRYGDAMDYWGQGTS | |
| | VTVSKISGGGGSGGGGS | |
| | GGGGSGGGGSGGGGSS | |
| | DIVLTQSPASLAVSLGQR | |
| | ATISCRASKSVSTSGYSY | |
| | LHWYQQKPGQPPKLLIY | |
| | LASNLESGVPARFSGSGS | |
| | GTDFTLNIHPVEEEDAA | |
| | TYYCQHSRELPFTFGSG | |
| | TKLEIK | |
| | (SEQ ID NO: 29) | |
| BC8-CD45-(vL- | DIVLTQSPASLAVSLGQR | GACATCGTCCTGACCCAGTCCCCAG |
| vH) | ATISCRASKSVSTSGYSY | CCAGCCTGGCGGTGTCCCTGGGTCA |
| | LHWYQQKPGQPPKLLIY | ACGAGCTACCATTTCCTGCAGAGCC |
| | LASNLESGVPARFSGSGS | TCTAAGTCCGTATCCACTTCCGGCTA |
| | GTDFTLNIHPVEEEDAA | TAGTTACCTGCATTGGTACCAACAG |
| | TYYCQHSRELPFTFGSG | AAACCAGGGCAGCCACCCAAGCTGC |
| | TKLEIKGGGGSGGGGSG | TCATCTATCTGGCCTCAAACCTGGA |
| | GGGSQVQLVESGGGLV | GTCCGGCGTGCCCGCCAGATTCTCC |
| | QPGGSLKLSCAASGFDF | GGAAGTGGGAGCGGCACCGACTTTA |
| | SRYWMSWVRQAPGKGL | CATTGAACATCCACCCAGTCGAAGA |
| | EWIGEINPTSSTINFTPSL | GGAAGATGCTGCCACTTATTACTGT |
| | KDKVFISRDNAKNTLYL | CAGCACTCCAGGGAGCTGCCATTTA |
| | QMSKVRSEDTALYYCA | CATTTGGCTCTGGCACAAAACTGGA |
| | RGNYYRYGDAMDYWG | GATCAAAGGCGGAGGCGGAAGTGG |
| | QGTSVTVS | AGGCGGAGGATCTGGCGGCGGAGG |
| | (SEQ ID NO: 30) | CTCTCAGGTTCAGCTGGTCGAGTCC |
| | | GGGGGAGGTCTGGTACAGCCGGGTG |
| | | GCAGCCTCAAGCTGTCATGTGCCGC |
| | | ATCTGGTTTTGACTTTAGCAGGTATT |
| | | GGATGAGCTGGGTCCGCCAGGCCCC |
| | | GGGCAAGGGACTTGAGTGGATTGGA |
| | | GAAATAAACCCTACTTCCAGCACAA |
| | | TCAACTTCACACCTTCTTTGAAAGAT |
| | | AAGGTTTTTATCTCTCGGGATAACG |
| | | CTAAAAACACACTGTACTTGCAGAT |
| | | GTCTAAGGTGCGAAGCGAAGACACC |
| | | GCCCTCTATTATTGCGCCCGGGGTA |
| | | ATTACTACCGCTATGGGGATGCGAT |
| | | GGACTATTGGGGGCAAGGTACCTCC |
| | | GTCACGGTCTCA |
| | | (SEQ ID NO: 31) |
| humanized9.4C | EVQLVQSGAEVKKPGAS | |
| D45antibody; | VKVSCKASGYTFTSYSI | |
| heavy-chain | QWVRQAPGQRLEWIGYI | |
| and light-chain | NPSSGYIKYNQHFRGRA | |
| variable domains | TLTADRSASTAYMELSS | |
| are genetically | LRSEDTAVYYCARGNSG | |
| fused using a | SFDYWGQGTLVTVSSG | |
| flexible linker | GGGSGGGGSGGGGSGG | |
| | GGSDIVMTQSPLSLPVTP | |
| | GEPASISCRSSQSLLHSS | |
| | GITYLYWFLQKPGQSPQ | |
| | LLIYRMSNLASGVPDRF | |
| | SGSGSGTDFTLKISR VEA | |
| | EDVGVYYCMQHLEYPF | |
| | TFGQGTKLEIK | |
| | (SEQ ID NO: 32) | |

In some embodiments, the anti-CD45 scFv domain comprises a sequence of any one of SEQ ID NOS: 29, 30, 31, or 32, or a sequence having at least 90%, at least 95%, at least 97%, at least 99%, or 100% identical thereto.

In some embodiments, the extracellular ligand binding domain comprises a binding agent for CD11a, such as an antibody or antigen-binding fragment thereof. In some embodiments, the anti-CD11a antibody is a human anti-CD11a antibody, a humanized anti-CD11a antibody, or a chimeric anti-CD11a antibody. In some embodiments, the anti-CD11a antibody is an anti-CD11a monoclonal antibody. Illustrative anti-CD11a antibodies include efalizumab. In some embodiments, the antibody molecule or antigen-binding fragment that binds to CD11a is specific to one CD11a isoform or binds to more than one CD11a isoform, e.g., is a pan-CD11a antibody or antigen-binding fragment.

In some embodiments, the full length VH and VL regions comprise SEQ ID NO: 179 and SEQ ID NO: 180, respectively. In some embodiments, the binding domain comprises the full length VH region and VL regions on a single polypeptide. In some embodiments, the polypeptide comprises from N-terminal to C-terminal the full length VH regions and the full length VL region. In some embodiments, the polypeptide comprises from N-terminal to C-terminal the full length VL region and the full length VH region. In some embodiments, the binding domain comprises SEQ ID NO: 179 and SEQ ID NO: 180. In some embodiments, the anti-CD11a VH domain comprises a sequence of SEQ ID NO: 179 or a sequence having at least 90%, at least 95%, at least 97%, at least 99%, or 100% identical thereto. In some embodiments, the anti-CD11a VL domain comprises a sequence of SEQ ID NO: 180 or a sequence having at least 90%, at least 95%, at least 97%, at least 99%, or 100% identical thereto.

In some embodiments, the anti-CD11a full length VH and VL regions comprise SEQ ID NO: 247 and SEQ ID NO: 248, respectively. In some embodiments, the binding domain comprises the full length VH region and VL regions on a single polypeptide. In some embodiments, the polypeptide comprises from N-terminal to C-terminal the full length VH regions and the full length VL region. In some embodiments, the polypeptide comprises from N-terminal to C-terminal the full length VL region and the full length VH region. In some embodiments, the binding domain comprises SEQ ID NO: 247 and SEQ ID NO: 248. In some embodiments, the anti-CD11a VH domain comprises a sequence of SEQ ID NO: 247 or a sequence having at least 90%, at least 95%, at least 97%, at least 99% identity, or 100% identical thereto. In some embodiments, the anti-CD11a VL domain comprises a sequence of SEQ ID NO: 248 or a sequence having at least 90%, at least 95%, at least 97%, at least 99% identity, or 100% identical thereto.

In some embodiments, the extracellular ligand binding domain comprises a binding agent for SPN, such as an antibody or antigen-binding fragment thereof. In some embodiments, the anti-SPN antibody is a human anti-SPN antibody, a humanized anti-SPN antibody, or a chimeric anti-SPN antibody. In some embodiments, the anti-SPN antibody is an anti-SPN monoclonal antibody. Illustrative anti-SPN antibodies include MAB2038 and OX-58. In some embodiments, the antibody molecule or antigen-binding fragment that binds to SPN is specific to one SPN isoform or binds to more than one SPN isoform, e.g., is a pan-SPN antibody or antigen-binding fragment.

In some embodiments, the anti-SPN VH domain comprises any one of SEQ ID NOs: 249-252, 257, 261-280 or a sequence having at least 90%, at least 95%, at least 97%, at least 99% identity, or 100% identical thereto. In some embodiments, the anti-SPN VL domain comprises any one of SEQ ID NOs: 253-256, 258, 281-303 or a sequence having at least 90%, at least 95%, at least 97%, at least 99% identity, or 100% identical thereto. In some embodiments, the anti-SPN HC domain comprises a sequence of SEQ ID NO: 259 or a sequence having at least 90%, at least 95%, at least 97%, at least 99% identity, or 100% identical thereto. In some embodiments, the anti-SPN LC domain comprises a sequence of SEQ ID NO: 260 or a sequence having at least 90%, at least 95%, at least 97%, at least 99% identity, or 100% identical thereto.

In some embodiments, the extracellular ligand binding domain comprises a binding agent for CD33, such as an antibody or antigen-binding fragment thereof. In some embodiments, the anti-CD33 antibody is a human anti-CD33 antibody, a humanized anti-CD33 antibody, or a chimeric anti-CD33 antibody. In some embodiments, the anti-CD33 antibody is an anti-CD33 monoclonal antibody. Illustrative anti-CD33 antibodies include Gemtuzumab ozogamicin and immunotoxin vadastuximab talirine. In some embodiments, the antibody molecule or antigen-binding fragment that binds to CD33 is specific to one CD33 isoform or binds to more than one CD33 isoform, e.g., is a pan-CD33 antibody or antigen-binding fragment.

In some embodiments, the anti-CD33 full length VH and VL regions comprise SEQ ID NO: 181 and SEQ ID NO: 182, respectively. In some embodiments, the binding domain comprises the full length VH region and VL regions on a single polypeptide. In some embodiments, the polypeptide comprises from N-terminal to C-terminal the full length VH regions and the full length VL region. In some embodiments, the polypeptide comprises from N-terminal to C-terminal the full length VL region and the full length VH region. In some embodiments, the binding domain comprises SEQ ID NO: 181 and SEQ ID NO: 182. In some embodiments, the anti-CD33 VH domain comprises a sequence of SEQ ID NO: 181 or a sequence having at least 90%, at least 95%, at least 97%, at least 99% identity, or 100% identical thereto. In some embodiments, the anti-CD33 VL domain comprises a sequence of SEQ ID NO: 182 or a sequence having at least 90%, at least 95%, at least 97%, at least 99% identity, or 100% identical thereto.

In some embodiments, the anti-CD33 full length VH and VL regions comprise SEQ ID NO: 336 and SEQ ID NO: 335, respectively. In some embodiments, the binding domain comprises SEQ ID NO: 336 and SEQ ID NO: 335. In some embodiments, the anti-CD33 VH domain comprises a sequence of SEQ ID NO: 336 or a sequence having at least 90%, at least 95%, at least 97%, at least 99% identity, or 100% identical thereto. In some embodiments, the anti-CD33 VL domain comprises a sequence of SEQ ID NO: 335 or a sequence having at least 90%, at least 95%, at least 97%, at least 99% identity, or 100% identical thereto.

In some embodiments, the anti-CD33 full length VH and VL regions comprise SEQ ID NO: 337 and SEQ ID NO: 338, respectively. In some embodiments, the binding domain comprises SEQ ID NO: 337 and SEQ ID NO: 338. In some embodiments, the anti-CD33 VH domain comprises a sequence of SEQ ID NO: 337 or a sequence having at least 90%, at least 95%, at least 97%, at least 99% identity, or 100% identical thereto. In some embodiments, the anti-CD33 VL domain comprises a sequence of SEQ ID NO: 338 or a sequence having at least 90%, at least 95%, at least 97%, at least 99% identity, or 100% identical thereto.

In some embodiments, the anti-CD33 full length VH and VL regions comprise SEQ ID NO: 339 and SEQ ID NO: 340, respectively. In some embodiments, the binding domain comprises SEQ ID NO: 339 and SEQ ID NO: 340. In some embodiments, the anti-CD33 VH domain comprises a sequence of SEQ ID NO: 339 or a sequence having at least 90%, at least 95%, at least 97%, at least 99% identity, or 100% identical thereto. In some embodiments, the anti-CD33 VL domain comprises a sequence of SEQ ID NO: 340 or a sequence having at least 90%, at least 95%, at least 97%, at least 99% identity, or 100% identical thereto.

In some embodiments, the anti-CD33 full length VH and VL regions comprise SEQ ID NO: 341 and SEQ ID NO: 342, respectively. In some embodiments, the binding domain comprises SEQ ID NO: 341 and SEQ ID NO: 342. In some embodiments, the anti-CD33 VH domain comprises a sequence of SEQ ID NO: 341 or a sequence having at least 90%, at least 95%, at least 97%, at least 99% identity, or 100% identical thereto. In some embodiments, the anti-CD33 VL domain comprises a sequence of SEQ ID NO: 342 or a sequence having at least 90%, at least 95%, at least 97%, at least 99% identity, or 100% identical thereto.

In some embodiments, the anti-CD33 full length HC and LC regions comprise SEQ ID NO: 331 and SEQ ID NO: 332, respectively. In some embodiments, the anti-CD33 HC domain comprises a sequence of SEQ ID NO: 331 or a sequence having at least 90%, at least 95%, at least 97%, at least 99% identity, or 100% identical thereto. In some embodiments, the anti-CD33 LC domain comprises a sequence of SEQ ID NO: 332 or a sequence having at least 90%, at least 95%, at least 97%, at least 99% identity, or 100% identical thereto.

In some embodiments, the anti-CD33 full length HC and LC regions comprise SEQ ID NO: 333 and SEQ ID NO: 334, respectively. In some embodiments, the anti-CD33 HC domain comprises a sequence of SEQ ID NO: 333 or a sequence having at least 90%, at least 95%, at least 97%, at least 99% identity, or 100% identical thereto. In some embodiments, the anti-CD33 LC domain comprises a sequence of SEQ ID NO: 334 or a sequence having at least 90%, at least 95%, at least 97%, at least 99% identity, or 100% identical thereto.

In some embodiments, the binding domain is an anti-CD33 scFv domain. In some embodiments, the anti-CD33 scFv domain comprises a polypeptide comprising any one of SEQ ID NOs: 304-312. In some embodiments, the anti-CD33 scFv domain comprises a sequence of any one of SEQ ID NOS: 304-312, or a sequence having at least 90%, at least 95%, at least 97%, at least 99% identity, or 100% identical thereto.

In some embodiments, the extracellular ligand binding domain comprises a binding agent for FLT3, such as an antibody or antigen-binding fragment thereof. In some embodiments, the anti-FLT3 antibody is a human anti-FLT3 antibody, a humanized anti-FLT3 antibody, or a chimeric anti-FLT3 antibody. In some embodiments, the anti-FLT3 antibody is an anti-FLT3 monoclonal antibody. Illustrative anti-FLT3 antibodies include LY3012218 (IMC-EB10) and 4G8SDIEM. In some embodiments, the antibody molecule or antigen-binding fragment that binds to FLT3 is specific to one FLT3 isoform or binds to more than one FLT3 isoform, e.g., is a pan-FLT3 antibody or antigen-binding fragment.

In some embodiments, the anti-FLT3 VH domain comprises any one of SEQ ID NOs: 319, 321, or a sequence having at least 90%, at least 95%, at least 97%, at least 99% identity, or 100% identical thereto. In some embodiments, the anti-FLT3 VL domain comprises any one of SEQ ID NOs: 320, 322, or a sequence having at least 90%, at least 95%, at least 97%, at least 99% identity, or 100% identical thereto.

In some embodiments, the anti-FLT3 HC domain comprises any one of SEQ ID NOs: 344, 346, 348, 350, 352, 353, 355, 357, 359, 361, 363, 365, 367, 369, 371, 373, 375, 377, or a sequence having at least 90%, at least 95%, at least 97%, at least 99% identity, or 100% identical thereto. In some embodiments, the anti-FLT3 LC domain comprises a sequence of SEQ ID NOs: 343, 345, 347, 349, 351, 354, 356, 358, 360, 362, 364, 366, 368, 370, 372, 374, 376, 378, or a sequence having at least 90%, at least 95%, at least 97%, at least 99% identity, or 100% identical thereto.

### Cytoplasmic Activation Domain

The cytoplasmic domain of CARs of the instant disclosure comprises at least one cytoplasmic activation domain. In some embodiments, the cytoplasmic activation domain ensures that there is T-cell receptor (TCR) signaling necessary to activate the effector functions of the CAR T-cell. In some embodiments, the at least one cytoplasmic activation is a CD247 molecule (CD3ζ) activation domain, a stimulatory killer immunoglobulin-like receptor (KIR) KIR2DS2 activation domain, or a DNAX-activating protein of 12 kDa (DAP12) activation domain. In some embodiments, the CD3ζ activation domain comprises an amino acid sequence having at least 80% identity, at least 90% identity, at least 95% identity, at least 99% identity, or 100% identical to a sequence of

(SEQ ID NO: 33)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CD3ζ activation domain comprises or consists essentially of SEQ ID NO: 33. In some embodiments, the CD3ζ activation domain is encoded by a nucleotide sequence having at least 80% identity, at least 90% identity, at least 95% identity, at least 99% identity, or 100% identical to a sequence of

(SEQ ID NO: 34)
AGAGTGAAGTTCAGCAGGAGCGCAGACGCCCCCGCGTACAAGCAGGGCCAGAA
CCAGCTCTATAACGAGCTCAATCTAGGACGAAGAGAGGAGTACGATGTTTTGGA
CAAGCGTAGAGGCCGGGACCCTGAGATGGGGGGAAAGCCGAGAAGGAAGAACC
CTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTT
TACCAGGGACTCAGTACAGCCACCAAGGACACCTACGACGCCCTTCACATGCAG
GCCCTGCCCCCTCGC. In some embodiments, the CD3ζ activation
domain is encoded by SEQ ID NO: 34.

### Chimeric Antigen Receptors (CARs)

In some embodiments, the either the first or the second engineered receptor is a chimeric antigen receptor (CAR). In some embodiments, the first and second engineered receptors are chimeric antigen receptors. All CAR architectures are envisaged as within the scope of the instant disclosure.

The term “chimeric antigen receptors” or “CARs” as used herein, may refer to artificial T-cell receptors, chimeric T-cell receptors, or chimeric immunoreceptors, for example, and encompass engineered receptors that graft an artificial specificity onto a particular immune effector cell, such as a helper T cell (CD4+), cytotoxic T cell (CD8+) or NK cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen such as a CD45 antigen. In some embodiments, CARs comprise an extracellular domain comprising an antigen-binding region, a transmembrane domain, and an intracellular signaling domain. In some embodiments, CARs comprise fusions of single-chain variable fragments (scFvs) or scFabs derived from monoclonal antibodies, fused to a transmembrane domain and intracellular signaling domain(s). The fusion may also comprise a hinge. Either heavy-light (H-L) and light-heavy (L-H) scFvs may be used. The specificity of CAR designs may be derived from ligands of receptors (e.g., peptides). Depending on the type of intracellular domain, a CAR can be an activator receptor or an inhibitor receptor. In some embodiments, for example, when the CAR is an activator receptor, the CAR comprises domains for additional co-stimulatory signaling, such as CD3, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, cytokines, and cytokine receptors. As used herein, characteristics attributed to a chimeric antigen receptor may be understood to refer to the receptor itself or to a host cell comprising the receptor.

### Extracellular Domains

In some embodiments, the extracellular domain of the CAR comprises the antigen binding domains described supra.

### Hinge Region

In some embodiments, the CARs of the present disclosure comprise an extracellular hinge region. Incorporation of a hinge region can affect cytokine production from CAR-T cells and improve expansion of CAR-T cells in vivo. Illustrative hinges can be isolated or derived from IgG, CD28 or CD8, among others, for example IgG1. Illustrative hinge domains are provided in Table 6A.

In some embodiments, the hinge is isolated or derived from CD8a or CD28. In some embodiments, the CD8a hinge comprises an amino acid sequence having at least 80% identity, at least 90% identity, at least 95% identity, at least 99% identity, or 100% identical to a sequence of TTTPAPRPPTPAPTIASQPLSLRPEACRPAAGGAVHTRGLDFACD (SEQ ID NO: 35). In some embodiments, the CD8a hinge comprises SEQ ID NO: 35. In some embodiments, the CD8a hinge consists essentially of SEQ ID NO: 35. In some embodiments, the CD8a hinge is encoded by a nucleotide sequence having at least 80% identity, at least 90% identity, at least 95% identity, at least 99% identity, or 100% identical to a sequence of

(SEQ ID NO: 36)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT.

In some embodiments, the CD8a hinge is encoded by SEQ ID NO: 36.

In some embodiments, the CD28 hinge comprises an amino acid sequence having at least 80% identity, at least 90% identity, at least 95% identity, at least 99% identity, or 100% identical to a sequence of CTIEVMYPPPYLDNEKSNGTIIHVKGKHLCPSPLFPGPSKP (SEQ ID NO: 37). In some embodiments, the CD28 hinge comprises or consists essentially of SEQ ID NO: 37. In some embodiments, the CD28 hinge is encoded by a nucleotide sequence having at least 80% identity, at least 90% identity, at least 95% identity, at least 99% identity, or 100% identical to a sequence of:

(SEQ ID NO: 38)
TGTACCATTGAAGTTATGTATCCTCCTCCTTACCTAGACAATGAGAAGA
GCAATGGAACCATTATCCATGTGAAAGGGAAACACCTTTGTCCAAGTCC
CCTATTTCCCGGACCTTCTAAGCCC.

In some embodiments, the CD28 hinge is encoded by SEQ ID NO: 38.

In some embodiments, the activator receptor comprises a hinge sequence isolated or derived from CD8, CD28, IgG1, or IgG4, or a synthetic hinge.

TABLE 6A
Hinge Domain Sequences and their use in CAR Constructs

| Hinge | Protein Sequence | DNA Sequence |
|---|---|---|
| CD8 | FVPVFLPAKPTT | TTCGTGCCGGTCTTCCTGCCAGCGAAGCCCA |
| | TPAPRPPTPAPTI | CCACGACGCCAGCGCCGCGACCACCAACAC |
| | ASQPLSLRPEAC | CGGCGCCCACCATCGCGTCGCAGCCCCTGTC |
| | RPAAGGAVHTR | CCTGCGCCCAGAGGCGTGCCGGCCAGCGGC |
| | GLDFACD | GGGGGGCGCAGTGCACACGAGGGGGCTGGA |
| | (SEQ ID NO: 39) | CTTCGCCTGTGAT |
| | | (SEQ ID NO: 40) |
| CD28 | IEVMYPPPYLDN | ATTGAAGTTATGTATCCTCCTCCTTACCTAG |
| | EKSNGTIIHVKG | ACAATGAGAAGAGCAATGGAACCATTATCC |
| | KHLCPSPLFPGP | ATGTGAAAGGGAAACACCTTTGTCCAAGTCC |
| | SKP | CCTATTTCCCGGACCTTCTAAGCCC |
| | (SEQ ID NO: 41) | (SEQ ID NO: 42) |
| Short | GGGSSGGGSG | GGTGGCGGTTCTTCCGGCGGTGGCTCTGGT |
| linker | (SEQ ID NO: 43) | (SEQ ID NO: 44) |
| IgG4 | ESKYGPPCPSCP | GAGTCCAAATATGGTCCCCCATGCCCATCAT |
| (EQ) | APEFEGGPSVFL | GCCCAGCACCTGAGTTCGAGGGGGGACCAT |
| | FPPKPKDTLMIS | CAGTCTTCCTGTTCCCCCCAAAACCCAAGGA |
| | RTPEVTCVVVD | CACTCTCATGATCTCCCGGACCCCTGAGGTC |
| | VSQEDPEVQFN | ACGTGCGTGGTGGTGGACGTGAGCCAGGAA |
| | WYVDGVEVHN | GACCCCGAGGTCCAGTTCAACTGGTACGTGG |
| | AKTKPREEQFQ | ATGGCGTGGAGGTGCATAATGCCAAGACAA |
| | STYRVVSVLTV | AGCCGCGGGAGGAGCAGTTCCAGAGCACGT |
| | LHQDWLNGKE | ACCGTGTGGTCAGCGTCCTCACCGTCCTGCA |
| | YKCKVSNKGLP | CCAGGACTGGCTGAACGGCAAGGAGTACAA |
| | SSIEKTISKAKG | GTGCAAGGTCTCCAACAAAGGCCTCCCGTCC |
| | QPREPQVYTLPP | TCCATCGAGAAAACCATCTCCAAAGCCAAA |
| | SQEEMTKNQVS | GGGCAGCCCCGAGAGCCACAGGTGTACACC |
| | LTCLVKGFYPS | CTGCCCCCATCCCAGGAGGAGATGACCAAG |
| | DIAVEWESNGQ | AACCAGGTCAGCCTGACCTGCCTGGTCAAA |
| | PENNYKTTPPVL | GGCTTCTACCCCAGCGACATCGCCGTGGAGT |
| | DSDGSFFLYSRL | GGGAGAGCAATGGGCAGCCGGAGAACAACT |
| | TVDKSRWQEGN | ACAAGACCACGCCTCCCGTGCTGGACTCCGA |
| | VFSCSVMHEAL | CGGCTCCTTCTTCCTCTACAGCAGGCTCACC |
| | HNHYTQKSLSL | GTGGACAAGAGCAGGTGGCAGGAGGGGAAT |
| | SLGK | GTCTTCTCATGCTCCGTGATGCATGAGGCTC |
| | (SEQ ID NO: 45) | TGCACAACCACTACACACAGAAGAGCCTCT |
| | | CCCTGTCTCTGGGTAAA |
| | | (SEQ ID NO: 46) |
| IgG1 | AEPKSPDKTHT | GCCGAGCCCAAGTCCCCTGATAAAACTCAC |
| | CPPCPKDPK | ACCTGCCCACCCTGTCCTAAGGACCCGAAG |
| | (SEQ ID NO: 47) | (SEQ ID NO: 48) |

In some embodiments, for example those embodiments where the CAR is an activator receptor, the hinge comprises a sequence of SEQ ID NO: 35, 36, 37, 38, 39, 40, 41, 42, 43, 44, 45, 46, 47, or 48, or a sequence having at least 80%, at least 85%, at least 90%, at least 95%, at least 97%, at least 99%, or that is identical thereto. In some embodiments, for example those embodiments where the CAR is an activator receptor, the hinge comprises a sequence of SEQ ID NO: 35, 36, 37, 38, 39, 40, 41, 42, 43, 44, 45, 46, 47, or 48.

### Transmembrane Domain

The CARs of the present disclosure can be designed to comprise a transmembrane domain that is fused to the extracellular domain of the CAR. In some embodiments, the transmembrane domain that naturally is associated with one of the domains in the CAR is used. For example, a CAR comprising a CD28 co-stimulatory domain might also use a CD28 transmembrane domain. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments of the CARs of the disclosure, the CARs comprise a CD28 transmembrane domain. In some embodiments, the CD28 transmembrane domain comprises an amino acid sequence having at least 80% identity, at least 90% identity, at least 95% identity, at least 99% identity, or 100% identical to a sequence of FWVLVVVGGVLACYSLLVTVAFIIFWV (SEQ ID NO: 49). In some embodiments, the CD28 transmembrane domain comprises or consists essentially of SEQ ID NO: 49. In some embodiments, the CD28 transmembrane domain is encoded by a nucleotide sequence having at least 80% identity, at least 90% identity, at least 95% identity, at least 99% identity, or 100% identical to a sequence of

(SEQ ID NO: 50)
TTCTGGGTGCTGGTCGTTGTGGGCGGCGTGCTGGCCTGCTACAGCCTGC
TGGTGACAGTGGCCTTCATCATCTTTTGGGTG.

In some embodiments, the CD28 transmembrane domain is encoded by SEQ ID NO: 50.

In some embodiments of the CARs of the disclosure, the CARs comprise an IL-2Rbeta transmembrane domain. In some embodiments, the IL-2Rbeta transmembrane domain comprises an amino acid sequence having at least 80% identity, at least 90% identity, at least 95% identity, at least 99% identity, or 100% identical to a sequence of IPWLGHLLVGLSGAFGFIILVYLLI (SEQ ID NO: 51). In some embodiments, the IL-2Rbeta transmembrane domain comprises or consists essentially of SEQ ID NO: 51. In some embodiments, the IL-2Rbeta transmembrane domain is encoded by a nucleotide sequence having at least 80% identity, at least 90% identity, at least 95% identity, at least 99% identity, or 100% identical to a sequence of

(SEQ ID NO: 52)
ATTCCGTGGCTCGGCCACCTCCTCGTGGGCCTCAGCGGGGCTTTTGGCT
TCATCATCTTAGTGTACTTGCTGATC.

In some embodiments, the IL-2Rbeta transmembrane domain is encoded by SEQ ID NO: 52.

In some embodiments, the CAR comprises a transmembrane domain isolated or derived from CD8 or CD28. In some embodiments, the CAR comprises a transmembrane domain sequence in Table 6B. In some embodiments, the CAR comprises a transmembrane domain sequence of SEQ ID NO: 49, 51, 53, or 55, or a sequence having at least 80% identity, at least 90% identity, at least 95% identity, at least 99% identity, or 100% identical thereto. In some embodiments, the CAR comprises a transmembrane domain sequence of SEQ ID NO: 49, 51, 53, or 55.

TABLE 6B
Transmembrane Domain Sequences and their use in CAR Constructs

| TM | | |
|---|---|---|
| Domain | Protein Sequence | DNA Sequence |
| CD8 | IYIWAPLAGTCGVL | ATCTACATCTGGGCGCCCTTGGCCG |
| | LLSLVIT | GGACTTGTGGGGTCCTTCTCCTGTC |
| | (SEQ ID NO: 53) | ACTGGTTATCACC |
| | | (SEQ ID NO: 54) |
| CD8 | IYIWAPLAGTCGVL | ATCTACATCTGGGCGCCCTTGGCCG |
| | LLSLVITLYCNHRN | GGACTTGTGGGGTCCTTCTCCTGTC |
| | (SEQ ID NO: 55) | ACTGGTTATCACCCTTTACTGCAAC |
| | | CACAGGAAC |
| | | (SEQ ID NO: 56) |
| CD28 | FWVLVVVGGVLAC | TTCTGGGTGCTGGTCGTTGTGGGCG |
| | YSLLVTVAFIIFWV | GCGTGCTGGCCTGCTACAGCCTGCT |
| | (SEQ ID NO: 49) | GGTGACAGTGGCCTTCATCATCTTT |
| | | TGGGTG |
| | | (SEQ ID NO: 50) |

### Cytoplasmic Domain

The disclosure provides an activator receptor comprising an extracellular ligand binding domain specific to an activator antigen. In some embodiments, the activator receptor is a T cell receptor (TCR) or a chimeric antigen receptor (CAR). In some embodiments, the CAR comprises an intracellular domain isolated or derived from CD28, 4-1BB or CD3z, or a combination thereof.

The cytoplasmic domain or otherwise the intracellular signaling domain of the CARs of the instant invention is responsible for activation of at least one of the normal effector functions of the immune cell in which the CAR has been placed. The term “effector function” refers to a specialized function of a cell. Effector functions of a regulatory T cell, for example, include the suppression or downregulation of induction or proliferation of effector T cells. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact chain as long as it transduces the effector function signal. In some cases, multiple intracellular domains can be combined to achieve the desired functions of the CAR-T cells of the instant disclosure. The term intracellular signaling domain is thus meant to include any truncated portion of one or more intracellular signaling domains sufficient to transduce the effector function signal.

Examples of intracellular signaling domains for use in the CARs of the instant disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are often insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. In some embodiments, the ITAM contains a tyrosine separated from a leucine or an isoleucine by any two other amino acids (YxxL) (SEQ ID NO: 57).

In some embodiments, the cytoplasmic domain contains 1, 2, or 3 ITAMs. In some embodiments, the cytoplasmic domain contains 1 ITAM. In some embodiments, the cytoplasmic domain contains 2 ITAMs. In some embodiments, the cytoplasmic domain contains 3 ITAMs. In some embodiments, the cytoplasmic domain contains 4 ITAMs. In some embodiments, the cytoplasmic domain contains 5 ITAMs.

In some embodiments, the cytoplasmic domain is a CD3ζ activation domain. In some embodiments, CD3ζ activation domain comprises a single ITAM. In some embodiments, CD3ζ activation domain comprises two ITAMs. In some embodiments, CD3ζ activation domain comprises three ITAMs.

In some embodiments, the CD3ζ activation domain comprising a single ITAM comprises an amino acid sequence having at least 80% identity, at least 90% identity, at least 95% identity, at least 99% identity, or 100% identical to a sequence of RVKFSRSADAPAYQQGQNQLYNELNLGRREEYDVLHMQALPPR (SEQ ID NO: 58). In some embodiments, the CD3ζ activation domain comprises SEQ ID NO: 58. In some embodiments, the CD3ζ activation domain comprising a single ITAM consists essentially of an amino acid sequence of SEQ ID NO: 58.

In some embodiments, the CD3ζ activation domain comprising a single ITAM is encoded by a nucleotide sequence having at least 80% identity, at least 90% identity, at least 95% identity, at least 99% identity, or 100% identical to a sequence of AGAGTGAAGTTCAGCAGGAGCGCAGACGCCCCCGCGTACCAGCAGGGCCAGAA CCAGCTCTATAACGAGCTCAATCTAGGACGAAGAGAGGAGTACGATG TTTTGCACATGCAGGCCCTGCCCCCTCGC (SEQ ID NO: 59). In some embodiments, the CD3ζ activation domain is encoded by SEQ ID NO: 59.

Further examples of ITAM containing primary cytoplasmic signaling sequences that can be used in the CARs of the instant disclosure include those derived from TCRζ, FcRγ, FcRβ, CD3γ, CD3δ, CD3ε, CD3ζ, CD5, CD22, CD79a, CD79b, and CD66d. It is particularly preferred that cytoplasmic signaling molecule in the CAR of the instant invention comprises a cytoplasmic signaling sequence derived from CD3ζ.

### CD45 Targeted CAR

The CD45 targeted CAR (also called “anti-CD45 CAR”) or CD45 targeted polypeptide described herein may include a CD45 targeting scFv.

In some embodiments, the first receptor is an anti-CD45 CAR comprising any one of SEQ ID NOS: 133-140, 142, 144, 146, 148, 151, 153, 155, 156-168, 328, 329, 330 or a sequence sharing at least 75%, at least 80%, at least 85%, at least 90%, at least 95%, or at least 98% identity thereto.

In some embodiments, nucleic acids (e.g., isolated nucleic acids) encoding one or more chimeric antigen receptors are provided. In some embodiments, the nucleic acid is an RNA construct, such as a messenger RNA (mRNA) transcript or a modified RNA. In some embodiments, the nucleic acid is a DNA construct.

In some embodiments, the first receptor is an anti-CD45 CAR encoded by a polynucleotide comprising SEQ ID NO: 141, 143 145, 147, 149, 150, 152, 154, or a sequence sharing at least 75%, at least 80%, at least 85%, at least 90%, at least 95%, or at least 98% identity thereto.

### CD33 Targeted CAR

The CD33 targeted CAR (also called “anti-CD33 CAR”) or CD33 targeted polypeptide described herein may include a CD33 targeting scFv.

In some embodiments, the first receptor is an anti-CD33 CAR comprising any one of SEQ ID NOS: 313-318 or a sequence sharing at least 75%, at least 80%, at least 85%, at least 90%, at least 95%, or at least 98% identity thereto.

### CD123 Targeted CAR

The CD123 targeted CAR (also called “anti-CD123 CAR”) or CD123 targeted polypeptide described herein may include a CD123 targeting scFv.

In some embodiments, the first receptor is an anti-CD123 CAR comprising any one of SEQ ID NOS: 420-423 or a sequence sharing at least 75%, at least 80%, at least 85%, at least 90%, at least 95%, or at least 98% identity thereto.

### Co-Stimulatory Domain

In some embodiments, the cytoplasmic domain of the CAR can be designed to comprise the CD3ζ signaling domain by itself or combined with any other desired cytoplasmic domain(s) useful in the context of the CAR of the instant disclosure. For example, the cytoplasmic domain of the CAR can comprise a CD3ζ chain portion and a co-stimulatory domain. The co-stimulatory domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include the co-stimulatory domain is selected from the group consisting of IL-2Rβ, Fc Receptor gamma (FcRγ), Fc Receptor beta (FcRβ), CD3g molecule gamma (CD3γ), CD3δ, CD3ε, CD5 molecule (CD5), CD22 molecule (CD22), CD79a molecule (CD79a), CD79b molecule (CD79b), carcinoembryonic antigen related cell adhesion molecule 3 (CD66d), CD27 molecule (CD27), CD28 molecule (CD28), TNF receptor superfamily member 9 (4-1BB), TNF receptor superfamily member 4 (OX40), TNF receptor superfamily member 8 (CD30), CD40 molecule (CD40), programmed cell death 1 (PD-1), inducible T cell costimulatory (ICOS), lymphocyte function-associated antigen-1 (LFA-1), CD2 molecule (CD2), CD7 molecule (CD7), TNF superfamily member 14 (LIGHT), killer cell lectin like receptor C2 (NKG2C) and CD276 molecule (B7-H3) c-stimulatory domains, or functional fragments thereof.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

In some embodiments, the intracellular domains of CARs of the instant disclosure comprise at least one co-stimulatory domain. In some embodiments, the co-stimulatory domain is isolated or derived from CD28. In some embodiments, the CD28 co-stimulatory domain comprises an amino acid sequence having at least 80% identity, at least 90% identity, at least 95% identity, at least 99% identity, or 100% identical to a sequence of

(SEQ ID NO: 60)
RSKRSRLLHSDYMNMTPRRPGPTRKHYQPYAPPRDFAAYRS.

In some embodiments, the CD28 co-stimulatory domain comprises or consists essentially of SEQ ID NO: 60. In some embodiments, the CD28 co-stimulatory domain is encoded by a nucleotide sequence having at least 80% identity, at least 90% identity, at least 95% identity, at least 99% identity, or 100% identical to a sequence of AGGAGCAAGCGGAGCAGACTGCTGCACAGCGACTACATGAACATGACCCCCCGG AGGCCTGGCCCCACCCGGAAGCACTACCAGCCCTACGCCCCTCCCAGGGATTTCG CCGCCTACCGGAGC (SEQ ID NO: 61). In some embodiments, the CD28 co-stimulatory domain is encoded by SEQ ID NO: 61.

In some embodiments, the intracellular domain of the CARs of the instant disclosure comprises an interleukin-2 receptor beta-chain (IL-2Rbeta or IL-2R-beta) cytoplasmic domain. In some embodiments, the IL-2Rbeta domain is truncated. In some embodiments, the IL-2Rbeta cytoplasmic domain comprises one or more STAT5-recruitment motifs. In some embodiments, the CAR comprises one or more STAT5-recruitment motifs outside the IL-2Rbeta cytoplasmic domain.

In some embodiments, the IL-2-Rbeta intracellular domain comprises an amino acid sequence having at least 80% identity, at least 90% identity, at least 95% identity, at least 99% identity, or 100% identical to a sequence of NCRNTGPWLKKVLKCNTPDPSKFFSQLSSEHGGDVQKWLSSPFPSSSFSPGGLAPEIS PLEVLERDKVTQLLPLNTDAYLSLQELQGQDPTHLV (SEQ ID NO: 62). In some embodiments, the IL2R-beta intracellular domain comprises or consists essentially of SEQ ID NO: 62. In some embodiments, the IL-2R-beta intracellular domain is encoded by a nucleotide sequence having at least 80% identity, at least 90% identity, at least 95% identity, at least 99% identity, or 100% identical to a sequence of AACTGCAGGAACACCGGGCCATGGCTGAAGAAGGTCCTGAAGTGTAACACCCCA GACCCCTCGAAGTTCTTTTCCCAGCTGAGCTCAGAGCATGGAGGCGACGTCCAGA AGTGGCTCTCTTCGCCCTTCCCCTCATCGTCCTTCAGCCCTGGCGGCCTGGCACCT GAGATCTCGCCACTAGAAGTGCTGGAGAGGGACAAGGTGACGCAGCTGCTCCCC CTGAACACTGATGCCTACTTGTCTCTCCAAGAACTCCAGGGTCAGGACCCAACTC ACTTGGTG (SEQ ID NO: 63). In some embodiments, the IL-2R-beta intracellular domain is encoded by SEQ ID NO: 63.

In some embodiments, the IL-2R-beta cytoplasmic domain comprises one or more STAT5-recruitment motifs. Illustrative STAT5-recruitment motifs are provided by Passerini et al. (International Immunology, Vol. 20, No. 3, pp. 421-431, 2008) and by Kagoya et al. (Nature Medicine doi:10.1038/nm.4478, 2018).

In some embodiments, the STAT5-recruitment motif(s) consists of the sequence Tyr-Leu-Ser-Leu (SEQ ID NO: 64).

In some embodiments, the CAR comprises an intracellular domain isolated or derived from CD28, 4-1BB and/or CD3ζ or a combination thereof. Illustrative domains derived from CD28, 4-1BB and/or CD3ζ are shown in Table 6C below. In some embodiments, the CAR comprises an intracellular domain comprising a sequence of SEQ ID NOS: 65, 67, or 69, or a sequence having at least 80%, at least 85%, at least 90%, at least 95%, at least 97%, at least 99%, or that is identical thereto. In some embodiments, the CAR comprises an intracellular domain comprising a sequence of SEQ ID NOS: 65, 67, or 69. In some embodiments, the CAR intracellular domain is encoded by the sequence set forth in any one of SEQ ID NOs: 66, 68, or 70.

TABLE 6C
Intracellular Domain Sequences and their use in CAR Constructs

| IC Domain | Protein Sequence | DNA Sequence |
|---|---|---|
| CD28-4- | RSKRSRLLHSD | AGGAGTAAGAGGAGCAGGCTCCTGC |
| 1BB-CD3z | YMNMTPRRPGP | ACAGTGACTACATGAACATGACTCCC |
| | TRKHYQPYAPP | CGCCGCCCCGGGCCCACCCGCAAGC |
| | RDFAAYRSRFS | ATTACCAGCCCTATGCCCCACCACGC |
| | VVKRGRKKLLY | GACTTCGCAGCCTATCGCTCCCGTTT |
| | IFKQPFMRPVQT | CTCTGTTGTTAAACGGGGCAGAAAGA |
| | TQEEDGCSCRFP | AGCTCCTGTATATATTCAAACAACCA |
| | EEEEGGCELRV | TTTATGAGACCAGTACAAACTACTCA |
| | KFSRSADAPAY | AGAGGAAGATGGCTGTAGCTGCCGA |
| | QQGQNQLYNEL | TTTCCAGAAGAAGAAGAAGGAGGAT |
| | NLGRREEYDVL | GTGAACTGAGAGTGAAGTTCAGCAG |
| | DKRRGRDPEMG | GAGCGCAGACGCCCCCGCGTACCAG |
| | GKPRRKNPQEG | CAGGGCCAGAACCAGCTCTATAACG |
| | LYNELQKDKM | AGCTCAATCTAGGACGAAGAGAGGA |
| | AEAYSEIGMKG | GTACGATGTTTTGGACAAGAGACGTG |
| | ERRRGKGHDGL | GCCGGGACCCTGAGATGGGGGGAAA |
| | YQGLSTATKDT | GCCGAGAAGGAAGAACCCTCAGGAA |
| | YDALHMQALPP | GGCCTGTACAATGAACTGCAGAAAG |
| | R | ATAAGATGGCGGAGGCCTACAGTGA |
| | (SEQ ID NO: 65) | GATTGGGATGAAAGGCGAGCGCCGG |
| | | AGGGGCAAGGGGCACGATGGCCTTT |
| | | ACCAGGGTCTCAGTACAGCCACCAA |
| | | GGACACCTACGACGCCCTTCACATGC |
| | | AGGCCCTGCCCCCTCGCTAG |
| | | (SEQ ID NO: 66) |
| CD28- | RSKRSRLLHSD | AGGAGCAAGCGGAGCAGACTGCTGC |
| CD3z | YMNMTPRRPGP | ACAGCGACTACATGAACATGACCCCC |
| | TRKHYQPYAPP | CGGAGGCCTGGCCCCACCCGGAAGC |
| | RDFAAYRSRVK | ACTACCAGCCCTACGCCCCTCCCAGG |
| | FSRSADAPAYK | GATTTCGCCGCCTACCGGAGCAGAGT |
| | QGQNQLYNELN | GAAGTTCAGCAGGAGCGCAGACGCC |
| | LGRREEYDVLD | CCCGCGTACAAGCAGGGCCAGAACC |
| | KRRGRDPEMGG | AGCTCTATAACGAGCTCAATCTAGGA |
| | KPRRKNPQEGL | CGAAGAGAGGAGTACGATGTTTTGG |
| | YNELQKDKMA | ACAAGCGTAGAGGCCGGGACCCTGA |
| | EAYSEIGMKGE | GATGGGGGGAAAGCCGAGAAGGAAG |
| | RRRGKGHDGLY | AACCCTCAGGAAGGCCTGTACAATG |
| | QGLSTATKDTY | AACTGCAGAAAGATAAGATGGCGGA |
| | DALHMQALPPR | GGCCTACAGTGAGATTGGGATGAAA |
| | (SEQ ID NO: 67) | GGCGAGCGCCGGAGGGGCAAGGGGC |
| | | ACGATGGCCTTTACCAGGGACTCAGT |
| | | ACAGCCACCAAGGACACCTACGACG |
| | | CCCTTCACATGCAGGCCCTGCCCCCT |
| | | CGCTAG (SEQ ID NO: 68) |
| 4-1BB- | KRGRKKLLYIF | AAACGGGGCAGAAAGAAGCTCCTGT |
| CD3z | KQPFMRPVQTT | ATATATTCAAACAACCATTTATGAGA |
| | QEEDGCSCRFPE | CCAGTACAAACTACTCAAGAGGAAG |
| | EEEGGCELGGG | ATGGCTGTAGCTGCCGATTTCCAGAA |
| | RVKFSRSADAP | GAAGAAGAAGGAGGATGTGAACTGG |
| | AYQQGQNQLY | GCGGTGGCAGAGTGAAGTTCAGCAG |
| | NELNLGRREEY | GAGCGCAGACGCCCCCGCGTACCAG |
| | DVLDKRRGRDP | CAGGGCCAGAACCAGCTCTATAACG |
| | EMGGKPRRKNP | AGCTCAATCTAGGACGAAGAGAGGA |
| | QEGLYNELQKD | GTACGATGTTTTGGACAAGAGACGTG |
| | KMAEAYSEIGM | GCCGGGACCCTGAGATGGGGGGAAA |
| | KGERRRGKGHD | GCCGAGAAGGAAGAACCCTCAGGAA |
| | GLYQGLSTATK | GGCCTGTACAATGAACTGCAGAAAG |
| | DTYDALHMQA | ATAAGATGGCGGAGGCCTACAGTGA |
| | LPPR | GATTGGGATGAAAGGCGAGCGCCGG |
| | (SEQ ID NO: 69) | AGGGGCAAGGGGCACGATGGCCTTT |
| | | ACCAGGGTCTCAGTACAGCCACCAA |
| | | GGACACCTACGACGCCCTTCACATGC |
| | | AGGCCCTGCCCCCTCGCTAG |
| | | (SEQ ID NO: 70) |

### T Cell Receptors (TCRs)

The disclosure provides a first activator receptor and immune cells comprising same. In some embodiments, the first receptor is a T cell receptor (TCR). Illustrative TCRs comprising cytoplasmic domains for use in the instant disclosure are described in PCT/US2020/045250, the contents of which are incorporated herein by reference.

As used herein, a “TCR,” sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an illustrative TCR complex, the TCR alpha and TCR beta polypeptides form a heterodimer, CD3 epsilon and CD3 delta form a heterodimer, CD3 epsilon and CD3 gamma form a heterodimer, and two CD3 zeta form a homodimer.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof that, when natively expressed by a T-cell, provides the primary cytoplasmic signaling sequence(s) that regulate activation of the TCR complex in a stimulatory way for at least some aspect of the T-cell signaling pathway. TCR alpha and/or TCR beta chains of wild type TCR complexes do not contain stimulatory domains and require association with CD3 subunits such as CD3 zeta to initiate signaling. In one aspect, the primary stimulatory signal is initiated by, for instance, binding of a TCR/CD3 complex with an a major histocompatibility complex (MHC) bound to peptide, and which leads to mediation of a T-cell response, including, but not limited to, proliferation, activation, differentiation, and the like. One or more stimulatory domains, as described herein, can be fused to the intracellular portion of any one or more subunits of the TCR complex, including TCR alpha, TCR beta, CD3 delta, CD3 gamma and CD3 epsilon.

As used herein, a “domain capable of providing a stimulatory signal” refers to any domain that, either directly or indirectly, can provide a stimulatory signal that enhances or increases the effectiveness of signaling mediated by the TCR complex to enhance at least some aspect of T-cell signaling. The domain capable of providing a stimulatory signal can provide this signal directly, for example with the domain capable of providing the stimulatory signal is a primary stimulatory domain or co-stimulatory domain. Alternatively, or in addition, the domain capable of providing the stimulatory signal can act indirectly. For example, the domain can be a scaffold that recruits stimulatory proteins to the TCR, or provide an enzymatic activity, such as kinase activity, that acts through downstream targets to provide a stimulatory signal.

As used herein, a “domain capable of providing an inhibitory signal” refers to any domain that, either directly or indirectly, can provide an inhibitory signal that inhibits or decreases the effectiveness of signaling mediated by the TCR complex. The domain capable of providing an inhibitory signal can reduce, or block, totally or partially, at least some aspect of T-cell signaling or function. The domain capable of providing an inhibitory signal can provide this signal directly, for example with the domain capable of providing the inhibitory signal provides a primary inhibitory signal. Alternatively, or in addition, the domain capable of providing the stimulatory signal can act indirectly. For example, the domain can recruit additional inhibitory proteins to the TCR, or can provide an enzymatic activity that acts through downstream targets to provide an inhibitory signal.

Any suitable ligand binding domain may be fused to an extracellular domain, hinge domain or transmembrane of the TCRs described herein. For example, the ligand binding domain can be an antigen binding domain of an antibody or TCR, or comprise an antibody fragment, a Vβ only domain, a linear antibody, a single-chain variable fragment (scFv), or a single domain antibody (sdAb).

In some embodiments, the ligand binding domain is fused to one or more extracellular domains or transmembrane domains of one or more TCR subunits. The TCR subunit can be TCR alpha, TCR beta, CD3 delta, CD3 epsilon, CD3 gamma or CD3 zeta. For example, the ligand binding domain can be fused to TCR alpha, or TCR beta, or portions of the ligand binding can be fused to two subunits, for example portions of the ligand binding domain can be fused to both TCR alpha and TCR beta.

TCR subunits include TCR alpha, TCR beta, CD3 zeta, CD3 delta, CD3 gamma and CD3 epsilon. Any one or more of TCR alpha, TCR beta chain, CD3 gamma, CD3 delta, CD3 epsilon, or CD3 zeta, or fragments or derivative thereof, can be fused to one or more domains capable of providing a stimulatory signal of the disclosure, thereby enhancing TCR function and activity.

TCR transmembrane domains isolated or derived from any source are envisaged as within the scope of the disclosure. The transmembrane domain may be derived either from a natural or from a recombinant source. Where the source is natural, the domain may be derived from any membrane-bound or transmembrane protein.

In some embodiments, the transmembrane domain is capable of signaling to the intracellular domain(s) whenever the TCR complex has bound to a target. A transmembrane domain of particular use in this disclosure may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the transmembrane domain can be attached to the extracellular region of a polypeptide of the TCR, e.g., the antigen binding domain of the TCR alpha or beta chain, via a hinge, e.g., a hinge from a human protein. For example, the hinge can be a human immunoglobulin (Ig) hinge, e.g., an IgG4 hinge, or a CD8a hinge. In some embodiments, the hinge is isolated or derived from CD8α or CD28.

In some embodiments, the extracellular ligand binding domain is attached to one or more transmembrane domains of the TCR. In some embodiments, the transmembrane domain comprises a TCR alpha transmembrane domain, a TCR beta transmembrane domain, or both. In some embodiments, the transmembrane comprises a CD3 zeta transmembrane domain.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region).

In some embodiments, the transmembrane domain can be selected or modified by amino acid substitution to avoid binding of such domains to the transmembrane domains of the same or different surface membrane proteins, e.g., to minimize interactions with other members of the receptor complex.

When present, the transmembrane domain may be a natural TCR transmembrane domain, a natural transmembrane domain from a heterologous membrane protein, or an artificial transmembrane domain. The transmembrane domain may be a membrane anchor domain. Without limitation, a natural or artificial transmembrane domain may comprise a hydrophobic a-helix of about 20 amino acids, often with positive charges flanking the transmembrane segment. The transmembrane domain may have one transmembrane segment or more than one transmembrane segment. Prediction of transmembrane domains/segments may be made using publicly available prediction tools (e.g. TMHMM, Krogh et al. Journal of Molecular Biology 2001; 305(3):567-580; or TMpred, Hofmann & Stoffel Biol. Chem. Hoppe-Seyler 1993; 347:166). Non-limiting examples of membrane anchor systems include platelet derived growth factor receptor (PDGFR) transmembrane domain, glycosylphosphatidylinositol (GPI) anchor (added post-translationally to a signal sequence) and the like.

In some embodiments, the transmembrane domain comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity, or 100% identical to a sequence of: VIGFRILLLKVAGFNLLMTLRLW (SEQ ID NO: 71). In some embodiments, the TCR alpha transmembrane domain comprises, or consists essentially of, SEQ ID NO: 71. In some embodiments, the TCR alpha transmembrane domain is encoded by a sequence of

(SEQ ID NO: 72)
GTGATTGGGTTCCGAATCCTCCTCCTGAAAGTGGCCGGGTTTAATCTGCT
CATGACGCTGCGGCTGTGG.

In some embodiments, the transmembrane domain comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity, or 100% identical to a sequence of: TILYEILLGKATLYAVLVSALVL (SEQ ID NO: 73). In some embodiments, the TCR beta transmembrane domain comprises, or consists essentially of, SEQ ID NO: 73. In some embodiments, the TCR beta transmembrane domain is encoded by a sequence of

(SEQ ID NO: 74)
ACCATCCTCTATGAGATCTTGCTAGGGAAGGCCACCTTGTATGCCGTGCT
GGTCAGTGCCCTCGTGCTG.

TCRs of the disclosure can comprise one or more intracellular domains. In some embodiments, the intracellular domain comprises one or more domains capable of providing a stimulatory signal to a transmembrane domain. In some embodiments, the intracellular domain comprises a first intracellular domain capable of providing a stimulatory signal and a second intracellular domain capable of providing a stimulatory signal. In other embodiments, the intracellular domain comprises a first, second and third intracellular domain capable of providing a stimulatory signal. The intracellular domains capable of providing a stimulatory signal are selected from the group consisting of a CD28 molecule (CD28) domain, a LCK proto-oncogene, Src family tyrosine kinase (Lck) domain, a TNF receptor superfamily member 9 (4-1BB) domain, a TNF receptor superfamily member 18 (GITR) domain, a CD4 molecule (CD4) domain, a CD8a molecule (CD8a) domain, a FYN proto-oncogene, Src family tyrosine kinase (Fyn) domain, a zeta chain of T cell receptor associated protein kinase 70 (ZAP70) domain, a linker for activation of T cells (LAT) domain, lymphocyte cytosolic protein 2 (SLP76) domain, (TCR) alpha, TCR beta, CD3 delta, CD3 gamma and CD3 epsilon intracellular domains.

In some embodiments, an intracellular domain comprises at least one intracellular signaling domain. An intracellular signaling domain generates a signal that promotes a function a cell, for example an immune effector function of a TCR containing cell, e.g., a TCR-expressing T-cell. In some embodiments, the intracellular domain of the first receptor of the disclosure includes at least one intracellular signaling domain. For example, the intracellular domains of CD3 gamma, delta or epsilon comprise signaling domains.

In some embodiments, the extracellular domain, transmembrane domain and intracellular domain are isolated or derived from the same protein, for example T-cell receptor (TCR) alpha, TCR beta, CD3 delta, CD3 gamma, CD3 epsilon or CD3 zeta.

Examples of intracellular domains for use in activator receptors of the disclosure include the cytoplasmic sequences of the TCR alpha, TCR beta, CD3 zeta, and 4-1BB, and the intracellular signaling co-receptors that act in concert to initiate signal transduction following antigen receptor engagement, as well as any derivative or variant of these sequences and any recombinant sequence that has the same functional capability.

In some embodiments, the intracellular signaling domain comprises a primary intracellular signaling domain. Illustrative primary intracellular signaling domains include those derived from the proteins responsible for primary stimulation, or antigen dependent stimulation.

In some embodiments, the intracellular domain comprises a CD3 delta intracellular domain, a CD3 epsilon intracellular domain, a CD3 gamma intracellular domain, a CD3 zeta intracellular domain, a TCR alpha intracellular domain or a TCR beta intracellular domain.

In some embodiments, the intracellular domain comprises a TCR alpha intracellular domain. In some embodiments, a TCR alpha intracellular domain comprises Ser-Ser. In some embodiments, a TCR alpha intracellular domain is encoded by a sequence of TCCAGC (SEQ ID NO: 75).

In some embodiments, the intracellular domain comprises a TCR beta intracellular domain. In some embodiments, the TCR beta intracellular domain comprises an amino acid sequence having at least 80% identity, at least 90% identity, or 100% identical to a sequence of: MAMVKRKDSR (SEQ ID NO: 76). In some embodiments, the TCR beta intracellular domain comprises, or consists essentially of SEQ ID NO: 76. In some embodiments, the TCR beta intracellular domain is encoded by a sequence of

| (SEQ ID NO: 77) | |
|---|---|
| | ATGGCCATGGTCAAGAGAAAGGATTCCAGA. |

In some embodiments, the intracellular signaling domain comprises at least one stimulatory intracellular domain. In some embodiments, the intracellular signaling domain comprises a primary intracellular signaling domain, such as a CD3 delta, CD3 gamma and CD3 epsilon intracellular domain, and one additional stimulatory intracellular domain, for example a co-stimulatory domain. In some embodiments, the intracellular signaling domain comprises a primary intracellular signaling domain, such as a CD3 delta, CD3 gamma and CD3 epsilon intracellular domain, and two additional stimulatory intracellular domains.

Illustrative co-stimulatory intracellular signaling domains include those derived from proteins responsible for co-stimulatory signals, or antigen independent stimulation. Co-stimulatory molecules include, but are not limited to an MHC class I molecule, BTLA, a Toll ligand receptor, as well as DAP10, DAP12, CD30, LIGHT, OX40, CD2, CD27, CDS, ICAM-1, LFA-1 (CD11a/CD18) 4-1BB (CD137, TNF receptor superfamily member 9), and CD28 molecule (CD28). A co-stimulatory protein can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, CD4, and the like. The co-stimulatory domain can comprise the entire intracellular portion, or the entire native intracellular signaling domain, of the molecule from which it is derived, or a functional variant thereof.

In some embodiments, the stimulatory domain comprises a co-stimulatory domain. In some embodiments, the co-stimulatory domain comprises a CD28 or 4-1BB co-stimulatory domain. CD28 and 4-1BB are well characterized co-stimulatory molecules required for full T cell activation and known to enhance T cell effector function. For example, CD28 and 4-1BB have been utilized in chimeric antigen receptors (CARs) to boost cytokine release, cytolytic function, and persistence over the first-generation CAR containing only the CD3 zeta signaling domain. Likewise, inclusion of co-stimulatory domains, for example CD28 and 4-1BB domains, in TCRs can increase T cell effector function and specifically allow co-stimulation in the absence of co-stimulatory ligand, which is typically down-regulated on the surface of tumor cells. In some embodiments, the stimulatory domain comprises a CD28 intracellular domain or a 4-1BB intracellular domain.

### Inhibitor Receptors

The disclosure provides a second receptor, comprising an extracellular ligand binding domain specific to a non-cancerous antigen that has been lost in a cancer cell. The antigen can be lost in the cancer cell through any mechanism, such as, without limitation, epigenetic changes that effect antigen expression, mutations to the gene encoding the non-cancerous antigen, disruption of cellular signaling that regulates expression of the non-cancerous antigen, chromosome loss, partial or complete deletion of the genomic locus, gene silencing through modification of nucleic acids or heterochromatin, or loss of expression through other mechanisms. In variations of the compositions and methods disclosed herein, the cells or subject treated may exhibit a loss of expression of the non-cancerous antigen because of non-genetic changes. Accordingly the disclosure provides compositions and methods for killing cells lacking expression of the non-cancerous antigen from any cause.

Illustrative inhibitory receptors are described in PCT/US2020/045228, PCT/US2020/064607, PCT/US2021/029907, and PCT/US2020/059856, the contents of each of which are incorporated herein by reference.

The term “inhibitor chimeric antigen receptor” or “inhibitor receptor” as used herein refers to an antigen-binding domain that is fused to an intracellular signaling domain capable of transducing an inhibitory signal that inhibits or suppresses the immune activity of an immune cell. Inhibitor receptors have immune cell inhibitory potential, and are distinct and distinguishable from CARs, which are receptors with immune cell activating potential. For example, CARs are activating receptors as they include intracellular stimulatory and/or co-stimulatory domains. Inhibitor receptors are inhibiting receptors that contain intracellular inhibitory domains.

As used herein “inhibitory signal” refers to signal transduction or changes in protein expression in an immune cell resulting in suppression of an immune response (e.g., decrease in cytokine production or reduction of immune cell activation). Inhibition or suppression of an immune cell can be selective and/or reversible, or not selective and/or reversible. Inhibitor receptors are responsive to non-cancerous or allogeneic donor cell antigens expressed on normal or allogeneic donor cells (e.g. LILRB1). For example, when a non-cancerous or allogeneic donor cell antigen (e.g. LILRB1) binds to or contacts the inhibitor receptor, the inhibitor receptor is responsive and initiates an inhibitory signal in the immune cell expressing the inhibitor receptor. Upon binding of the non-cancerous or allogeneic donor cell antigen by the extracellular ligand binding domain of the inhibitor receptor, the inhibitor receptor inhibits activation of an immune cell expressing the engineered inhibitor receptor.

Inhibitory receptors of the disclosure may comprise an extracellular ligand binding domain. Any type of ligand binding domain that can regulate the activity of a receptor in a ligand dependent manner is envisaged as within the scope of the instant disclosure. Inhibitory receptors are responsive to allogeneic donor cell antigens (e.g. HLA-A*02). For example, when an allogeneic donor cell antigen (e.g. HLA-A*02) binds to or contacts the inhibitory receptor, the inhibitory receptor is responsive and activates an inhibitory signal in the immune cell expressing the inhibitory receptor upon binding of the allogeneic donor cell antigen by the extracellular ligand binding domain of the inhibitory receptor.

A “non-cancerous antigen” or “non-cancerous ligand” as used herein refers to a gene whose expression is reduced or eliminated in a cancer cell, but is expressed or detectably expressed in a normal or healthy cell. For example, a subject may have a cancer in which the cancer calls express SPN. In such a subject, because the inhibitor receptor is designed to be specific to PECAM-1 (or other non-cancerous antigen), the inhibitor receptor of immune cells encountering the cancer cell does not release an inhibitory signal. In normal non-cancerous cells, PECAM-1 (or other non-cancerous antigen) is present and inhibits activation of the engineered immune cell. By this mechanism, the immune cell is selectively activated, and selectively kills, cancer cells expressing SPN but having lost PECAM-1 (or another non-cancerous antigen). In some embodiments, the non-cancerous antigen is not expressed in a cancer cell of the subject. In some embodiments, the non-cancerous antigen is not expressed in a fraction of the cells in a tumor in the subject. In some embodiments, the non-cancerous antigen is expressed by a cancer cell at a lower expression level relative to the level of expression in a non-cancerous cell.

In some embodiments, a cancer cell in a subject has lost expression of the non-cancerous antigen. Loss of expression or lack of expression of the non-cancerous antigen in a cell can be by any mechanism, such as, without limitation, epigenetic changes that effect non-cancerous gene expression, mutations to the gene encoding the non-cancerous antigen, genetic deletion, or disruption of cellular signaling that regulates expression of the non-cancerous gene.

An “allogeneic donor cell antigen” or “allogeneic donor cell ligand” as used herein refers to a gene whose expression is reduced or eliminated in an allogeneic donor cell, but is expressed or detectably expressed in a subject's cell. For example, a subject may have a cancer in which the cancer calls express SPN and non-cancerous blood cells which express SPN. In such a subject, because the inhibitor receptor is designed to be specific to HLA-A*02 (or other allogeneic donor cell antigen), the inhibitor receptor of immune cells encountering the subject's cell does not release an inhibitory signal. In allogeneic donor cells, HLA-A*02 (or other allogeneic donor cell antigen) is present and inhibits activation of the engineered immune cell. By this mechanism, the immune cell is selectively activated, and selectively kills, a subject's cells expressing SPN but not expressing HLA-A*02 (or another allogeneic donor cell antigen). The allogeneic donor cell may be a stem cell. The stem cell may be a HSC.

In some embodiments, the second receptor is an inhibitor chimeric antigen receptor (i.e. inhibitor receptor). In some embodiments, the second receptor is an inhibitor receptor. In some embodiments, the second receptor is humanized.

The disclosure provides a second receptor, which is an inhibitory receptor, comprising an extracellular ligand binding domain that can discriminate between different levels of expression of a non-cancerous or allogeneic donor cell antigen. This allows the second receptor to inhibit activation of immune cells comprising the second receptor when contacted by non-cancerous or allogeneic donor cells that express the ligand for the second receptor, but to allow activation of immune cells that are contacted by cancer or the subject's cells that express low levels, or have no expression, of the ligand for the second receptor.

### Inhibitor Ligands

The disclosure provides a second ligand and a second inhibitor receptor comprising a second ligand binding domain that binds to the second ligand. In some embodiments, the second ligand is an inhibitor ligand. In some embodiments, the second ligand is an antigen, and the second inhibitor receptor comprises an antigen binding domain that specifically recognizes the second ligand antigen.

The disclosure provides a second inhibitor receptor comprising an extracellular region, the extracellular region comprising a second ligand binding domain capable of specifically binding to a second ligand that inhibits activation of effector cells expressing the first and second receptors, wherein the effector cells are activated by binding only the first ligand to the first activator receptor.

As used herein an “inhibitor” or “inhibitor ligand” or “inhibitor antigen” refers to a second ligand that binds to a second ligand binding domain (inhibitor LBD) of an engineered receptor of the disclosure, but inhibits activation of an immune cell expressing the engineered inhibitor receptor. The inhibitor is not expressed by the cancerous cells. The inhibitor ligand is expressed in a plurality of normal, non-cancerous cells, including normal, non-cancerous cells that express the activator ligand, thereby protecting these cells from the cytotoxic effects of the adoptive cell therapy. Without wishing to be bound by theory, inhibitor ligands can block activation of the effector cells through a variety of mechanisms. For example, binding of the inhibitor ligand to the inhibitor LBD can block transmission of a signal that occurs upon binding of the activator ligand to the activator LBD that would, in the absence of the inhibitor, lead to activation of the immune cell expressing the engineered receptors described herein.

In some embodiments, the inhibitor is not expressed by the subject's cells. The inhibitor ligand is expressed in a plurality of allogeneic donor cells, including allogeneic donor cells that express the A antigen, thereby protecting these cells from the cytotoxic effects of the adoptive cell therapy. Without wishing to be bound by theory, inhibitor ligands can block activation of the effector cells through a variety of mechanisms. For example, binding of the inhibitor ligand to the inhibitor LBD can block transmission of a signal that occurs upon binding of the A antigen to the activator LBD that would, in the absence of the inhibitor, lead to activation of the immune cell expressing the engineered receptors described herein.

Alternatively, or in addition, binding of the inhibitor ligand to the second engineered receptor can cause loss of cell surface expression the first, activator receptor from the surface of the immune cells comprising the two receptor system described herein. Without wishing to be bound by theory, it is thought that immune cell engagement of activator and inhibitor ligands on normal cells acts in part by causing the inhibitory receptor to cause removal of nearby activator receptor molecules from the immune cell surface. This process locally desensitizes the immune cell, reversibly raising its activation threshold. Immune cells that engage only the activator ligand on a cancer cell cause local activation signals which are unimpeded by signals from the second, inhibitory receptor. This local activation increases until release of cytotoxic granules leads to cancer cell selective cell death. However, modulation of surface receptor expression levels may not be the only mechanism by which inhibitory receptors inhibit activation of immune cells by the first activator receptor. Without wishing to be bound by theory, other mechanisms may come into play, including, but not limited to, cross-talk between activator and inhibitory receptor signaling pathways.

In some embodiments, the second ligand is not expressed by the cancer cells, and is expressed by the non-cancerous cells. In some embodiments, the non-cancerous antigen is not expressed by the cancer cells, and is expressed by non-cancerous cells. In some embodiments, the non-cancerous antigen is expressed by healthy cells, i.e. cells that are not cancer cells. In some embodiments, the cancer cells have lost expression of the non-cancerous antigen. In some embodiments, the non-cancerous cells are a plurality of healthy cells (i.e., normal, or healthy cells), that express both the cancer antigen and the non-cancerous antigen.

In some embodiments, the inhibitor ligand is an antigen and the second ligand binding domain comprises a scFv domain.

In some embodiments, the inhibitor ligand binding domain comprises a VB-only ligand binding domain.

In some embodiments, the inhibitor ligand binding domain comprises an antigen binding domain isolated or derived from a T cell receptor (TCR). For example, the inhibitor ligand binding domain comprises TCR α and β chain variable domains.

In some embodiments, the inhibitor ligand binding domain is an antigen binding domain. Suitable antigen-binding domains include, but are not limited to, antigen-binding domains from antibodies, antibody fragments, scFv, antigen-binding domains derived from T cell receptors, and the like.

Any cell surface molecule expressed by the non-cancerous cells that is not expressed by cancer cells may be a suitable non-cancerous antigen for the second receptor extracellular ligand binding domain. For example, a cell adhesion molecule, a cell-cell signaling molecule, an extracellular domain, a molecule involved in chemotaxis, a glycoprotein, a G protein-coupled receptor, a transmembrane domain, a receptor for a neurotransmitter, or a voltage gated ion channel can be used as a non-cancerous antigen.

Alternatively, or in addition, expression of activator and inhibitor antigens may be correlated, i.e. the two are expressed at similar levels on non-cancerous cells.

In some embodiments, the inhibitor ligand is a peptide ligand. In some embodiments, the inhibitor ligand is a peptide antigen.

In some embodiments, the non-cancerous antigen is lost in the cancer cells. In some embodiments, the inhibitor ligand is encoded by a gene that is absent or polymorphic in blood cancer cells.

Methods of distinguishing the differential expression of inhibitor ligands between cancerous and non-cancerous cells will be readily apparent to the person or ordinary skill in the art. For example, the presence or absence of inhibitor ligands in non-cancerous and cancerous cells can be assayed by immunohistochemistry with an antibody that binds to the inhibitor ligand, followed by microscopy or FACS, RNA expression profiling of cancer cells and non-cancerous cells, or DNA sequencing of non-cancerous and cancerous cells to determine if the genomic locus of the inhibitor ligand comprises mutations or deletions in either the cancerous or non-cancerous cells.

### CDCP1

In some embodiments, the non-cancerous antigen is CUB domain-containing protein 1 (CDCP1). In some embodiments, the non-cancerous antigen comprises a peptide of CDCP1. In some embodiments, the CDCP1 non-cancerous cell antigen is expressed by healthy cells of a subject.

In some embodiments, the inhibitor ligand is CDPC1.

The term “CDCP1” (also known as SIMA135, TRASK, CD318 and gp140) refers to a 140 kD transmembrane glycoprotein with a large extracellular domain containing three CUB domains, and a smaller intracellular domain. CDCP1 may be cleaved by serine proteases at the extracellular domain to generate a truncated molecule of 80 kDa size. Reference to CDCP1 is also intended to include a further isoform (UniProt number Q9H5V8-3) having an NK to SE substitution at 342 and 343 and amino acids 344-836 missing.

In vivo, CDCP1 is not cleaved during normal physiological circumstances, but its cleavage may be induced during tumorigenesis or tissue injury.

Western blot analysis has demonstrated that CDCP1 is a phosphotyrosine protein, consistent with the presence of 5 intracellular tyrosine residues. In addition, the inhibitor PP2 has been used to demonstrate that a Src kinase family member acts to phosphorylate the tyrosines of CDCP1. CDCP1 may contribute to metastasis, in part, by allowing cancer cells to survive and metastasize in the absence of attachment. In the MDA-MB-468 breast cancer cell line, enforced CDCP1 expression induced cell detachment and growth in suspension even in the presence of a suitable adhesive substrate. However, CDCP1-mediated cell detachment is not observed universally, and how CDCP1 induces suspension growth in specific circumstances is unknown.

In some embodiments, the inhibitor ligand comprises CDCP1. There are several CDCP1 variants encoding distinct isoforms, all of which fall within the scope of the instant disclosure.

### ABCG2

In some embodiments, the inhibitor ligand is ABCG2.

The term “ABCG2” (also known as ATP-binding cassette super-family G member 2, CD338, and CDw338) refers to an ATP-binding cassette (ABC) transporter protein. ABCG2 actively pumps drugs and other compounds against their concentration gradient using the bonding and hydrolysis of ATP as the energy source. The expression of this transport protein is highly conserved throughout the animal kingdom. ABCG2 forms into a homodimer to assume its active transport conformation.

Substrate binding with compounds occurs in the large central cavity. ABCG2 can bind to a broad range of compounds but binds strongest to flat, polycyclic chemicals with lots of hydrophobic character. In some embodiments, the inhibitor ligand comprises ABCG2. There are several ABCG2 variants encoding distinct isoforms, all of which fall within the scope of the instant disclosure.

### FCGR3A

In some embodiments, the inhibitor ligand is FCGR3A.

The term “FCGR3A” (also known as low affinity immunoglobulin gamma Fc region receptor III-A, FcγRIIIA, CD16, and CD16a) refers to a receptor for the invariable Fc fragment of immunoglobulin gamma (IgG). FCGR3A is optimally activated upon binding of clustered antigen-IgG complexes displayed on cell surfaces, and triggers lysis of antibody-coated cells, a process known as antibody-dependent cellular cytotoxicity (ADCC). FCGR3A does not bind free monomeric IgG, thus avoiding inappropriate effector cell activation. FCGR3A binds antigen-IgG complexes generated upon infection and triggers NK cell-dependent cytokine production and degranulation to limit viral load and propagation. FCGR3A is involved in the generation of memory-like adaptive NK cells capable to produce high amounts of IFNG and to efficiently eliminate virus-infected cells via ADCC. FCGR3A is expressed on NK cells, monocytes, and macrophages. The major polymorphism in FCGR3A is a point mutation (T>G) at nucleotide 559 (rs396991), which results in either a valine (V158) or phenylalanine (F158) at amino acid position 158. These alleles are also co-dominantly expressed. In some embodiments, the inhibitor ligand comprises FCGR3A. There are several FCGR3A variants encoding distinct isoforms, all of which fall within the scope of the instant disclosure.

### PECAM-1

In some embodiments, the inhibitor ligand is PECAM-1.

The term “PECAM-1” (also known as Platelet endothelial cell adhesion molecule and CD31) refers to a hemophilic cell adhesion molecule. PECAM-1 is a type I transmembrane protein with six C2-set Ig domains. Homophilic interactions between leukocyte PECAM-1 and endothelial PECAM-1 are required for the trans-endothelial migration (TEM) of leukocytes. Leukocyte PECAM-1 engages endothelial PECAM-1 upon reaching the apical side of the endothelial border, and when this interaction is disrupted through genetic ablation or function-blocking reagents, leukocytes are arrested on the apical surface of the endothelial cell, above the junction. There are several PECAM-1 variants encoding distinct isoforms, all of which fall within the scope of the instant disclosure.

### HLA-G

In some embodiments, the inhibitor ligand is HLA-G.

The term “HLA-G” (also known as HLA-G histocompatibility antigen class I G and human leukocyte antigen G) refers to a non-classical HLA class I heavy chain paralogue. HLA-G shares 86% sequence similarity with classical HLA class I genes; however, a stop codon in exon 6 produces a mature HLA-G molecule that is shorter than its classical class I counterparts. Alternative splicing of the primary transcript produces seven different isoforms of HLA-G. All isoforms are equally tolerogenic. Typically, HLA-G forms a MHC class I complex together with β2 microglobulin (B2M or β2m). In one embodiment, HLA-G refers to the MHC class I complex of HLA-G and β2 microglobulin. There are several HLA-G variants encoding distinct isoforms, all of which fall within the scope of the instant disclosure.

### HLA-G Targeted CAR

The HLA-G targeted CAR (also called “anti-HLA-G CAR”) or HLA-G targeted polypeptide described herein may include a HLA-G targeting scFv.

In some embodiments, the second receptor is an anti-HLA-G CAR comprising any one of SEQ ID NOS: 391-393 or a sequence sharing at least 75%, at least 80%, at least 85%, at least 90%, at least 95%, or at least 98% identity thereto.

### SIGLEC5

In some embodiments, the inhibitor ligand is SIGLEC5.

The term “SIGLEC5” (also known as Sialic acid-binding Ig-like lectin 5 and CD170) refers to a cell surface lectin characterized by structural motifs in the immunoglobulin (Ig)-like domains and sialic acid recognition sites in the first Ig V set domain. The sialic acid-binding immunoglobulin-like lectins (SIGLECs), such as SIGLEC5, are a subgroup of the immunoglobulin superfamily that mediate protein-carbohydrate interactions. They specifically interact with sialic acids in glycoproteins and glycolipids, with each SIGLEC having a particular preference for both the nature of the sialic acid and its glycosidic linkage to adjacent sugars. There are SIGLEC5 variants encoding distinct isoforms, all of which fall within the scope of the instant disclosure.

### PDZK1IP1

In some embodiments, the inhibitor ligand is PDZK1IP1.

The term “PDZK1IP1” (also known as PDZK1 Interacting Protein 1, MAP17, DD96, and SPAP) refers to a protein which interacts with several PDZ domain-containing molecules, including sodium-hydrogen antiporter regulators. PDZK1IP1 contributes to the internalization of sodium-dependent phosphate transport protein 2b the trans-Golgi network and participates in enhancement of the endogenous uphill transport system in the kidney. There are PDZK1IP1 variants encoding distinct isoforms, all of which fall within the scope of the instant disclosure.

### MME

In some embodiments, the inhibitor ligand is membrane metallo-endopeptidase (MME). The term “MME” (also known as neutral endopeptidase (NEP), CD10, common acute lymphoblastic leukemia antigen (CALLA), and Neprilysin) refers to a zinc-dependent metalloprotease that cleaves peptides at the amino side of hydrophobic residues and inactivates several peptide hormones. There are MME variants encoding distinct isoforms, all of which fall within the scope of the instant disclosure.

In some embodiments, the anti-MME VH region comprises any one of SEQ ID NOs: 394, 397, 400-402, 404, or 406 or a sequence having at least 90%, at least 95%, at least 97%, at least 99% identity, or 100% identical thereto. In some embodiments, the anti-MME VL region comprises any one of SEQ ID NOs: 395, 396, 398, 399, 403, 405, or 407 or a sequence having at least 90%, at least 95%, at least 97%, at least 99% identity, or 100% identical thereto. In some embodiments, the binding domain comprises the full length VH region and VL regions on a single polypeptide. In some embodiments, the polypeptide comprises from N-terminal to C-terminal the full length VH regions and the full length VL region. In some embodiments, the polypeptide comprises from N-terminal to C-terminal the full length VL region and the full length VH region.

### Extracellular Ligand Binding Domains

Inhibitor receptors of the disclosure may comprise an extracellular ligand binding domain. Any type of ligand binding domain that can regulate the activity of a receptor in a ligand dependent manner is envisaged as within the scope of the instant disclosure.

In some embodiments, the ligand binding domain is an antigen binding domain. Illustrative antigen binding domains include, inter alia, scFv, SdAb, VB-only domains, and TCR antigen binding domains derived from the TCR α and B chain variable domains.

Any type of antigen binding domain is envisaged as within the scope of the instant disclosure.

Illustrative anti-CDCP1 antibodies include CUB1, REA194, OTI2B2, OTI2C1, OTI2B8, OTI4G5, OTI5B3, CSTEM26, 309116, or 309121.

In some embodiments, the CDCP1 ligand binding domain comprises a scFv domain. Various single variable domains known in the art or disclosed herein that bind to and recognize CDCP1 are suitable for use in embodiments. Such scFvs include, for example and without limitation, the following mouse and humanized scFv antibodies that bind CDCP1 in a peptide-independent way shown in Table 9 below (complementarity determining regions underlined):

TABLE 9
anti-CDCP1 VH and VL Sequences

| Construct | Heavy Chain | Light Chain |
|---|---|---|
| VH; VL | Variable Heavy Chain: | Variable Light Chain: |
| | EVQLQQFGAELVKPGASVKIS | NIVMTQSPQSMSMSVGERVTLSCKA |
| | CKASGYSFSDFNIEWLKQSHG | SENVGAYVSWFQQKPDQSPKLLILA |
| | KSLEWIGDINPNYDSTNYNQK | ASNRYTGVPARFIGSGSATDFTLTI |
| | FKGRATLTVDKSSSTAYMEVR | SSVQAEDLADYHCGQSYTYPYTFGG |
| | SLTSEDTAVYYCARLGYGYAM | GTKLEIKRADAAPTVS (SEQ ID |
| | DYWGQGTSVTVSS (SEQ ID | NO: 79) |
| | NO: 78) | |
| | heavy chain variable | light chain variable and |
| | and constant region: | constant region: |
| | EVQLQQFGAELVKPGASVKIS | IVMTQSPQSMSMSVGERVTLSCKAS |
| | CKASGYSFSDFNIEWLKQSHG | ENVGAYVSWFQQKPDQSPKLLILAA |
| | KSLEWIGDINPNYDSTNYNQK | SNRYTGVPARFIGSGSATDFTLTIS |
| | FKGRATLTVDKSSSTAYMEVR | SVQAEDLADYHCGQSYTYPYTFGGG |
| | SLTSEDTAVYYCARLGYGYAM | TKLEIKRADAAPTVSIFPPSDEQLK |
| | DYWGQGTSVTVSSASTKGPSV | SGTASVVCLLNNFYPREAKVQWKVD |
| | FPLAPSSKSTSGGTAALGCLV | NALQSGNSQESVTEQDSKDSTYSLS |
| | KDYFPEPVTVSWNSGALTSGV | STLTLSKADYEKHKVYACEVTHQGL |
| | HTFPAVLQSSGLYSLSSVVTV | SSPVTKSFNRGEC (SEQ ID NO: |
| | PSSSLGTQTYICNVNHKPSNT | 81) |
| | KVDKKVEPKSCDKTHTCPPCP | |
| | APELLGGPSVFLFPPKPKDTL | |
| | MISRTPEVTCVVVDVSHEDPE | |
| | VKFNWYVDGVEVHNAKTKPRE | |
| | EQYNSTYRVVSVLTVLHQDWL | |
| | NGKEYKCKVSNKALPAPIEKT | |
| | ISKAKGQPREPQVYTLPPSRE | |
| | EMTKNQVSLTCLVKGFYPSDI | |
| | AVEWESNGQPENNYKTTPPVL | |
| | DSDGSFFLYSKLTVDKSRWQQ | |
| | GNVFSCSVMHEALHNHYTQKS | |
| | LSLSPGK (SEQ ID NO: | |
| | 80) | |

In some embodiments, the second antigen binding domain comprises a scFv domain that binds to a CDCP1 antigen.

In some embodiments, the non-cancerous antigen comprises CDCP1. Various single variable domains known in the art or disclosed herein that bind to and recognize CDCP1 are suitable for use in embodiments.

Illustrative heavy chain and light chain CDRs (CDR-H1, CDR-H2 and CDR-H3, or CDR-L1, CDR-L2 and CDR-L3, respectively) for CDCP1 antigen binding domains are shown in Table 10 below.

TABLE 10
anti-CDCP1 CDR Sequences

| CDR-L1 | CDR-L2 | CDR-L3 | CDR-H1 | CDR-H2 | CDR-H3 |
|---|---|---|---|---|---|
| ENVGAY | AAS | GQSYTY | GYSFSDFN | INPNYDST | ARLGYGYAM |
| (SEQ ID | (SEQ ID | PYT | (SEQ ID | (SEQ ID | DY |
| NO: 85) | NO: 86) | (SEQ ID | NO: 82) | NO: 83) | (SEQ ID NO: |
| | | NO: 87) | | | 84) |

In some embodiments, the scFv comprises the complementarity determined regions (CDRs) of any one of SEQ ID NOS: 82-87. In some embodiments, the scFv comprises a sequence at least 95% identical to any one of SEQ ID NOS: 82-87. In some embodiments, the scFv comprises a sequence identical to any one of SEQ ID NOS: 78-87. In some embodiments, the heavy chain of the antibody comprises the heavy chain CDRs of any one of SEQ ID NOS: 82-84, and wherein the light chain of the antibody comprises the light chain CDRs of any one of SEQ ID NOS: 85-87. In some embodiments, the heavy chain of the antibody comprises a sequence at least 95% identical to the heavy chain portion of any one of SEQ ID NOS: 78 or 80, and wherein the light chain of the antibody comprises a sequence at least 95% identical to the light chain portion of any one of SEQ ID NOS: 79 or 81. In some embodiments, the heavy chain of the antibody is encoded by a polynucleotide sequence at least 95% identical to SEQ ID NO: 131 and the light chain of the antibody is encoded by a polynucleotide sequence at least 95% identical to SEQ ID NO: 132.

In some embodiments, the second, inhibitory ligand is CDCP1, and the inhibitory ligand binding domain comprises a CDCP1 antigen binding domain. In some embodiments, the CDCP1 antigen binding domain comprises a scFv domain. In some embodiments, the CDCP1 antigen binding domain comprises a sequence of any one of SEQ ID NOs: 78, 79, 80, or 81.

In some embodiments, the ABCG2 ligand binding domain comprises a scFv domain. Various single variable domains known in the art or disclosed herein that bind to and recognize ABCG2 are suitable for use in embodiments. Such scFvs include, for example and without limitation, the following mouse and humanized scFv antibodies that bind ABCG2 in a peptide-independent way shown in Table 11 below:

TABLE 11
anti-ABCG2 VH and VL Sequences

| Heavy Chain | Light Chain |
|---|---|
| EVMLVESGGALVKPGGSLKLS | DVVMTQTPLSLPVSLGDQASISCRSS |
| CAASGFTFSNNAMSWVRQTPE | QSLVHSDVNTYLHWYLQRPGQSPKLL |
| TRLEWVATITGGGSYTYYPDS | IYKVSNRFSGVPDRFSGSGSGTDFTL |
| VKGRFTISRDNARNTLYLQMS | KISRVESEDLGIYFCSQTTHVPYTFG |
| SLRSEDTATYYCASPDGNYEG | GGTKLEIK (SEQ ID NO: 194) |
| VLAYWGQGTLVTVSA (SEQ | |
| ID NO: 183) | |
| QVQLQQSGADLVRPGASVKLS | DVVMTQSPLSLPVTLGQPASISCRSS |
| CTASGFNIKDDYVHWVKQRPE | QSLVHSDVNTYLHWYQQRPGQSPRLL |
| QGLEWIGRIDPANGNTRYAPK | IYKVSNRFSGVPDRFSGSGSGTDFTL |
| FRGKATMTADTSSNTAYLQLS | KISRVEAEDVGVYFCSQTTHVPYTFG |
| SLTSADTAVYYCSPPLWVGGF | GGTKLEIK (SEQ ID NO: 195) |
| AYWGQGTLVTVSS (SEQ ID | |
| NO: 187) | |
| EVQLVQSGAEVKKPGASVKVS | DIQMTQSSSYLSVSVGGRVTITCKAS |
| CKASGFNIKDDYVHWVRQAPG | DQINYWLAWYQQKPGNAPRLLISGAT |
| QGLEWIGRIDPANGNTRYAPK | SLETGVPSRFSGSGSGKDYTLSITSF |
| FRGRATMTADTSISTAYMELS | QTEDVATYYCQQYWTTPYTFGGGTKV |
| RLRSDDTAVYYCSPPLWVGGF | EIK (SEQ ID NO: 203) |
| AYWGQGTLVTVSS (SEQ ID | |
| NO: 188) | |
| EVQLVQSGAEVKKPGASVKVS | |
| CKASGFNIKDDYVHWVRQAPG | |
| QGLEWIGRIDPAQGNTRYAPK | |
| FRGRATMTADTSISTAYMELS | |
| RLRSDDTAVYYCSPPLWVGGF | |
| AYWGQGTLVTVSS (SEQ ID | |
| NO: 189) | |
| EVQLVQSGAEVKKPGASVKVS | |
| CKASGFNIKDDYVHWVRQAPG | |
| QGLEWIGRIDPASGNTRYAPK | |
| FRGRATMTADTSISTAYMELS | |
| RLRSDDTAVYYCSPPLWVGGF | |
| AYWGQGTLVTVSS (SEQ ID | |
| NO: 190) | |
| QGQMHQSGAELVKPGASVKLS | |
| CKTSGFTFNSGYISWLKQKPR | |
| QSLEWIAWIYAGTGISNFNQK | |
| FTGKAQLTVDTSSSTAYMQLS | |
| SLTSADSAIYFCASGARKTLD | |
| FWGQGTSVTVSS (SEQ ID | |
| NO: 199) | |
| QVQLQESGPGLVKPSQSLSLT | IVLTQSPSSFSVSLGDRVTISCKASG |
| CTVTGFSITSDYAWNWIRQFP | YILNRLAWYQQKPGNAPRLLISGATS |
| GKKLEWMGYINFDGGTTYNPS | LETGFPSRFSGTGSGKDYTLSISSLQ |
| LRGRISITRDTSKNQFFLQLR | TEDVGTYYCQQYWSTPWTFGGGTKLE |
| SVTPEDTATYYCATFYGAKGT | IRRADAAPTVSIFPPSSEQLTSGGAS |
| LDYWGQGTSVTVSSAKTTPPS | VVCFLNNFYPKDINVKWKIDGSERQN |
| VYPLAPVCGDTSGSSVTLGCL | GVLNSWTDQDSKDSTYSMSSTLTLTK |
| VKGYFPEPVTLTWNSGSLSSG | DEYERHNSYTCEATHKTSTSPIVKSF |
| VHTFPAVLQSDLYTLSSSVTV | NRNE (SEQ ID NO: 234) |
| TSSTWPSQSITCNVAHPASST | |
| KVDKKIEPRGP (SEQ ID | |
| NO: 233) | |

In some embodiments, the second antigen binding domain comprises a scFv domain that binds to an ABCG2 antigen.

In some embodiments, the non-cancerous antigen comprises ABCG2. Various single variable domains known in the art or disclosed herein that bind to and recognize ABCG2 are suitable for use in embodiments.

Illustrative heavy chain and light chain CDRs (CDR-H1, CDR-H2 and CDR-H3, or CDR-L1, CDR-L2 and CDR-L3, respectively) for ABCG2 antigen binding domains are shown in Table 12 below.

TABLE 12
anti-ABCG2 CDR Sequences

| CDR-L1 | CDR-L2 | CDR-L3 | CDR-H1 | CDR-H2 | CDR-H3 |
|---|---|---|---|---|---|
| RSSQSL | KVSNRF | SQTTHVP | NNAMS | TITGGGSY | PDGNYEGVLA |
| VHSDVN | S (SEQ | YT (SEQ | (SEQ ID | TYYPDSVK | Y (SEQ ID |
| TYLH | ID NO: | ID NO: | NO: 184) | G (SEQ | NO: 186) |
| (SEQ ID | 197) | 198) | | ID NO: | |
| NO: 196) | | | | 185) | |
| KASDQI | GATSLE | QQYWTTP | DDYVH | RIDPANGN | PLWVGGFAY |
| NYWLA | T (SEQ | YT (SEQ | (SEQ ID | TRYAPKFR | (SEQ ID |
| (SEQ ID | ID NO: | ID NO: | NO: 191) | G (SEQ | NO: 193) |
| NO: 204) | 205) | 206) | | ID NO: | |
| | | | | 192) | |
| | | | SGYIS | WIYAGTGI | GARKTLDF |
| | | | (SEQ ID | SNFNQKFT | (SEQ ID |
| | | | NO: 200) | G (SEQ | NO: 202) |
| | | | | ID NO: | |
| | | | | 201) | |

In some embodiments, the scFv comprises the complementarity determined regions (CDRs) of any one of SEQ ID NOS: 184-186, 191-193, 196-198, 200-202, or 204-206. In some embodiments, the heavy chain of the antibody comprises the heavy chain CDRs of any one of SEQ ID NOS: 184-186, 191-193, or 200-202, and wherein the light chain of the antibody comprises the light chain CDRs of any one of SEQ ID NOS: 196-198 or 204-206. In some embodiments, the heavy chain of the antibody comprises a sequence at least 95% identical to the heavy chain portion of any one of SEQ ID NOS: 187-190 or 199, and wherein the light chain of the antibody comprises a sequence at least 95% identical to the light chain portion of any one of SEQ ID NOS: 194, 195, or 203.

In some embodiments, the second, inhibitory ligand is ABCG2, and the inhibitory ligand binding domain comprises an ABCG2 antigen binding domain. In some embodiments, the ABCG2 antigen binding domain comprises a scFv domain.

In some embodiments, the FCGR3A ligand binding domain comprises a scFv domain. Various single variable domains known in the art or disclosed herein that bind to and recognize FCGR3A are suitable for use in embodiments. Such scFvs include, for example and without limitation, the following mouse and humanized scFv antibodies that bind FCGR3A in a peptide-independent way. In some embodiments, the second antigen binding domain comprises a scFv domain that binds to a FCGR3A antigen.

In some embodiments, the non-cancerous antigen comprises FCGR3A. Various single variable domains known in the art or disclosed herein that bind to and recognize FCGR3A are suitable for use in embodiments.

In some embodiments, the anti-FCGR3A scFv comprises a sequence at least 95% identical to any one of SEQ ID NOS: 207-208. In some embodiments, the scFv comprises a sequence identical to any one of SEQ ID NOS: 207-208.

In some embodiments, the anti-FCG3A VH domain comprises any one of SEQ ID NOs: 235, 237, 245 or a sequence having at least 90%, at least 95%, at least 97%, at least 99% identity, or 100% identical thereto. In some embodiments, the anti-FCGR3A VL domain comprises any one of SEQ ID NOs: 236, 238-244, 246 or a sequence having at least 90%, at least 95%, at least 97%, at least 99% identity, or 100% identical thereto.

In some embodiments, the second, inhibitory ligand is PECAM-1, and the inhibitory ligand binding domain comprises a PECAM-1 antigen binding domain. In some embodiments, the PECAM-1 antigen binding domain comprises a scFv domain. Various single variable domains known in the art or disclosed herein that bind to and recognize PECAM-1 are suitable for use in embodiments. In some embodiments, the non-cancerous antigen comprises PECAM-1. In some embodiments, the second, inhibitory ligand is PECAM-1, and the inhibitory ligand binding domain comprises a PECAM-1 antigen binding domain.

In some embodiments, the HLA-G ligand binding domain comprises a scFv domain. Various single variable domains known in the art or disclosed herein that bind to and recognize HLA-G are suitable for use in embodiments. Such scFvs include, for example and without limitation, the following mouse and humanized scFv antibodies that bind HLA-G in a peptide-independent way shown in Table 13 below (complementarity determining regions underlined):

TABLE 13
anti-HLA-G VH and VL Sequences

| Heavy Chain | Light Chain |
|---|---|
| QVQLQQSGAELVKPGSSVKIS | QIVLTQSPALMSASPGEKVTMTCSA |
| CKASGYSFPSYDMHWIKQQPG | SSSVNYMHWYQQKPRSSPKPWIYLT |
| NGLEWIGWIYPGNGNTKYNQK | SNLASGVPARFSGSGSGTSYSLTIS |
| FNGKASLTADKSSSTAYMQLS | TMEAEDAATYYCQQWSSNLPITFGS |
| SLTSEDSAVYFCAKPTVATRY | GTKLEIK (SEQ ID NO: 222) |
| NWFAYWGQGTLVTVSS (SEQ | |
| ID NO: 221) | |
| QVQLKESGPGLVQPSQTLSLT | DTVLTQSPTLAVSPGEKITISCRAS |
| CSVSGFSLTTYSVSWVRQPPG | ETVSTMLHWYQQKPGQQPKLLISLA |
| KGLEWIGAIWSGGSTDYNSAL | SHLESGVPARFTGSGSGTDFTLTID |
| KSRLRISRDTSKSQVLLKMNS | PVEADDTATYYCQQTWNDPLTFGSG |
| LQTEDRAMYFCVRMNNKFGYW | TKLEIK (SEQ ID NO: 224) |
| YFDLWGPGTMVTVSS (SEQ | |
| ID NO: 223) | |
| QVQLQQPGAELVRPGSSVKLS | DVLMTQIPFSLPVSLGDQASISCRS |
| CKASGYTFTDYWMDWVKQRPG | SQSIVHRSGNTYLEWYLQKPGQSPK |
| QGLEWIGTIYPSDSSTHYNQE | LLIYKVSNRFSGVPDRFSGSGSGTD |
| FKGKATMTVDKSSSTAYMHLS | FTLKISRVEAEDLGVYYCFQGSHLP |
| SLTSEDSAVYYCAREGLAGVF | PTFGGTTLEIK (SEQ ID NO: |
| YFDYWGQGTTLTVSS (SEQ | 232) |
| ID NO: 231) | |
| QIQLVQSGPELKKPGETVKIS | DIHMTQSPSSLSASLGGKVTITCKA |
| CKASGYTFTTYGMSWVKQAPG | SQDINRYIAWYQHKPGKGPRLLIHF |
| KGLKWMGWIYTYSGVPTYADD | TSTLQSGIPSRFSGSGSGRDYSFSI |
| LEGRFAFSLETSASTAYLQIN | SNLEPEDIATYYCLQYDDLRTFGGG |
| NLKNEDTATYFCARVRDGYYR | TKLEIK (SEQ ID NO: 324) |
| YAMDYWGQGTSVTVSSC | |
| (SEQ ID NO: 323) | |
| QVQLQQPGAELVRPGSSVKLS | DDVLMTQIPFSLPVSLGDQASISCR |
| CKASGYTFTDYWMDWVKQRPG | SSQSIVHRSGNTYLEWYLQKPGQSP |
| QGLEWIGTIYPSDSSTHYNQE | KLLIYKVSNRFSGVPDRFSGSGSGT |
| FKGKATMTVDKSSSTAYMHLS | DFTLKISRVEAEDLGVYYCFQGSHL |
| SLTSEDSAVYYCAREGLAGVF | PPTFGGGTTLEIK (SEQ ID NO: |
| YFDYWGQGTTLTVSS (SEQ | 326) |
| ID NO: 325) | |
| QVQLQQPGAELVRPGSSVKLS | DVLMTQIPFSLPVSLGDQASISCRS |
| CKASGYTFTDYWMDWVKQRPG | SQSIVHRSGNTYLEWYLQKPGQSPK |
| QGLEWIGTIYPSDSSTHYNQE | LLIYKVSNRFSGVPDRFSGSGSGTD |
| FKGKATMTVDKSSSTAYMHLS | FTLKISRVEAEDLGVYYCFQGSHLP |
| SLTSEDSAVYYCAREGLAGVF | PTFGGTTLEIK (SEQ ID NO: |
| YFDYWGQGTTLTVSS (SEQ | 380) |
| ID NO: 379) | |
| QIQLVQSGPELKKPGETVKIS | DIHMTQSPSSLSASLGGKVTITCKA |
| CKASGYTFTTYGMSWVKQAPG | SQDINRYIAWYQHKPGKGPRLLIHF |
| KGLKWMGWIYTYSGVPTYADD | TSTLQSGIPSRFSGSGSGRDYSFSI |
| LEGRFAFSLETSASTAYLQIN | SNLEPEDIATYYCLQYDDLRTFGGG |
| NLKNEDTATYFCARVRDGYYR | TKLEIK (SEQ ID NO: 382) |
| YAMDYWGQGTSVTVSSC | |
| (SEQ ID NO: 381) | |
| QVQLQESGPGLVKPSETLSLT | EIVLTQSPGTLSLSPGERATLSCRA |
| CAVSGYSILSGYYWFWIRQPP | SNAVSSSYLAWYQQKPGQAPRLLIY |
| GKGLEWIGGIYHSGSTAYNPS | GASYRATGIPDRFSGSGSGTDFTLT |
| LKSRVTISVDTSKNQFSLKLS | ISRLEPEDFAVYYCQQHSLYPPTFG |
| SVTAADTAVYYCARGGEVTYS | GGTKVEIK (SEQ ID NO: 384) |
| RGPLDVWGQGTTVTVSS | |
| (SEQ ID NO: 383) | |

In some embodiments, the anti-HLA-G VH domain comprises any one of SEQ ID NOs: 221, 223, 231, 323, 325, 379, 381, 383, 385, 387, 388, or a sequence having at least 90%, at least 95%, at least 97%, at least 99% identity, or 100% identical thereto. In some embodiments, the anti-HLA-G VL domain comprises any one of SEQ ID NOs: 222, 224, 232, 324, 326, 380, 382, 384, 386, 389, or a sequence having at least 90%, at least 95%, at least 97%, at least 99% identity, or 100% identical thereto.

In some embodiments, the second antigen binding domain comprises a scFv domain that binds to a HLA-G antigen. In some embodiments, the anti-HLA-G scFv domain comprises SEQ ID NO: 390 or a sequence having at least 90%, at least 95%, at least 97%, at least 99% identity, or 100% identical thereto.

In some embodiments, the non-cancerous antigen comprises HLA-G. Various single variable domains known in the art or disclosed herein that bind to and recognize HLA-G are suitable for use in embodiments.

Illustrative heavy chain and light chain CDRs (CDR-H1, CDR-H2 and CDR-H3, or CDR-L1, CDR-L2 and CDR-L3, respectively) for HLA-G antigen binding domains are shown in Table 14 below.

TABLE 14
anti-HLA-G CDR Sequences

| CDR-L1 | CDR-L2 | CDR-L3 | CDR-H1 | CDR-H2 | CDR-H3 |
|---|---|---|---|---|---|
| SASSSV | LTSNLAS | QQWSSNLP | SYDMH | WIYPGNGN | PTVATRYNWF |
| NYMH | (SEQ ID | IT (SEQ | (SEQ ID | TKYNQKEN | AY (SEQ ID |
| (SEQ ID | NO: 213) | ID NO: | NO: 209) | G (SEQ | NO: 211) |
| NO: 212) | | 214) | | ID NO: | |
| | | | | 210) | |
| RASETV | LASHLES | QQTWNDPL | TYSVS | AIWSGGST | MNNKFGYWYF |
| STMLH | (SEQ ID | T (SEQ | (SEQ ID | DYNSALKS | DL (SEQ ID |
| (SEQ ID | NO: 219) | ID NO: | NO: 215) | (SEQ ID | NO: 217) |
| NO: 218 | | 220) | | NO: 216) | |
| QSIVHRS | KVS | FQGSHLPP | GYTFTDYW | IYPSDSST | AREGLAGVFY |
| GNTY | (SEQ ID | T (SEQ | (SEQ ID | (SEQ ID | FDY (SEQ |
| (SEQ ID | NO: 229) | ID NO: | NO: 225) | NO: 226) | ID NO: |
| NO: 228 | | 230) | | | 227) |

In some embodiments, the scFv comprises the complementarity determined regions (CDRs) of any one of SEQ ID NOS: 209-220 or 225-230. In some embodiments, the heavy chain of the antibody comprises the heavy chain CDRs of any one of SEQ ID NOS: 209-211, 215-217, or 225-227, and wherein the light chain of the antibody comprises the light chain CDRs of any one of SEQ ID NOS: 212-214, 218-220, or 228-230. In some embodiments, the heavy chain of the antibody comprises a sequence at least 95% identical to the heavy chain portion of any one of SEQ ID NOS: 221, 223, or 231, and wherein the light chain of the antibody comprises a sequence at least 95% identical to the light chain portion of any one of SEQ ID NOS: 222, 224, or 232. In some embodiments, the second, inhibitory ligand is HLA-G, and the inhibitory ligand binding domain comprises a HLA-G antigen binding domain. In some embodiments, the HLA-G antigen binding domain comprises a scFv domain.

In further embodiments of any of the ligand binding domains, each CDR sequence may have 1, 2, 3 or more substitutions, insertions, or deletions. CDR sequences may tolerate substitutions, deletions, or insertions. Using sequence alignment tools, routine experimentation, and known assays, those of skill in the art may generate and test variant sequences having 1, 2, 3, or more substitutions, insertions, or deletions in CDR sequences without undue experimentation.

### Extracellular Hinge Region and Transmembrane Domains

In some embodiments, the inhibitor receptors of the present disclosure comprise an extracellular hinge region. Illustrative hinges can be isolated or derived from IgD and CD8 domains, for example IgG1. In some embodiments, the hinge is isolated or derived from CD8α or CD28.

The inhibitor receptors of the present disclosure can be designed to comprise a transmembrane domain that is fused to the extracellular domain of the inhibitor receptors. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular domain of the inhibitory receptors. A glycine-serine doublet provides a particularly suitable linker.

### Intracellular Domain

The disclosure provides an inhibitor receptor comprising an intracellular domain. The intracellular domain of the inhibitor receptors of the instant disclosure is responsible for inhibiting activation of the immune cells comprising the inhibitor receptors, which would otherwise be activated in response to activation signals by the first receptor. In some embodiments, for example in the second inhibitory receptors of the disclosure which provide an inhibitory signal, the inhibitory signal is transmitted through the intracellular domain of the receptor. In some embodiments, the inhibitor receptor comprises an inhibitor intracellular domain. In some embodiments, the inhibitor engineered receptor is a CAR comprising an inhibitory intracellular domain (an inhibitory CAR).

In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

Inhibitory domains can be isolated from human tumor necrosis factor related apoptosis inducing ligand (TRAIL) receptor and CD200 receptor 1. In some embodiments, the TRAIL receptor comprises TR10A, TR10B or TR10D.

In some embodiments, the inhibitory domain comprises an intracellular domain, a transmembrane domain or a combination thereof. In some embodiments, the inhibitory intracellular domain is isolated from phosphoprotein membrane anchor with glycosphingolipid microdomains 1 (PAG1). In some embodiments, the inhibitory intracellular domain is isolated from leukocyte immunoglobulin like receptor B1 (LILRB1). In some embodiments, the inhibitory domain is isolated or derived from a human protein, for example a human TRAIL receptor, CTLA-4, or PD-1, PAG1 or LILRB1 protein.

In some embodiments, the inhibitory domain comprises an intracellular domain, a transmembrane domain, a hinge region or a combination thereof. In some embodiments, the inhibitory domain comprises an immunoreceptor tyrosine-based inhibitory motif (ITIM). In some embodiments, the inhibitory domain comprising an ITIM can be isolated or derived from an immune checkpoint inhibitor such as CTLA-4 and PD-1. Illustrative hinge, transmembrane, and intracellular domains that may be used in inhibitory receptors described herein are shown in Table 15.

Inhibitory domains can be isolated from human tumor necrosis factor related apoptosis inducing ligand (TRAIL) receptor and CD200 receptor 1. In some embodiments, the inhibitory domain is isolated or derived from a human protein, for example a human TRAIL receptor, CTLA-4, or PD-1 protein. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the engineered receptor comprises an inhibitory domain isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein. In some embodiments, the inhibitor receptor comprises an inhibitory domain. In some embodiments, the inhibitor receptor comprises an inhibitory intracellular domain and/or an inhibitory domain and transmembrane domain. In some embodiments, the inhibitory intracellular domain is fused to an intracellular domain of an inhibitor receptor. In some embodiments, the inhibitory intracellular domain is fused to the transmembrane domain of an inhibitor receptors.

In some embodiments, the inhibitor receptor comprises a cytoplasmic domain and transmembrane domain isolated or derived from the same protein, for example an ITIM containing protein. In some embodiments, the inhibitor receptor comprises a cytoplasmic domain, a transmembrane domain, and an extracellular domain or a portion thereof isolated or derived isolated or derived from the same protein, for example an ITIM containing protein. In some embodiments, the inhibitor receptor comprises a hinge region isolated or derived from isolated or derived from the same protein as the intracellular domain and/or transmembrane domain, for example an ITIM containing protein. In some embodiments, the inhibitor receptor comprises an inhibitory domain. In some embodiments, the inhibitor receptor comprises an inhibitory intracellular domain and/or an inhibitory transmembrane domain. In some embodiments, the inhibitor receptor is a CAR comprising an inhibitory domain (an inhibitory CAR). In some embodiments, the inhibitory intracellular domain is fused to the intracellular domain of a CAR. In some embodiments, the inhibitory intracellular domain is fused to the transmembrane domain of a CAR.

The disclosure provides inhibitor receptors comprising an intracellular domain. In some embodiments, the inhibitor receptor comprises a LILRB1 intracellular domain or a functional variant thereof.

As used herein, “intracellular domain” refers to the cytoplasmic or intracellular domain of a protein, such as a receptor, that interacts with the interior of the cell, and carries out a cytosolic function. As used herein, “cytosolic function” refers to a function of a protein or protein complex that is carried out in the cytosol of a cell. For example, intracellular signal transduction cascades are cytosolic functions.

As used herein an “immunoreceptor tyrosine-based inhibitory motif” or “ITIM” refers to a conserved sequence of amino acids with a consensus sequence of S/I/V/LxYxxI/V/L (SEQ ID NO: 88), or the like, that is found in the cytoplasmic tails of many inhibitor receptors of the immune system. After ITIM-possessing inhibitory receptors interact with their ligand, the ITIM motif is phosphorylated, allowing the inhibitory receptor to recruit other enzymes, such as the phosphotyrosine phosphatases SHP-1 and SHP-2, or the inositol-phosphatase called SHIP.

In some embodiments, the polypeptide comprises an intracellular domain comprising at least one immunoreceptor tyrosine-based inhibitory motif (ITIM), at least two ITIMs, at least 3 ITIMs, at least 4 ITIMs, at least 5 ITIMs or at least 6 ITIMs. In some embodiments, the intracellular domain has 1, 2, 3, 4, 5, or 6 ITIMs.

In some embodiments, the polypeptide comprises an intracellular domain comprising at least one ITIM selected from the group of ITIMs consisting of NLYAAV (SEQ ID NO: 89), VTYAEV (SEQ ID NO: 90), VTYAQL (SEQ ID NO: 91), and SIYATL (SEQ ID NO: 92).

In further particular embodiments, the polypeptide comprises an intracellular domain comprising at least two immunoreceptor tyrosine-based inhibitory motifs (ITIMs), wherein each ITIM is independently selected from NLYAAV (SEQ ID NO: 89), VTYAEV (SEQ ID NO: 90), VTYAQL (SEQ ID NO: 91), and SIYATL (SEQ ID NO: 92).

In some embodiments, the intracellular domain comprises both ITIMs NLYAAV (SEQ ID NO: 89) and VTYAEV (SEQ ID NO: 90). In some embodiments, the intracellular domain comprises a sequence at least 95% identical to SEQ ID NO: 88. In some embodiments, the intracellular domain comprises or consists essentially of a sequence identical to SEQ ID NO: 93.

In some embodiments, the intracellular domain comprises both ITIMs VTYAEV (SEQ ID NO: 90) and VTYAQL (SEQ ID NO: 91). In some embodiments, the intracellular domain comprises a sequence at least 95% identical to SEQ ID NO: 88. In some embodiments, the intracellular domain comprises or consists essentially of a sequence identical to SEQ ID NO: 88.

In some embodiments, the intracellular domain comprises both ITIMs VTYAQL (SEQ ID NO: 91) and SIYATL (SEQ ID NO: 92). In some embodiments, the intracellular domain comprises a sequence at least 95% identical to SEQ ID NO: 94. In some embodiments, the intracellular domain comprises or consists essentially of a sequence identical to SEQ ID NO: 94.

In some embodiments, the intracellular domain comprises the ITIMs NLYAAV (SEQ ID NO: 89), VTYAEV (SEQ ID NO: 90), and VTYAQL (SEQ ID NO: 91). In some embodiments, the intracellular domain comprises a sequence at least 95% identical to SEQ ID NO: 95. In some embodiments, the intracellular domain comprises or consists essentially of a sequence identical to SEQ ID NO: 95.

In some embodiments, the intracellular domain comprises the ITIMs VTYAEV (SEQ ID NO: 90), VTYAQL (SEQ ID NO: 91), and SIYATL (SEQ ID NO: 92). In some embodiments, the intracellular domain comprises a sequence at least 95% identical to SEQ ID NO: 96. In some embodiments, the intracellular domain comprises or consists essentially of a sequence identical to SEQ ID NO: 96.

In some embodiments, the intracellular domain comprises the ITIMs NLYAAV (SEQ ID NO: 89), VTYAEV (SEQ ID NO: 90), VTYAQL (SEQ ID NO: 91), and SIYATL (SEQ ID NO: 92). In embodiments, the intracellular domain comprises a sequence at least 95% identical to SEQ ID NO: 97. In some embodiments, the intracellular domain comprises or consists essentially of a sequence identical to SEQ ID NO: 97.

In some embodiments, the intracellular domain comprises a sequence at least 95% identical to the LILRB1 intracellular domain (SEQ ID NO: 98). In some embodiments, the intracellular domain comprises or consists essentially of a sequence identical to the LILRB1 intracellular domain (SEQ ID NO: 98).

LILRB1 intracellular domains or functional variants thereof of the disclosure can have at least 1, at least 2, at least 4, at least 4, at least 5, at least 6, at least 7, or at least 8 ITIMs. In some embodiments, the LILRB1 intracellular domain or functional variant thereof has 2, 3, 4, 5, or 6 ITIMs.

In particular embodiments, the polypeptide comprises an intracellular domain comprising two, three, four, five, or six immunoreceptor tyrosine-based inhibitory motifs (ITIMs), wherein each ITIM is independently selected from NLYAAV (SEQ ID NO: 89), VTYAEV (SEQ ID NO: 90), VTYAQL (SEQ ID NO: 91), and SIYATL (SEQ ID NO: 92).

In particular embodiments, the polypeptide comprises an intracellular domain comprising at least three immunoreceptor tyrosine-based inhibitory motifs (ITIMs), wherein each ITIM is independently selected from NLYAAV (SEQ ID NO: 89), VTYAEV (SEQ ID NO: 90), VTYAQL (SEQ ID NO: 91), and SIYATL (SEQ ID NO: 92).

In some embodiments, the intracellular domain comprises a TCR alpha intracellular domain. In some embodiments, the intracellular domain comprises a TCR alpha intracellular domain and an LILRB1 intracellular domain, as described herein. In some embodiments, a TCR alpha intracellular domain comprises Ser-Ser. In some embodiments, a TCR alpha intracellular domain is encoded by a sequence of TCCAGC (SEQ ID NO: 75).

In some embodiments, the intracellular domain comprises a TCR beta intracellular domain. In some embodiments, the intracellular domain comprises a TCR beta intracellular domain and an LILRB1 intracellular domain, as described herein. In some embodiments, the TCR beta intracellular domain comprises an amino acid sequence having at least 80% identity, at least 90% identity, or 100% identical to a sequence of: MAMVKRKDSR (SEQ ID NO: 76). In some embodiments, the TCR beta intracellular domain comprises, or consists essentially of MAMVKRKDSR (SEQ ID NO: 76). In some embodiments, the TCR beta intracellular domain is encoded by a sequence of ATGGCCATGGTCAAGAGAAAGGATTCCAGA (SEQ ID NO: 77).

The LILRB1 protein has four immunoglobulin (Ig) like domains termed D1, D2, D3 and D4. In some embodiments, the LILRB1 hinge domain comprises an LILRB1 D3D4 domain or a functional variant thereof. In some embodiments, the LILRB1 D3D4 domain comprises a sequence at least 95%, at least 96%, at least 97%, at least 98%, at least 99% or identical to SEQ ID NO: 99. In some embodiments, the LILRB1 D3D4 domain comprises, or consists essentially of SEQ ID NO: 99.

### LILRB1 Inhibitor Domain

The present disclosure provides an inhibitor receptor comprising a LILRB1 inhibitory domain, and optionally, a LILRB1 transmembrane and/or hinge domain, or functional variants thereof. The inclusion of the LILRB1 transmembrane domain and/or the LILRB1 hinge domain in the inhibitor receptor may increase the inhibitory signal generated by the inhibitor receptor compared to a reference inhibitor receptor having another transmembrane domain or another hinge domain. The inhibitor receptor comprising the LILRB1 inhibitory domain may be a CAR or TCR, as described herein. Any suitable ligand binding domain, as described herein, may be fused to the LILRB1-based inhibitory receptors having one or more domains from Leukocyte immunoglobulin-like receptor subfamily B member 1 (LILRB1, or LIR1). In some embodiments, the inhibitor receptor comprises a LILRB1 intracellular domain or a functional variant thereof.

Leukocyte immunoglobulin-like receptor subfamily B member 1 (LILRB1), also known as Leukocyte immunoglobulin-like receptor B₁, as well as ILT2, LIR1, MIR7, PIRB, CD85J, ILT-2 LIR-1, MIR-7 and PIR-B, is a member of the leukocyte immunoglobulin-like receptor (LIR) family. The LILRB1 protein belongs to the subfamily B class of LIR receptors. These receptors contain two to four extracellular immunoglobulin domains, a transmembrane domain, and two to four cytoplasmic immunoreceptor tyrosine-based inhibitory motifs (ITIMs). The LILRB1 receptor is expressed on immune cells, where it binds to MHC class I molecules on antigen-presenting cells and transduces a negative signal that inhibits stimulation of an immune response. LILRB1 is thought to regulate inflammatory responses, as well as cytotoxicity, and to play a role in limiting auto-reactivity. Multiple transcript variants encoding different isoforms of LILRB1 exist, all of which are contemplated as within the scope of the instant disclosure.

In some embodiments of the inhibitor receptors described herein, the inhibitor receptor comprises one or more domains isolated or derived from LILRB1. In some embodiments of the receptors having one or more domains isolated or derived from LILRB1, the one or more domains of LILRB1 comprise an amino acid sequence that is at least 80%, at least 90%, at least 95%, at least 96%, at least 97%, at least 98%, at least 99%, or 100% identical to a sequence or subsequence of SEQ ID NO: 100. In some embodiments, the one or more domains of LILRB1 comprise an amino acid sequence that is identical to a sequence or subsequence of SEQ ID NO: 100. In some embodiments, the one or more domains of LILRB1 consist of an amino acid sequence that is at least 80%, at least 90%, at least 95%, at least 96%, at least 97%, at least 98%, at least 99%, or 100% identical to a sequence or subsequence of SEQ ID NO: 100. In some embodiments, the one or more domains of LILRB1 consist of an amino acid sequence that is identical to a sequence or subsequence of SEQ ID NO: 100.

In some embodiments of the receptors having one or more domains isolated or derived from LILRB1, the one or more domains of LILRB1 are encoded by a polynucleotide sequence that is at least 80%, at least 90%, at least 95%, at least 96%, at least 97%, at least 98%, at least 99%, or 100% identical to a sequence or subsequence of SEQ ID NO: 101.

In some embodiments of the receptors having one or more domains of LILRB1, the one or more domains of LILRB1 are encoded by a polynucleotide sequence that is identical to a sequence or subsequence of SEQ ID NO: 101. In various embodiments, a chimeric antigen receptor is provided, comprising a polypeptide, wherein the polypeptide comprises one or more of: an LILRB1 hinge domain or functional fragment or variant thereof; an LILRB1 transmembrane domain or a functional variant thereof; and an LILRB1 intracellular domain or an intracellular domain comprising at least one, or at least two immunoreceptor tyrosine-based inhibitory motifs (ITIMs), wherein each ITIM is independently selected from NLYAAV (SEQ ID NO: 89), VTYAEV (SEQ ID NO: 90), VTYAQL (SEQ ID NO: 91), and SIYATL (SEQ ID NO: 92).

### Transmembrane Domain

The disclosure provides inhibitor receptors comprising a transmembrane domain. In some embodiments, the inhibitor receptor comprises a LILRB1 hinge domain, or a functional variant thereof. In some embodiments, the inhibitor receptor comprises a LILRB1 transmembrane domain, or a functional variant thereof. In some embodiments, the inhibitor receptor comprises LILRB1 hinge and transmembrane domains, or functional variants thereof.

A “transmembrane domain,” as used herein, refers to a domain of a protein that spans the membrane of the cell. Transmembrane domains typically consist predominantly of non-polar amino acids, and may traverse the lipid bilayer once or several times. Transmembrane domains usually comprise alpha helices, a configuration which maximizes internal hydrogen bonding.

Transmembrane domains isolated or derived from any source are envisaged as within the scope of the fusion proteins of the disclosure.

In particular embodiments, the polypeptide comprises an LILRB1 transmembrane domain or a functional variant thereof.

In some embodiments, the LILRB1 transmembrane domain or a functional variant thereof comprises a sequence at least 95% identical, at least 96% identical, at least 97% identical, at least 98% identical or at least 99% to SEQ ID NO: 102. In some embodiments, the LILRB1 transmembrane domain or a functional variant thereof comprises a sequence at least 95% identical to SEQ ID NO: 102. In some embodiments, the LILRB1 transmembrane domain comprises a sequence identical to SEQ ID NO: 102. In embodiments, the LILRB1 transmembrane domain consists essentially of a sequence identical to SEQ ID NO: 102.

In some embodiments of the chimeric antigen receptors of the disclosure, the transmembrane domain is not a LILRB1 transmembrane domain. In some embodiments, the transmembrane domain is one that is associated with one of the other domains of the fusion protein, or isolated or derived from the same protein as one of the other domains of the fusion protein.

The transmembrane domain may be derived either from a natural or from a recombinant source. Where the source is natural, the domain may be derived from any membrane-bound or transmembrane protein. Illustrative transmembrane domains may include at least the transmembrane region(s) of e.g., the alpha, beta or zeta chain of the TCR, CD3 delta, CD3 epsilon or CD3 gamma, CD28, CD3 epsilon, CD45, CD4, CD5, CD8, CD9, CD16, CD22, CD33, CD37, CD64, CD80, CD86, CD134, CD137, CD154.

In some embodiments, the transmembrane domain comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity, or 100% identical to a sequence of: VIGFRILLLKVAGFNLLMTLRLW (SEQ ID NO: 71). In some embodiments, the TCR alpha transmembrane domain comprises, or consists essentially of SEQ ID NO: 71. In some embodiments, the TCR alpha transmembrane domain is encoded by a sequence of:

(SEQ ID NO: 72)
GTGATTGGGTTCCGAATCCTCCTCCTGAAAGTGGCCGGGTTTAATCTGCT
CATGACGCTGCGGCTGTGG.

In some embodiments, the transmembrane comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity, or 100% identical to a sequence of: TILYEILLGKATLYAVLVSALVL (SEQ ID NO: 73). In some embodiments, the TCR beta transmembrane domain comprises, or consists essentially of SEQ ID NO: 73. In some embodiments, the TCR beta transmembrane domain is encoded by a sequence of

(SEQ ID NO: 74)
ACCATCCTCTATGAGATCTTGCTAGGGAAGGCCACCTTGTATGCCGTGCT
GGTCAGTGCCCTCGTGCTG.

In some embodiments, the TCR alpha and/or TCR beta transmembrane domain comprises one or more mutations that attenuate or abolish interaction of the TCR with the TCR CD3 subunit. In some embodiments, the TCR alpha transmembrane domain comprises a R253L mutation. In some embodiments, the TCR beta transmembrane domain comprises a K288L mutation.

In some embodiments the transmembrane domain comprise a CD28 transmembrane domain. In some embodiments, the CD28 transmembrane domain comprises an amino acid sequence having at least 80% identity, at least 90% identity, at least 95% identity, at least 99% identity, or 100% identical to a sequence of FWVLVVVGGVLACYSLLVTVAFIIFWV (SEQ ID NO: 49). In some embodiments, the CD28 transmembrane domain comprises or consists essentially of SEQ ID NO: 49. In some embodiments, the CD28 transmembrane domain is encoded by a nucleotide sequence having at least 80% identity, at least 90% identity, at least 95% identity, at least 99% identity, or 100% identical to a sequence of

(SEQ ID NO: 50)
TTCTGGGTGCTGGTCGTTGTGGGCGGCGTGCTGGCCTGCTACAGCCTGCT
GGTGACAGTGGCCTTCATCATCTTTTGGGTG.

In some embodiments, the transmembrane domain can be attached to the extracellular region chimeric antigen receptor, e.g., the antigen-binding domain or ligand binding domain, via a hinge, e.g., a hinge from a human protein. For example, in some embodiments, the hinge can be a human immunoglobulin (Ig) hinge, e.g., an IgG4 hinge, a CD8a hinge or an LILRB1 hinge.

### Hinge Domain

The disclosure provides inhibitor receptors comprising a hinge domain. In some embodiments, the hinge domain is a LILRB1 hinge domain or a functional variant thereof.

The LILRB1 protein has four immunoglobulin (Ig) like domains termed D1, D2, D3 and D4. In some embodiments, the LILRB1 hinge domain comprises an LILRB1 D3D4 domain or a functional variant thereof. In some embodiments, the LILRB1 D3D4 domain comprises a sequence at least 95%, at least 96%, at least 97%, at least 98%, at least 99% or identical to SEQ ID NO: 99. In some embodiments, the LILRB1 D3D4 domain comprises or consists essentially of SEQ ID NO: 99.

In some embodiments, the inhibitor receptor comprises a LILRB1 hinge domain or functional fragment or variant thereof. In embodiments, the LILRB1 hinge domain or functional fragment or variant thereof comprises a sequence at least 95%, at least 96%, at least 97%, at least 98%, at least 99% identical or identical to SEQ ID NO: 103, SEQ ID NO: 99, or SEQ ID NO: 104. In embodiments, the LILRB1 hinge domain or functional fragment or variant thereof comprises a sequence at least 95% identical to SEQ ID NO: 103, SEQ ID NO: 99, or SEQ ID NO: 104.

In some embodiments, the LILRB1 hinge domain comprises a sequence identical to SEQ ID NO: 103, SEQ ID NO: 99, or SEQ ID NO: 104.

In some embodiments, the LILRB1 hinge domain consists essentially of a sequence identical to SEQ ID NO: 103, SEQ ID NO: 99, or SEQ ID NO: 104.

In some embodiments or the chimeric antigen receptors of the disclosure, the receptor comprises a hinge that is not isolated or derived from LILRB1.

In some embodiments, the hinge is isolated or derived from CD8a or CD28. In some embodiments, the CD8a hinge comprises an amino acid sequence having at least 80% identity, at least 90% identity, at least 95% identity, at least 99% identity, or 100% identical to a sequence of TTTPAPRPPTPAPTIASQPLSLRPEACRPAAGGAVHTRGLDFACD (SEQ ID NO: 35). In some embodiments, the CD8a hinge comprises SEQ ID NO: 35. In some embodiments, the CD8a hinge consists essentially of SEQ ID NO: 35. In some embodiments, the CD8a hinge is encoded by a nucleotide sequence having at least 80% identity, at least 90% identity, at least 95% identity, at least 99% identity, or 100% identical to a sequence of

(SEQ ID NO: 36)
accacgacgccagcgccgcgaccaccaacaccggcgcccaccatcgcgtc
gcagcccctgtccctgcgcccagaggcgtgccggccagcggggggggcgc
agtgcacacgagggggctggacttcgcctgtgat.

In some embodiments, the CD28 hinge comprises an amino acid sequence having at least 80% identity, at least 90% identity, at least 95% identity, at least 99% identity, or 100% identical to a sequence of CTIEVMYPPPYLDNEKSNGTIIHVKGKHLCPSPLFPGPSKP (SEQ ID NO: 37). In some embodiments, the CD28 hinge comprises or consists essentially of SEQ ID NO: 37. In some embodiments, the CD28 hinge is encoded by a nucleotide sequence having at least 80% identity, at least 90% identity, at least 95% identity, at least 99% identity, or 100% identical to a sequence of

(SEQ ID NO: 38)
tgtaccattgaagttatgtatcctcctccttacctagacaatgagaagag
caatggaaccattatccatgtgaaagggaaacacctttgtccaagtcccc
tatttcccggaccttctaagccc.

### Combinations of LILRB1 Domains

In some embodiments, the inhibitor receptors of the disclosure comprise a polypeptide comprising more than one LILRB1 domain or functional equivalent thereof. For example, in some embodiments, the polypeptide comprises a LILRB1 transmembrane domain and intracellular domain, or a LILRB1 hinge domain, transmembrane domain and intracellular domain.

In particular embodiments, the polypeptide comprises an LILRB1 hinge domain or functional fragment or variant thereof, and the LILRB1 transmembrane domain or a functional variant thereof. In some embodiments, the polypeptide comprises a sequence at least 95% identical, at least 96% identical, at least 97% identical, at least 98% identical, at least 99% identical or identical to SEQ ID NO: 105. In some embodiments, the polypeptide comprises a sequence at least 95% identical to SEQ ID NO: 105. In some embodiments, the polypeptide comprises a sequence identical to SEQ ID NO: 105.

In further embodiments, the polypeptide comprises: the LILRB1 transmembrane domain or a functional variant thereof, and an LILRB1 intracellular domain and/or an intracellular domain comprising at least one immunoreceptor tyrosine-based inhibitory motif (ITIM), wherein the ITIM is selected from NLYAAV (SEQ ID NO: 89), VTYAEV (SEQ ID NO: 90), VTYAQL (SEQ ID NO: 91), and SIYATL (SEQ ID NO: 92). In some embodiments, the polypeptide comprises the LILRB1 transmembrane domain or a functional variant thereof, and an LILRB1 intracellular domain and/or an intracellular domain comprising at least two ITIMs, wherein each ITIM is independently selected from NLYAAV (SEQ ID NO: 89), VTYAEV (SEQ ID NO: 90), VTYAQL (SEQ ID NO: 91), and SIYATL (SEQ ID NO: 92).

In some embodiments, the polypeptide comprises a LILRB1 transmembrane domain and intracellular domain. In some embodiments, the polypeptide comprises a sequence at least 95% identical, at least 96% identical, at least 97% identical, at least 98% identical, at least 99% identical or identical to SEQ ID NO: 106. In some embodiments, the polypeptide comprises a sequence at least 95% identical to SEQ ID NO: 106. In some embodiments, the polypeptide comprises a sequence identical to SEQ ID NO: 106.

In preferred embodiments, the polypeptide comprises: a LILRB1 hinge domain or functional fragment or variant thereof; a LILRB1 transmembrane domain or a functional variant thereof; and a LILRB1 intracellular domain and/or an intracellular domain comprising at least two immunoreceptor tyrosine-based inhibitory motifs (ITIMs), wherein each ITIM is independently selected from NLYAAV (SEQ ID NO: 89), VTYAEV (SEQ ID NO: 90), VTYAQL (SEQ ID NO: 91), and SIYATL (SEQ ID NO: 92).

In some embodiments, the polypeptide comprises a sequence at least 95% identical, at least 99% identical, or identical to SEQ ID NO: 108 or SEQ ID NO: 107.

In some embodiments, the polypeptide comprises a sequence at least 95% identical, at least 99% identical, or identical to SEQ ID NO: 105.

In some embodiments, the polypeptide comprises a sequence at least 95% identical, at least 99% identical, or identical to SEQ ID NO: 106.

In some embodiments, the inhibitor receptor comprises a LIRLRB1 hinge, transmembrane domain, and intracellular domain. In some embodiments, the LIRLRB1 hinge, transmembrane domain, and intracellular domain comprises a sequence of SEQ ID NO: 108, or a sequence at 90%, at least 95% identical, at least 96% identical, at least 97% identical, at least 98% identical, at least 99% identical or identical thereto.

TABLE 15
Inhibitory receptor Hinge, Transmembrane
and Intracellular Domains

| Protein | |
|---|---|
| Sequence | DNA Sequence |
| YGSQSSKPYLLT | TACGGCTCACAGAGCTCCAAACCCTACCTGC |
| HPSDPLELVVSG | TGACTCACCCCAGTGACCCCCTGGAGCTCGT |
| PSGGPSSPTTGP | GGTCTCAGGACCGTCTGGGGGCCCCAGCTCC |
| TSTSGPEDQPLT | CCGACAACAGGCCCCACCTCCACATCTGGCC |
| PTGSDPQSGLGR | CTGAGGACCAGCCCCTCACCCCCACCGGGTC |
| HLGVVIGILVAV | GGATCCCCAGAGTGGTCTGGGAAGGCACCTG |
| ILLLLLLLLLFL | GGGGTTGTGATCGGCATCTTGGTGGCCGTCA |
| ILRHRRQGKHWT | TCCTACTGCTCCTCCTCCTCCTCCTCCTCTT |
| STQRKADFQHPA | CCTCATCCTCCGACATCGACGTCAGGGCAAA |
| GAVGPEPTDRGL | CACTGGACATCGACCCAGAGAAAGGCTGATT |
| QWRSSPAADAQE | TCCAACATCCTGCAGGGGCTGTGGGGCCAGA |
| ENLYAAVKHTQP | GCCCACAGACAGAGGCCTGCAGTGGAGGTCC |
| EDGVEMDTRSPH | AGCCCAGCTGCCGATGCCCAGGAAGAAAACC |
| DEDPQAVTYAEV | TCTATGCTGCCGTGAAGCACACACAGCCTGA |
| KHSRPRREMASP | GGATGGGGTGGAGATGGACACTCGGAGCCCA |
| PSPLSGEFLDTK | CACGATGAAGACCCCCAGGCAGTGACGTATG |
| DRQAEEDRQMDT | CCGAGGTGAAACACTCCAGACCTAGGAGAGA |
| EAAASEAPQDVT | AATGGCCTCTCCTCCTTCCCCACTGTCTGGG |
| YAQLHSLTLRRE | GAATTCCTGGACACAAAGGACAGACAGGCGG |
| ATEPPPSQEGPS | AAGAGGACAGGCAGATGGACACTGAGGCTGC |
| PAVPSIYATLAI | TGCATCTGAAGCCCCCCAGGATGTGACCTAC |
| H (SEQ ID | GCCCAGCTGCACAGCTTGACCCTCAGACGGG |
| NO: 108) | AGGCAACTGAGCCTCCTCCATCCCAGGAAGG |
| | GCCCTCTCCAGCTGTGCCCAGCATCTACGCC |
| | ACTCTGGCCATCCACTAG (SEQ ID NO: |
| | 109) |

Additional sequences for the LILRB1 based inhibitor receptors of the disclosure are shown in Table 16 below, with D3D4 domain and ITIMs underlined.

TABLE 16
Polypeptide Sequences for Elements of
Illustrative Inhibitory Receptors

| Name | Sequence |
|---|---|
| LILRB1 | MTPILTVLICLGLSLGPRTHVQAGHLPKPTLWAEPGSVIT |
| | QGSPVTLRCQGGQETQEYRLYREKKTALWITRIPQELVKK |
| | GQFPIPSITWEHAGRYRCYYGSDTAGRSESSDPLELVVTG |
| | AYIKPTLSAQPSPVVNSGGNVILQCDSQVAFDGFSLCKEG |
| | EDEHPQCLNSQPHARGSSRAIFSVGPVSPSRRWWYRCYAY |
| | DSNSPYEWSLPSDLLELLVLGVSKKPSLSVQPGPIVAPEE |
| | TLTLQCGSDAGYNRFVLYKDGERDFLQLAGAQPQAGLSQA |
| | NFTLGPVSRSYGGQYRCYGAHNLSSEWSAPSDPLDILIAG |
| | QFYDRVSLSVQPGPTVASGENVTLLCQSQGWMQTFLLTKE |
| | GAADDPWRLRSTYQSQKYQAEFPMGPVTSAHAGTYRCYGS |
| | QSSKPYLLTHPSDPLELVVSGPSGGPSSPTTGPTSTSGPE |
| | DQPLTPTGSDPQSGLGRHLGVVIGILVAVILLLLLLLLLF |
| | LILRHRRQGKHWTSTQRKADFQHPAGAVGPEPTDRGLQWR |
| | SSPAADAQEENLYAAVKHTQPEDGVEMDTRSPHDEDPQAV |
| | TYAEVKHSRPRREMASPPSPLSGEFLDTKDRQAEEDRQMD |
| | TEAAASEAPQDVTYAQLHSLTLRREATEPPPSQEGPSPAV |
| | PSIYATLAIH SEQ ID NO: 110 |
| LILRB1 hinge- | YGSQSSKPYLLTHPSDPLELVVSGPSGGPSSPTTGPTSTS |
| transmembrane- | GPEDQPLTPTGSDPQSGLGRHLGVVIGILVAVILLLLLLL |
| intracellular | LLFLILRHRRQGKHWTSTQRKADFQHPAGAVGPEPTDRGL |
| domain | QWRSSPAADAQEENLYAAVKHTQPEDGVEMDTRSPHDEDP |
| | QAVTYAEVKHSRPRREMASPPSPLSGEFLDTKDRQAEEDR |
| | QMDTEAAASEAPQDVTYAQLHSLTLRREATEPPPSQEGPS |
| | PAVPSIYATLAIH SEQ ID NO: 108 |
| LILRB1 hinge- | VVSGPSGGPSSPTTGPTSTSGPEDQPLTPTGSDPQSGLGR |
| transmembrane- | HLGVVIGILVAVILLLLLLLLLFLILRHRRQGKHWTSTQR |
| intracellular | KADFQHPAGAVGPEPTDRGLQWRSSPAADAQEENLYAAVK |
| domain (w/o | HTQPEDGVEMDTRSPHDEDPQAVTYAEVKHSRPRREMASP |
| YGSQSSKPYLL | PSPLSGEFLDTKDRQAEEDRQMDTEAAASEAPQDVTYAQL |
| THPSDPLEL, | HSLTLRREATEPPPSQEGPSPAVPSIYATLAIH SEQ ID |
| SEQ ID NO: 111) | NO: 112 |
| LILRB1 hinge | YGSQSSKPYLLTHPSDPLELVVSGPSGGPSSPTTGPTSTS |
| domain | GPEDQPLTPTGSDPQSGLGRHLG SEQ ID NO: 103 |
| LILRB1 | VVIGILVAVILLLLLLLLLFLIL SEQ ID NO: 102 |
| transmembrane | |
| domain | |
| LILRB1 | RHRRQGKHWTSTQRKADFQHPAGAVGPEPTDRGLQWRSSP |
| intracellular | AADAQEENLYAAVKHTQPEDGVEMDTRSPHDEDPQAVTYA |
| domain | EVKHSRPRREMASPPSPLSGEFLDTKDRQAEEDRQMDTEA |
| | AASEAPQDVTYAQLHSLTLRREATEPPPSQEGPSPAVPSI |
| | YATLAIH SEQ ID NO: 113 |
| ITIM1 | NLYAAV SEQ ID NO: 89 |
| ITIM2 | VTYAEV SEQ ID NO: 90 |
| ITIM3 | VTYAQL SEQ ID NO: 91 |
| ITIM4 | SIYATL SEQ ID NO: 92 |
| ITIM1-2 | NLYAAVKHTQPEDGVEMDTRSPHDEDPQAVTYAEV SEQ |
| | ID NO: 93 |
| ITIM2-3 | VTYAEVKHSRPRREMASPPSPLSGEFLDTKDRQAEEDRQM |
| | DTEAAASEAPQDVTYAQL SEQ ID NO: 114 |
| ITIM3-4 | VTYAQLHSLTLRREATEPPPSQEGPSPAVPSIYATL SEQ |
| | ID NO: 94 |
| ITIM1-3 | NLYAAVKHTQPEDGVEMDTRSPHDEDPQAVTYAEVKHSRP |
| | RREMASPPSPLSGEFLDTKDRQAEEDRQMDTEAAASEAPQ |
| | DVTYAQL SEQ ID NO: 95 |
| ITIM2-4 | VTYAEVKHSRPRREMASPPSPLSGEFLDTKDRQAEEDRQM |
| | DTEAAASEAPQDVTYAQLHSLTLRREATEPPPSQEGPSPA |
| | VPSIYATL SEQ ID NO: 96 |
| ITIM1-4 | NLYAAVKHTQPEDGVEMDTRSPHDEDPQAVTYAEVKHSRP |
| | RREMASPPSPLSGEFLDTKDRQAEEDRQMDTEAAASEAPQ |
| | DVTYAQLHSLTLRREATEPPPSQEGPSPAVPSIYATL |
| | SEQ ID NO: 97 |
| D3D4 domain | YGSQSSKPYLLTHPSDPLEL SEQ ID NO: 99 |
| Short hinge | VVSGPSGGPSSPTTGPTSTSGPEDQPLTPTGSDPQSGLGR |
| | HLG SEQ ID NO: 104 |
| Hinge (iTIM | YGSQSSKPYLLTHPSDPLELVVSGPSGGPSSPTTGPTSTS |
| hinge) | GPEDQPLTPTGSDPQSGLGRHLGV (SEQ ID NO: |
| | 115) |
| Short hinge 2 | VVSGPSGGPSSPTTGPTSTSGPEDQPLTPTGSDPQSGLGR |
| | HLGV (SEQ ID NO: 116) |
| Long hinge 1 | AGSGGSGGSGGSPVPSTPPTPSPSTPPTPSPSGGSGNSSG |
| | SGGSPVPSTPPTPSPSTPPTPSPSASV (SEQ ID NO: |
| | 117) |
| Long hinge 2 | AGSGGSGGSGGSPVPSTPPTNSSSTPPTPSPSPVPSTPPT |
| | NSSSTPPTPSPSPVPSTPPTNSSSTPPTPSPSASV (SEQ |
| | ID NO: 118) |
| 2X short hinge | VVSGPSGGPSSPTTGPTSTSGPEDQPLTPTGSDPQSGLGR |
| | HVVSGPSGGPSSPTTGPTSTSGPEDQPLTPTGSDPQSGLG |
| | RHLGV (SEQ ID NO: 119) |
| Hinge | TTGPTSTSGPEDQPLTPTGSDPQSGLGRHLGV (SEQ ID |
| (truncated) | NO: 120) |
| Hinge- | YGSQSSKPYLLTHPSDPLELVVSGPSGGPSSPTTGPTSTS |
| transmembrane | GPEDQPLTPTGSDPQSGLGRHLGVVIGILVAVILLLLLLL |
| | LLFLIL SEQ ID NO: 121 |
| Transmembrane- | VVIGILVAVILLLLLLLLLFLILRHRRQGKHWTSTQRKAD |
| intracellular | FQHPAGAVGPEPTDRGLQWRSSPAADAQEENLYAAVKHTQ |
| domain | PEDGVEMDTRSPHDEDPQAVTYAEVKHSRPRREMASPPSP |
| | LSGEFLDTKDRQAEEDRQMDTEAAASEAPQDVTYAQLHSL |
| | TLRREATEPPPSQEGPSPAVPSIYATLAIH SEQ ID |
| | NO: 122 |

For example, the non-target antigen comprises HLA-A*02, and the second inhibitory receptor comprises a sequence of: DVLMTQTPLSLPVSLGDQASISCRSSQSIVHSNGNTYLEWYLQKPGQSPKLLIYKVSN RFSGVPDRFSGSGSGTDFTLKISRVEAEDLGVYYCFQGSHVPRTSGGGTKLEIKGGGG SGGGGSGGGGSGGQVQLQQSGPELVKPGASVRISCKASGYTFTSYHIHWVKQRPGQ GLEWIGWIYPGNVNTEYNEKFKGKATLTADKSSSTAYMHLSSLTSEDSAVYFCAREE ITYAMDYWGQGTSVTVSSYGSQSSKPYLLTHPSDPLELVVSGPSGGPSSPTTGPTSTS GPEDQPLTPTGSDPQSGLGRHLGVVIGILVAVILLLLLLLLLFLILRHRRQGKHWTSTQ RKADFQHPAGAVGPEPTDRGLQWRSSPAADAQEENLYAAVKHTQPEDGVEMDTRS PHDEDPQAVTYAEVKHSRPRREMASPPSPLSGEFLDTKDRQAEEDRQMDTEAAASE APQDVTYAQLHSLTLRREATEPPPSQEGPSPAVPSIYATLAIH (SEQ ID NO: 677), or a sequence having at least 90%, at least 95%, at least 97%, at least 99%, or is identical thereto. In some embodiments, the non-target antigen comprises HLA-A*02 and the second inhibitory receptor comprises a sequence of SEQ ID NO: 677.

### Assays

Provided herein are assays that can be used to measure the activity of the engineered receptors of the disclosure.

The activity of engineered receptors can be assayed using a cell line engineered to express a reporter of receptor activity such as a luciferase reporter. Illustrative cell lines include Jurkat T cells, although any suitable cell line known in the art may be used. For example, Jurkat cells expressing a luciferase reporter under the control of an NFAT promoter can be used as effector cells. Expression of luciferase by this cell line reflects TCR-mediated signaling.

The reporter cells can be transfected with each of the various fusion protein constructs, combinations of fusion protein constructs or controls described herein.

Expression of the fusion proteins in reporter cells can be confirmed by using fluorescently labeled MHC tetramers to detect expression of the fusion protein.

To assay the activity of engineered receptors, target cells are loaded with antigen prior to exposure to the effector cells comprising the reporter and the engineered receptor. For example, target cells can be loaded with antigen at least 12, 14, 16, 18, 20, 22 or 24 hours prior to exposure to effector cells. Illustrative target cells include A375 cells, although any suitable cells known in the art may be used. In some cases, target cancer cells can be loaded with serially diluted concentrations of an antigen, such as a CD45 antigen. The effector cells can then be co-cultured with target cancer cells for a suitable period of time, for example 6 hours. Luciferase is then measured by luminescence reading after co-culture. Luciferase luminescence can be normalized to maximum and minimum intensity to allow comparison of activating peptide concentrations for each engineered receptor construct.

Provided herein are methods of determining the relative EC50 of engineered receptors of the disclosure. As used herein, “EC50” refers to the concentration of an inhibitor or agent where the response (or binding) is reduced by half. EC50s of engineered receptors of the disclosure refer to concentration of antigen where binding of the engineered receptor to the antigen is reduced by half. Binding of the antigen, or probe to the engineered receptor can be measured by staining with labeled peptide or labeled peptide-MHC complex. EC50 can be obtained by nonlinear regression curve fitting of reporter signal with peptide titration. Probe binding and EC50 can be normalized to the levels of benchmark TCR without a fusion protein, e.g. SPN.

### Polynucleotide Gene Transfer

The disclosure provides a polynucleotide system, comprising one or more polynucleotides comprising polynucleotide sequences encoding: an activator receptor comprising an extracellular ligand binding domain specific to a SPN antigen and an inhibitor receptor comprising an extracellular ligand binding domain specific to a non-cancerous antigen (e.g., PECAM-1) that is not expressed by the cancer cell.

In some embodiments, the disclosure provides a polynucleotide system, comprising one or more polynucleotides comprising polynucleotide sequences encoding: an activator receptor comprising an extracellular ligand binding domain specific to an A antigen antigen and an inhibitor receptor comprising an extracellular ligand binding domain specific to a B antigen that is not expressed by the subject's cell but is expressed by the donor stem cell.

The disclosure provides plasmids comprising the polynucleotide system described herein. The disclosure provides plasmids comprising the polynucleotides described herein.

The disclosure provides polynucleotides encoding the sequence(s) of the activator and inhibitor receptors described herein. In some embodiments, the sequence of the activating and/or inhibitor receptor, or a fusion protein of the activator and/or inhibitor receptor is operably linked to a promoter. In some embodiments, the sequence encoding the activator receptor, or a polypeptide thereof, is operably linked to a first promoter, and the sequence encoding an inhibitor receptor, or a fusion protein thereof, is operably linked to a second promoter.

The disclosure provides plasmids encoding the coding sequence or sequences of any of the engineered receptors described herein. In some embodiments, the sequence of the activator and/or inhibitor receptor is operably linked to a promoter. In some embodiments, the sequence encoding the activator receptor is operably linked to a first promoter, and the sequence encoding an inhibitor receptor is operably linked to a second promoter.

In some embodiments, the activator receptor is encoded by a first plasmid and the inhibitor receptor is encoded by second plasmid. In some embodiments, both engineered receptors are encoded by a single plasmid.

In some embodiments, the activator and inhibitor receptors are encoded by a single plasmid. Methods of encoding multiple polypeptides using a single plasmid will be known to persons of ordinary skill in the art, and include, inter alia, encoding multiple polypeptides under the control of different promoters, or, if a single promoter is used to control transcription of multiple polypeptides, use of sequences encoding internal ribosome entry sites (IRES) and/or self-cleaving peptides. Illustrative self-cleaving peptides include T2A, P2A, E2A and F2A self-cleaving peptides. In some embodiments, the T2A self-cleaving peptide comprises a sequence of EGRGSLLTCGDVEENPGP (SEQ ID NO: 123). In some embodiments, the P2A self-cleaving peptide comprises a sequence of ATNFSLLKQAGDVEENPGP (SEQ ID NO: 124). In some embodiments, the E2A self-cleaving peptide comprises a sequence of QCTNYALLKLAGDVESNPGP (SEQ ID NO: 125). In some embodiments, the F2A self-cleaving peptide comprises a sequence of VKQTLNFDLLKLAGDVESNPGP (SEQ ID NO: 126). Any of the foregoing can also include an N terminal GSG linker. For example, a T2A self-cleaving peptide can also comprise a sequence of GSGEGRGSLLTCGDVEENPGP (SEQ ID NO: 127), which can be encoded by a sequence of

(SEQ ID NO: 128)
GGATCCGGAGAGGGCAGAGGCAGCCTGCTGACATGTGGCGACGTGGAAG
AGAACCCTGGCCCC.

In some embodiments, a polynucleotide sequence provided herein is codon-optimized. Codon-optimization is the process of introducing silent nucleotide changes into a nucleic acid sequence which increase the production of the protein encoded by the nucleic acid sequence without changing the amino acid sequence of the protein.

In some embodiments, the plasmid is an expression plasmid, i.e. for the expression of the engineered receptors of the disclosure in a suitable cell.

### Polynucleotides

The present disclosure provides nucleic acid molecules, specifically polynucleotides, and/or messenger RNAs (mRNAs) which encode one or more activator and/or inhibitor receptors. The term “nucleic acid,” in its broadest sense, includes any compound and/or substance that comprises a polymer of nucleotides. These polymers are often referred to as polynucleotides. Exemplary nucleic acids or polynucleotides of the disclosure include, but are not limited to, ribonucleic acids (RNAs), mRNAs, modified mRNAs (mmRNAs), deoxyribonucleic acids (DNAs), threose nucleic acids (TNAs), glycol nucleic acids (GNAs), peptide nucleic acids (PNAs), and locked nucleic acids (LNAs). In some embodiments, the polynucleotide is an mRNA. In some embodiments, the mRNA which encodes one or more activator and/or inhibitor receptors is capable of being translated to produce the encoded receptors in vitro, in vivo, in situ or ex vivo.

The present disclosure encompasses the delivery of polynucleotides or mRNA for any of therapeutic, pharmaceutical, diagnostic or imaging by any appropriate route. Delivery may be naked or formulated.

### Naked Delivery

The polynucleotides or mRNA of the present disclosure may be delivered to a cell naked. As used herein in, “naked” refers to delivering polynucleotides or mRNA free from agents which promote transfection. For example, the polynucleotides or mRNA delivered to the cell may contain no modifications. The naked polynucleotides or mRNA may be delivered to the cell using routes of administration described herein.

In some embodiments, the mRNA can be delivered to an immune cell through naked mRNA injection. In some embodiments, intracellular delivery of naked mRNA may be carried out in vitro, in vivo, in situ or ex vivo.

### Formulated Delivery

The polynucleotides or mRNA of the present disclosure may be formulated using the methods described herein. The formulations may contain polynucleotides or mRNA which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated polynucleotide or mRNA may be delivered to the cell using routes of administration described herein.

The compositions may also be formulated for direct delivery to an organ or tissue in any of several ways including, but not limited to, direct soaking or bathing, via a catheter, by gels, powder, ointments, creams, gels, lotions, and/or drops, and the like.

### Viral Vectors

A number of viral based systems have been developed for gene transfer into mammalian cells. For example, retroviruses provide a convenient platform for gene delivery systems. A selected gene can be inserted into a plasmid and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Viral vectors derived from retroviruses such as the lentivirus are suitable tools to achieve long-term gene transfer since they allow long-term, stable integration of a transgene and its propagation in daughter cells. Lentiviral vectors have the added advantage over viral vectors derived from onco-retroviruses such as murine leukemia viruses in that they can transduce non-proliferating cells, such as hepatocytes. They also have the added advantage of low immunogenicity.

### Nanocarriers

Nanocarriers may be used to deliver nucleic acids encoding an activator receptor (or receptors) and an inhibitor receptor (or receptors) to immune cells, either ex vivo or in vivo. For example, a nanocarrier may be used to deliver a messenger RNA (mRNA), or set of mRNAs, encoding the receptors to immune cells.

Nanocarriers include micelles, polymers, liposomes, and lipid nanoparticles (LNPs). Nanoparticles are composed of lipids, polymers, or both that encapsulate the nucleic acid. Examples of nanoparticle-type nanocarriers include ionizable lipid nanoparticles (LNP), solid lipid nanoparticles (SLN). Illustrative LNPs that may be used to deliver nucleic acids to immune cells are described in, e.g., Billingsley et al. (2020, Ionizable Lipid Nanoparticle-Mediated mRNA Delivery for Human CAR T Cell Engineering, Nano Letters). In general, suitable LNPs contain an ionizable lipid core that remains neutral in a physiologically relevant pH but builds charge in acidic environments, such as the endosome, to aid in endosomal escape and enable intracellular nucleic acid delivery. (e.g., WO 2019/067999 and WO 2021/055892).

In some embodiments, a LNP may be used to deliver an mRNA or mmRNA ex vivo.

A number of nanoparticle-based systems have been developed for gene transfer into mammalian cells. For example, lipid nanoparticles provide a convenient platform for gene delivery systems. A selected mRNA (or other nucleic acid) can be packaged in various LNP formulations. The mRNA-containing LNP can then be delivered to cells of the subject in vivo.

Nanocarriers other than LNPs may also be used to deliver nucleic acids to immune cells, both ex vivo and in vivo. For example, Parayanth et al. Nat. Commun. 11:6080 (2020) describes an injectable nanocarrier that delivers in vitro-transcribed (IVT) receptor-encoding mRNA. Illustrative nanocarriers of the disclosure may comprise poly(beta-amino ester) (PBAE) and a coating comprising polyglutamic acid (PGA). Nanocarriers of the disclosure may comprise positively charged lipids comprising poly(beta-amino ester), poly(L-lysine), poly(ethylene imine) (PEI), poly-(amidoamine) dendrimers (PAMAMs), poly(amine-co-esters), poly(dimethylaminoethyl methacrylate) (PDMAEMA), chitosan, poly-(L-lactide-co-L-lysine), poly[a-(4-aminobutyl)-L-glycolic acid] (PAGA), or poly(4-hydroxy-L-proline ester) (PHP).

Viral vectors derived from retroviruses such as the lentivirus are suitable tools to achieve long-term gene transfer since they allow long-term, stable integration of a transgene and its propagation in daughter cells. Lentiviral vectors have the added advantage over viral vectors derived from onco-retroviruses such as murine leukemia viruses in that they can transduce non-proliferating cells, such as hepatocytes. They also have the added advantage of low immunogenicity.

The expression of natural or synthetic nucleic acids encoding engineered receptors is typically achieved by operably linking a nucleic acid encoding the fusion protein or portions thereof to a promoter, and incorporating the construct into an expression plasmid. The plasmids can be suitable for replication and integration in eukaryotes. Typical cloning plasmid contain transcription and translation terminators, initiation sequences, and promoters useful for regulation of the expression of the desired nucleic acid sequence.

The polynucleotides encoding fusion proteins can be cloned into a number of types of plasmids. For example, the polynucleotides can be cloned into a plasmid including, but not limited to a phagemid, a phage derivative, an animal virus, and a cosmid. Plasmids of particular interest include expression plasmid, replication plasmid, probe generation plasmid, and sequencing plasmid.

Further, the expression plasmid may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as viral vectors include, but are not limited to, retroviruses, adenoviruses, adeno-associated viruses, herpes viruses, and lentiviruses. In general, a suitable viral vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a plasmid and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcription initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). Another example of a suitable promoter is the U6 promoter. Another example of a suitable promoter is the H1 promoter. However, other constitutive promoter sequences may also be used, including, but not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters including, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. An illustrative sequence of the U6 promoter is set forth in SEQ ID NO: 129. An illustrative sequence of the H1 promoter is set forth in SEQ ID NO: 130.

In certain aspect, a plasmid comprises a termination sequence, for example, a T6 or T7 termination sequence.

In order to assess the expression of an engineered receptor, the expression plasmid to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression plasmid, the plasmid can be readily introduced into a host cell, e.g., mammalian, bacterial, yeast, or insect cell by any method in the art. For example, the expression plasmid can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising plasmid and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA plasmid. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An illustrative colloidal system for use as a delivery vehicle in vitro and in vivo is a liposome (e.g., an artificial membrane vesicle).

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Immune Cells

Provided herein are immune cells comprising the polynucleotides, plasmids, fusion proteins and engineered receptors described herein.

The disclosure provides an immune cell responsive to a cancer cell, comprising: an activator receptor comprising an extracellular ligand binding domain specific to a SPN antigen; and an inhibitor receptor comprising an extracellular ligand binding domain specific to a PECAM-1 antigen. In some embodiments, the cancer cell expresses SPN.

In some embodiments, the PECAM-1 antigen is expressed by healthy cells of a subject. In some embodiments, the healthy cells of the subject express both the SPN antigen and the PECAM-1 antigen. In some embodiments, the activator receptor and the inhibitor receptor specifically activate the immune cell when contacted by the cancer cell. In some embodiments, the activator receptor and the inhibitor receptor specifically inhibit the immune cell when contacted by the non-cancerous cell.

As used herein, the term “specifically activate” refers to the activation of an immune cell, for example, wherein binding of the activator ligand or “A antigen” to the activator receptor ligand binding domain leads to activation of at least one of the normal effector functions of the immune cell.

In some embodiments, the disclosure provides an immune cell responsive to a cancer cell of a subject, comprising: an activator receptor comprising an extracellular ligand binding domain specific to an A antigen; and an inhibitor receptor comprising an extracellular ligand binding domain specific to a B antigen. In some embodiments, the cancer cell expresses an A antigen of the present disclosure.

The disclosure provides an immune cell responsive to a blood cell of a subject, comprising: an activator receptor comprising an extracellular ligand binding domain specific to an A antigen; and an inhibitor receptor comprising an extracellular ligand binding domain specific to a B antigen. In some embodiments, the blood cell expresses an A antigen of the present disclosure.

In some embodiments, the B antigen is expressed by the allogeneic donor stem cells. In some embodiments, the cells of the subject express the A antigen but do not express the B antigen. In some embodiments, the activator receptor and the inhibitor receptor specifically activate the immune cell when contacted by the cancer cell or subject's blood cell. In some embodiments, the activator receptor and the inhibitor receptor specifically inhibit the immune cell when contacted by the allogeneic donor cell.

As used herein, the term “specifically inhibit” refers to the inhibition on of an immune cell, for example, wherein binding of the inhibitor ligand to the inhibitor receptor ligand binding domain blocks transmission of a signal that occurs upon binding of the activator ligand or “A antigen” to the activator ligand or “A antigen” binding domain that would, in the absence of the inhibitor ligand, lead to activation of the immune cell.

In some embodiments, the immune cell is a T cell. In some embodiments, the T cell is a CD8+ CD4− T cell. In some embodiments, the T cell is a CD8− CD4+ helper T cell. In some embodiments, the T cell is a CD8+ CD4+ T cell. In some embodiments, the immune cell is autologous. In some embodiments, the immune cell is allogeneic.

As used herein, the term “immune cell” refers to a cell involved in the innate or adaptive (acquired) immune systems. Illustrative innate immune cells include phagocytic cells such as neutrophils, monocytes and macrophages, Natural Killer (NK) cells, polymophonuclear leukocytes such as neutrophils, eosinophils, and basophils and mononuclear cells such as monocytes, macrophages, and mast cells. Immune cells with roles in acquired immunity include lymphocytes such as T cells and B cells.

As used herein, a “T cell” refers to a type of lymphocyte that originates from a bone marrow precursor that develops in the thymus gland. There are several distinct types of T cells which develop upon migration to the thymus, which include, helper CD4+ T cells, cytotoxic CD8+ T cells, memory T cells, regulatory CD4+ T cells, NK T cells, γδ T cells, Mucosal-associated invariant T (MAIT) cells, and stem memory T cells. Different types of T cells can be distinguished by the ordinarily skilled artisan based on their expression of markers. Methods of distinguishing between T cell types will be readily apparent to the ordinarily skilled artisan.

In some embodiments, the first receptor and the second receptor together specifically activate the immune cell in the presence of the target cell.

In some embodiments, the immune cell is CD4+, CD8+, a gamma delta T cell, an invariant T cells, an iNK cell, a NK cell, a macrophages, or combinations thereof. In some embodiments, the immune cell is a gamma delta (γδ) T cell. In some embodiments, the immune cell is an invariant T cell. In some embodiments, the immune cell is an invariant natural killer T cell (INKT cell). In some embodiments, the immune cell is a T cell. In some embodiments, the immune cell is a B cell. In some embodiments, the immune cell is a Natural Killer (NK) cell. In some embodiments, the immune cell is CD8−. In some embodiments, the immune cell is CD8+. In some embodiments, the immune cell is CD4+. In some embodiments, the immune cell is CD4−. In some embodiments, the immune cell is CD8−/CD4+. In some embodiments, the immune cell is a CD8+ CD4− T cell.

In some embodiments, the immune cell is non-natural. In some embodiments, the immune cell is isolated.

In some embodiments, the engineered immune cell expresses the first and second receptors at a ratio of about 100:1 to 1:100 of first receptor to second receptor. In some embodiments, the engineered immune cell expresses the first and second receptors at a ratio of about 50:1 to 1:50 of first receptor to second receptor. In some embodiments, the engineered immune cell expresses the first and second receptors at a ratio of about 10:1 to 1:10 of first receptor to second receptor. In some embodiments, the engineered immune cell expresses the first and second receptors at a ratio of about 5:1 to 1:5 of first receptor to second receptor. In some embodiments, the engineered immune cell expresses the first and second receptors at a ratio of about 3:1 to 1:3 of first receptor to second receptor. In some embodiments, the engineered immune cell expresses the first and second receptors at a ratio of about 2:1 to 1:2 of first receptor to second receptor. In some embodiments, the engineered immune cell expresses the first and second receptors at a ratio of about 1:1.

In some embodiments, the immune cell further comprises a T2A self-cleaving peptide, wherein the T2A self-cleaving peptide comprises SEQ ID NO: 123 or 127, or a sequence sharing at least 75%, at least 80%, at least 85%, at least 90%, at least 95%, or at least 98% identity thereto.

In some embodiments, the engineered immune cell comprising the engineered receptors of the disclosure is a T cell. In some embodiments, the T cell is an effector T cell or a regulatory T cell.

In some embodiments, the immune cell is selected form the group consisting of T cells, B cells and Natural Killer (NK) cells. In some embodiments, the immune cell is a T cell. In some embodiments, the immune cell is a B cell. In some embodiments, the immune cell is a Natural Killer (NK) cell. In some embodiments, the immune cell is CD8−. In some embodiments, the immune cell is CD8+. In some embodiments, the immune cell is CD4+. In some embodiments, the immune cell is CD4−. In some embodiments, the immune cell is CD8−/CD4+. In some embodiments, the immune cell is a CD8+/CD4− T cell.

In some embodiments, the immune cell is a gamma delta (γδ) T cell. In some embodiments, the immune cell is an invariant T cell. In some embodiments, the immune cell is an invariant natural killer T cell (INKT cell).

In some embodiments, the immune cell comprises a polynucleotide or polynucleotide system described herein. In some embodiments, the polynucleotide or polynucleotide system comprises a sequence encoding a first receptor described herein. In some embodiments, the polynucleotide or polynucleotide system comprises a sequence encoding a second receptor described herein. In some embodiments, the polynucleotide or polynucleotide systems encodes a shRNA described herein. In some embodiments, the polynucleotide or polynucleotide system comprises a sequence encoding a first receptor described herein and a second receptor described herein. In some embodiments, the polynucleotide or polynucleotide system comprises a sequence encoding a first receptor described herein, a second receptor described herein, and a shRNA described herein.

Methods transforming populations of immune cells, such as T cells, with the viral vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Methods of activating and culturing populations of T cells comprising the engineered TCRs, CARs, fusion proteins or vectors encoding the fusion proteins of the instant disclosure, will be readily apparent to the person of ordinary skill in the art.

Whether prior to or after genetic modification of T cells to express an engineered TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10,040,846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640™ or, X-vivo 15, (Lonza®)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% CO2).

In some embodiments, the T cells comprising engineered TCRs, CARs, and/or inhibitory receptors of the disclosure are autologous. Prior to expansion and genetic modification, a source of T cells is obtained from a subject. Immune cells such as T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11b, CD 16, HLA-DR, and CD8.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, the immune cell is autologous. For example, the immune cells is isolated or derived from same subject who will receive the cell as part of a therapeutic regimen.

In some embodiments, the immune cell is all allogeneic. Allogeneic immune cells can be derived from a donor other than the subject to which the immune cells will be administered. Allogeneic immune cells have been commonly referred to in cell therapy as “off-the-shelf” or “universal” because of the possibility for allogeneic cells to be prepared and stored for use in subjects of a variety of genotypes.

In an embodiment, an immune cell comprises:
(a) an activator receptor comprising an extracellular ligand binding domain specific to a SPN antigen, wherein the extracellular ligand binding domain of the activator receptor is an scFv; and
(b) an inhibitor receptor comprising an extracellular ligand binding domain specific to a PECAM-1 antigen that is not expressed by a blood cancer cell, wherein the extracellular ligand binding domain of the inhibitor receptor is an scFv.

In an embodiment, an immune cell comprises:
(a) an activator receptor comprising an extracellular ligand binding domain specific to a SPN antigen, wherein the extracellular ligand binding domain of the activator receptor is an scFv; and
(b) an inhibitor receptor comprising an extracellular ligand binding domain specific to a PECAM-1 antigen that is expressed by a blood cancer cell at a lower expression level than normal, non-cancerous, cells (e.g., stem cells), wherein the extracellular ligand binding domain of the inhibitor receptor is an scFv.

### Immune Cells with Reduced MHC Class I Polypeptide Expression

In some embodiments, the immune cells described herein are modified to inactivate, or reduce or eliminate expression or function of an endogenous gene encoding an allele of an endogenous MHC class I polypeptide. In some embodiments, the gene encoding the MHC class I polypeptide is HLA-A, HLA-B, and/or HLA-C. HLA-A, HLA-B and HLA-C are encoded by the HLA-A, HLA-B and HLA-C loci. Each of HLA-A, HLA-B and HLA-C includes many variant alleles, all of which are envisaged as within the scope of the instant disclosure. In some embodiments, the gene encoding the MHC class I polypeptide is HLA-A. In some embodiments, the gene encoding the MHC class I polypeptide is HLA-A*02. In some embodiments, the gene encoding the MHC class I polypeptide is HLA-A*02:01. In some embodiments, the gene encoding the MHC class I polypeptide is HLA-A*02:01:01. In some embodiments, the gene encoding the MHC class I polypeptide is HLA-A*02:01:01:01.

In some embodiments, the genetically engineered immune cells described herein are modified to reduce or eliminate expression of the B2M gene product. The beta-2 microglobulin (B2M) gene encodes a protein that associates with the major histocompatibility complex (MHC) class I, i.e. MHC-I complex. The MHC-I complex is required for presentation of antigens on the cell surface. The MHC-I complex is disrupted and non-functional when the B2M is deleted (Wang D et al. Stem Cells Transl Med. 4:1234-1245 (2015)). Furthermore, the B2M gene can be disrupted with high efficiency using gene editing techniques known in the art (Ren et al. Clin. Cancer Res. 23:2255-2266 (2017)). Reducing or eliminating B2M can reduce, or eliminate functional MHC I on the surface of the immune cell.

The disclosure provides gene editing systems for editing an endogenous target gene in an immune cell. The disclosure provides interfering RNAs specific to sequences of target genes. Gene editing systems such as CRISPR/Cas systems, TALENs and zinc fingers can be used to generate double strand breaks, which, through gene repair mechanisms such as homology directed repair or non-homologous end joining (NHEJ), can be used to introduce mutations. NHEJ after resection of the ends of the break, or improper end joining, can be used to introduce deletions. In some embodiments, the target gene comprises a gene encoding a subunit of the MHC-I complex.

Target gene sequences include, but are not limited to, promoters, enhancers, introns, exons, intron/exon junctions, transcription products (pre-mRNA, mRNA, and splice variants), and/or 3′ and 5′ untranslated regions (UTRs). Any gene element or combination of gene elements may be targeted for the purpose of genetic editing in the immune cells described herein. Modifications to the target genes can be accomplished using any method known in the art to edit the target gene that results in altered or disrupted expression or function the target gene or gene product.

In some embodiments, modifying the gene encoding the MHC class I polypeptide comprises deleting all or a portion of the gene. In some embodiments, modifying the gene encoding the MHC class I polypeptide comprises introducing a mutation in the gene. In some embodiments, the mutation comprises a deletion, insertion, substitution, or frameshift mutation. In some embodiments, modifying the gene comprises using a nucleic acid guided endonuclease.

Gene sequences for the target genes described herein are known in the art. The sequences can be found at public databases, such as NCBI GenBank or the NCBI nucleotide database. Sequences may be found using gene identifiers, for example, the HLA-A gene has NCBI Gene ID: 3105, the HLA-B gene has NCBI Gene ID: 3106, the HLA-C gene has NCBI Gene ID: 3107, and the B2M gene has NCBI Gene ID: 567 and NCBI Reference Sequence: NC_000015.10. Gene sequences may also be found by searching public databases using keywords. For example, HLA-A alleles may be found in the NCBI nucleotide database by searching keywords, “HLA-A*02”, “HLA-A*02:01”, “HLA-A*02:01:01”, or “HLA-A*02:01:01:01.” These sequences can be used for targeting in various gene editing techniques known in the art. Table 10 provides non-limiting illustrative sequences of HLA-A allele and B2M gene sequences targeted for modification as described herein.

TABLE 10
Illustrative Target Gene Sequences

| | |
|---|---|
| B2M mRNA | SEQ ID NO: 678 |
| B2M Gene (GenBank: 567) | SEQ ID NO: 679 |
| HLA-A*02:01:01:01 sequence encoding mRNA | SEQ ID NO: 680 |
| HLA-A*02 (GenBank: LK021978.1) | SEQ ID NO: 681 |

The person of ordinary skill in the art will appreciate that T can be substituted for U to convert an RNA sequence to a DNA sequence and vice versa, and both are envisaged as target gene sequences of the disclosure.

### Pharmaceutical Compositions

The disclosure provides pharmaceutical compositions comprising immune cells expressing the engineered receptors of the disclosure and a pharmaceutically acceptable diluent, carrier or excipient. In some embodiments, the pharmaceutical composition is for use as a medicament in the treatment of cancer. In some embodiments, the cancer is blood cancer. In some embodiments, the cancer is a hematologic cancer. In some embodiments, the cancer is leukemia, lymphoma, myeloma, acute myeloid leukemia (AML), acute lymphocytic leukemia (ALL), chronic myelogenous leukemia (CML), chronic lymphocytic leukemia (CLL), myeloid leukemia, Non-Hodgkin lymphoma, Waldenstrom macroglobulinemia, Hodgkin lymphoma, multiple myeloma, myeloproliferative neoplasms (MPNs), juvenile myelomonocytic leukaemia (JMML), Juvenile Chronic Myeloid Leukaemia (JCML), or Chronic Myelomonocytic Leukaemia of Infancy. In some embodiments, the cancer is AML. Illustrative AML subtypes include myeloblastic-undifferentiated (MO), myeloblastic-minimal maturation (M1), myeloblastic-full maturation (M2), promyeloctic (M3), myelomonocytic (M4), monocytic (M5), erythroleukemia (M6), and megakaryocyte (M7).

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

In some embodiments, the immune cell expresses both the first receptor and the second receptor. In some embodiments, at least about 50%, about 55%, about 60%, about 65%, about 70%, about 75%, about 80%, about 85%, about 90%, or about 95% of the immune cells express both the first receptor and the second receptor. In some embodiments, at least 90% of the immune cells express both the first receptor and the second receptor.

The disclosure provides pharmaceutical compositions comprising a plurality of immune cells of the disclosure, and a pharmaceutically acceptable carrier, diluent or excipient.

Formulations of a pharmaceutical composition suitable for parenteral administration typically generally comprise of immune cells combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Parenteral formulations also include aqueous solutions which may contain excipients such as salts, carbohydrates and buffering agents. Illustrative parenteral administration forms include solutions or suspensions in sterile aqueous solutions, for example, aqueous propylene glycol or dextrose solutions. Such dosage forms can be suitably buffered, if desired. Formulations for parenteral administration may be formulated to be immediate and/or modified release. Modified release formulations include delayed-, sustained-, pulsed-, controlled-, targeted and programmed release.

In some embodiments, the formulated composition comprising the immune cells is suitable for administration via injection. In some embodiments, the formulated composition comprising the immune cells is suitable for administration via infusion.

The pharmaceutical compositions of the present disclosure, which may conveniently be presented in unit dosage form, may be prepared according to conventional techniques well known in the pharmaceutical industry. Such techniques include the step of bringing into association the immune cells with the pharmaceutical carrier(s) or excipient(s), such as liquid carriers.

Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, preservatives, antioxidants, opacifiers, thickening agents and stabilizers. However, such materials, when added, should not unduly interfere with the biological activities of the immune cells of the compositions of the present disclosure.

The formulation or composition may also contain more than one active ingredient useful for the particular indication, disease, or condition being treated with the immune cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

Administration can be effected in one dose, continuously or intermittently throughout the course of treatment. Single or multiple administrations can be carried out with the dose level and pattern being selected by the treating physician.

The pharmaceutical composition in some embodiments contains the immune cells in amounts effective to treat or prevent a cancer, such as a therapeutically effective or prophylactically effective amount. Therapeutic or prophylactic efficacy in some embodiments is monitored by periodic assessment of treated subjects. For repeated administrations over days, weeks or months, depending on the condition, the treatment can be repeated until a desired suppression of cancer signs or symptoms occurs. However, other dosage regimens may be useful and can be determined. The desired dosage can be delivered by a single bolus administration or infusion of the composition or by multiple bolus administrations or infusions of the composition.

### Methods of Use

Provided herein are methods for selectively killing SPN-positive blood cancer cells, comprising contacting the SPN-positive blood cancer cells with an immune cell comprising the engineered receptors of the disclosure. The cells may be, for example, in a tissue (e.g., in vivo) or in a mixed culture. In some embodiments, the non-cancerous antigen is a PECAM-1 antigen.

Provided herein are methods of treating a subject in need thereof, comprising administering to the subject a therapeutically effective amount of a composition comprising immune cells comprising the engineered receptors of the disclosure.

In some embodiments, the method comprises treating a SPN+ blood cancer in a subject, comprising administering to the subject the immune cells described herein. In some embodiments, the immune cells are autologous. In some embodiments, the immune cells are allogeneic. In some embodiments, the non-cancerous antigen is a PECAM-1 antigen.

The disclosure provides methods of making an immune cell therapy. In some embodiments, the method comprises transforming immune cells with the polynucleotide system described herein. In some embodiments, the polynucleotide system comprises one or more polynucleotides comprising polynucleotide sequences encoding: an activator receptor comprising an extracellular ligand binding domain specific to a SPN antigen; and an inhibitor receptor comprising an extracellular ligand binding domain specific to a PECAM-1 antigen that is not expressed by the cancer cell.

In some embodiments, the method of treating a subject comprises providing immune cells from a subject suffering from or at risk for a SPN positive blood cancer. In some embodiments, the method comprises transducing the immune cell with the polynucleotide system described herein.

Provided herein are methods for selectively sparing donor stem cells, comprising contacting blood cancer cells with an immune cell comprising the engineered receptors of the disclosure. The cells may be, for example, in a tissue (e.g., in vivo) or in a mixed culture.

Provided herein are methods of treating a subject in need thereof, comprising administering to the subject a therapeutically effective amount of a composition comprising immune cells comprising the engineered receptors of the disclosure.

In some embodiments, the method comprises treating a blood cancer in a subject, comprising administering to the subject the immune cells described herein. In some embodiments, the method comprises conditioning a subject for stem cell transplant, comprising administering to the subject the immune cells described herein. In some embodiments, the immune cells are autologous. In some embodiments, the immune cells are allogeneic. In some embodiments, the donor stem cells comprise the B antigen and are spared by the immune cells described herein.

The disclosure provides methods of making an immune cell therapy. In some embodiments, the method comprises transforming immune cells with the polynucleotide system described herein. In some embodiments, the polynucleotide system comprises one or more polynucleotides comprising polynucleotide sequences encoding: an activator receptor comprising an extracellular ligand binding domain specific to an A antigen; and an inhibitor receptor comprising an extracellular ligand binding domain specific to a B antigen that is not expressed by the donor stem cell.

In some embodiments, the method of treating a subject comprises providing immune cells to a subject suffering from or at risk for blood cancer. In some embodiments, the method comprises transducing the immune cell with the polynucleotide system described herein.

As used herein, a “blood cell” refers to a cell produced through hematopoiesis. Illustrative blood cells include, but are not limited to, myeloid cells, hematopoietic stem cells (blood stem cells), myeloid stem cells, myeloid progenitor cells, and myeloblasts. Myeloid cells are a type of blood cell that originates in the bone marrow. In some embodiments, the myeloid cells are differentiated. Illustrative differentialed myeloid cells include, but are not limited to, erythrocytes, neutrophils, basophils, eosinophils, monocytes, and platelets.

As used herein, a “blood cancer cell” refers to a cancer of blood cells and is characterized by the rapid growth of abnormal leukocytes which accumulate in the bone marrow and disrupt the production of normal blood cells. Blood cancer begins in blood-forming tissue, such as the bone marrow, or in the cells of the immune system. Illustrative blood cancer cells include, but are not limited to, hematopoietic cancer cells, acute myeloid leukemia (AML) cells, and hematologic cancer cells.

As used herein, a “non-cancerous blood cell” refers to a cell produced through hematopoiesis that is not malignant, i.e., the cell is without evidence of cancer. Illustrative non-cancerous blood cells include, but are not limited to, myeloid cells, hematopoietic stem cells (blood stem cells), myeloid stem cells, and myeloblasts. Myeloid cells are a type of blood cell that originates in the bone marrow. In some embodiments, the myeloid cells are differentiated. Illustrative differentialed myeloid cells include, but are not limited to, erythrocytes, neutrophils, basophils, eosinophils, monocytes, and platelets.

As used herein, a “donor stem cell” refers to stem cells derived another person (the donor), such as through stem cell mobilization methods, which are known in the art, or through bone marrow harvesting. The donor stem cells are thus allogeneic and may be transplanted to a subject suffering from or at risk for blood cancer. The donor stem cell transplants replace the cancerous bone marrow of the subject with healthy donor stem cells.

As used herein, “sparing” refers to refraining from killing, injuring, or distressing selective cells. For example, immune cells of the present disclosure are engineered to spare donor stem cells expressing the B antigen of the inhibitor receptor.

As used herein, “autologous” refers to any material derived from the same individual to which it is later to be re-introduced. For example, an engineered autologous cell therapy method involves collection of lymphocytes from a patient, which are then engineered to express, e.g., a CAR construct, and then administered back to the same patient.

As used herein, “allogeneic” refers to any material derived from one individual which is then introduced to another individual of the same species where the donor and recipient are not genetically identical (i.e., the transplant is not derived from the same individual as the recipient or an identical twin of the recipient), e.g., allogeneic stem cell transplantation. As used herein, “haploidentical” refers to a subset of allogeneic where material derived from a half-identical haplotype donor is introduced to a recipient, e.g., haploidentical stem cell transplantation.

The current method for adoptive cell therapy using autologous cells includes isolating immune cells from patient blood, performing a series of modifications on the isolated cells, and administering the cells to a patient (Papathanasiou et al. Cancer Gene Therapy. 27:799-809 (2020)). Providing immune cells from a subject suffering from or at risk for cancer or a hematological malignancy requires isolation of immune cell from the patient's blood, and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g. the modifications described herein.

In some embodiments, methods for adoptive cell therapy include isolating immune cells from donor blood, performing a series of modifications on the isolated cells, and administering the cells to a patient (Papathanasiou et al. Cancer Gene Therapy. 27:799-809 (2020)). Providing immune cells from a subject suffering from or at risk for cancer or a hematological malignancy requires isolation of immune cell from the donor blood, and can be accomplished through methods known in the art, for example, by leukapheresis.

In some embodiments, the method of treating a subject described herein comprises modifications to immune cells from the subject comprising a series of modifications comprising enrichment, activation, genetic modification, expansion, formulation, and cryopreservation.

The disclosure provides enrichment steps that can be, for example, washing and fractionating methods known in the art for preparation of subject PBMCs for downstream procedures, e.g. the modifications described herein. For example, without limitation, methods can include devices to remove gross red blood cells and platelet contaminants, systems for size-based cell fractionation for the depletion of monocytes and the isolation of lymphocytes, and/or systems that allow the enrichment of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g. T cells, required for their ex vivo expansion. Immune cell activation can be achieved, for example, by contacting the subject immune cells with dendritic cells, contacting the subject immune cells with artificial antigen-presenting cells (AAPCs), or contacting the immune cells with irradiated K562-derived AAPCs. Other methods for activating subject immune cells can be, for example, contacting the immune cells with isolated activating factors and compositions, e.g. beads, surfaces, or particles functionalized with activating factors. Activating factors can include, for example, antibodies, e.g. anti-CD3 and/or anti-CD28 antibodies. Activating factors can also be, for example, cytokines, e.g. interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

The disclosure provides genetic modification steps for modifying the subject immune cells. In some embodiments, the genetic modification comprises transducing the immune cell with an engineered receptor. In some embodiments, the method comprises transducing the immune cell with a first viral vector comprising a sequence encoding the activator receptor and a second viral vector comprising a sequence encoding the inhibitor receptor, thereby producing an immune cell expressing the activator and inhibitor receptors.

The disclosure provides expansion steps for the genetically modified subject immune cells. Genetically modified subject immune cells can be expanded in any immune cell expansion system known in the art to generate therapeutic doses of immune cells for administration. For example, bioreactor bags for use in a system comprising controller pumps, and probes that allow for automatic feeding and waste removal can be used for immune cell expansion. Cell culture flasks with gas-permeable membranes at the base may be used for immune cell expansion. Any such system known in the art that enables expansion of immune cells for clinical use is encompassed by the expansion step provided herein. Immune cells are expanded in culture systems in media formulated specifically for expansion. Expansion can also be facilitated by contacting the immune cell of the disclosure with activation factors as described herein. Those skilled in the art will appreciate that expansion steps, as provided herein, may also encompass any newly discovered culture systems, media, or activating factors that can be used to expand immune cells.

The disclosure provides formulation and cryopreservation steps for the expanded genetically modified subject immune cells. Formulation steps provided include, for example, washing away excess components used in the preparation and expansion of immune cells of the methods of treatment described herein. Any pharmaceutically acceptable formulation medium or wash buffer compatible with immune cell known in the art may be used to wash, dilute/concentration immune cells, and prepare doses for administration. Formulation medium can be acceptable for administration of the immune cells, such as, for example crystalloid solutions for intravenous infusion. Cryopreservation can optionally be used to store immune cells long-term. Cryopreservation can be achieved using known methods in the art, including for example, storing cells in a cryopreservation medium containing cryopreservation components. Cryopreservation components can include, for example, dimethyl sulfoxide or glycerol. Immune cells stored in cryopreservation medium can be cryopreserved by reducing the storage temperature to −80° C. to −180° C.

In some embodiments, the method comprises administering immune cells described herein. In some embodiments, the method comprises administering a conditioning regimen prior to administering the immune cells described herein. In some embodiments, the conditioning regimen is lymphodepletion. A lymphodepletion regimen can include, for example, administration of alemtuzumab, cyclophosphamide, benduamustin, rituximab, pentostatin, and/or fludarabine. Lymphodepletion regimen can be administered in one or more cycles until the desired outcome of reduced circulating immune cells.

In some embodiments, the conditioning regimen comprises administering an agent that specifically targets, and reduces or eliminates CD52+ cells in the subject, and the immune cells are modified to reduce or eliminate CD52 expression.

In some embodiments, the method of treatment comprises determining the level of expression of an activator ligand, e.g. a CD45 antigen. In some embodiments, the level of expression of an activator ligand is determined in tumor tissue samples from the subject. In some embodiments, the expression level of an activator ligand is determined using next generation sequencing. In some embodiments, the expression level of an activator ligand is determined using RNA sequencing. In some embodiments, the level of an activator ligand is determined using immunohistochemistry.

In some embodiments, the method of treatment comprises determining the level of expression of an A antigen. In some embodiments, the level of expression of an A antigen is determined in tumor tissue samples from the subject. In some embodiments, the expression level of an A antigen is determined using next generation sequencing. In some embodiments, the expression level of an A antigen is determined using RNA sequencing. In some embodiments, the level of an A antigen is determined using immunohistochemistry.

In some embodiments, the method of treatment comprises administering a therapeutically effective dose of immune cells in a subject in need thereof.

In some embodiments, a therapeutically effective dose of the immune cells described herein are administered. In some embodiments, the immune cells of the disclosure are administered by intravenous injection. In some embodiments, the immune cells of the disclosure are administered by intraperitoneal injection. In some embodiments, a therapeutically effective dose comprises about 0.5×10⁶ cells, about 1×10⁶ cells, about 2×10⁶ cells, about 3×10⁶ cells, 4×10⁶ cells, about 5×10⁶ cells, about 6×10⁶ cells, about 7×10⁶ cells, about 8×10⁶ cells, about 9×10⁶ cells, about 1×10⁷, about 2×10⁷, about 3×10⁷, about 4×10⁷, about 5×10⁷, about 6×10⁷, about 7×10⁷, about 8×10⁷, about 9×10⁷, 1×10⁸ cells, about 2×10⁸ cells, about 3×10⁸ cells, about 4×10⁸ cells, about 5×10⁸ cells, or about 6×10⁸ cells. In some embodiments, a therapeutically effective dose comprises about 0.5×10⁶ cells to about 6×10⁸ cells, about 1×10⁶ cells to about 5×10⁸ cells, about 2×10⁶ cells to about 5×10⁸ cells, about 3×10⁶ cells to about 4×10⁸ cells, about 4×10⁶ cells to about 3×10⁸ cells, about 5×10⁶ cells to about 2×10⁸ cells, about 6×10⁶ cells to about 1×10⁸ cells, about 7×10⁶ cells to about 9×10⁷ cells, about 8×10⁶ cells to about 8×10⁷ cells, about 9×10⁶ cells to about 7×10⁷ cells, about 1×10⁷ cells to about 6×10⁷ cells, or about 2×10⁷ cells to about 5×10⁷ cells. In some embodiments, a therapeutically effective dose comprises about 0.5×10⁶ cells to about 6×10⁸ cells. The term “about” as referred to in a therapeutically dose, can be, for example, ±0.5×10⁶ cells, +0.5×10⁷ cells, or +0.5×10⁸ cells.

In some embodiments, the subject in need thereof has cancer. In some embodiments, the cancer is a CD45 positive cancer. Cancer is a disease in which abnormal cells divide without control and spread to nearby tissue. In some embodiments, the cancer comprises a liquid tumor. Illustrative liquid tumors include leukemias and lymphomas.

Any cancer wherein a plurality of the cancer cells expresses the activator ligand and do not express the inhibitor ligand is envisaged as within the scope of the instant disclosure. For example, CD45 positive cancers that can be treated using the methods described herein include a hematologic cancer, such as blood cancer. In some embodiments, the cancer is leukemia, lymphoma, myeloma, acute myeloid leukemia (AML), acute lymphocytic leukemia (ALL), chronic lymphocytic leukemia (CLL), myeloid leukemia, Non-Hodgkin lymphoma, Waldenstrom macroglobulinemia, Hodgkin lymphoma, multiple myeloma, myeloproliferative neoplasms (MPNs), juvenile myelomonocytic leukaemia (JMML), Juvenile Chronic Myeloid Leukaemia (JCML), or Chronic Myelomonocytic Leukaemia of Infancy.

In some embodiments, a plurality of cancer cells do not express PECAM-1.

In some embodiments, the subject in need thereof has cancer. Cancer is a disease in which abnormal cells divide without control and spread to nearby tissue. In some embodiments, the cancer comprises a liquid tumor. Illustrative liquid tumors include leukemias and lymphomas.

A plurality of cells which express the A antigen and do not express the inhibitor ligand is envisaged as within the scope of the instant disclosure. For example, cancers that can be treated using the methods described herein include a hematologic cancer, such as blood cancer. In some embodiments, the cancer is leukemia, lymphoma, myeloma, acute myeloid leukemia (AML), acute lymphocytic leukemia (ALL), chronic lymphocytic leukemia (CLL), myeloid leukemia, Non-Hodgkin lymphoma, Waldenstrom macroglobulinemia, Hodgkin lymphoma, multiple myeloma, myeloproliferative neoplasms (MPNs), juvenile myelomonocytic leukaemia (JMML), Juvenile Chronic Myeloid Leukaemia (JCML), or Chronic Myelomonocytic Leukaemia of Infancy.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

The disclosure provides methods of treating a cancer in a subject comprising: (a) determining the genotype of normal cells and a plurality of cancer cells of the subject; (b) determining the expression of SPN in a plurality of cancer cells; and (c) administering a plurality of immune cells to the subject, wherein the plurality of immune cells comprise: (i) a first receptor, optionally a chimeric antigen receptor (CAR) or T cell receptor (TCR), comprising an extracellular ligand binding domain specific to SPN, or a peptide antigen thereof; and (ii) a second receptor, optionally an inhibitory chimeric antigen receptor (i.e. inhibitory receptors), comprising an extracellular ligand binding domain specific to a PECAM-1 antigen.

The disclosure provides methods of treating a cancer in a subject comprising: (a) determining the genotype of normal cells and a plurality of cancer cells of the subject; (b) determining the expression of an A antigen in a plurality of cancer cells; and (c) administering a plurality of immune cells to the subject, wherein the plurality of immune cells comprise: (i) a first receptor, optionally a chimeric antigen receptor (CAR) or T cell receptor (TCR), comprising an extracellular ligand binding domain specific to the A antigen, or a peptide antigen thereof; and (ii) a second receptor, optionally an inhibitory chimeric antigen receptor (i.e. inhibitory receptors), comprising an extracellular ligand binding domain specific to a B antigen expressed by the donor stem cells.

Methods of genotyping cancer cells and normal cells from a subject for the presence or absence of SNPs will be readily apparent to persons of ordinary skill in the art. SNP genotyping methods include, inter alia, PCR based methods such as dual-probe TaqMan™ assays, array based hybridization methods and sequencing.

Methods of measuring the expression of the target antigen in cancer or normal cells from a subject will be readily apparent to persons of ordinary skill in the art. These include, inter alia, methods of measuring RNA expression such as RNA sequencing and reverse transcription polymerase chain reaction (RT-PCR), as well as methods of measuring protein expression such as immunohistochemistry based methods.

Methods of measuring the expression of the target antigen in cancer or normal cells from a subject will be readily apparent to persons of ordinary skill in the art. These include, inter alia, methods of measuring RNA expression such as RNA sequencing and reverse transcription polymerase chain reaction (RT-PCR), as well as methods of measuring protein expression such as immunohistochemistry based methods.

In some embodiments, the immune cells are T cells.

In some embodiments, the immune cells are allogeneic or autologous.

In some embodiments, the second receptor increases the specificity of the immune cells for the CD45-positive cancer cells compared to immune cells that express the first receptor but do not express the second receptor. In some embodiments, the immune cells have reduced side effects compared to immune cells that express the first receptor but do not express the second receptor.

### Dosage and Administration

The immune cells and of the present disclosure may be administered in a number of ways depending upon whether local or systemic treatment is desired.

In general, administration may be parenteral.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al and U.S. Pat. No. 4,690,915 to Rosenberg.

The compositions of the disclosure are suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In some embodiments, parenteral administration of the compositions of the present disclosure comprises intravenous or intraarterial administration.

The cells or population of cells can be administrated in one or more doses. In some embodiments, an effective amount of cells can be administrated as a single dose. In some embodiments, an effective amount of cells can be administrated as more than one doses over a period time. Timing of administration is within the judgment of a managing physician and depends on the clinical condition of the patient.

The cells or population of cells may be obtained from any source, such as a blood bank or a donor, or the patient themselves.

An effective amount means an amount which provides a therapeutic or prophylactic benefit. The dosage administered will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

For purposes of the disclosure, an assay, which comprises, for example, comparing the extent to which target cells are lysed or one or more cytokines are secreted by immune cells expressing the receptors, upon administration of a given dose of such immune cells to a mammal, among a set of mammals of which is each given a different dose of the immune cells, can be used to determine a starting dose to be administered to a mammal.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The immune cells of the disclosure are in some embodiments are co-administered with one or more additional therapeutic agents or in connection with another therapeutic intervention, either simultaneously or sequentially in any order. In some contexts, the immune cells are co-administered with another therapy sufficiently close in time such that the immune cell populations enhance the effect of one or more additional therapeutic agents, or vice versa. In some embodiments, the immune cells are administered prior to the one or more additional therapeutic agents. In some embodiments, the immune cells are administered after to the one or more additional therapeutic agents.

In embodiments, a lymphodepleting chemotherapy is administered to the subject prior to, concurrently with, or after administration (e.g., infusion) of adoptive immune cells. In an example, the lymphodepleting chemotherapy is administered to the subject prior to administration of the immune cells. For example, the lymphodepleting chemotherapy ends 1-4 days (e.g., 1, 2, 3, or 4 days) prior to adoptive cell infusion. In embodiments, multiple doses of adoptive cells are administered, e.g., as described herein. In embodiments, a lymphodepleting chemotherapy is administered to the subject prior to, concurrently with, or after administration (e.g., infusion) of the immune cells described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, antithymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD 154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-1H, anti-CD25 antibodies, calcineurin inhibitors, mycophenolate, and steroids, which may be used alone or in combination. As a further example, a lymphodepletion regimen can include, administration of alemtuzumab, cyclophosphamide, benduamustin, rituximab, pentostatin, and/or fludarabine. Lymphodepletion regimen can be administered in one or more cycles until the desired outcome of reduced circulating immune cells. In some embodiments, the lymphodepletion comprises administering an agent that specifically targets, and reduces or eliminates CD52+ cells in the subject, and the immune cells are modified to reduce or eliminate CD52 expression.

In some embodiments, an immune stimulating therapy is administered to the subject prior to, concurrently with, or after administration (e.g. infusion) of adoptive immune cells. In some embodiments, the immune stimulating therapy comprises homeostatic cytokines. In some embodiments, the immune stimulating therapy comprises immune-stimulatory molecules. In some embodiments, the immune stimulating therapy comprises IL-2, IL-7, IL-12, IL-15, IL-21, IL-9, or a functional fragment thereof. In some embodiments, the immune stimulating therapy comprises IL-2, IL-7, IL-12, IL-15, IL-21, IL-9, or combinations thereof. In some embodiments, the immune stimulating therapy comprises IL-2, or a functional fragment thereof.

Methods for adoptive cell therapy using autologous cells includes isolating immune cells from patient blood, performing a series of modifications on the isolated cells including transducing the cells with one or more vectors encoding the dual receptor system described herein, and administering the cells to a patient. Providing immune cells from a subject suffering from or at risk for cancer or a hematological malignancy requires isolation of immune cell from the patient's blood, and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g. the modifications described herein.

In some embodiments, the method of treating a subject described herein comprises modifications to immune cells from the subject comprising a series of modifications comprising enrichment and/or depletion, activation, genetic modification, expansion, formulation, and cryopreservation.

The disclosure provides enrichment and/or depletion steps that can be, for example, washing and fractionating methods known in the art for preparation of subject PBMCs for downstream procedures, e.g. the modifications described herein. For example, without limitation, methods can include devices to remove gross red blood cells and platelet contaminants, systems for size-based cell fractionation for the depletion of monocytes and the isolation of lymphocytes, and/or systems that allow the enrichment or depletion of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g. T cells, required for their ex vivo expansion. Immune cell activation can be achieved, for example, by contacting the subject immune cells with dendritic cells, contacting the subject immune cells with artificial antigen-presenting cells (AAPCs), or contating the immune cells with irradiated K562-derived AAPCs. Other methods for activating subject immune cells can be, for example, contacting the immune cells with isolated activating factors and compositions, e.g. beads, surfaces, or particles functionalized with activating factors. Activating factors can include, for example, antibodies, e.g. anti-CD3 and/or anti-CD28 antibodies. Activating factors can also be, for example, cytokines, e.g. interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

In some embodiments, the method comprises transducing the immune cell with one or more plasmids encoding the activator and inhibitory receptors, thereby producing immune cells expressing the activator and inhibitory receptors.

The disclosure provides expansion steps for the genetically modified subject immune cells. Genetically modified subject immune cells can be expanded in any immune cell expansion system known in the art to generate therapeutic doses of immune cells for administration. For example, bioreactor bags for use in a system comprising controller pumps, and probes that allow for automatic feeding and waste removal can be used for immune cell expansion. Cell culture flasks with gas-permeable membranes at the base may be used for immune cell expansion. Any such system known in the art that enables expansion of immune cells for clinical use is encompassed by the expansion step provided herein. Immune cells are expanded in culture systems in media formulated specifically for expansion. Expansion can also be facilitated by contacting the immune cell of the disclosure with activation factors as described herein. Those skilled in the art will appreciate that expansion steps, as provided herein, may also encompass any newly discovered culture systems, media, or activating factors that can be used to expand immune cells.

The disclosure provides formulation and cryopreservation steps for the expanded genetically modified subject immune cells. Formulation steps provided include, for example, washing away excess components used in the preparation and expansion of immune cells of the methods of treatment described herein. Any pharmaceutically acceptable formulation medium or wash buffer compatible with immune cell known in the art may be used to wash, dilute/concentration immune cells, and prepare doses for administration. Formulation medium can be acceptable for administration of the immune cells, such as, for example crystalloid solutions for intravenous infusion.

Cryopreservation can optionally be used to store immune cells long-term. Cryopreservation can be achieved using known methods in the art, including for example, storing cells in a cryopreservation medium containing cryopreservation components. Cryopreservation components can include, for example, dimethyl sulfoxide or glycerol. Immune cells stored in cryopreservation medium can be cryopreserved by reducing the storage temperature to −80° C. to −196° C.

In some embodiments, the level of expression of an A antigen is determined in tumor tissue samples from the subject. In some embodiments, the expression level of an A antigen is determined using next generation sequencing. In some embodiments, the expression level of an A antigen is determined using RNA sequencing. In some embodiments, the level of an A antigen is determined using immunohistochemistry.

In some embodiments, the method of treatment comprises administering a therapeutically effective dose of immune cells comprising an inhibitor receptor to a subject in need thereof.

In some embodiments, the level of expression of SPN is determined in tumor tissue samples from the subject. In some embodiments, the expression level of SPN is determined using next generation sequencing. In some embodiments, the expression level of SPN is determined using RNA sequencing. In some embodiments, the level of SPN is determined using immunohistochemistry.

In some embodiments, the level of expression of CD45 is determined in tumor tissue samples from the subject. In some embodiments, the expression level of CD45 is determined using next generation sequencing. In some embodiments, the expression level of CD45 is determined using RNA sequencing. In some embodiments, the level of CD45 is determined using immunohistochemistry.

In some embodiments, the method of treatment comprises administering a therapeutically effective dose of immune cells comprising a PECAM-1 inhibitor receptor to a subject in need thereof. In some embodiments, the method of treatment comprises administering a therapeutically effective dose of immune cells comprising a PECAM-1 inhibitory receptor to a subject in need thereof.

In some embodiments, a therapeutically effective dose of the immune cells described herein are administered. In some embodiments, the immune cells of the disclosure are administered by intravenous injection. In some embodiments, the immune cells of the disclosure are administered by intraperitoneal injection. In some embodiments, a therapeutically effective dose comprises about 0.5×10⁶ cells, about 1×10⁶ cells, about 2×10⁶ cells, about 3×10⁶ cells, 4×10⁶ cells, about 5×10⁶ cells, about 6×10⁶ cells, about 7×10⁶ cells, about 8×10⁶ cells, about 9×10⁶ cells, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸ cells, about 2×10⁸ cells, about 3×10⁸ cells, about 4×10⁸ cells, about 5×10⁸ cells, about 6×10⁸ cells, about 7×10⁸ cells, about 8×10⁸ cells, about 9×10⁸ cells, about 1×10⁹ cells, about 2×10⁹ cells, about 3×10⁹ cells, about 3×10⁹ cells, about 4×10⁹ cells, about 5×10⁹ cells, about 5×10⁹ cells, about 6×10⁹ cells, about 7×10⁹ cells, about 8×10⁹ cells, about 9×10⁹ cells, about 1×10¹⁰ cells, about 2×10¹⁰ cells, about 3×10¹⁰ cells, about 4×10¹⁰ cells, about 5×10¹⁰ cells, about 6×10¹⁰ cells, about 7×10¹⁰ cells, about 8×10¹⁰ cells, or about 9×10¹⁰ cells.

In some embodiments, a therapeutically effective dose comprises about 0.5×10⁶ cells to about 9×10¹⁰ cells, about 1×10⁶ cells to about 5×10¹⁰ cells, about 2×10⁶ cells to about 5×10⁹ cells, about 3×10⁶ cells to about 5×10⁹ cells, about 4×10⁶ cells to about 3×10⁹ cells, about 5×10⁶ cells to about 2×10⁹ cells, about 6×10⁶ cells to about 1×10⁹ cells, 0.5×10⁶ cells to about 6×10⁹ cells, about 1×10⁶ cells to about 5×10⁹ cells, about 2×10⁶ cells to about 5×10⁹ cells, about 3×10⁶ cells to about 4×10⁹ cells, about 4×10⁶ cells to about 3×10⁹ cells, about 5×10⁶ cells to about 2×10⁹ cells, about 6×10⁶ cells to about 1×10⁹ cells, 0.5×10⁶ cells to about 6×10⁸ cells, about 1×10⁶ cells to about 5×10⁸ cells, about 2×10⁶ cells to about 5×10⁸ cells, about 3×10⁶ cells to about 4×10⁸ cells, about 4×10⁶ cells to about 3×10⁸ cells, about 5×10⁶ cells to about 2×10⁸ cells, about 6×10⁶ cells to about 1×10⁸ cells, about 7×10⁶ cells to about 9×10⁸ cells, about 8×10⁶ cells to about 8×10⁸ cells, about 9×10⁶ cells to about 7×10⁸ cells, about 1×10⁷ cells to about 6×10⁸ cells, about 2×10⁷ cells to about 5×10⁸ cells, about 7×10⁶ cells to about 9×10⁷ cells, about 8×10⁶ cells to about 8×10⁷ cells, about 9×10⁶ cells to about 7×10⁷ cells, about 1×10⁷ cells to about 6×10⁷ cells, or about 2×10⁷ cells to about 5×10⁷ cells.

In some embodiments, a therapeutically effective dose comprises about 0.5×10⁵ cells to about 9×10¹⁰ cells. In some embodiments, a therapeutically effective dose comprises about 0.5×10⁶ cells to about 1×10¹⁰ cells. In some embodiments, a therapeutically effective dose comprises about 0.5×10⁶ cells to about 5×10⁹ cells. In some embodiments, a therapeutically effective dose comprises about 0.5×10⁶ cells to about 1×10⁹ cells. In some embodiments, a therapeutically effective dose comprises about 0.5×10⁶ cells to about 6×10⁸ cells. In some embodiments, a therapeutically effective dose comprises about 0.5×10⁶ cells to about 9×10¹⁰ cells. In some embodiments, a therapeutically effective dose comprises about 0.5×10⁷ cells to about 1×10¹⁰ cells. In some embodiments, a therapeutically effective dose comprises about 0.5×10⁷ cells to about 5×10⁹ cells. In some embodiments, a therapeutically effective dose comprises about 0.5×10⁷ cells to about 1×10⁹ cells. In some embodiments, a therapeutically effective dose comprises about 0.5×10⁷ cells to about 6×10⁸ cells. In some embodiments, a therapeutically effective dose comprises about 0.5×10⁸ cells to about 9×10¹⁰ cells. In some embodiments, a therapeutically effective dose comprises about 0.5×10⁸ cells to about 1×10¹⁰ cells. In some embodiments, a therapeutically effective dose comprises about 0.5×10⁸ cells to about 5×10⁹ cells. In some embodiments, a therapeutically effective dose comprises about 0.5×10⁸ cells to about 1×10⁹ cells. The term “about” as referred to in a therapeutically dose, can be, for example, ±0.5×10⁶ cells, ±0.5×10⁷ cells, or ±0.5×10⁸ cells.

### Kits and Articles of Manufacture

The disclosure provides kits and articles of manufacture comprising the polynucleotides and plasmids encoding the engineered receptors described herein, and immune cells edited using gene editing systems described herein and comprising the engineered receptors described herein. In some embodiments, the kit comprises articles such as vials, syringes and instructions for use.

In some embodiments, the kit comprises a polynucleotide or vector comprising a sequence encoding one or more engineered receptors of the disclosure.

In some embodiments, the kit comprises a plurality of immune cells comprising an engineered receptor as described herein. In some embodiments, the plurality of immune cells comprises a plurality of T cells.

### Definitions

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

As used herein, a “subsequence” refers to a length of contiguous amino acids or nucleotides that form a part of a sequence described herein. A subsequence may be identical to a part of a full length sequence when aligned to the full length sequence, or less than 100% identical to the part of the full length sequence to which it aligns (e.g., 90% identical to 50% of the full sequence, or the like).

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

A polynucleotide is “operably linked” to another polynucleotide when it is placed into a functional relationship with the other polynucleotide. For example, a promoter or enhancer is operably linked to a coding sequence if it affects the transcription of the sequence. A peptide is “operably linked” to another peptide when the polynucleotides encoding them are operably linked, preferably they are in the same open reading frame.

A “promoter” is a sequence of DNA needed to turn a gene on or off. Promoters are located immediately upstream and/or overlapping the transcription start site, and are usually between about one hundred to several hundred base pairs in length.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about,” when immediately preceding a number or numeral, means that the number or numeral ranges plus or minus 10%.

### Stem Cell Transplant

Stem cell transplant is a procedure designed to restore the blood-forming cells of a patient. Allogeneic stem cell transplant refers to treatment with stem cells derived from a donor, other than the patient (or a patient's identical twin). As preparation for transplant, conditioning therapy is administered to deplete the patient's stem and immune cells. In cancer patients, the conditioning therapy also serves to kill cancer cells. Then the stem cell transplantation is administered. The transplanted donor stem cells replace the stem cells of the patient and begin to generate blood and immune cells. These donor-derived cells restore the patient's blood and immune system. The transplanted cells or their progeny may also have graft versus tumor effects. For example, donor-derived T cells may kill cancer cells. Stem cell transplant may fail because (1) the donor cells fail to engraft; (2) the engrafted donor cells attack healthy cells of the patient, a syndrome called graft-versus-host disease (GvHD); or (3) cancer cells survive the therapy, resulting in relapse.

The present disclosure provides immune cells engineered to target patient blood cells and spare from killing donor allogeneic stem cells (termed “transplant-sparing immune cells”). The present disclosure provides methods of use of the engineered immune cells in combination with stem cell transplant.

In some embodiments, the engineered immune cells are manipulated ex vivo or in vivo. Immunce cells manipulation for allogenic stem cell transplantation is known in the art and is described, for example, in de Witte et al., Allogeneic Stem Cell Transplantation Platforms With Ex Vivo and In Vivo Immune Manipulations: Count and Adjust, HemaSphere, e580 (2021).

The transplant-sparing immune cells of the present disclosure are engineered to express at least one activator receptor as described herein that specifically binds at least one activator antigen (“A antigen”) present on patient blood cells. The transplant-sparing immune cells of the present disclosure are engineered to also express at least one inhibitor receptor as described herein that specifically binds at least one blocker antigen (“B antigen”) present on the donor allogenic stem cells cells and absent on patient blood cells. Gene typing approaches using next-generation sequencing short read technology is standard clinical care, as described in Furst, et al. (2019). Other methods for haplotyping prior to stem cell transplant are well known in the art and may be used in the practice of the presently disclosed methods.

In some embodiments, an allelic variant of a HLA protein may be used as the B antigen. Inhibitor receptors specific for HLA variants are known in the art and stem cell transplant recipients are routinely tested for HLA variants.

In some embodiments, the A antigen may be SPN, CD45, ITGAL, CD33, or FLT3.

In some embodiments, the B antigen may be an allelic variant of HLA (e.g., HLA-A*02). In some embodiments, allelic variants of HLA loci may also be a B antigen (HLA-A, -B, -C, -DRB1, -DQB1, -DPB1, etc).

The present disclosure provides an immune cell comprising an activator receptor comprising an extracellular ligand-binding domain specific to an activator antigen expressed by blood cells, and an inhibitor receptor comprising an extracellular ligand-binding domain specific to an inhibitor antigen, wherein the inhibitor antigen is an allelic variant of a Human Leukocyte Antigen (HLA).

In some embodiments, the activator antigen is expressed by hematopoietic cells, myeloid cells, or hematopoietic stem cells, or a combination thereof.

Provided herein are immune cells engineered to detect the haplotype mismatch between a subject's blood cells and donor stem cells, and uses thereof in, for example and without limitation, treating or preventing cancer. The immune cells are engineered to express a receptor system that activates the immune cell selectively when the immune cell contacts an activator antigen (“A antigen”) only when the immune cell fails to contact a blocker antigen (“B antigen”). The receptor system may be formed from one, two, or more receptors, and it may detect one, two or more A antigens as well as one, two or more B antigens.

In a two receptor system, the first receptor (an activator receptorrr, or “A module”) activates the immune cells, while the second receptor (an inhibitor receptorrrrr, or “B module”) inhibits activation of the immune cells by the first receptor. Each receptor contains a ligand-binding domain (LBD) that binds the A antigen or the B antigen, respectively. Signals from the two receptors upon ligand binding are integrated by the immune cell. Presence of the B antigen on cells of a stem cell transplant prevents killing of the stem cells by the immune cells, even when the cells of the stem cell transplant also express the A antigen. The blood cells of the subject are targeted by their expression of the A antigen, and not protected by expression of the B antigen, as the immune cells are engineered such that they recognize a variant of the B antigen not present on the subject's blood cells.

In some embodiments, the B antigen is an allelic variant Human Leukocyte Antigen (HLA). In such embodiments, the immune cell comprises an activator receptor comprising an extracellular ligand-binding domain specific to an activator antigen expressed by blood cells; and an inhibitor receptor comprising an extracellular ligand-binding domain specific to an inhibitor antigen, wherein the inhibitor antigen is an allelic variant of a Human Leukocyte Antigen (HLA).

In known stem cell transplant methods, recipients of stem cell transplant are tested to determine their haplotype; and the stem cell transplant is matched at as many HLA loci as possible. The present inventors have recognized that the stem cell transplant may be selected to have a mismatch at a predetermined HLA locus. The immune cells may then be engineered to use the allelic variant of this HLA present in the stem cell transplant, but absent in the recipient; or an off-the-shelf immune cell where the selected B antigen specificity may be chosen. Thereby, a system of the subject's cells, donor's stem cells, and engineered immune cells may be configured to permit selective killing of the subject's blood cells, while sparing the donor stem cells.

The compositions and methods provided herein comprise immune cells which target and kill blood cells, such as cancerous cells, as well as, permissively, other blood cells (e.g., bone marrow resident blood cells such as bone marrow stem cells). Because the stem cell transplant replaces the other blood cells of the subject, the subject tolerates killing of its blood cells, which would otherwise be poorly tolerated or even lethal. In variations, the engineered immune cells may be administered before, after, or concurrently with the stem cell transplant.

In some embodiments, the stem cell transplant comprises hematopeotic stem cells.

In some embodiments, the hematopeotic stem cells are CD34+ positively selected hematopeotic stem cells.

In some embodiments, the hematopeotic stem cells are CD3− negatively selected hematopeotic stem cells.

In some embodiments, the hematopeotic stem cells are CD19− negatively selected hematopeotic stem cells.

Stem cell transplant technology is known in the art and is described, for example, in Roldan et al., Allogeneic Stem Cell Transplantation with CD34. Cell Selection, Clinical Hematology International, 154-160 (2019).

The present disclosure provides a biobank, comprising a collection of immune cells according to the present disclosure and a collection of allogeneic stem cell transplants, wherein each allogeneic stem cell transplant is positive for the allelic variant of an HLA and each immune cell comprises an inhibitor receptor specific to one of the alleleic variants.

In some embodiments, the biobank comprises an immune cell whose inhibitor antigen is HLA-A*02 and the allogeneic stem cell transplant that are HLA-A*02+.

In some embodiments, the biobank comprises an immune cell whose inhibitor antigen is HLA-B*07−/−, the inhibitor antigen is HLA-B*07 and the allogeneic stem cell transplant are HLA-B*07+.

In some embodiments, the biobank comprises an immune cell whose inhibitor antigen is HLA-C*07−/−, the inhibitor antigen is HLA-C*07 and the allogeneic stem cell transplant are HLA-C*07+.

In some embodiments, the biobank comprises an immune cell whose inhibitor antigen is HLA-A*69−/−, the inhibitor antigen is HLA-A*69 and the allogeneic stem cell transplant are HLA-A*69+.

### Inhibitor Receptors

The disclosure provides a second receptor, comprising an extracellular ligand binding domain specific to an allogeneic donor cell antigenencoding the non-cancerous antigen, disruption of cellular signaling that regulates expression, such as an allelic variant of a gene. The allogeneic donor cell allelic variant can be absent in the patient cell. In compositions and methods disclosed herein, the cells or subject treated exhibit allelic variance with the allogeneic donor cells. Accordingly, the disclosure provides compositions and methods for killing cells and/or treating subject exhibiting allelic variation with the allogeneic donor cellantigen from any cause.

The allogeneic donor cell antigen can be a protein, or an antigen peptide thereof in a complex with a major histocompatibility complex class I (MHC-I), where the allogeneic donor cell antigen comprises a polymorphism. Because the allogeneic donor cell antigen is polymorphic, allelic variation of a single copy of the gene encoding the allogeneic donor cell antigen, yields a subject cell that retains the other polymorphic variant of gene. For example, an allogeneic donor cell may have HLA-A*02 and HLA-A*01 alleles at the HLA locus and a subject may have only the HLA-A*01 allele (the HLA-A*02 allele is absent). In such a subject, the HLA-A*01 protein remains present, but is not recognized by the inhibitory receptor of immune cells encountering the subject cell, because the inhibitor receptor is designed to be specific to the HLA-A*02 (or other allogeneic donor cell antigen). In allogeneic donor cells, the HLA-A*02 (or other allogeneic donor cell antigen) is present and inhibits activation of the engineered immune cell. In subject cells, the HLA-A*02 allelic variant (or other allogeneic donor cell antigen) is absent. Immune cells engineered to express the inhibitory receptor do not receive an inhibitory signal from the inhibitory receptor, as the inhibitory receptor only responds to the HLA-A*02 (or other allogeneic donor cell antigen), which is absent on subject cells. By this mechanism, the immune cell is selectively activated, and selectively kills, subject cells expressing an A antigen and missing HLA-A*02 (or another allogeneic donor cell antigen) thereby sparing allogeneic donor stem cells in allogeneic stem cell transplantation. HLA-A is used here as an example. Similar polymorphic variation occurs in the population at other MHC genes and in other non-MHC genes as well. Accordingly, the disclosure provides a second receptor, comprising an extracellular ligand binding domain specific to an allogeneic donor cell antigen selected from intercellular adhesion molecule 1 (ICAM1), catechol-O-methyltransferase (COMT), C-X-C motif chemokine ligand 16 (CXCL16), leucine rich repeat neuronal 4 (LRRN4) and uroplakin 3B (UPK3B), or an antigen peptide thereof in a complex with a major histocompatibility complex class I (MHC-I), wherein the allogeneic donor cell antigen may comprise a nonsynonymous, extracellular-domain polymorphism (e.g., in an extracellular domain of ICAM1, COMT, CXCL16), and immune cells comprising same. In some embodiments, the second receptor is an inhibitory chimeric antigen receptor.

Illustrative inhibitory receptors are described in PCT/US2020/045228 filed on Sep. 6, 2020, PCT/US2020/064607, filed on Dec. 11, 2020, PCT/US2021/029907, filed on Apr. 29, 2021 and PCT/US2020/059856 filed on Nov. 10, 2020, the contents of each of which are incorporated herein by reference.

In some embodiments, the second receptor is humanized.

The disclosure provides a second receptor, which is an inhibitory receptor, comprising an extracellular ligand binding that can discriminate between single amino-acid variant alleles of an antigen. This ability to discriminate between allelic variants of an antigen allows the second receptor to inhibit activation of immune cells comprising the second receptor in the presence of allogeneic donor cells that express that the allele recognized by the ligand binding domain. However, activation of immune cells is not inhibited in the presence of subject cells from which the allele is absent. The disclosure provides a second receptor, which is an inhibitory receptor, comprising an extracellular ligand binding that can discriminate between different levels of expression of an allogeneic donor cell antigen. This allows the second receptor to inhibit activation of immune cells comprising the second receptor in the presence of allogeneic donor cells that express the ligand for the second receptor, but to allow activation of immune cells in the presence of subject cells that express low levels, or have no expression, of the ligand for the second receptor.

### Allogeneic Donor Cell Antigens

In some embodiments, the allogeneic donor cell antigen is not expressed by the subject's cells, and is expressed by allogeneic donor cells. In some embodiments, the target cells are a plurality of subject cells that do not express the allogeneic donor cell antigen. In some embodiments, the allogeneic donor cell cells are a plurality of allogeneic donor cells that express both the A and B antigens.

Any cell surface molecule expressed by the allogeneic donor cells that is not expressed by the subject's cells may be a suitable allogeneic donor cell antigen for the second receptor extracellular ligand binding domain. For example, a cell adhesion molecule, a cell-cell signaling molecule, an extracellular domain, a molecule involved in chemotaxis, a glycoprotein, a G protein-coupled receptor, a transmembrane, a receptor for a neurotransmitter, or a voltage gated ion channel can be used as an allogeneic donor cell antigen.

Illustrative allogeneic donor cell antigens absent in the subject's cells include ICAM1, COMT and CXCL16. In some embodiments, the allogeneic donor cell antigen is selected from the group consisting of a polymorphic variant of ICAM1, COMT and CXCL16. In some embodiments, the allogeneic donor cell antigen is an antigen peptide comprising a polymorphic residue of ICAM1, COMT or CXCL16 in a complex with a major histocompatibility complex class I (MHC-I).

Allogeneic donor cell major histocompatibility complex class I MHC-I (or pMHC-I) antigens comprising any of HLA-A, HLA-B, HLA-C or HLA-E are envisaged as within the scope of the disclosure. In some embodiments, the allogeneic donor cell antigen comprises a Major Histocompatibility Complex (MHC) protein. In some embodiments, the MHC is MHC class I. In some embodiments, the MHC class I protein comprises a human leukocyte antigen (HLA) protein. In some embodiments, the allogeneic donor cell antigen comprises an allele of an HLA Class I protein selected from the group consisting of HLA-A, HLA-B, HLA-C, or HLA-E. In some embodiments, the HLA-A allele comprises HLA-A*01, HLA-A*02, HLA-A*03 or HLA-A*11. In some embodiments, the HLA-B allele comprises HLA-B*07. In some embodiments, the HLA-C allele comprises HLA-C*07.

In some embodiments, the inhibitor ligand is encoded by a gene that is absent or polymorphic. In some embodiments, the allogeneic donor cell antigen comprises HLA-A. In some embodiments, the allogeneic donor cell antigen comprises an allele of HLA-A. In some embodiments, the allele of HLA-A comprises HLA-A*01, HLA-A*02, HLA-A*03 or HLA-A*11. In some embodiments, the allogeneic donor cell antigen comprises HLA-A*69. In some embodiments, the allogeneic donor cell antigen comprises a human leukocyte antigen A*02 allele (HLA-A*02).

In some embodiments, the allogeneic donor cell antigen comprises an allele of HLA-B. In some embodiments, the allele of HLA-B comprises HLA-B*07.

In some embodiments, the allogeneic donor cell antigen comprises HLA-C. In some embodiments, the HLA-C allele comprises HLA-C*07.

In some embodiments, the allogeneic donor cell antigen comprises ICAM1 or an antigen peptide thereof in a complex with MHC-I. Human ICAM1 is frequently lost through LOH in cancer cells.

A wild type Human ICAM1 is described in NCBI record number NP_000192.2 the contents of which are incorporated by reference herein in their entirety. In some embodiments, ICAM1 comprises an amino acid sequence of: MAPSSPRPAL

(SEQ ID NO: 452)
PALLVLLGAL FPGPGNAQTS VSPSKVILPR GGSVLVTCST
SCDQPKLLGIETPLPKKELL LPGNNRKVYE LSNVQEDSQP
MCYSNCPDGQ STAKTFLTVY WTPER VELAPLPSWQPVGKN
LTLRCQVEGG APRANLTVVL LRGEKELKRE
PAVGEPAEVT TTVLVRRDHHGANFSCRTEL DLRPQGLELF
ENTSAPYQLQ TFVLPATPPQ LVSPRVLEVD
TQGTVVCSLDGLFPVSEAQV
HLALGDQRLN PTVTYGNDSF SAKASVSVTA EDEGTQRLTC
AVILGNQSQEEETLQTVTIYSF PAPNVILTKP EVSEGTEVTV
KCEAHPRAKV TLNGVPAQPL
GPRAQLLLKATPEDNGRSFS CSATLEVAGQ LIHKNQTREL
RVLYGPRLDE RDCPGNWTWP ENSQQTPMCQAWGNPLPELK
CLKDGTFPLP IGESVTVTRD
LEGTYLCRAR STQGEVTRKV TVNVLSPRYE IVIITVVAAA
VIMGTAGLST YLYNRQRKIK KYRLQQAQKG TPMKPNTQAT PP.

In some embodiments, ICAM1 comprises a sequence that shares at least 80%, at least 85%, at least 90%, at least 95%, at least 96%, at least 97%, at least 98%, at least 99% identity, or is 100% identical to SEQ ID NO: 452. Polymorphic residues of ICAM1 are marked as bold and underlined in SEQ ID NO: 452. For example, rs5498 is a polymorphism at position 469 of SEQ ID NO: 452, which can be a K or an E.

In some embodiments, the allogeneic donor cell antigen comprises a polymorphism of ICAM1. For example, the allogeneic donor cell antigen comprises a peptide derived from ICAM1 comprising a polymorphic residue of ICAM1. Polymorphic residues of ICAM1 include amino acid residue 469 of SEQ ID NO: 452. In some embodiments, the allogeneic donor cell antigen comprises a K at position 469 of SEQ ID NO: 452. In some embodiments, the allogeneic donor cell antigen comprises an E at position 469 of SEQ ID NO: 452.

In some embodiments, the allogeneic donor cell antigen comprises COMT or an antigen peptide thereof in a complex with MHC-I. Human COMT is frequently lost through LOH in cancer cells.

A wild type Human COMT is described in NCBI record number NP_000192.2, the contents of which are incorporated by reference herein in their entirety. In some embodiments, COMT comprises an amino acid sequence of: MPEAPPLLLA

(SEQ ID NO: 453)
AVLLGLVLLV VLLLLLRHWG WGLCLIGWNE FILQPIHNLL
MGDTKEQRIL NHVLQHAEPG NAQSVLEAID
TYCEQKEWAM NVGDKKGKIV DAVIQEHQPS
VLLELGAYCGYSAVRMARLL SPGARLITIE INPDCAAITQ
RMVDFAGVKD KVTLVVGASQ
DIIPQLKKKYDVDTLDMVFL DHWKDRYLPD TLLLEECGLL
RKGTVLLADN VICPGAPDFL AHVRGSSCFECTHYQSFLEY
REVVDGLEKA IYKGPGSEAG P.

In some embodiments, COMT comprises a sequence that shares at least 80%, at least 85%, at least 90%, at least 95%, at least 96%, at least 97%, at least 98%, at least 99% identity, or is 100% identical to SEQ ID NO: 453. Polymorphic residues of COMT include, but are not limited to, for example, V158M is a polymorphism at position 158 of SEQ ID NO: 453, which can be a V or an M.

Polymorphic residues of COMT 1 include amino acid residue 158 of SEQ ID NO: 453. In some embodiments, the allogeneic donor cell antigen comprises a peptide of COMT comprising amino acid 158 of SEQ ID NO: 453. In some embodiments, the allogeneic donor cell antigen comprises a V at position 158 of SEQ ID NO: 453. In some embodiments, the allogeneic donor cell antigen comprises an M at position 158 of SEQ ID NO: 453.

In some embodiments, the allogeneic donor cell antigen comprises C-X-C motif chemokine ligand 16 (CXCL16) or an antigen peptide thereof in a complex with MHC-I. Human CXCL16 precursor is described in NCBI record number NP_001094282.1, the contents of which are incorporated by reference herein in their entirety. In some embodiments, CXCL16 comprises an amino acid sequence of: MSGSQSEVAP SPQSPRSPEM GRDLRPGSRV LLLLLLLLLV YLTQPGNGNE GSVTGSCYCGKRISSDSPPS VQFMNRLRKH LRAYHRCLYY TRFQLLSWSV CGGNKDPWVQ ELMSCLDLKECGHAYSGIVA HQKHLLPTSP PISQASEGAS SDIHTPAQML LSTLQSTQRP TLPVGSLSSDKELTRPNETT IHTAGHSLAA GPEAGENQKQ PEKNAGPTAR TSATVPVLCL LAIIFILTAALSYVLCKRRR GQSPQSSPDL PVHYIPVAPD SNT (SEQ ID NO: 454).

In some embodiments, the allogeneic donor cell antigen comprises a polymorphism of CXCL16. For example, the allogeneic donor cell antigen comprises a peptide derived from CXCL16 comprising a polymorphic residue of CXCL16. Polymorphic residues of CXCL16 include positions 142 and 200 of SEQ ID NO: 454. In some embodiments, the allogeneic donor cell antigen comprises a peptide of CXCL16 comprising amino acid 142 or 200 of SEQ ID NO: 454. In some embodiments, the allogeneic donor cell antigen comprises a peptide of CXCL16 comprising an A at amino acid 200 of SEQ ID NO: 454. In some embodiments, the allogeneic donor cell antigen comprises a peptide of CXCL16 comprising a V at amino acid 200 of SEQ ID NO: 454. In some embodiments, the allogeneic donor cell antigen comprises a peptide of CXCL16 comprising an I at amino acid 142 of SEQ ID NO: 454. In some embodiments, the allogeneic donor cell antigen comprises a peptide of CXCL16 comprising a T at amino acid 142 of SEQ ID NO: 454.

In some embodiments, the allogeneic donor cell antigen comprises HLA-A*01, HLA-A*02, HLA-A*03, HLA-A*11, HLA-B*07 or HLA-C*07. Various single variable domains that bind to or recognize the specified HLA alleles, for use in embodiments described herein, are described in Table 7A. (complementarity determining regions underlined):

TABLE 7A
HLA scFv binding domains
HLA-A*02 antigen binding domains

| | |
|---|---|
| DVLMTQTPLSLPVSL | GATGTTTTGATGACCCAAACTCCACTCTCCCTGCCT |
| GDQASISCRSSQSIVH | GTCAGTCTTGGAGATCAAGCCTCCATCTCTTGCAG |
| SNGNTYLEWYLQKP | ATCTAGTCAGAGCATTGTACATAGTAATGGAAACA |
| GQSPKLLIYKVSNRF | CCTATTTAGAATGGTACCTGCAGAAACCAGGCCAG |
| SGVPDRFSGSGSGTD | TCTCCAAAGCTCCTGATCTACAAAGTTTCCAACCG |
| FTLKISRVEAEDLGV | ATTTTCTGGGGTCCCAGACAGGTTCAGTGGCAGTG |
| YYCFQGSHVPRTSGG | GATCAGGGACAGATTTCACACTCAAGATCAGTAGA |
| GTKLEIKGGGGSGGG | GTGGAGGCTGAGGATCTGGGAGTTTATTACTGCTT |
| GSGGGGSGGQVQLQ | TCAAGGTTCACATGTTCCTCGGACGTCCGGTGGAG |
| QSGPELVKPGASVRI | GCACCAAGCTGGAAATCAAAGGCGGAGGTGGAAG |
| SCKASGYTFTSYHIH | CGGAGGGGGAGGATCTGGCGGCGGAGGAAGCGGA |
| WVKQRPGQGLEWIG | GGCCAGGTCCAGCTGCAGCAGTCTGGACCTGAGCT |
| WIYPGNVNTEYNEK | GGTGAAGCCTGGGGCTTCAGTGAGGATATCCTGCA |
| FKGKATLTADKSSST | AGGCTTCTGGCTACACCTTCACAAGTTACCATATA |
| AYMHLSSLTSEDSAV | CATTGGGTGAAGCAGAGGCCTGGACAGGGACTTG |
| YFCAREEITYAMDY | AGTGGATTGGATGGATTTATCCTGGAAATGTTAAT |
| WGQGTSVTVSS (SEQ | ACTGAGTACAATGAGAAGTTCAAGGGCAAGGCCA |
| ID NO: 455) | CACTGACTGCAGACAAATCGTCCAGCACAGCCTAC |
| | ATGCACCTCAGCAGCCTGACCTCTGAGGACTCTGC |
| | GGTCTATTTCTGTGCCAGAGAGGAGATTACCTATG |
| | CTATGGACTACTGGGGTCAAGGAACCTCAGTCACC |
| | GTGTCCTCA (SEQ ID NO: 456) |
| QVQLVQSGAEVKKP | CAGGTGCAGCTGGTGCAGTCTGGGGCTGAGGTGAA |
| GSSVKVSCKASGYTF | GAAGCCTGGGTCCTCAGTGAAGGTTTCCTGCAAGG |
| TSYHIHWVRQAPGQ | CTTCTGGATACACCTTCACTAGCTATCATATACATT |
| GLEWMGWIYPGNVN | GGGTGCGCCAGGCCCCCGGACAAGGGCTTGAGTG |
| TEYNEKFKGKATITA | GATGGGATGGATCTACCCTGGCAATGTTAACACAG |
| DKSTSTAYMELSSLR | AATATAATGAGAAGTTCAAGGGCAAAGCCACCATT |
| SEDTAVYYCAREEIT | ACCGCGGACAAATCCACGAGCACAGCCTACATGG |
| YAMDYWGQGTTVT | AGCTGAGCAGCCTGAGATCTGAAGACACGGCTGTG |
| VSSGGGGSGGGGSG | TATTACTGTGCGAGGGAGGAAATTACCTACGCTAT |
| GGGSGGEIVLTQSPG | GGACTACTGGGGCCAGGGAACCACAGTCACCGTGT |
| TLSLSPGERATLSCRS | CCTCAGGCGGAGGTGGAAGCGGAGGGGGAGGATC |
| SQSIVHSNGNTYLEW | TGGCGGCGGAGGAAGCGGAGGCGAGATTGTATTG |
| YQQKPGQAPRLLIYK | ACCCAGAGCCCAGGCACCCTGAGCCTCTCTCCAGG |
| VSNRFSGIPDRFSGSG | AGAGCGGGCCACCCTCAGTTGTAGATCCAGTCAGA |
| SGTDFTLTISRLEPED | GTATTGTACACAGTAATGGGAACACCTATTTGGAA |
| FAVYYCFQGSHVPRT | TGGTATCAGCAGAAACCAGGTCAAGCCCCAAGATT |
| FGGGTKVEIK (SEQ | GCTCATCTACAAAGTCTCTAACAGATTTAGTGGTA |
| ID NO: 457) | TTCCAGACAGGTTCAGCGGTTCCGGAAGTGGTACT |
| | GATTTCACCCTCACGATCTCCAGGCTCGAGCCAGA |
| | AGATTTCGCCGTTTATTACTGTTTTCAAGGTTCACA |
| | TGTGCCGCGCACATTCGGTGGGGGTACTAAAGTAG |
| | AAATCAAA (SEQ ID NO: 458) |
| QVQLVQSGAEVKKP | CAGGTGCAGCTGGTGCAGTCTGGGGCTGAGGTGAA |
| GSSVKVSCKASGYTF | GAAGCCTGGGTCCTCAGTGAAGGTTTCCTGCAAGG |
| TSYHIHWVRQAPGQ | CTTCTGGATACACCTTCACTAGCTATCATATACATT |
| GLEWMGWIYPGNVN | GGGTGCGCCAGGCCCCCGGACAAGGGCTTGAGTG |
| TEYNEKFKGKATITA | GATGGGATGGATCTACCCTGGCAATGTTAACACAG |
| DKSTSTAYMELSSLR | AATATAATGAGAAGTTCAAGGGCAAAGCCACCATT |
| SEDTAVYYCAREEIT | ACCGCGGACAAATCCACGAGCACAGCCTACATGG |
| YAMDYWGQGTTVT | AGCTGAGCAGCCTGAGATCTGAAGACACGGCTGTG |
| VSSGGGGSGGGGSG | TATTACTGTGCGAGGGAGGAAATTACCTACGCTAT |
| GGGSGGDIVMTQTPL | GGACTACTGGGGCCAGGGAACCACAGTCACCGTGT |
| SLPVTPGEPASISCRS | CCTCAGGCGGAGGTGGAAGCGGAGGGGGAGGATC |
| SQSIVHSNGNTYLEW | TGGCGGCGGAGGAAGCGGAGGCGACATTGTAATG |
| YLQKPGQSPQLLIYK | ACCCAGACCCCACTCAGCCTGCCCGTCACTCCAGG |
| VSNRFSGVPDRFSGS | AGAGCCGGCCAGCATCAGTTGTAGATCCAGTCAGA |
| GSGTDFTLKISRVEA | GTATTGTACACAGTAATGGGAACACCTATTTGGAA |
| EDVGVYYCFQGSHV | TGGTATCTGCAGAAACCAGGTCAATCCCCACAATT |
| PRTFGGGTKVEIK | GCTCATCTACAAAGTCTCTAACAGATTTAGTGGTG |
| (SEQ ID NO: 459) | TACCAGACAGGTTCAGCGGTTCCGGAAGTGGTACT |
| | GATTTCACCCTCAAGATCTCCAGGGTCGAGGCAGA |
| | AGATGTCGGCGTTTATTACTGTTTTCAAGGTTCACA |
| | TGTGCCGCGCACATTCGGTGGGGGTACTAAAGTAG |
| | AAATCAAA (SEQ ID NO: 460) |
| EVQLVESGGGLVKP | GAGGTGCAGCTGGTGGAGTCTGGGGGTGGGCTGGT |
| GGSLRLSCAASGYTF | GAAGCCTGGGGGCTCACTGAGGCTTTCCTGCGCGG |
| TSYHIHWVRQAPGK | CTTCTGGATACACCTTCACTAGCTATCATATACATT |
| GLEWVGWIYPGNVN | GGGTGCGCCAGGCCCCCGGAAAAGGGCTTGAGTG |
| TEYNEKFKGRFTISR | GGTGGGATGGATCTACCCTGGCAATGTTAACACAG |
| DDSKNTLYLQMNSL | AATATAATGAGAAGTTCAAGGGCAGATTCACCATT |
| KTEDTAVYYCAREEI | AGCAGGGACGATTCCAAGAACACACTCTACCTGCA |
| TYAMDYWGQGTTV | GATGAACAGCCTGAAAACTGAAGACACGGCTGTGT |
| TVSSGGGGSGGGGS | ATTACTGTGCGAGGGAGGAAATTACCTACGCTATG |
| GGGGSGGDIQMTQS | GACTACTGGGGCCAGGGAACCACAGTCACCGTGTC |
| PSSLSASVGDRVTITC | CTCAGGCGGAGGTGGAAGCGGAGGGGGAGGATCT |
| RSSQSIVHSNGNTYL | GGCGGCGGAGGAAGCGGAGGCGACATTCAAATGA |
| EWYQQKPGKAPKLL | CCCAGAGCCCATCCAGCCTGAGCGCATCTGTAGGT |
| IYKVSNRFSGVPSRFS | GACCGGGTCACCATCACTTGTAGATCCAGTCAGAG |
| GSGSGTDFTLTISSLQ | TATTGTACACAGTAATGGGAACACCTATTTGGAAT |
| PEDFATYYCFQGSHV | GGTATCAGCAGAAACCAGGTAAAGCCCCAAAATT |
| PRTFGGGTKVEIK | GCTCATCTACAAAGTCTCTAACAGATTTAGTGGTG |
| (SEQ ID NO: 461) | TACCAAGCAGGTTCAGCGGTTCCGGAAGTGGTACT |
| | GATTTCACCCTCACGATCTCCTCTCTCCAGCCAGAA |
| | GATTTCGCCACTTATTACTGTTTTCAAGGTTCACAT |
| | GTGCCGCGCACATTCGGTGGGGGTACTAAAGTAGA |
| | AATCAAA (SEQ ID NO: 462) |
| QVQLVQSGAEVKKP | CAGGTGCAGCTGGTGCAGTCTGGGGCTGAGGTGAA |
| GSSVKVSCKASGYTF | GAAGCCTGGGTCCTCAGTGAAGGTTTCCTGCAAGG |
| TSYHIHWVRQAPGQ | CTTCTGGATACACCTTCACTAGCTATCATATACATT |
| GLEWIGWIYPGNVN | GGGTGCGCCAGGCCCCCGGACAAGGGCTTGAGTG |
| TEYNEKFKGKATITA | GATCGGATGGATCTACCCTGGCAATGTTAACACAG |
| DESTNTAYMELSSLR | AATATAATGAGAAGTTCAAGGGCAAAGCCACCATT |
| SEDTAVYYCAREEIT | ACCGCGGACGAATCCACGAACACAGCCTACATGG |
| YAMDYWGQGTLVT | AGCTGAGCAGCCTGAGATCTGAAGACACGGCTGTG |
| VSSGGGGSGGGGSG | TATTACTGTGCGAGGGAGGAAATTACCTACGCTAT |
| GGGSGGDIQMTQSPS | GGACTACTGGGGCCAGGGAACCCTGGTCACCGTGT |
| TLSASVGDRVTITCR | CCTCAGGCGGAGGTGGAAGCGGAGGGGGAGGATC |
| SSQSIVHSNGNTYLE | TGGCGGCGGAGGAAGCGGAGGCGACATTCAAATG |
| WYQQKPGKAPKLLI | ACCCAGAGCCCATCCACCCTGAGCGCATCTGTAGG |
| YKVSNRFSGVPARFS | TGACCGGGTCACCATCACTTGTAGATCCAGTCAGA |
| GSGSGTEFTLTISSLQ | GTATTGTACACAGTAATGGGAACACCTATTTGGAA |
| PDDFATYYCFQGSH | TGGTATCAGCAGAAACCAGGTAAAGCCCCAAAATT |
| VPRTFGQGTKVEVK | GCTCATCTACAAAGTCTCTAACAGATTTAGTGGTG |
| (SEQ ID NO: 463) | TACCAGCCAGGTTCAGCGGTTCCGGAAGTGGTACT |
| | GAATTCACCCTCACGATCTCCTCTCTCCAGCCAGAT |
| | GATTTCGCCACTTATTACTGTTTTCAAGGTTCACAT |
| | GTGCCGCGCACATTCGGTCAGGGTACTAAAGTAGA |
| | AGTCAAA (SEQ ID NO: 464) |
| QVQLVQSGAEVKKP | CAGGTGCAGCTGGTGCAGTCTGGGGCTGAGGTGAA |
| GSSVKVSCKASGYTF | GAAGCCTGGGTCCTCAGTGAAGGTTTCCTGCAAGG |
| TSYHMHWVRQAPG | CTTCTGGATACACCTTCACTAGCTATCATATGCATT |
| QGLEWIGYIYPGNVN | GGGTGCGCCAGGCCCCCGGACAAGGGCTTGAGTG |
| TEYNEKFKGKATLT | GATCGGATACATCTACCCTGGCAATGTTAACACAG |
| ADKSTNTAYMELSSL | AATATAATGAGAAGTTCAAGGGCAAAGCCACCCTT |
| RSEDTAVYFCAREEI | ACCGCGGACAAATCCACGAACACAGCCTACATGG |
| TYAMDYWGQGTLV | AGCTGAGCAGCCTGAGATCTGAAGACACGGCTGTG |
| TVSSGGGGSGGGGS | TATTTCTGTGCGAGGGAGGAAATTACCTACGCTAT |
| GGGGSGGDVQMTQS | GGACTACTGGGGCCAGGGAACCCTGGTCACCGTGT |
| PSTLSASVGDRVTITC | CCTCAGGCGGAGGTGGAAGCGGAGGGGGAGGATC |
| SSSQSIVHSNGNTYM | TGGCGGCGGAGGAAGCGGAGGCGACGTTCAAATG |
| EWYQQKPGKAPKLL | ACCCAGAGCCCATCCACCCTGAGCGCATCTGTAGG |
| IYKVSNRFSGVPDRF | TGACCGGGTCACCATCACTTGTAGCTCCAGTCAGA |
| SGSGSGTEFTLTISSL | GTATTGTACACAGTAATGGGAACACCTATATGGAA |
| QPDDFATYYCHQGS | TGGTATCAGCAGAAACCAGGTAAAGCCCCAAAATT |
| HVPRTFGQGTKVEV | GCTCATCTACAAAGTCTCTAACAGATTTAGTGGTG |
| K (SEQ ID NO: 465) | TACCAGACAGGTTCAGCGGTTCCGGAAGTGGTACT |
| | GAATTCACCCTCACGATCTCCTCTCTCCAGCCAGAT |
| | GATTTCGCCACTTATTACTGTCATCAAGGTTCACAT |
| | GTGCCGCGCACATTCGGTCAGGGTACTAAAGTAGA |
| | AGTCAAA (SEQ ID NO: 466) |
| QVQLQQSGPELVKP | CAGGTGCAGCTGCAGCAGTCTGGGCCTGAGCTGGT |
| GASVKMSCKASGYT | GAAGCCTGGGGCCTCAGTGAAGATGTCCTGCAAGG |
| FTSYHIQWVKQRPG | CTTCTGGATACACCTTCACTAGCTATCATATCCAGT |
| QGLEWIGWIYPGDGS | GGGTGAAGCAGAGGCCTGGACAAGGGCTTGAGTG |
| TQYNEKFKGKTTLT | GATCGGATGGATCTACCCTGGCGATGGTAGTACAC |
| ADKSSSTAYMLLSSL | AGTATAATGAGAAGTTCAAGGGCAAAACCACCCTT |
| TSEDSAIYFCAREGT | ACCGCGGACAAATCCTCCAGCACAGCCTACATGTT |
| YYAMDYWGQGTSV | GCTGAGCAGCCTGACCTCTGAAGACTCTGCTATCT |
| TVSSGGGGSGGGGS | ATTTCTGTGCGAGGGAGGGGACCTACTACGCTATG |
| GGGGSGGDVLMTQT | GACTACTGGGGCCAGGGAACCTCAGTCACCGTGTC |
| PLSLPVSLGDQVSISC | CTCAGGCGGAGGTGGAAGCGGAGGGGGAGGATCT |
| RSSQSIVHSNGNTYL | GGCGGCGGAGGAAGCGGAGGCGATGTTTTGATGA |
| EWYLQKPGQSPKLLI | CCCAGACTCCACTCTCCCTGCCTGTCTCTCTTGGAG |
| YKVSNRFSGVPDRFS | ACCAAGTCTCCATCTCTTGTAGATCCAGTCAGAGT |
| GSGSGTDFTLKISRV | ATTGTACACAGTAATGGGAACACCTATTTAGAATG |
| EAEDLGVYYCFQGS | GTATCTGCAGAAACCAGGTCAGTCTCCAAAGTTGC |
| HVPRTFGGGTKLEIK | TCATCTACAAAGTCTCTAACAGATTTAGTGGTGTA |
| (SEQ ID NO: 467) | CCAGACAGGTTCAGCGGTTCCGGAAGTGGTACTGA |
| | TTTCACCCTCAAGATCTCGAGAGTGGAGGCTGAGG |
| | ATCTGGGAGTTTATTACTGTTTTCAAGGTTCACATG |
| | TGCCGCGCACATTCGGTGGAGGTACTAAACTGGAA |
| | ATCAAA (SEQ ID NO: 468) |
| QLQLQESGPGLVKPS | CAGCTGCAGCTGCAGGAGTCTGGGCCCGGGCTGGT |
| ETLSLTCTVSGYTFTS | GAAGCCTTCGGAAACGCTGAGCCTCACCTGCACGG |
| YHIQWIRQPPGKGLE | TTTCTGGATACACCTTCACCAGCTATCATATCCAGT |
| WIGWIYPGDGSTQY | GGATCCGACAGCCCCCTGGAAAAGGGCTTGAGTGG |
| NEKFKGRATISVDTS | ATCGGATGGATCTACCCTGGCGATGGTTCAACACA |
| KNQFSLNLDSVSAAD | GTACAATGAGAAGTTCAAGGGCAGAGCCACGATT |
| TAIYYCAREGTYYA | AGCGTGGACACATCCAAGAACCAATTCTCCCTGAA |
| MDYWGKGSTVTVSS | CCTGGACAGCGTGAGTGCTGCGGACACGGCCATTT |
| GGGGSGGGGSGGGG | ATTACTGTGCGAGAGAGGGAACTTACTACGCTATG |
| SGGDIQMTQSPSSLS | GACTACTGGGGCAAAGGGAGCACGGTCACCGTGTC |
| ASVGDRVTITCRSSQ | CTCAGGCGGAGGTGGAAGCGGAGGGGGAGGATCT |
| SIVHSNGNTYLEWY | GGCGGCGGAGGAAGCGGAGGCGACATCCAGATGA |
| QQKPGKAPKLLIYKV | CCCAGAGCCCAAGCTCCCTGAGTGCGTCCGTGGGC |
| SNRFSGVPSRFSGSGS | GACCGCGTGACCATCACTTGCAGATCCTCTCAGTC |
| GTDFTFTISSLQPEDI | CATCGTGCACTCCAACGGCAACACGTACCTCGAGT |
| ATYYCFQGSHVPRTF | GGTACCAGCAGAAGCCCGGGAAGGCCCCGAAACT |
| GPGTKVDIK (SEQ ID | GCTCATCTACAAGGTGAGCAACCGGTTCTCCGGCG |
| NO: 469) | TCCCCAGCCGCTTCTCAGGGTCCGGCTCGGGGACG |
| | GATTTCACCTTCACGATTAGCAGCTTGCAGCCCGA |
| | AGACATCGCCACGTACTACTGCTTTCAGGGAAGTC |
| | ACGTGCCGCGTACCTTCGGGCCGGGCACGAAAGTG |
| | GATATTAAG (SEQ ID NO: 470) |
| EVQLVQSGAELKKP | GAGGTGCAGCTGGTGCAGTCTGGGGCCGAGCTGAA |
| GSSVKVSCKASGYTF | GAAGCCTGGGTCCTCGGTGAAGGTGTCCTGCAAGG |
| TSYHIQWVKQAPGQ | CTTCTGGATACACCTTCACCAGCTATCATATCCAGT |
| GLEWIGWIYPGDGST | GGGTAAAACAGGCCCCTGGACAAGGGCTTGAGTG |
| QYNEKFKGKATLTV | GATCGGATGGATCTACCCTGGCGATGGTTCAACAC |
| DKSTNTAYMELSSLR | AGTACAATGAGAAGTTCAAGGGCAAAGCCACGCTT |
| SEDTAVYYCAREGT | ACCGTGGACAAATCCACGAACACAGCCTACATGGA |
| YYAMDYWGQGTLV | GCTGAGCAGCCTGAGATCTGAGGACACGGCCGTAT |
| TVSSGGGGSGGGGS | ATTACTGTGCGAGAGAGGGAACTTACTACGCTATG |
| GGGGSGGDIQMTQS | GACTACTGGGGCCAAGGGACCCTGGTCACCGTGTC |
| PSTLSASVGDRVTITC | CTCAGGCGGAGGTGGAAGCGGAGGGGGAGGATCT |
| RSSQSIVHSNGNTYL | GGCGGCGGAGGAAGCGGAGGCGACATCCAGATGA |
| EWYQQKPGKAPKLL | CCCAGAGCCCATCCACCCTGAGTGCGTCCGTGGGC |
| IYKVSNRFSGVPSRFS | GACCGCGTGACCATCACTTGCAGATCCTCTCAGTC |
| GSGSGTDFTLTISSLQ | CATCGTGCACTCCAACGGCAACACGTACCTCGAGT |
| PDDFATYYCFQGSH | GGTACCAGCAGAAGCCCGGGAAGGCCCCGAAACT |
| VPRTFGQGTKVEVK | GCTCATCTACAAGGTGAGCAACCGGTTCTCCGGCG |
| (SEQ ID NO: 471) | TCCCCAGCCGCTTCTCAGGGTCCGGCTCGGGGACG |
| | GATTTCACCCTCACGATTAGCAGCTTGCAGCCCGA |
| | TGACTTCGCCACGTACTACTGCTTTCAGGGAAGTC |
| | ACGTGCCGCGTACCTTCGGGCAGGGCACGAAAGTG |
| | GAAGTTAAG (SEQ ID NO: 472) |
| QVQLVQSGAEVKKP | CAGGTGCAGCTGGTGCAGTCTGGGGCCGAGGTGAA |
| GSSVKVSCKASGYTF | GAAGCCTGGGTCCTCGGTGAAGGTGTCCTGCAAGG |
| TSYHIQWVRQAPGQ | CTTCTGGATACACCTTCACCAGCTATCATATCCAGT |
| GLEWMGWIYPGDGS | GGGTACGACAGGCCCCTGGACAAGGGCTTGAGTG |
| TQYNEKFKGRVTITA | GATGGGATGGATCTACCCTGGCGATGGTTCAACAC |
| DKSTSTAYMELSSLR | AGTACAATGAGAAGTTCAAGGGCAGAGTCACGATT |
| SEDTAVYYCAREGT | ACCGCGGACAAATCCACGAGCACAGCCTACATGG |
| YYAMDYWGQGTTV | AGCTGAGCAGCCTGAGATCTGAGGACACGGCCGTA |
| TVSSGGGGSGGGGS | TATTACTGTGCGAGAGAGGGAACTTACTACGCTAT |
| GGGGSGGEIVLTQSP | GGACTACTGGGGCCAAGGGACCACGGTCACCGTGT |
| GTLSLSPGERATLSC | CCTCAGGCGGAGGTGGAAGCGGAGGGGGAGGATC |
| RSSQSIVHSNGNTYL | TGGCGGCGGAGGAAGCGGAGGCGAGATCGTCCTG |
| EWYQQKPGQAPRLLI | ACCCAGAGCCCAGGGACCCTGAGTTTGTCCCCGGG |
| YKVSNRFSGIPDRFS | CGAGCGCGCGACCCTCAGTTGCAGATCCTCTCAGT |
| GSGSGTDFTLTISRLE | CCATCGTGCACTCCAACGGCAACACGTACCTCGAG |
| PEDFAVYYCFQGSH | TGGTACCAGCAGAAGCCCGGGCAGGCCCCGCGACT |
| VPRTFGGGTKVEIK | GCTCATCTACAAGGTGAGCAACCGGTTCTCCGGCA |
| (SEQ ID NO: 473) | TCCCCGACCGCTTCTCAGGGTCCGGCTCGGGGACG |
| | GATTTCACCCTCACGATTAGCCGCTTGGAGCCCGA |
| | AGACTTCGCCGTGTACTACTGCTTTCAGGGAAGTC |
| | ACGTGCCGCGTACCTTCGGGGGGGGCACGAAAGTG |
| | GAAATTAAG (SEQ ID NO: 474) |
| QVTLKQSGAEVKKP | CAGGTGACCCTGAAGCAGTCTGGGGCCGAGGTGA |
| GSSVKVSCTASGYTF | AGAAGCCTGGGTCCTCGGTGAAGGTGTCCTGCACG |
| TSYHVSWVRQAPGQ | GCTTCTGGATACACCTTCACCAGCTATCATGTCAGC |
| GLEWLGRIYPGDGST | TGGGTACGACAGGCCCCTGGACAAGGGCTTGAGTG |
| QYNEKFKGKVTITAD | GTTGGGAAGGATCTACCCTGGCGATGGTTCAACAC |
| KSMDTSFMELTSLTS | AGTACAATGAGAAGTTCAAGGGCAAAGTCACGATT |
| EDTAVYYCAREGTY | ACCGCGGACAAATCCATGGACACATCCTTCATGGA |
| YAMDLWGQGTLVT | GCTGACCAGCCTGACATCTGAGGACACGGCCGTAT |
| VSSGGGGSGGGGSG | ATTACTGTGCGAGAGAGGGAACTTACTACGCTATG |
| GGGSGGEIVLTQSPG | GACCTCTGGGGCCAAGGGACCCTGGTCACCGTGTC |
| TLSLSPGERATLSCRS | CTCAGGCGGAGGTGGAAGCGGAGGGGGAGGATCT |
| SQSIVHSNGNTYLAW | GGCGGCGGAGGAAGCGGAGGCGAGATCGTCCTGA |
| YQQKPGQAPRLLISK | CCCAGAGCCCAGGGACCCTGAGTTTGTCCCCGGGC |
| VSNRFSGVPDRFSGS | GAGCGCGCGACCCTCAGTTGCAGATCCTCTCAGTC |
| GSGTDFTLTISRLEPE | CATCGTGCACTCCAACGGCAACACGTACCTCGCGT |
| DFAVYYCQQGSHVP | GGTACCAGCAGAAGCCCGGGCAGGCCCCGCGACT |
| RTFGGGTKVEIK | GCTCATCTCCAAGGTGAGCAACCGGTTCTCCGGCG |
| (SEQ ID NO: 475) | TCCCCGACCGCTTCTCAGGGTCCGGCTCGGGGACG |
| | GATTTCACCCTCACGATTAGCCGCTTGGAGCCCGA |
| | AGACTTCGCCGTGTACTACTGCCAACAGGGAAGTC |
| | ACGTGCCGCGTACCTTCGGGGGGGGCACGAAAGTG |
| | GAAATTAAG (SEQ ID NO: 476) |
| QVQLVQSGAEVKKP | CAGGTGCAGCTGGTGCAGTCTGGGGCCGAGGTGAA |
| GASVKVSCKASGYT | GAAGCCTGGGGCCTCGGTGAAGGTGTCCTGCAAGG |
| FTSYHMHWVRQAPG | CTTCTGGATACACCTTCACCAGCTATCATATGCACT |
| QRLEWMGWIYPGDG | GGGTACGACAGGCCCCTGGACAAAGGCTTGAGTG |
| STQYNEKFKGKVTIT | GATGGGATGGATCTACCCTGGCGATGGTTCAACAC |
| RDTSASTAYMELSSL | AGTACAATGAGAAGTTCAAGGGCAAAGTCACGATT |
| RSEDTAVYYCAREG | ACCCGGGACACATCCGCGAGCACAGCCTACATGGA |
| TYYAMDYWGQGTL | GCTGAGCAGCCTGAGATCTGAGGACACGGCCGTAT |
| VTVSSGGGGSGGGG | ATTACTGTGCGAGAGAGGGAACTTACTACGCTATG |
| SGGGGSGGDIVMTQ | GACTACTGGGGCCAAGGGACCCTGGTCACCGTGTC |
| TPLSLPVTPGEPASIS | CTCAGGCGGAGGTGGAAGCGGAGGGGGAGGATCT |
| CRSSQSIVHSNGNTY | GGCGGCGGAGGAAGCGGAGGCGACATCGTCATGA |
| LDWYLQKPGQSPQL | CCCAGACCCCACTGTCCCTGCCTGTGACCCCGGGC |
| LIYKVSNRFSGVPDR | GAGCCCGCGAGCATCAGTTGCAGATCCTCTCAGTC |
| FSGSGSGTDFTLKISR | CATCGTGCACTCCAACGGCAACACGTACCTCGACT |
| VEAEDVGVYYCMQ | GGTACCTGCAGAAGCCCGGGCAGTCCCCGCAACTG |
| GSHVPRTFGGGTKVE | CTCATCTACAAGGTGAGCAACCGGTTCTCCGGCGT |
| IK (SEQ ID NO: 477) | CCCCGACCGCTTCTCAGGGTCCGGCTCGGGGACGG |
| | ATTTCACCCTCAAGATTAGCCGCGTGGAGGCCGAA |
| | GACGTCGGCGTGTACTACTGCATGCAGGGAAGTCA |
| | CGTGCCGCGTACCTTCGGGGGGGGCACGAAAGTGG |
| | AAATTAAG (SEQ ID NO: 478) |

HLA-B*07 antigen binding domains

| | |
|---|---|
| 1.10_scFv | QVQLQESGPGLVKPSQTLSLTCTVSGYSITSGYSWHW |
| | IRQPPGKGLEWIGYIHFSGSTHYHPSLKSRVTISVDTS |
| | KNQFSLKLSSVTAADTAVYYCARGGVVSHYAMDCW |
| | GQGTTVTVSSGGGGSGGGGSGGGGSGGDIQMTQSPS |
| | SLSASVGDRVTITCRASENIYSNLAWYQQKPGKAPKL |
| | LIYAATYLPDGVPSRFSGSGSGTDFTLTISSLQPEDFA |
| | TYYCQHFWVTPYTFGGGTKVEIK (SEQ ID NO: 479) |
| 1.9_scFv | EVQLVESGGGLVKPGGSLRLSCAASGYSITSGYSWH |
| | WVRQAPGKGLEWVSYIHFSGSTHYHPSLKSRFTISRD |
| | NAKNSLYLQMNSLRAEDTAVYYCARGGVVSHYAM |
| | DCWGQGTTVTVSSGGGGSGGGGSGGGGSGGDIQMT |
| | QSPSSVSASVGDRVTITCRASENIYSNLAWYQQKPGK |
| | APKLLIYAATYLPDGVPSRFSGSGSGTDFTLTISSLQP |
| | EDFATYYCQHFWVTPYTFGGGTKVEIK (SEQ ID NO: |
| | 480) |
| 1.8_scFv | EVQLVESGGGLVKPGGSLRLSCAASGYSITSGYSWH |
| | WVRQAPGKGLEWVGYIHFSGSTHYHPSLKSRFTISRD |
| | DSKNTLYLQMNSLKTEDTAVYYCARGGVVSHYAMD |
| | CWGQGTTVTVSSGGGGSGGGGSGGGGSGGEIVLTQS |
| | PATLSLSPGERATLSCRASENIYSNLAWYQQKPGQAP |
| | RLLIYAATYLPDGIPARFSGSGSGTDFTLTISSLEPEDF |
| | AVYYCQHFWVTPYTFGGGTKVEIK (SEQ ID NO: 481) |
| 1.7 scFv | QVQLQQSGPGLVKPSQTLSLTCAISGYSITSGYSWHW |
| | IRQSPSRGLEWLGYIHFSGSTHYHPSLKSRITINPDTSK |
| | NQFSLQLNSVTPEDTAVYYCARGGVVSHYAMDCWG |
| | QGTTVTVSSGGGGSGGGGSGGGGSGGEIVLTQSPAT |
| | LSLSPGERATLSCRASENIYSNLAWYQQKPGQAPRLL |
| | IYAATYLPDGIPARFSGSGSGTDFTLTISRLEPEDFAVY |
| | YCQHFWVTPYTFGGGTKVEIK (SEQ ID NO: 482) |
| 1.6_scFv | EVQLVESGGGLVKPGGSLRLSCAASGYSITSGYSWH |
| | WVRQAPGKGLEWVGYIHFSGSTHYHPSLKSRFTISRD |
| | DSKNTLYLQMNSLKTEDTAVYYCARGGVVSHYAMD |
| | CWGQGTTVTVSSGGGGSGGGGSGGGGSGGDIQMTQ |
| | SPSSVSASVGDRVTITCRASENIYSNLAWYQQKPGKA |
| | PKLLIYAATYLPDGVPSRFSGSGSGTDFTLTISSLQPE |
| | DFATYYCQHFWVTPYTFGGGTKVEIK (SEQ ID NO: |
| | 483) |
| 1.5_scFv | EVQLVESGGGLVQPGGSLRLSCAASGYSITSGYSWH |
| | WVRQAPGKGLEWVSYIHFSGSTHYHPSLKSRFTISRD |
| | NSKNTLYLQMNSLRAEDTAVYYCARGGVVSHYAM |
| | DCWGQGTTVTVSSGGGGSGGGGSGGGGSGGDIQMT |
| | QSPSSLSASVGDRVTITCRASENIYSNLAWYQQKPGK |
| | APKLLIYAATYLPDGVPSRFSGSGSGTDFTLTISSLQP |
| | EDFATYYCQHFWVTPYTFGGGTKVEIK (SEQ ID NO: |
| | 484) |
| 1.4_scFv | EVQLVESGGGLVKPGGSLRLSCAASGYSITSGYSWH |
| | WVRQAPGKGLEWVGYIHFSGSTHYHPSLKSRFTISRD |
| | DSKNTLYLQMNSLKTEDTAVYYCARGGVVSHYAMD |
| | CWGQGTTVTVSSGGGGSGGGGSGGGGSGGDIQMTQ |
| | SPSSLSASVGDRVTITCRASENIYSNLAWYQQKPGKA |
| | PKLLIYAATYLPDGVPSRFSGSGSGTDFTLTISSLQPE |
| | DFATYYCQHFWVTPYTFGGGTKVEIK (SEQ ID NO: |
| | 485) |
| 1.3_scFv | QVQLQQWGAGLLKPSETLSLTCAVYGYSITSGYSWH |
| | WIRQPPGKGLEWIGYIHFSGSTHYHPSLKSRVTISVDT |
| | SKNQFSLKLSSVTAADTAVYYCARGGVVSHYAMDC |
| | WGQGTTVTVSSGGGGSGGGGSGGGGSGGDIQMTQS |
| | PSSLSASVGDRVTITCRASENIYSNLAWYQQKPGKAP |
| | KLLIYAATYLPDGVPSRFSGSGSGTDFTLTISSLQPED |
| | FATYYCQHFWVTPYTFGGGTKVEIK (SEQ ID NO: |
| | 486) |
| 1.2_scFv | QVQLQESGPGLVKPSQTLSLTCTVSGYSITSGYSWHW |
| | IRQHPGKGLEWIGYIHFSGSTHYHPSLKSRVTISVDTS |
| | KNQFSLKLSSVTAADTAVYYCARGGVVSHYAMDCW |
| | GQGTTVTVSSGGGGSGGGGSGGGGSGGDIQMTQSPS |
| | SLSASVGDRVTITCRASENIYSNLAWYQQKPGKAPKL |
| | LIYAATYLPDGVPSRFSGSGSGTDFTLTISSLQPEDFA |
| | TYYCQHFWVTPYTFGGGTKVEIK (SEQ ID NO: 487) |
| 1.1_scFv | QVQLQQSGPGLVKPSQTLSLTCAISGYSITSGYSWHW |
| | IRQSPSRGLEWLGYIHFSGSTHYHPSLKSRITINPDTSK |
| | NQFSLQLNSVTPEDTAVYYCARGGVVSHYAMDCWG |
| | QGTTVTVSSGGGGSGGGGSGGGGSGGDIQMTQSPSS |
| | LSASVGDRVTITCRASENIYSNLAWYQQKPGKAPKLL |
| | IYAATYLPDGVPSRFSGSGSGTDFTLTISSLQPEDFAT |
| | YYCQHFWVTPYTFGGGTKVEIK (SEQ ID NO: 488) |

HLA-A*11 antigen binding domains

| | |
|---|---|
| QVQLQESGPGLVKPS | CAGGTGCAGCTGCAGGAAAGCGGCCCTGGCCTGGT |
| QTLSLTCTVSGGSISS | GAAACCCAGCCAGACCCTGAGCCTGACCTGCACAG |
| GGYYWSWIRQPPGK | TGTCCGGCGGCTCGATCAGCAGCGGCGGCTACTAC |
| GLEWIGYIYYSGSTY | TGGTCCTGGATCAGACAGCCCCCTGGCAAGGGCCT |
| YNPSLKSRVTISVDTS | GGAATGGATCGGCTACATCTACTACAGCGGCAGCA |
| KNQFSLKLSSVTAAD | CCTACTACAACCCCAGCCTGAAGTCCAGAGTGACC |
| TAVYYCARHYYYYS | ATCAGCGTGGACACCAGCAAGAACCAGTTCAGCCT |
| MDVWGKGTTVTVSS | GAAGCTGAGCAGCGTGACAGCCGCCGACACCGCT |
| GGGGSGGGGSGGGG | GTGTATTACTGTGCGAGACACTACTACTACTACTCC |
| SGGDIQMTQSPSSLS | ATGGACGTCTGGGGCAAAGGGACCACGGTCACCGT |
| ASVGDRVTITCRASQ | GTCCTCAGGCGGAGGTGGAAGCGGAGGGGGAGGA |
| SISSYLNWYQQKPGK | TCTGGCGGCGGAGGAAGCGGAGGCGACATCCAGA |
| APKLLIYAASSLQSG | TGACCCAGTCTCCATCCTCCCTGTCTGCATCTGTAG |
| VPSRFSGSGSGTDFT | GAGACAGAGTCACCATCACTTGCCGGGCAAGTCAG |
| LTISSLQPEDFATYYC | AGCATTAGCAGCTATTTAAATTGGTATCAGCAGAA |
| QQSYSTPLTFGGGTK | ACCAGGGAAAGCCCCTAAGCTCCTGATCTATGCTG |
| VEIK (SEQ ID NO: | CATCCAGTTTGCAAAGTGGGGTCCCATCAAGGTTC |
| 489) | AGTGGCAGTGGATCTGGGACAGATTTCACTCTCAC |
| | CATCAGCAGTCTGCAACCTGAAGATTTTGCAACTT |
| | ACTACTGTCAACAGAGTTACAGTACCCCTCTCACTT |
| | TCGGCGGCGGAACAAAGGTGGAGATCAAG (SEQ ID |
| | NO: 490) |
| QITLKESGPTLVKPT | CAGATCACCCTGAAAGAGTCCGGCCCCACCCTGGT |
| QTLTLTCTFSGFSLST | GAAACCCACCCAGACCCTGACCCTGACATGCACCT |
| SGVGVGWIRQPPGK | TCAGCGGCTTCAGCCTGAGCACCTCTGGCGTGGGC |
| ALEWLALIYWNDDK | GTGGGCTGGATCAGACAGCCTCCCGGCAAGGCCCT |
| RYSPSLKSRLTITKDT | GGAATGGCTGGCCCTGATCTACTGGAACGACGACA |
| SKNQVVLTMTNMDP | AGCGGTACAGCCCCAGCCTGAAGTCCCGGCTGACC |
| VDTATYYCAHRHMR | ATCACCAAGGACACCTCGAAGAACCAGGTGGTGCT |
| LSCFDYWGQGTLVT | GACCATGACAAACATGGACCCCGTGGACACCGCCA |
| VSSGGGGSGGGGSG | CATATTACTGTGCACACAGACACATGCGTTTAAGC |
| GGGSGGDIQMTQSPS | TGTTTTGACTACTGGGGCCAGGGAACCCTGGTCAC |
| SLSASVGDRVTITCR | CGTGTCCTCAGGCGGAGGTGGAAGCGGAGGGGGA |
| ASQSISSYLNWYQQK | GGATCTGGCGGCGGAGGAAGCGGAGGCGACATCC |
| PGKAPKLLIYAASSL | AGATGACCCAGTCTCCATCCTCCCTGTCTGCATCTG |
| QSGVPSRFSGSGSGT | TAGGAGACAGAGTCACCATCACTTGCCGGGCAAGT |
| DFTLTISSLQPEDFAT | CAGAGCATTAGCAGCTATTTAAATTGGTATCAGCA |
| YYCQQSYSTPLTFGG | GAAACCAGGGAAAGCCCCTAAGCTCCTGATCTATG |
| GTKVEIK (SEQ ID | CTGCATCCAGTTTGCAAAGTGGGGTCCCATCAAGG |
| NO: 491) | TTCAGTGGCAGTGGATCTGGGACAGATTTCACTCT |
| | CACCATCAGCAGTCTGCAACCTGAAGATTTTGCAA |
| | CTTACTACTGTCAACAGAGTTACAGTACCCCTCTCA |
| | CTTTCGGCGGCGGAACAAAGGTGGAGATCAAG |
| | (SEQ ID NO: 492) |
| QVQLVQSGAEVKKP | CAGGTGCAGCTGGTGCAGTCTGGCGCCGAAGTGAA |
| GASVKVSCKASGYT | GAAACCTGGCGCCTCCGTGAAGGTGTCCTGCAAGG |
| FTSYAMHWVRQAPG | CCAGCGGCTACACCTTCACCAGCTACGCCATGCAC |
| QRLEWMGWINAGN | TGGGTTCGACAGGCCCCTGGCCAGAGACTGGAATG |
| GNTKYSQKFQGRVTI | GATGGGCTGGATCAACGCCGGCAACGGCAACACC |
| TRDTSASTAYMELSS | AAGTACAGCCAGAAATTCCAGGGCAGAGTGACCA |
| LRSEDTAVYYCARE | TCACCCGGGACACCAGCGCCAGCACCGCCTACATG |
| GNGANPDAFDIWGQ | GAACTGAGCAGCCTGCGGAGCGAGGACACCGCTG |
| GTMVTVSSGGGGSG | TGTATTACTGTGCGAGAGAAGGAAATGGTGCCAAC |
| GGGSGGGGSGGDIQ | CCTGATGCTTTTGATATCTGGGGCCAAGGGACAAT |
| MTQSPSSLSASVGDR | GGTCACCGTGTCCTCAGGCGGAGGTGGAAGCGGA |
| VTITCRASQSISSYLN | GGGGGAGGATCTGGCGGCGGAGGAAGCGGAGGCG |
| WYQQKPGKAPKLLI | ACATCCAGATGACCCAGTCTCCATCCTCCCTGTCTG |
| YAASSLQSGVPSRFS | CATCTGTAGGAGACAGAGTCACCATCACTTGCCGG |
| GSGSGTDFTLTISSLQ | GCAAGTCAGAGCATTAGCAGCTATTTAAATTGGTA |
| PEDFATYYCQQSYST | TCAGCAGAAACCAGGGAAAGCCCCTAAGCTCCTGA |
| PLTFGGGTKVEIK | TCTATGCTGCATCCAGTTTGCAAAGTGGGGTCCCA |
| (SEQ ID NO: 493) | TCAAGGTTCAGTGGCAGTGGATCTGGGACAGATTT |
| | CACTCTCACCATCAGCAGTCTGCAACCTGAAGATT |
| | TTGCAACTTACTACTGTCAACAGAGTTACAGTACC |
| | CCTCTCACTTTCGGCGGCGGAACAAAGGTGGAGAT |
| | CAAG (SEQ ID NO: 494) |
| EVQLVESGGGLVQP | GAAGTGCAGCTGGTGGAAAGCGGCGGAGGCCTGG |
| GGSLRLSCAASGFTF | TGCAGCCTGGCGGCAGCCTGAGACTGTCTTGCGCC |
| SSYDMHWVRQATG | GCCAGCGGCTTCACCTTCAGCAGCTACGACATGCA |
| KGLEWVSAIGTAGD | CTGGGTCCGCCAGGCCACCGGCAAGGGACTGGAAT |
| TYYPGSVKGRFTISR | GGGTGTCCGCCATCGGCACAGCCGGCGACACTTAC |
| ENAKNSLYLQMNSL | TACCCCGGCAGCGTGAAGGGCCGGTTCACCATCAG |
| RAGDTAVYYCARDL | CAGAGAGAACGCCAAGAACAGCCTGTACCTGCAG |
| PGSYWYFDLWGRGT | ATGAACAGCCTTCGAGCCGGCGATACCGCCGTGTA |
| LVTVSSGGGGSGGG | TTACTGTGCAAGAGATCTCCCTGGTAGCTACTGGT |
| GSGGGGSGGDIQMT | ACTTCGATCTCTGGGGCCGTGGCACCCTGGTCACT |
| QSPSSLSASVGDRVTI | GTGTCCTCAGGCGGAGGTGGAAGCGGAGGGGGAG |
| TCRASQSISSYLNWY | GATCTGGCGGCGGAGGAAGCGGAGGCGACATCCA |
| QQKPGKAPKLLIYAA | GATGACCCAGTCTCCATCCTCCCTGTCTGCATCTGT |
| SSLQSGVPSRFSGSGS | AGGAGACAGAGTCACCATCACTTGCCGGGCAAGTC |
| GTDFTLTISSLQPEDF | AGAGCATTAGCAGCTATTTAAATTGGTATCAGCAG |
| ATYYCQQSYSTPLTF | AAACCAGGGAAAGCCCCTAAGCTCCTGATCTATGC |
| GGGTKVEIK (SEQ ID | TGCATCCAGTTTGCAAAGTGGGGTCCCATCAAGGT |
| NO: 495) | TCAGTGGCAGTGGATCTGGGACAGATTTCACTCTC |
| | ACCATCAGCAGTCTGCAACCTGAAGATTTTGCAAC |
| | TTACTACTGTCAACAGAGTTACAGTACCCCTCTCAC |
| | TTTCGGCGGCGGAACAAAGGTGGAGATCAAG (SEQ |
| | ID NO: 496) |
| QVQLQESGPGLVKPS | CAGGTGCAGCTGCAGGAAAGCGGCCCTGGCCTGGT |
| QTLSLTCTVSGGSISS | GAAACCCAGCCAGACCCTGAGCCTGACCTGCACAG |
| GGYYWSWIRQPPGK | TGTCCGGCGGCTCGATCAGCAGCGGCGGCTACTAC |
| GLEWIGYIYYSGSTY | TGGTCCTGGATCAGACAGCCCCCTGGCAAGGGCCT |
| YNPSLKSRVTISVDTS | GGAATGGATCGGCTACATCTACTACAGCGGCAGCA |
| KNQFSLKLSSVTAAD | CCTACTACAACCCCAGCCTGAAGTCCAGAGTGACC |
| TAVYYCARHYYYYY | ATCAGCGTGGACACCAGCAAGAACCAGTTCAGCCT |
| LDVWGKGTTVTVSS | GAAGCTGAGCAGCGTGACAGCCGCCGACACCGCT |
| GGGGSGGGGSGGGG | GTGTATTACTGTGCGAGACACTACTACTACTACTA |
| SGGDIQMTQSPSSLS | CCTGGACGTCTGGGGCAAAGGGACCACGGTCACCG |
| ASVGDRVTITCRASQ | TGTCCTCAGGCGGAGGTGGAAGCGGAGGGGGAGG |
| SISSYLNWYQQKPGK | ATCTGGCGGCGGAGGAAGCGGAGGCGACATCCAG |
| APKLLIYAASSLQSG | ATGACCCAGTCTCCATCCTCCCTGTCTGCATCTGTA |
| VPSRFSGSGSGTDFT | GGAGACAGAGTCACCATCACTTGCCGGGCAAGTCA |
| LTISSLQPEDFATYYC | GAGCATTAGCAGCTATTTAAATTGGTATCAGCAGA |
| QQSYSTPLTFGGGTK | AACCAGGGAAAGCCCCTAAGCTCCTGATCTATGCT |
| VEIK (SEQ ID NO: | GCATCCAGTTTGCAAAGTGGGGTCCCATCAAGGTT |
| 497) | CAGTGGCAGTGGATCTGGGACAGATTTCACTCTCA |
| | CCATCAGCAGTCTGCAACCTGAAGATTTTGCAACT |
| | TACTACTGTCAACAGAGTTACAGTACCCCTCTCACT |
| | TTCGGCGGCGGAACAAAGGTGGAGATCAAG (SEQ |
| | ID NO: 498) |
| EVQLVESGGGLVQP | GAAGTGCAGCTGGTGGAAAGCGGCGGAGGCCTGG |
| GGSLRLSCAASGFTF | TGCAGCCTGGCGGCAGCCTGAGACTGTCTTGCGCC |
| SSYWMHWVRQAPG | GCCAGCGGCTTCACCTTCAGCAGCTACTGGATGCA |
| KGLVWVSRINSDGSS | CTGGGTCCGCCAGGCCCCTGGCAAGGGACTGGTCT |
| TSYADSVKGRFTISR | GGGTGTCTCGAATCAACAGCGACGGCAGCAGCACC |
| DNAKNTLYLQMNSL | AGCTACGCCGACAGCGTGAAGGGCCGGTTCACCAT |
| RAEDTAVYYCCLGV | CAGCCGGGACAACGCCAAGAACACCCTGTACCTGC |
| LLYNWFDPWGQGTL | AGATGAACAGCCTGCGGGCCGAGGACACCGCCGT |
| VTVSSGGGGSGGGG | GTATTACTGTTGTTTGGGTGTTTTATTATACAACTG |
| SGGGGSGGDIQMTQ | GTTCGACCCCTGGGGCCAGGGAACCCTGGTCACCG |
| SPSSLSASVGDRVTIT | TGTCCTCAGGCGGAGGTGGAAGCGGAGGGGGAGG |
| CRASQSISSYLNWYQ | ATCTGGCGGCGGAGGAAGCGGAGGCGACATCCAG |
| QKPGKAPKLLIYAAS | ATGACCCAGTCTCCATCCTCCCTGTCTGCATCTGTA |
| SLQSGVPSRFSGSGS | GGAGACAGAGTCACCATCACTTGCCGGGCAAGTCA |
| GTDFTLTISSLQPEDF | GAGCATTAGCAGCTATTTAAATTGGTATCAGCAGA |
| ATYYCQQSYSTPLTF | AACCAGGGAAAGCCCCTAAGCTCCTGATCTATGCT |
| GGGTKVEIK (SEQ ID | GCATCCAGTTTGCAAAGTGGGGTCCCATCAAGGTT |
| NO: 499) | CAGTGGCAGTGGATCTGGGACAGATTTCACTCTCA |
| | CCATCAGCAGTCTGCAACCTGAAGATTTTGCAACT |
| | TACTACTGTCAACAGAGTTACAGTACCCCTCTCACT |
| | TTCGGCGGCGGAACAAAGGTGGAGATCAAG (SEQ |
| | ID NO: 500) |
| QVQLQESGPGLVKPS | CAGGTGCAGCTGCAGGAAAGCGGCCCTGGCCTGGT |
| QTLSLTCTVSGGSISS | GAAACCCAGCCAGACCCTGAGCCTGACCTGCACAG |
| GGYYWSWIRQPPGK | TGTCCGGCGGCTCGATCAGCAGCGGCGGCTACTAC |
| GLEWIGYIYYSGSTY | TGGTCCTGGATCAGACAGCCCCCTGGCAAGGGCCT |
| YNPSLKSRVTISVDTS | GGAATGGATCGGCTACATCTACTACAGCGGCAGCA |
| KNQFSLKLSSVTAAD | CCTACTACAACCCCAGCCTGAAGTCCAGAGTGACC |
| TAVYYCARHYYYY | ATCAGCGTGGACACCAGCAAGAACCAGTTCAGCCT |
| MDVWGKGTTVTVSS | GAAGCTGAGCAGCGTGACAGCCGCCGACACCGCT |
| GGGGSGGGGSGGGG | GTGTATTACTGTGCGAGACACTACTACTACTACAT |
| SGGDIQMTQSPSSLS | GGACGTCTGGGGCAAAGGGACCACGGTCACCGTGT |
| ASVGDRVTITCRASQ | CCTCAGGCGGAGGTGGAAGCGGAGGGGGAGGATC |
| SISSYLNWYQQKPGK | TGGCGGCGGAGGAAGCGGAGGCGACATCCAGATG |
| APKLLIYAASSLQSG | ACCCAGTCTCCATCCTCCCTGTCTGCATCTGTAGGA |
| VPSRFSGSGSGTDFT | GACAGAGTCACCATCACTTGCCGGGCAAGTCAGAG |
| LTISSLQPEDFATYYC | CATTAGCAGCTATTTAAATTGGTATCAGCAGAAAC |
| QQSYSTPLTFGGGTK | CAGGGAAAGCCCCTAAGCTCCTGATCTATGCTGCA |
| VEIK (SEQ ID NO: | TCCAGTTTGCAAAGTGGGGTCCCATCAAGGTTCAG |
| 501) | TGGCAGTGGATCTGGGACAGATTTCACTCTCACCA |
| | TCAGCAGTCTGCAACCTGAAGATTTTGCAACTTAC |
| | TACTGTCAACAGAGTTACAGTACCCCTCTCACTTTC |
| | GGCGGCGGAACAAAGGTGGAGATCAAG (SEQ ID |
| | NO: 502) |
| QITLKESGPTLVKPT | CAGATCACCCTGAAAGAGTCCGGCCCCACCCTGGT |
| QTLTLTCTFSGFSLST | GAAACCCACCCAGACCCTGACCCTGACATGCACCT |
| SGVGVGWIRQPPGK | TCAGCGGCTTCAGCCTGAGCACCTCTGGCGTGGGC |
| ALEWLALIYWNDDK | GTGGGCTGGATCAGACAGCCTCCCGGCAAGGCCCT |
| RYSPSLKSRLTITKDT | GGAATGGCTGGCCCTGATCTACTGGAACGACGACA |
| SKNQVVLTMTNMDP | AGCGGTACAGCCCCAGCCTGAAGTCCCGGCTGACC |
| VDTATYYCAHKTTS | ATCACCAAGGACACCTCGAAGAACCAGGTGGTGCT |
| FYFDYWGQGTLVTV | GACCATGACAAACATGGACCCCGTGGACACCGCCA |
| SSGGGGSGGGGSGG | CATATTACTGTGCACACAAAACGACGTCGTTTTAC |
| GGSGGDIQMTQSPSS | TTTGACTACTGGGGCCAGGGAACCCTGGTCACCGT |
| LSASVGDRVTITCRA | GTCCTCAGGCGGAGGTGGAAGCGGAGGGGGAGGA |
| SQSISSYLNWYQQKP | TCTGGCGGCGGAGGAAGCGGAGGCGACATCCAGA |
| GKAPKLLIYAASSLQ | TGACCCAGTCTCCATCCTCCCTGTCTGCATCTGTAG |
| SGVPSRFSGSGSGTD | GAGACAGAGTCACCATCACTTGCCGGGCAAGTCAG |
| FTLTISSLQPEDFATY | AGCATTAGCAGCTATTTAAATTGGTATCAGCAGAA |
| YCQQSYSTPLTFGGG | ACCAGGGAAAGCCCCTAAGCTCCTGATCTATGCTG |
| TKVEIK (SEQ ID NO: | CATCCAGTTTGCAAAGTGGGGTCCCATCAAGGTTC |
| 503) | AGTGGCAGTGGATCTGGGACAGATTTCACTCTCAC |
| | CATCAGCAGTCTGCAACCTGAAGATTTTGCAACTT |
| | ACTACTGTCAACAGAGTTACAGTACCCCTCTCACTT |
| | TCGGCGGCGGAACAAAGGTGGAGATCAAG (SEQ ID |
| | NO: 504) |
| QVQLQESGPGLVKPS | CAGGTGCAGCTGCAGGAAAGCGGCCCTGGCCTGGT |
| QTLSLTCTVSGGSISS | GAAACCCAGCCAGACCCTGAGCCTGACCTGCACAG |
| GGYYWSWIRQPPGK | TGTCCGGCGGCTCGATCAGCAGCGGCGGCTACTAC |
| GLEWIGYIYYSGSTY | TGGTCCTGGATCAGACAGCCCCCTGGCAAGGGCCT |
| YNPSLKSRVTISVDTS | GGAATGGATCGGCTACATCTACTACAGCGGCAGCA |
| KNQFSLKLSSVTAAD | CCTACTACAACCCCAGCCTGAAGTCCAGAGTGACC |
| TAVYYCARHYYYYY | ATCAGCGTGGACACCAGCAAGAACCAGTTCAGCCT |
| MDVWGKGTTVTVSS | GAAGCTGAGCAGCGTGACAGCCGCCGACACCGCT |
| GGGGSGGGGSGGGG | GTGTATTACTGTGCGAGACACTACTACTACTACTA |
| SGGDIQMTQSPSSLS | CATGGACGTCTGGGGCAAAGGGACCACGGTCACC |
| ASVGDRVTITCRASQ | GTGTCCTCAGGCGGAGGTGGAAGCGGAGGGGGAG |
| SISSYLNWYQQKPGK | GATCTGGCGGCGGAGGAAGCGGAGGCGACATCCA |
| APKLLIYAASSLQSG | GATGACCCAGTCTCCATCCTCCCTGTCTGCATCTGT |
| VPSRFSGSGSGTDFT | AGGAGACAGAGTCACCATCACTTGCCGGGCAAGTC |
| LTISSLQPEDFATYYC | AGAGCATTAGCAGCTATTTAAATTGGTATCAGCAG |
| QQSYSTPLTFGGGTK | AAACCAGGGAAAGCCCCTAAGCTCCTGATCTATGC |
| VEIK (SEQ ID NO: | TGCATCCAGTTTGCAAAGTGGGGTCCCATCAAGGT |
| 505) | TCAGTGGCAGTGGATCTGGGACAGATTTCACTCTC |
| | ACCATCAGCAGTCTGCAACCTGAAGATTTTGCAAC |
| | TTACTACTGTCAACAGAGTTACAGTACCCCTCTCAC |
| | TTTCGGCGGCGGAACAAAGGTGGAGATCAAG (SEQ |
| | ID NO: 506) |

HLA-A*03 scFv Sequences

| | |
|---|---|
| 15 | QVQLVQSGAEVKKPGASVKVSCKASGYTFTSYGISW |
| | VRQAPGQGLEWMGWISAYNGNTNYAQKLQGRVTM |
| | TTDTSTSTAYMELRSLRSDDTAVYYCARERVSQRGA |
| | FDIWGQGTMVTVSSGGGGSGGGGSGGGGSGGDIQM |
| | TQSPSSLSASVGDRVTITCRASQSISSYLNWYQQKPG |
| | KAPKLLIYAASSLQSGVPSRFSGSGSGTDFTLTISSLQP |
| | EDFATYYCQQSYSTPLTFGGGTKVEIK (SEQ ID NO: |
| | 507) |
| 16 | EVQLVESGGGLVQPGGSLRLSCAASGFTFSSYSMNW |
| | VRQAPGKGLEWVSYISSSSSTIYYADSVKGRFTISRD |
| | NAKNSLYLQMNSLRAEDTAVYYCARGNPDKDPFDY |
| | WGQGTLVTVSSGGGGSGGGGSGGGGSGGDIQMTQS |
| | PSSLSASVGDRVTITCRASQSISSYLNWYQQKPGKAP |
| | KLLIYAASSLQSGVPSRFSGSGSGTDFTLTISSLQPEDF |
| | ATYYCQQSYSTPLTFGGGTKVEIK (SEQ ID NO: 508) |
| 17 | QVQLQESGPGLVKPSETLSLTCTVSGGSVSSGSYYWS |
| | WIRQPPGKGLEWIGYIYYSGSTNYNPSLKSRVTISVDT |
| | SKNQFSLKLSSVTAADTAVYYCARDFYCTNWYFDL |
| | WGRGTLVTVSSGGGGSGGGGSGGGGSGGDIQMTQS |
| | PSSLSASVGDRVTITCRASQSISSYLNWYQQKPGKAP |
| | KLLIYAASSLQSGVPSRFSGSGSGTDFTLTISSLQPEDF |
| | ATYYCQQSYSTPLTFGGGTKVEIK (SEQ ID NO: 509) |
| 18 | QVQLQESGPGLVKPSETLSLTCTVSGGSISSYYWSWI |
| | RQPPGKGLEWIGYIYYSGSTNYNPSLKSRVTISVDTSK |
| | NQFSLKLSSVTAADTAVYYCARESSSGSYWYFDLWG |
| | RGTLVTVSSGGGGSGGGGSGGGGSGGDIQMTQSPSS |
| | LSASVGDRVTITCRASQSISSYLNWYQQKPGKAPKLL |
| | IYAASSLQSGVPSRFSGSGSGTDFTLTISSLQPEDFATY |
| | YCQQSYSTPLTFGGGTKVEIK (SEQ ID NO: 510) |
| 19 | EVQLVQSGAEVKKPGESLKISCKGSGYSFTSYWIGW |
| | VRQMPGKGLEWMGIIYPGDSDTRYSPSFQGQVTISAD |
| | KSISTAYLQWSSLKASDTAMYYCARDSGYKYNLYY |
| | YYYYMDVWGKGTTVTVSSGGGGSGGGGSGGGGSG |
| | GDIQMTQSPSSLSASVGDRVTITCRASQSISSYLNWY |
| | QQKPGKAPKLLIYAASSLQSGVPSRFSGSGSGTDFTL |
| | TISSLQPEDFATYYCQQSYSTPLTFGGGTKVEIK (SEQ |
| | ID NO: 511) |
| 20 | QVQLVQSGAEVKKPGASVKVSCKASGYTFTSYGISW |
| | VRQAPGQGLEWMGWISAYNGNTNYAQKLQGRVTM |
| | TTDTSTSTAYMELRSLRSDDTAVYYCARGGDLSHYY |
| | YYMDVWGKGTTVTVSSGGGGSGGGGSGGGGSGGQ |
| | TVVTQEPSLTVSPGGTVTLTCASSTGAVTSGYYPNWF |
| | QQKPGQAPRALIYSTSNKHSWTPARFSGSLLGGKAA |
| | LTLSGVQPEDEAEYYCLLYYGGAQWVFGGGTKLTV |
| | L (SEQ ID NO: 512) |
| 21 | QVQLVQSGAEVKKPGASVKVSCKASGYTFTSYGISW |
| | VRQAPGQGLEWMGWISAYNGNTNYAQKLQGRVTM |
| | TTDTSTSTAYMELRSLRSDDTAVYYCARENRRYNSC |
| | YYFDYWGQGTLVTVSSGGGGSGGGGSGGGGSGGDI |
| | QMTQSPSSLSASVGDRVTITCRASQSISSYLNWYQQK |
| | PGKAPKLLIYAASSLQSGVPSRFSGSGSGTDFTLTISSL |
| | QPEDFATYYCQQSYSTPLTFGGGTKVEIK (SEQ ID |
| | NO: 513) |
| 22 | QVQLVQSGAEVKKPGASVKVSCKASGYTFTSYGISW |
| | VRQAPGQGLEWMGWISAYNGNTNYAQKLQGRVTM |
| | TTDTSTSTAYMELRSLRSDDTAVYYCARGGDLSHYY |
| | YYLDVWGKGTTVTVSSGGGGSGGGGSGGGGSGGQT |
| | VVTQEPSLTVSPGGTVTLTCASSTGAVTSGYYPNWF |
| | QQKPGQAPRALIYSTSNKHSWTPARFSGSLLGGKAA |
| | LTLSGVQPEDEAEYYCLLYYGGAQWVFGGGTKLTV |
| | L (SEQ ID NO: 514) |
| 23 | EVQLVESGGGLVQPGGSLRLSCAASGFTVSSNYMSW |
| | VRQAPGKGLEWVSVIYSGGSTYYADSVKGRFTISRD |
| | NSKNTLYLQMNSLRAEDTAVYYCARATLLSLSYDAF |
| | DIWGQGTMVTVSSGGGGSGGGGSGGGGSGGDIQMT |
| | QSPSSLSASVGDRVTITCRASQSISSYLNWYQQKPGK |
| | APKLLIYAASSLQSGVPSRFSGSGSGTDFTLTISSLQPE |
| | DFATYYCQQSYSTPLTFGGGTKVEIK (SEQ ID NO: |
| | 515) |
| 24 | QVQLVQSGAEVKKPGASVKVSCKASGYTFTSYGISW |
| | VRQAPGQGLEWMGWISAYNGNTNYAQKLQGRVTM |
| | TTDTSTSTAYMELRSLRSDDTAVYYCARGGDLSHYY |
| | YMDVWGKGTTVTVSSGGGGSGGGGSGGGGSGGQT |
| | VVTQEPSLTVSPGGTVTLTCASSTGAVTSGYYPNWF |
| | QQKPGQAPRALIYSTSNKHSWTPARFSGSLLGGKAA |
| | LTLSGVQPEDEAEYYCLLYYGGAQWVFGGGTKLTV |
| | L (SEQ ID NO: 516) |
| 25 | EVQLVQSGAEVKKPGESLKISCKGSGYSFTSYWIGW |
| | VRQMPGKGLEWMGIIYPGDSDTRYSPSFQGQVTISAD |
| | KSISTAYLQWSSLKASDTAMYYCARERDRWFDPWG |
| | QGTLVTVSSGGGGSGGGGSGGGGSGGDIQMTQSPSS |
| | LSASVGDRVTITCRASQSISSYLNWYQQKPGKAPKLL |
| | IYAASSLQSGVPSRFSGSGSGTDFTLTISSLQPEDFATY |
| | YCQQSYSTPLTFGGGTKVEIK (SEQ ID NO: 517) |
| 26 | QVQLVQSGAEVKKPGASVKVSCKASGYTFTSYGISW |
| | VRQAPGQGLEWMGWISAYNGNTNYAQKLQGRVTM |
| | TTDTSTSTAYMELRSLRSDDTAVYYCARETPPSLGAF |
| | DIWGQGTMVTVSSGGGGSGGGGSGGGGSGGQSALT |
| | QPPSASGSPGQSVTISCTGTSSDVGGYNYVSWYQQHP |
| | GKAPKLMIYEVSKRPSGVPDRFSGSKSGNTASLTVSG |
| | LQAEDEADYYCSSYAGSNNWVFGGGTKLTVL (SEQ |
| | ID NO: 518) |
| 27 | QLQLQESGPGLVKPSETLSLTCTVSGGSISSSSYYWG |
| | WIRQPPGKGLEWIGSIYYSGSTYYNPSLKSRVTISVDT |
| | SKNQFSLKLSSVTAADTAVYYCAREAYCLSDSYWYF |
| | DLWGRGTLVTVSSGGGGSGGGGSGGGGSGGQSVLT |
| | QPPSASGTPGQRVTISCSGSSSNIGSNTVNWYQQLPG |
| | TAPKLLIYSNNQRPSGVPDRFSGSKSGTSASLAISGLQ |
| | SEDEADYYCAAWDDSLNGWVFGGGTKLTVL (SEQ |
| | ID NO: 519) |
| 28 | QVQLQESGPGLVKPSQTLSLTCTVSGGSISSGGYYWS |
| | WIRQPPGKGLEWIGYIYYSGSTYYNPSLKSRVTISVDT |
| | SKNQFSLKLSSVTAADTAVYYCARESWKYFYPRGY |
| | MDVWGKGTTVTVSSGGGGSGGGGSGGGGSGGDIQ |
| | MTQSPSSLSASVGDRVTITCRASQSISSYLNWYQQKP |
| | GKAPKLLIYAASSLQSGVPSRFSGSGSGTDFTLTISSL |
| | QPEDFATYYCQQSYSTPLTFGGGTKVEIK (SEQ ID |
| | NO: 520) |

In some embodiments, the ligand binding domain of the second, inhibitory receptor comprises a scFv. In some embodiments, the scFv binds to HLA-A*01, HLA-A*02, HLA-A*3, HLA-A*11, HLA-B*07 or HLA-C*07.

Illustrative heavy chain and light chain CDRs (CDR-H1, CDR-H2 and CDR-H3, or CDR-L1, CDR-L2 and CDR-L3, respectively) for HLA-A*02, HLA-A*03, HLA-A*11, and HLA-B*07 ligand binding domains are shown in Table 7B below.

TABLE 7B
CDRs corresponding to HLA antigen binding domains

| | | | | | |
|---|---|---|---|---|---|
| CDR-L1 | CDR-L2 | CDR-L3 | CDR-H1 | CDR-H2 | CDR-H3 |

HLA-A*02 CDRs

| | | | | | |
|---|---|---|---|---|---|
| RSSQSIVH | KVSNRFS | FQGSHVP | ASGYTFTS | WIYPGNV | EEITYAM |
| SNGNTYL | GVPDR | RT (SEQ ID | YHIH (SEQ | NTEYNEK | DY |
| E (SEQ ID | (SEQ ID | NO: 523) | ID NO: | FKGK | (SEQ ID |
| NO: 521) | NO: 522) | | 524) | (SEQ ID | NO: 526) |
| | | | | NO: 525) | |
| RSSQSIVH | KVSNRFS | MQGSHVP | SGYTFTSY | WIYPGDG | EGTYYAM |
| SNGNTYL | GVPDR | RT (SEQ ID | HMH (SEQ | STQYNEK | DY (SEQ |
| D (SEQ ID | (SEQ ID | NO: 529) | ID NO: | FKG (SEQ | ID NO: |
| NO: 527) | NO: 528) | | 530) | ID NO: | 532) |
| | | | | 531) | |

HLA-A*03 CDRs

| | | | | | |
|---|---|---|---|---|---|
| RASQSISS | AASSLQS | QQSYSTPL | SYGIS | WISAYNG | ERVSQRG |
| YLN (SEQ | (SEQ ID | T (SEQ ID | (SEQ ID | NTNYAQK | AFDI (SEQ |
| ID NO: | NO: 534) | NO: 535) | NO: 536) | LQG (SEQ | ID NO: |
| 533) | | | | ID NO: | 538) |
| | | | | 537) | |
| RASQSISS | AASSLQS | QQSYSTPL | SYSMN | YISSSSSTI | GNPDKDP |
| YLN(SEQ | (SEQ ID | T (SEQ ID | (SEQ ID | YYADSVK | FDY (SEQ |
| ID NO: | NO: 534) | NO: 535) | NO: 542) | G (SEQ ID | ID NO: |
| 533) | | | | NO: 543) | 544) |
| RASQSISS | AASSLQS | QQSYSTPL | SGSYYWS | YIYYSGST | DFYCTNW |
| YLN(SEQ | (SEQ ID | T (SEQ ID | (SEQ ID | NYNPSLK | YFDL (SEQ |
| ID NO: | NO: 534) | NO: 535) | NO: 548) | S (SEQ ID | ID NO: |
| 533) | | | | NO: 549) | 550) |
| RASQSISS | AASSLQS | QQSYSTPL | SYYWS | YIYYSGST | ESSSGSY |
| YLN(SEQ | (SEQ ID | T (SEQ ID | (SEQ ID | NYNPSLK | WYFDL |
| ID NO: | NO: 534) | NO: 535) | NO: 554) | S (SEQ ID | (SEQ ID |
| 533) | | | | NO: 555) | NO: 556) |
| RASQSISS | AASSLQS | QQSYSTPL | SYWIG | IIYPGDSD | DSGYKYN |
| YLN(SEQ | (SEQ ID | T (SEQ ID | (SEQ ID | TRYSPSFQ | LYYYYYY |
| ID NO: | NO: 534) | NO: 535) | NO: 560) | G (SEQ ID | MDV (SEQ |
| 533) | | | | NO: 561) | ID NO: |
| | | | | | 562) |
| ASSTGAV | STSNKHS | LLYYGGA | SYGIS | WISAYNG | GGDLSHY |
| TSGYYPN | (SEQ ID | QWV (SEQ | (SEQ ID | NTNYAQK | YYYMDV |
| (SEQ ID | NO: 564) | ID NO: | NO: 536) | LQG (SEQ | (SEQ ID |
| NO: 563) | | 565) | | ID NO: | NO: 568) |
| | | | | 567) | |
| RASQSISS | AASSLQS | QQSYSTPL | SYGIS | WISAYNG | ENRRYNS |
| YLN (SEQ | (SEQ ID | T (SEQ ID | (SEQ ID | NTNYAQK | CYYFDY |
| ID NO: | NO: 534) | NO: 535) | NO: 536) | LQG (SEQ | (SEQ ID |
| 533) | | | | ID NO: | NO: 574) |
| | | | | 573) | |
| ASSTGAV | STSNKHS | LLYYGGA | SYGIS | WISAYNG | GGDLSHY |
| TSGYYPN | (SEQ ID | QWV (SEQ | (SEQ ID | NTNYAQK | YYYLDV |
| (SEQ ID | NO: 576) | ID NO: | NO: 536) | LQG (SEQ | (SEQ ID |
| NO: 575) | | 577) | | ID NO: | NO: 580) |
| | | | | 579) | |
| RASQSISS | AASSLQS | QQSYSTPL | SNYMS | VIYSGGST | ATLLSLSY |
| YLN(SEQ | (SEQ ID | T (SEQ ID | (SEQ ID | YYADSVK | DAFDI |
| ID NO: | NO: 534) | NO: 535) | NO: 584) | G (SEQ ID | (SEQ ID |
| 533) | | | | NO: 585) | NO: 586) |
| ASSTGAV | STSNKHS | LLYYGGA | SYGIS | WISAYNG | GGDLSHY |
| TSGYYPN | (SEQ ID | QWV (SEQ | (SEQ ID | NTNYAQK | YYMDV |
| (SEQ ID | NO: 588) | ID NO: | NO: 590) | LQG (SEQ | (SEQ ID |
| NO: 587) | | 589) | | ID NO: | NO: 592) |
| | | | | 591) | |
| RASQSISS | AASSLQS | QQSYSTPL | SYWIG | IIYPGDSD | ERDRWFD |
| YLN (SEQ | (SEQ ID | T (SEQ ID | (SEQ ID | TRYSPSFQ | P (SEQ ID |
| ID NO: | NO: 594) | NO: 595) | NO: 596) | G (SEQ ID | NO: 598) |
| 593) | | | | NO: 597) | |
| TGTSSDV | EVSKRPS | SSYAGSN | SYGIS | WISAYNG | ETPPSLGA |
| GGYNYVS | (SEQ ID | NWV (SEQ | (SEQ ID | NTNYAQK | FDI (SEQ |
| (SEQ ID | NO: 600) | ID NO: | NO: 602) | LQG (SEQ | ID NO: |
| NO: 599) | | 601) | | ID NO: | 604) |
| | | | | 603) | |
| SGSSSNIG | SNNQRPS | AAWDDSL | SSSYYWG | SIYYSGST | EAYCLSD |
| SNTVN | (SEQ ID | NGWV | (SEQ ID | YYNPSLK | SYWYFDL |
| (SEQ ID | NO: 606) | (SEQ ID | NO: 608) | S (SEQ ID | (SEQ ID |
| NO: 605) | | NO: 607) | | NO: 609) | NO: 610) |
| RASQSISS | AASSLQS | QQSYSTPL | SGGYYWS | YIYYSGST | ESWKYFY |
| YLN (SEQ | (SEQ ID | T (SEQ ID | (SEQ ID | YYNPSLK | PRGYMDV |
| ID NO: | NO: 612) | NO: 613) | NO: 614) | S (SEQ ID | (SEQ ID |
| 611) | | | | NO: 615) | NO: 616) |

HLA-B*07 CDRs

| | | | | | |
|---|---|---|---|---|---|
| RASENIYS | AATYLPD | QHFWVTP | SGYSWH | YIHFSGST | GGVVSHY |
| NLA (SEQ | (SEQ ID | YT (SEQ | (SEQ ID | HYHPSLK | AMDC |
| ID NO: | NO: 618) | ID NO: | NO: 620) | S (SEQ ID | (SEQ ID |
| 617) | | 619) | | NO: 621) | NO: 622) |

In some embodiments, the extracellular ligand binding domain of the second receptor specifically binds an allelic variant of an HLA-A, HLA-B, or HLA-C protein. In some embodiments, the extracellular ligand binding domain of the second receptor specifically binds to HLA-A*01, HLA-A*02, HLA-A*03, HLA-A*11, HLA-B*07, or HLA-C*07.

In some embodiments, the extracellular ligand binding domain of the second receptor specifically binds to HLA-A*02. In some embodiments, the extracellular ligand binding domain of the second receptor comprises HLA-A*02 complementarity determining regions (CDRs) CDR-L1, CDR-L2, CDR-L3, CDR-H1, CDR-H2, CDR-H3 as disclosed Table 7B; or CDR sequences having at most 1, 2, or 3 substitutions, deletions, or insertions relative to the HLA-A*02 CDRs of Table 7B. In some embodiments, the extracellular ligand binding domain of the second receptor comprises complementarity determining regions (CDRs) CDR-L1, CDR-L2, CDR-L3, CDR-H1, CDR-H2, CDR-H3 of SEQ ID NOS: 521-526 or of SEQ ID NOS: 527-532; or CDR sequences having at most 1, 2, or 3 substitutions, deletions, or insertion relative to the CDRs of SEQ ID NOS: 521-526 or SEQ ID NOS: 527-532.

In some embodiments, the extracellular ligand binding domain of the second receptor specifically binds to HLA-A*03. In some embodiments, the extracellular ligand binding domain of the second receptor comprises HLA-A*03 complementarity determining regions (CDRs) CDR-L1, CDR-L2, CDR-L3, CDR-H1, CDR-H2, CDR-H3 as disclosed Table 7B; or CDR sequences having at most 1, 2, or 3 substitutions, deletions, or insertions relative to the HLA-A*03 CDRs of Table 7B.

In some embodiments, the extracellular ligand binding domain of the second receptor specifically binds to HLA-B*07. In some embodiments, the extracellular ligand binding domain of the second receptor comprises HLA-B*07 complementarity determining regions (CDRs) CDR-L1, CDR-L2, CDR-L3, CDR-H1, CDR-H2, CDR-H3 as disclosed Table 7B; or CDR sequences having at most 1, 2, or 3 substitutions, deletions, or insertions relative to the HLA-B*07 CDRs of Table 7B.

In further embodiments of any of the ligand binding domains, each CDR sequence may have 1, 2, 3 or more substitutions, insertions, or deletions. CDR sequences may tolerate substitutions, deletions, or insertions. Using sequence alignment tools, routine experimentation, and known assays, those of skill in the art may generate and test variant sequences having 1, 2, 3, or more substitutions, insertions, or deletions in CDR sequences without undue experimentation.

In some embodiments, the allogeneic donor cell antigen comprising HLA-A*02, and the ligand binding domain of the second receptor comprises an HLA-A*02 ligand binding domain. In some embodiments, the ligand binding domain binds HLA-A*02 independent of the peptide in a pMHC complex comprising HLA-A*02. In some embodiments, the HLA-A*02 ligand binding domain comprises an scFv domain. In some embodiments, the HLA-A*02 ligand binding domain comprises a sequence of any one of SEQ ID NOs: 455, 457, 459, 461, 463, 465, 467, 469, 471, 473, 475, and 477. In some embodiments, the HLA-A*02 ligand binding domain comprises a sequence at least 90%, at least 95%, at least 97% at least 99%, or 100% identical to a sequence of any one of SEQ ID NOs: 455, 457, 459, 461, 463, 465, 467, 469, 471, 473, 475, and 477. In some embodiments, the allogeneic donor cell antigen comprises HLA-A*02, and the extracellular ligand binding domain of the second receptor comprises a sequence of SEQ ID NO: 455, or a sequence having at least 90%, at least 95%, at least 97%, at least 99% identity, or 100% identical thereto. In some embodiments, the allogeneic donor cell antigen comprises HLA-A*02, and the extracellular ligand binding domain of the second receptor comprises a sequence of SEQ ID NO: 455.

In some embodiments, the allogeneic donor cell antigen comprises HLA-A*02, and the extracellular ligand binding domain of the second receptor comprises a VL comprising a sequence of DVLMTQTPLSLPVSLGDQASISCRSSQSIVHSNGNTYLEWYLQKPGQSPKLLIYKVSN RFSGVPDRFSGSGSGTDFTLKISR VEAEDLGVYYCFQGSHVPRTSGGGTKLEIK (SEQ ID NO: 623), or a sequence having at least 90%, at least 95%, at least 97%, at least 99%, or is 100% identical thereto. In some embodiments, the extracellular ligand binding domain of the second receptor comprises a VH comprising a sequence of QVQLQQSGPELVKPGASVRISCKASGYTFTSYHIHWVKQRPGQGLEWIGWIYPGNV NTEYNEKFKGKATLTADKSSSTAYMHLSSLTSEDSAVYFCAREEITYAMDYWGQGT SVTVSS (SEQ ID NO: 624), or a sequence having at least 90%, at least 95%, at least 97%, at least 99%, or is 100% identical thereto. In some embodiments, the VH and VL are separated by a linker, for example GGGGSGGGGSGGGGSGG (SEQ ID NO: 625). In some embodiments, the VH and VL are ordered, from N to C terminal, VH, linker and VL. In some embodiments, the VH and VL are ordered, from N to C terminal, VL, linker and VH. In some embodiments, the HLA-A*02 scFv comprises the complementarity determined regions (CDRs) of any one of SEQ ID NOS: 521-532. In some embodiments, the scFv comprises a sequence at least 95% identical to any one of SEQ ID NOS: 521-532. In some embodiments, the scFv comprises a sequence identical to any one of SEQ ID NOS: 521-532. In some embodiments, the heavy chain of the antigen binding domain comprises the heavy chain CDRs of any one of SEQ ID NOS: 521-532, and wherein the light chain of the antigen binding domain comprises the light chain CDRs of any one of SEQ ID NOS: 521-532. In some embodiments, the HLA-A*02 antigen binding domain comprises a heavy chain and a light chain, and the heavy chain comprises CDRs selected from SEQ ID NOs: 524-526 and 530-532 and the light chain comprises CDRs selected from SEQ ID NOs: 521-523 and 527-529.

In some embodiments, the heavy chain comprises a sequence identical to the heavy chain portion of any one of SEQ ID NOS: 455, 457, 459, 461, 463, 465, 467, 469, 471, 473, 475, and 477, and wherein the light chain of comprises a sequence identical to the light chain portion of any one of SEQ ID NOS: 455, 457, 459, 461, 463, 465, 467, 469, 471, 473, 475, and 477.

In some embodiments, the allogeneic donor cell antigen comprises HLA-A*03, and the extracellular ligand binding domain of the second receptor comprises an HLA-A*03 ligand binding domain.

In some embodiments, the allogeneic donor cell antigen comprises HLA-A*11, and the extracellular ligand binding domain of the second receptor comprises an HLA-A*11 ligand binding domain.

In some embodiments, the allogeneic donor cell antigen comprises HLA-B*07, and the extracellular ligand binding domain of the second receptor comprises an HLA-B*07 ligand binding domain.

In some embodiments, the allogeneic donor cell antigen comprises HLA-A*11. Various single variable domains known in the art or disclosed herein that bind to and recognize HLA-A*11 are suitable for use in embodiments.

Illustrative heavy chain and light chain CDRs (CDR-H1, CDR-H2 and CDR-H3, or CDR-L1, CDR-L2 and CDR-L3, respectively) for HLA-A*11 ligand binding domains are shown in Table 8 below. Any of the VH CDRs in Table 8 and may be combined with the VL CDRs disclosed in Table 8.

TABLE 8
Illustrative anti-HLA-A*11 CDR Sequences

| CDR H1 | CDR H2 | CDR H3 |
|---|---|---|
| SGGYYWS (SEQ ID | YIYYSGSTYYNPSLKS | HYYYYYMDV (SEQ |
| NO: 626) | (SEQ ID NO: 627) | ID NO: 628) |
| TSGVGVG (SEQ ID | LIYWNDDKRYSPSLKS | KTTSFYFDY (SEQ ID |
| NO: 629) | (SEQ ID NO: 630) | NO: 631) |
| SGGYYWS (SEQ ID | YIYYSGSTYYNPSLKS | HYYYYMDV (SEQ ID |
| NO: 632) | (SEQ ID NO: 633) | NO: 634) |
| SYWMH (SEQ ID NO: | RINSDGSSTSYADSVKG | GVLLYNWFDP (SEQ |
| 635) | (SEQ ID NO: 636) | ID NO: 637) |
| SGGYYWS (SEQ ID | YIYYSGSTYYNPSLKS | HYYYYYLDV (SEQ |
| NO: 638) | (SEQ ID NO: 639) | ID NO: 640) |
| SYDMH (SEQ ID NO: | AIGTAGDTYYPGSVKG | DLPGSYWYFDL (SEQ |
| 641) | (SEQ ID NO: 642) | ID NO: 643) |
| SYAMH (SEQ ID NO: | WINAGNGNTKYSQKFQ | EGNGANPDAFDI |
| 644) | G (SEQ ID NO: 645) | (SEQ ID NO: 646) |
| TSGVGVG (SEQ ID | LIYWNDDKRYSPSLKS | RHMRLSCFDY (SEQ |
| NO: 647) | (SEQ ID NO: 648) | ID NO: 649) |
| SGGYYWS (SEQ ID | YIYYSGSTYYNPSLKS | HYYYYSMDV (SEQ |
| NO: 650) | (SEQ ID NO: 651) | ID NO: 652) |
| CDR1 LC | CDR2 LC | CDR3 LC |
| RASQSISSYLN (SEQ | AASSLQS (SEQ ID NO: | QQSYSTPLT (SEQ ID |
| ID NO: 653) | 654) | NO: 655) |

In some embodiments, the HLA-A*11 ligand binding domain comprises a sequence of any one of SEQ ID NOs: 489, 491, 493, 495, 497, 499, 501, 503, and 505. In some embodiments, the HLA-A*11 ligand binding domain comprises a sequence at least 90%, at least 95% at least 99%, or 100% identical to a sequence of any one of SEQ ID NOs: 489, 491, 493, 495, 497, 499, 501, 503, and 505.

In some embodiments, the HLA-A*11 scFv comprises the complementarity determined regions (CDRs) of any one of SEQ ID NOS: 489, 491, 493, 495, 497, 499, 501, 503, and 505. In some embodiments, the scFv comprises a sequence at least 95% identical to any one of SEQ ID NOS: 489, 491, 493, 495, 497, 499, 501, 503, and 505. In some embodiments, the scFv comprises a sequence identical to any one of SEQ ID NOS: 489, 491, 493, 495, 497, 499, 501, 503, and 505. In some embodiments, the heavy chain of the antigen binding domain comprises the heavy chain CDRs of any one of SEQ ID NOS: 132-140, and wherein the light chain of the antigen binding domain comprises the light chain CDRs of SEQ ID NO: 141. In some embodiments, the HLA-A*11 antigen binding domain comprises a heavy chain and a light chain, and the heavy chain comprises one, two, or three CDRs selected from SEQ ID NOs: 626-652 and the light chain comprises one, two or three CDRs selected from SEQ ID NOs: 653-655.

Illustrative heavy and light chain sequences for HLA-A*11 antigen binding domains are provided in Table 9, below. In some embodiments, the HLA-A*11 antigen binding domain comprises a heavy chain and a light chain, and the heavy chain comprises a sequence at least 95% identical to the heavy chain portion of any one of SEQ ID NOS: 656, 658, 660, 662, 664, 666, 668, 670, and 672 and the light chain comprises a sequence at least 95% identical to the light chain portion of SEQ ID NO: 674.

In some embodiments, the heavy chain comprises a sequence identical to the heavy chain portion of any one of SEQ ID NOS: 489, 491, 493, 495, 497, 499, 501, 503, and 505, and wherein the light chain of comprises a sequence identical to the light chain portion of any one of SEQ ID NOS: 489, 491, 493, 495, 497, 499, 501, 503, and 505.

TABLE 9
Illustrative anti-HLA-A*11 heavy and light chain sequences

| | | |
|---|---|---|
| scFv | Protein Sequence | DNA Sequence |

Heavy Chain Sequences

| | | |
|---|---|---|
| 9 | QVQLQESGPGLVKPSQT | CAGGTGCAGCTGCAGGAAAGCGGCCCTGGCCTGGTGA |
| | LSLTCTVSGGSISSGGYY | AACCCAGCCAGACCCTGAGCCTGACCTGCACAGTGTC |
| | WSWIRQPPGKGLEWIGY | CGGCGGCTCGATCAGCAGCGGCGGCTACTACTGGTCC |
| | IYYSGSTYYNPSLKSRVT | TGGATCAGACAGCCCCCTGGCAAGGGCCTGGAATGGA |
| | ISVDTSKNQFSLKLSSVT | TCGGCTACATCTACTACAGCGGCAGCACCTACTACAA |
| | AADTAVYYCARHYYYY | CCCCAGCCTGAAGTCCAGAGTGACCATCAGCGTGGAC |
| | SMDVWGKGTTVTVSS | ACCAGCAAGAACCAGTTCAGCCTGAAGCTGAGCAGCG |
| | (SEQ ID NO: 656) | TGACAGCCGCCGACACCGCTGTGTATTACTGTGCGAG |
| | | ACACTACTACTACTACTCCATGGACGTCTGGGGCAAA |
| | | GGGACCACGGTCACCGTGTCCTCA (SEQ ID NO: 657) |
| 8 | QITLKESGPTLVKPTQTL | CAGATCACCCTGAAAGAGTCCGGCCCCACCCTGGTGA |
| | TLTCTFSGFSLSTSGVGV | AACCCACCCAGACCCTGACCCTGACATGCACCTTCAG |
| | GWIRQPPGKALEWLALI | CGGCTTCAGCCTGAGCACCTCTGGCGTGGGCGTGGGC |
| | YWNDDKRYSPSLKSRLT | TGGATCAGACAGCCTCCCGGCAAGGCCCTGGAATGGC |
| | ITKDTSKNQVVLTMTN | TGGCCCTGATCTACTGGAACGACGACAAGCGGTACAG |
| | MDPVDTATYYCAHRHM | CCCCAGCCTGAAGTCCCGGCTGACCATCACCAAGGAC |
| | RLSCFDYWGQGTLVTVS | ACCTCGAAGAACCAGGTGGTGCTGACCATGACAAACA |
| | S (SEQ ID NO: 658) | TGGACCCCGTGGACACCGCCACATATTACTGTGCACA |
| | | CAGACACATGCGTTTAAGCTGTTTTGACTACTGGGGCC |
| | | AGGGAACCCTGGTCACCGTGTCCTCA (SEQ ID NO: 659) |
| 7 | QVQLVQSGAEVKKPGA | CAGGTGCAGCTGGTGCAGTCTGGCGCCGAAGTGAAGA |
| | SVKVSCKASGYTFTSYA | AACCTGGCGCCTCCGTGAAGGTGTCCTGCAAGGCCAG |
| | MHWVRQAPGQRLEWM | CGGCTACACCTTCACCAGCTACGCCATGCACTGGGTTC |
| | GWINAGNGNTKYSQKF | GACAGGCCCCTGGCCAGAGACTGGAATGGATGGGCTG |
| | QGRVTITRDTSASTAYM | GATCAACGCCGGCAACGGCAACACCAAGTACAGCCA |
| | ELSSLRSEDTAVYYCAR | GAAATTCCAGGGCAGAGTGACCATCACCCGGGACACC |
| | EGNGANPDAFDIWGQG | AGCGCCAGCACCGCCTACATGGAACTGAGCAGCCTGC |
| | TMVTVSS (SEQ ID NO: | GGAGCGAGGACACCGCTGTGTATTACTGTGCGAGAGA |
| | 660) | AGGAAATGGTGCCAACCCTGATGCTTTTGATATCTGG |
| | | GGCCAAGGGACAATGGTCACCGTGTCCTCA (SEQ ID |
| | | NO: 661) |
| 6 | EVQLVESGGGLVQPGGS | GAAGTGCAGCTGGTGGAAAGCGGCGGAGGCCTGGTG |
| | LRLSCAASGFTFSSYDM | CAGCCTGGCGGCAGCCTGAGACTGTCTTGCGCCGCCA |
| | HWVRQATGKGLEWVSA | GCGGCTTCACCTTCAGCAGCTACGACATGCACTGGGT |
| | IGTAGDTYYPGSVKGRF | CCGCCAGGCCACCGGCAAGGGACTGGAATGGGTGTCC |
| | TISRENAKNSLYLQMNS | GCCATCGGCACAGCCGGCGACACTTACTACCCCGGCA |
| | LRAGDTAVYYCARDLP | GCGTGAAGGGCCGGTTCACCATCAGCAGAGAGAACGC |
| | GSYWYFDLWGRGTLVT | CAAGAACAGCCTGTACCTGCAGATGAACAGCCTTCGA |
| | VSS (SEQ ID NO: 662) | GCCGGCGATACCGCCGTGTATTACTGTGCAAGAGATC |
| | | TCCCTGGTAGCTACTGGTACTTCGATCTCTGGGGCCGT |
| | | GGCACCCTGGTCACTGTGTCCTCA (SEQ ID NO: 663) |
| 5 | QVQLQESGPGLVKPSQT | CAGGTGCAGCTGCAGGAAAGCGGCCCTGGCCTGGTGA |
| | LSLTCTVSGGSISSGGYY | AACCCAGCCAGACCCTGAGCCTGACCTGCACAGTGTC |
| | WSWIRQPPGKGLEWIGY | CGGCGGCTCGATCAGCAGCGGCGGCTACTACTGGTCC |
| | IYYSGSTYYNPSLKSRVT | TGGATCAGACAGCCCCCTGGCAAGGGCCTGGAATGGA |
| | ISVDTSKNQFSLKLSSVT | TCGGCTACATCTACTACAGCGGCAGCACCTACTACAA |
| | AADTAVYYCARHYYYY | CCCCAGCCTGAAGTCCAGAGTGACCATCAGCGTGGAC |
| | YLDVWGKGTTVTVSS | ACCAGCAAGAACCAGTTCAGCCTGAAGCTGAGCAGCG |
| | (SEQ ID NO: 664) | TGACAGCCGCCGACACCGCTGTGTATTACTGTGCGAG |
| | | ACACTACTACTACTACTACCTGGACGTCTGGGGCAAA |
| | | GGGACCACGGTCACCGTGTCCTCA (SEQ ID NO: 665) |
| 4 | EVQLVESGGGLVQPGGS | GAAGTGCAGCTGGTGGAAAGCGGCGGAGGCCTGGTG |
| | LRLSCAASGFTFSSYWM | CAGCCTGGCGGCAGCCTGAGACTGTCTTGCGCCGCCA |
| | HWVRQAPGKGLVWVSR | GCGGCTTCACCTTCAGCAGCTACTGGATGCACTGGGT |
| | INSDGSSTSYADSVKGRF | CCGCCAGGCCCCTGGCAAGGGACTGGTCTGGGTGTCT |
| | TISRDNAKNTLYLQMNS | CGAATCAACAGCGACGGCAGCAGCACCAGCTACGCC |
| | LRAEDTAVYYCCLGVLL | GACAGCGTGAAGGGCCGGTTCACCATCAGCCGGGACA |
| | YNWFDPWGQGTLVTVS | ACGCCAAGAACACCCTGTACCTGCAGATGAACAGCCT |
| | S (SEQ ID NO: 666) | GCGGGCCGAGGACACCGCCGTGTATTACTGTTGTTTG |
| | | GGTGTTTTATTATACAACTGGTTCGACCCCTGGGGCCA |
| | | GGGAACCCTGGTCACCGTGTCCTCA (SEQ ID NO: 667) |
| 3 | QVQLQESGPGLVKPSQT | CAGGTGCAGCTGCAGGAAAGCGGCCCTGGCCTGGTGA |
| | LSLTCTVSGGSISSGGYY | AACCCAGCCAGACCCTGAGCCTGACCTGCACAGTGTC |
| | WSWIRQPPGKGLEWIGY | CGGCGGCTCGATCAGCAGCGGCGGCTACTACTGGTCC |
| | IYYSGSTYYNPSLKSRVT | TGGATCAGACAGCCCCCTGGCAAGGGCCTGGAATGGA |
| | ISVDTSKNQFSLKLSSVT | TCGGCTACATCTACTACAGCGGCAGCACCTACTACAA |
| | AADTAVYYCARHYYYY | CCCCAGCCTGAAGTCCAGAGTGACCATCAGCGTGGAC |
| | MDVWGKGTTVTVSS | ACCAGCAAGAACCAGTTCAGCCTGAAGCTGAGCAGCG |
| | (SEQ ID NO: 668) | TGACAGCCGCCGACACCGCTGTGTATTACTGTGCGAG |
| | | ACACTACTACTACTACATGGACGTCTGGGGCAAAGGG |
| | | ACCACGGTCACCGTGTCCTCA (SEQ ID NO: 669) |
| 2 | QITLKESGPTLVKPTQTL | CAGATCACCCTGAAAGAGTCCGGCCCCACCCTGGTGA |
| | TLTCTFSGFSLSTSGVGV | AACCCACCCAGACCCTGACCCTGACATGCACCTTCAG |
| | GWIRQPPGKALEWLALI | CGGCTTCAGCCTGAGCACCTCTGGCGTGGGCGTGGGC |
| | YWNDDKRYSPSLKSRLT | TGGATCAGACAGCCTCCCGGCAAGGCCCTGGAATGGC |
| | ITKDTSKNQVVLTMTN | TGGCCCTGATCTACTGGAACGACGACAAGCGGTACAG |
| | MDPVDTATYYCAHKTT | CCCCAGCCTGAAGTCCCGGCTGACCATCACCAAGGAC |
| | SFYFDYWGQGTLVTVSS | ACCTCGAAGAACCAGGTGGTGCTGACCATGACAAACA |
| | (SEQ ID NO: 670) | TGGACCCCGTGGACACCGCCACATATTACTGTGCACA |
| | | CAAAACGACGTCGTTTTACTTTGACTACTGGGGCCAG |
| | | GGAACCCTGGTCACCGTGTCCTCA (SEQ ID NO: 671) |
| 1 | QVQLQESGPGLVKPSQT | CAGGTGCAGCTGCAGGAAAGCGGCCCTGGCCTGGTGA |
| | LSLTCTVSGGSISSGGYY | AACCCAGCCAGACCCTGAGCCTGACCTGCACAGTGTC |
| | WSWIRQPPGKGLEWIGY | CGGCGGCTCGATCAGCAGCGGCGGCTACTACTGGTCC |
| | IYYSGSTYYNPSLKSRVT | TGGATCAGACAGCCCCCTGGCAAGGGCCTGGAATGGA |
| | ISVDTSKNQFSLKLSSVT | TCGGCTACATCTACTACAGCGGCAGCACCTACTACAA |
| | AADTAVYYCARHYYYY | CCCCAGCCTGAAGTCCAGAGTGACCATCAGCGTGGAC |
| | YMDVWGKGTTVTVSS | ACCAGCAAGAACCAGTTCAGCCTGAAGCTGAGCAGCG |
| | (SEQ ID NO: 672) | TGACAGCCGCCGACACCGCTGTGTATTACTGTGCGAG |
| | | ACACTACTACTACTACTACATGGACGTCTGGGGCAAA |
| | | GGGACCACGGTCACCGTGTCCTCA (SEQ ID NO: 673) |

Light Chain Sequence

| | | |
|---|---|---|
| 1-9 | DIQMTQSPSSLSASVGD | GACATCCAGATGACCCAGTCTCCATCCTCCCTGTCTGC |
| | RVTITCRASQSISSYLNW | ATCTGTAGGAGACAGAGTCACCATCACTTGCCGGGCA |
| | YQQKPGKAPKLLIYAAS | AGTCAGAGCATTAGCAGCTATTTAAATTGGTATCAGC |
| | SLQSGVPSRFSGSGSGTD | AGAAACCAGGGAAAGCCCCTAAGCTCCTGATCTATGC |
| | FTLTISSLQPEDFATYYC | TGCATCCAGTTTGCAAAGTGGGGTCCCATCAAGGTTC |
| | QQSYSTPLTFGGGTKVEI | AGTGGCAGTGGATCTGGGACAGATTTCACTCTCACCA |
| | K (SEQ ID NO: 674) | TCAGCAGTCTGCAACCTGAAGATTTTGCAACTTACTAC |
| | | TGTCAACAGAGTTACAGTACCCCTCTCACTTTCGGCGG |
| | | CGGAACAAAGGTGGAGATCAAG (SEQ ID NO: 675) |

### Differentially Expressed Inhibitor Ligands

The disclosure provides inhibitor ligands (allogeneic donor cell antigens) that are differentially expressed between the subject's cells and allogeneic donor cells.

Activation of the inhibitory receptor is mediated by the presence of the allogeneic donor cell antigen on the surface of a cell. A cell that expresses the allogeneic donor cell antigen will activate the inhibitory receptor based on the level of expression of the allogeneic donor cell antigen. In some embodiments, the allogeneic donor cell antigen is expressed by both the subject's cells and allogeneic donor cell cells. However, in these embodiments, the allogeneic donor cell antigen is expressed by allogeneic donor cells at a higher level than in the subject's cells. The higher levels of allogeneic donor cell antigen expressed by the allogeneic donor cells activate the inhibitory receptor, thereby preventing activation of the immune cell. In contrast, the lower levels of allogeneic donor cell antigen expressed by the subject's cells are not sufficient to activate the inhibitory receptor, leading to activation of the immune cell.

In alternative embodiments, the allogeneic donor cell antigen is expressed by allogeneic donor cells but not by the subject's cells. In the absence of expression of the allogeneic donor cell antigen, the subject's cells stimulate the activator receptor, thereby activating the immune cells.

Differential expression can be determined by any techniques known in the art used to measure expression. These include, inter alia, techniques for measuring mRNA and/or protein levels of a target gene in a cell. Methods of measuring protein levels in samples include immunohistochemistry, enzyme-linked immunosorbent assays (ELISA), and analytical methods such as liquid chromatography-mass spectrometry (LC-MS). Methods of measuring mRNA levels include real time quantitative reverse transcription PCR (qRT-PCR), as well as high throughput sequencing. Expression differences can be observed between, for example, a subject cell and an allogeneic donor cell.

Activation of the inhibitory receptor by an allogeneic donor cell antigen can occur according to various modalities known in the art. Activation of the inhibitory receptor by an allogeneic donor cell antigen can be determined by methods known in the art. For example, the level of downstream intracellular signaling in a cell expressing the inhibitory receptor can be measured through the use of a reporter gene.

Without wishing to be bound by theory, whether or not expression of an allogeneic donor cell antigen inhibits activation of an immune cell via activation of the inhibitory receptor can occur according to the ratio of the allogeneic donor cell antigen to the inhibitor receptor. The expression levels of the allogeneic donor cell antigen and the inhibitory receptor, and the ratio thereof, can be determined by methods known in the art, including, inter alia, immunohistochemistry and fluorescence activated cell sorting (FACS). Analysis of the expression levels of the allogeneic donor cell antigen on subject and allogeneic donor cells can be used to predict selective targeting of the immune cells expressing the inhibitory receptor. Low or no expression of the allogeneic donor cell antigen on a subject or allogeneic donor cell can indicate, for example, that the inhibitory receptor will not be activated in an immune cell of the disclosure.

In some embodiments, the allogeneic donor cellantigen is selected from the group consisting of leucine rich repeat neuronal 4 (LRRN4) and uroplakin B3 (UPKB3), or a peptide antigen of any of these in a complex with a major histocompatibility complex class I (MHC-I). In some embodiments, the allogeneic donor cell antigen is LRRN4 or a peptide antigen thereof in a complex with MHC-I. In some embodiments, the allogeneic donor cell antigen is UPKB3 or a peptide antigen thereof in a complex with MHC-I.

In some embodiments, the A antigen is a peptide antigen of a cancer cell-specific antigen in a complex with a major histocompatibility complex class I (MHC-I).

Allogeneic donor cell MHC-I (pMHC) antigens comprising any of HLA-A, HLA-B or HLA-C are envisaged as within the scope of the disclosure. In some embodiments, the allogeneic donor cell antigen comprises HLA-A. In some embodiments, the allogeneic donor cell antigen comprises HLA-B. In some embodiments, the allogeneic donor cell antigen comprises HLA-C.

In some embodiments, the allogeneic donor cellallogeneic donor cell antigen comprises LRRN4 or an antigen peptide thereof in a complex with MHC-I. A human LRRN4 is described in NCBI record number NP_689824.2, the contents of which are incorporated by reference herein in their entirety. In some embodiments, the non-target antigen comprises UPK3B or an antigen peptide thereof in a complex with MHC-I. All isoforms of UPK3B are envisaged as within the scope of the instant disclosure. A human UPK3B isoform a precursor is described in NCBI record number NP_085047.1, the contents of which are incorporated by reference herein in their entirety. A human UPK3B isoform b precursor is described in NCBI record number NP_872625.1, the contents of which are incorporated by reference herein in their entirety. A human UPK3B isoform c precursor is described in NCBI record number NP 872624.1, the contents of which are incorporated by reference herein in their entirety. A human UPK3B isoform d precursor is described in NCBI record number NP_001334613.1, the contents of which are incorporated by reference herein in their entirety.

### Conditioning Regimen

The present disclosure provides a method of killing blood cells. In some embodiments, the method comprises contacting blood cells with the immune cell of the present disclosure. In some embodiments, the inhibitor receptor of the engineered immune cell is specific to an inhibitor antigen expressed by the allogenic stem cells, such that the allogenic stem cells are spared from killing by the immune cells. In some embodiments, the blood cells lack the inhibitor antigen, such that the immune cell kills the blood cells.

The present disclosure provides a method of conditioning a subject for allogeneic stem cell transplant. In some embodiments, the method comprises administering to the subject the immune cell of the present disclosure.

In some embodiments, the immune cell of the present disclosure is administered in an amount effective to condition the subject for stem cell transplant without a second conditioning therapy.

In some embodiments, the immune cell is administered in an amount effective to treat blood cancer in the subject without a second conditioning therapy.

In some embodiments, the method comprises administering a second conditioning therapy.

In some embodiments, the second conditioning therapy is chemotherapy.

In some embodiments, the second conditioning therapy includes immunosuppressive drugs including fludarabine, cyclophosphamide, busulfan, and/or thiotepa.

In some embodiments, the second conditioning therapy is radiation therapy.

In some embodiments, the second conditioning therapy is administered in an amount less than an amount effective to condition the subject for stem cell transplant without the immune cell.

In some embodiments, the second conditioning therapy is administered in an amount less than an amount effective to treat blood cancer in the subject without the immune cell.

The present disclosure provides a method of allogeneic stem cell transplant in a subject in need thereof. In some embodiments, the method comprises administering to the subject the immune cell of the present disclosure and an allogeneic stem cell transplant. In this embodiment, the inhibitor receptor of the immune cell is specific to an inhibitor antigen expressed by the cells of the stem cell transplant, such that the immune cells spare the stem cell transplant, and the blood cells of the subject lack the inhibitor antigen, such that the immune cell or pharmaceutical composition kills blood cells in the subject to condition the subject for the stem cell transplant.

### Treating Cancer

The present disclosure provides a method of treating or preventing relapse of blood cancer in a subject treated for blood cancer with allogeneic stem cell transplant. In some embodiments, the method comprises administering to the subject the immune cell of the present disclosure.

The present disclosure provides a method of treating blood cancer in a subject in need thereof. In some embodiments, the method comprises administering to the subject the immune cell of the present disclosure and an allogeneic stem cell transplant. In this embodiment, the inhibitor receptor of the immune cell is specific to an inhibitor antigen expressed by the cells of the stem cell transplant, such that the immune cells spare the stem cell transplant, and the cells of the blood cancer lack the inhibitor antigen, such that the immune cell kills blood cancer cells in the subject.

Cancer treatment combination of allogeneic stem cell transplant and immunotherapy is known in the art is described, for example, in Gottlieb et al., Combining CD34+ stem cell selection with prophylactic pathogen and leukemia directed T-cell immunotherapy to simultaneously reduce graft versus host disease, infection, and leukemia recurrence after allogeneic stem cell transplant, Am J Hematol 1-7 (2022).

In some embodiments, the blood cancer is leukemia.

In some embodiments, the blood cancer acute myeloid leukemia (AML).

In some embodiments, wherein the blood cancer is a lymphoma.

HLA matching is known in the art and is described, for example, in Furst et al., HLA Matching in Unrelated Stem Cell Transplantation up to Date, Transfus Med Hemother, 326-336 (2019).

The present disclosure provides a method of treating or preventing relapse of blood cancer in a subject wherein the immune cell and allogeneic stem cell transplant are selected such that if the subject is homozygous null HLA-A*02−/−, the inhibitor antigen is HLA-A*02 and the allogeneic stem cell transplant is HLA-A*02+.

The present disclosure provides a method of treating or preventing relapse of blood cancer in a subject wherein the immune cell and allogeneic stem cell transplant are selected such that if the subject is homozygous null HLA-B*07−/−, the inhibitor antigen is HLA-B*07 and the allogeneic stem cell transplant is HLA-B*07+.

The present disclosure provides a method of treating or preventing relapse of blood cancer in a subject wherein the immune cell and allogeneic stem cell transplant are selected such that if the subject is homozygous null HLA-C*07−/−, the inhibitor antigen is HLA-C*07 and the allogeneic stem cell transplant is HLA-C*07+.

The present disclosure provides a method of treating or preventing relapse of blood cancer in a subject wherein the immune cell and allogeneic stem cell transplant are selected such that if the subject is homozygous null HLA-A*69−/−, the inhibitor antigen is HLA-A*69 and the allogeneic stem cell transplant is HLA-A*69+.

The present disclosure provides a method of allogeneic stem cell transplant, comprising identifying a subject as homozygous null for an allelic variant of an HLA; and matching the subject to an immune cell comprising an inhibitor receptor specific to the allelic variant and an allogeneic stem cell positive for the allelic variant.

In some embodiments, the method comprises identifying the subject as homozygous null for the allelic variant of the HLA to which the inhibitor receptor of the immune cell is specific.

In some embodiments, the method comprises selecting the allogeneic stem cell transplant as positive for the allelic variant.

### ENUMERATED EMBODIMENTS

### Enumerated Embodiments Set I

Embodiment I-1 provides an immune cell for treatment of blood cancer, comprising:
a) an activator receptor comprising an extracellular ligand binding domain specific to a first activator antigen expressed by blood cancer cells; and
b) an inhibitor receptor comprising an extracellular ligand binding domain specific to a first inhibitor antigen expressed by non-cancerous blood cells.

Embodiment I-2 provides the immune cell of embodiment 1, wherein the first activator antigen is expressed by hematopoietic cells.

Embodiment I-3 provides the immune cell of any one of the preceding embodiments, wherein the first inhibitor antigen is expressed by myeloid cells.

Embodiment I-4 provides the immune cell of any one of the preceding embodiments, wherein the myeloid cells are differentiated.

Embodiment I-5 provides the immune cell of any one of the preceding embodiments, wherein the first inhibitor antigen is expressed by hematopoietic stem cells.

Embodiment I-6 provides the immune cell of any one of the preceding embodiments, wherein the first activator antigen is SPN or a peptide antigen of SPN.

Embodiment I-7 provides the immune cell of any one of the preceding embodiments, wherein the first inhibitor antigen is PECAM-1 or a peptide antigen of PECAM-1.

Embodiment I-8 provides the immune cell of any one of the preceding embodiments, wherein the first activator antigen is SPN and the first inhibitor antigen is PECAM-1.

Embodiment I-9 provides the immune cell of any one of the preceding embodiments, wherein the cancer cell expresses SPN.

Embodiment I-10 provides the immune cell of any one of the preceding embodiments, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-11 provides the immune cell of any one of the preceding embodiments, wherein the blood cancer cell is a leukemia cell or a lymphoma cell.

Embodiment I-12 provides the immune cell of any one of embodiments 7-11, wherein the PECAM-1 antigen is expressed by non-cancerous blood cells of a subject.

Embodiment I-13 provides the immune cell of any one of the preceding embodiments, wherein the non-cancerous blood cells of the subject express both the first activator antigen and the first inhibitor antigen.

Embodiment I-14 provides the immune cell of any one of the preceding embodiments, wherein the activator receptor and the inhibitor receptor specifically activate the immune cell when in contact with the blood cancer cell.

Embodiment I-15 provides the immune cell of any one of the preceding embodiments, wherein the activator receptor specifically activates the immune cell when in contact with the blood cancer cell.

Embodiment I-16 provides the immune cell of any one of the preceding embodiments, wherein the inhibitor receptor specifically inhibits the immune cell when in contact with the non-cancerous blood cell.

Embodiment I-17 provides the immune cell of any one of the preceding embodiments, wherein the immune cell is a T cell.

Embodiment I-18 provides the immune cell of Embodiment I-17, wherein the T cell is a CD8+ CD4− T cell.

Embodiment I-19 provides the immune cell of any one of the preceding embodiments, wherein the SPN antigen comprises a sequence or subsequence at least 95% identical to a sequence or subsequence of any one of SEQ ID NOs: 172-173.

Embodiment I-20 provides the immune cell of any one of the preceding embodiments, wherein the activator receptor is a T cell receptor (TCR) or a chimeric antigen receptor (CAR).

Embodiment I-21 provides the immune cell of Embodiment I-20, wherein the extracellular ligand binding domain of the activator receptor comprises an antibody fragment, a single chain Fv antibody fragment (scFv), a β chain variable domain (Vβ), or a TCR α chain variable domain and a TCR β chain variable domain.

Embodiment I-22 provides the immune cell of any one of embodiments 20-21, wherein the extracellular ligand binding domain of the activator receptor comprises a heavy chain variable (VH) region and a light chain variable (VL) region.

Embodiment I-23 provides the immune cell of Embodiment I-22, wherein the VH region comprises the sequence of any one of SEQ ID NOs: 249-252, 257, 259, or 261-280; and the VL region comprises the sequence of any one of SEQ ID NOs: 253-256, 258, 260, or 281-303.

Embodiment I-24 provides the immune cell of any one of embodiments 20-23, wherein the CAR comprises a hinge sequence isolated or derived from CD8, CD28, IgG1, or IgG4, or a synthetic hinge.

Embodiment I-25 provides the immune cell of any one of embodiments 20-24, wherein the CAR comprises a transmembrane domain isolated or derived from CD8 or CD28.

Embodiment I-26 provides the immune cell of any one of embodiments 20-25, wherein the CAR comprises an intracellular domain isolated or derived from CD28, 4-1BB or CD3z, or a combination thereof.

Embodiment I-27 provides the immune cell of any one of the preceding embodiments, wherein the inhibitor receptor is a TCR or a CAR.

Embodiment I-28 provides the immune cell of Embodiment I-27, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-29 provides the immune cell of any one of the embodiments 27-28, wherein the extracellular ligand binding domain of the inhibitor receptor comprises a heavy chain variable (VH) region and a light chain variable (VL) region.

Embodiment I-30 provides the immune cell of any one of the preceding embodiments, wherein the inhibitor receptor comprises a LILRB1 intracellular domain or a functional variant thereof.

Embodiment I-31 provides the immune cell of any one of the preceding embodiments, wherein the inhibitor receptor comprises LILRB1 hinge and transmembrane domains, or functional variants thereof.

Embodiment I-32 provides a pharmaceutical composition, comprising an effective amount of the immune cells of any one of embodiments 1-31.

Embodiment I-33 provides a pharmaceutical composition of Embodiment I-32, further comprising a pharmaceutically acceptable carrier, diluent or excipient.

Embodiment I-34 provides a pharmaceutical composition of any one of embodiments 32 or 33, for use as a medicament in the treatment of cancer.

Embodiment I-35 provides a polynucleotide system, comprising one or more polynucleotides comprising polynucleotide sequences encoding:
a) an activator receptor comprising an extracellular ligand binding domain specific to a SPN antigen; and
b) an inhibitor receptor comprising an extracellular ligand binding domain specific to a PECAM-1 antigen.

Embodiment I-36 provides a method of making an immune cell therapy, comprising transforming immune cells with the polynucleotide system of Embodiment I-35.

Embodiment I-37 provides a kit comprising the immune cell of any one of embodiments 1-31 or the pharmaceutical composition of any one of embodiments 32-34.

Embodiment I-38 provides an immune cell comprising:
a) an activator receptor comprising an extracellular ligand binding domain specific to a SPN antigen, wherein the extracellular ligand binding domain of the activator receptor is an scFv; and
b) an inhibitor receptor comprising an extracellular ligand binding domain specific to a PECAM-1 antigen that is not expressed by a blood cancer cell, wherein the extracellular ligand binding domain of the inhibitor receptor is an scFv.

Embodiment I-39 provides a method of treating cancer in a subject, comprising administering to the subject an immune cell comprising
a) an activator receptor comprising an extracellular ligand binding domain specific to a SPN antigen, wherein the extracellular ligand binding domain of the activator receptor is an scFv; and
b) an inhibitor receptor comprising an extracellular ligand binding domain specific to a PECAM-1 antigen that is not expressed by a blood cancer cell, wherein the extracellular ligand binding domain of the inhibitory receptor is an scFv.

Embodiment I-40 provides a method of identifying antigens for use in treating blood cancers with chimeric antigen receptor immune cell therapy, comprising:
a) selecting an antigen expressed on blood cancer cells; and
b) selecting an antigen expressed on non-cancerous blood cells.

Embodiment I-41 provides the method of Embodiment I-40, comprising generating an immune cell comprising two or more receptors comprising antigen-binding domains, wherein one receptor binds to a first antigen expressed on blood cancer cells and one receptor binds to the second antigen expressed on non-cancerous blood cells.

Embodiment I-42 provides the immune cell of any one of embodiments 1-5, wherein the first activator antigen is CD45 or a peptide antigen of CD45.

Embodiment I-43 provides the immune cell of Embodiment I-42, wherein the first inhibitor antigen is PECAM-1 or a peptide antigen of PECAM-1.

Embodiment I-44 provides the immune cell of Embodiment I-42, wherein the first activator antigen is CD45 and the first inhibitor antigen is PECAM-1.

Embodiment I-45 provides the immune cell of Embodiment I-42, wherein the cancer cell expresses CD45.

Embodiment I-46 provides the immune cell of any one of embodiments 42-45, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-47 provides the immune cell of any one of embodiments 42-45, wherein the blood cancer cell is a leukemia cell or a lymphoma cell.

Embodiment I-48 provides the immune cell of any one of embodiments 42-47, wherein the PECAM-1 antigen is expressed by non-cancerous blood cells of a subject.

Embodiment I-49 provides the immune cell of any one of embodiments 42-48, wherein the immune cell is a T cell.

Embodiment I-50 provides the immune cell of any one of embodiments 42-49, wherein the CD45 antigen comprises a sequence or subsequence at least 95% identical to a sequence or subsequence of any one of SEQ ID NOs: 1-9 or 327.

Embodiment I-51 provides the immune cell of any one of embodiments 42-50, wherein the activator receptor is a TCR or a CAR.

Embodiment I-52 provides the immune cell of Embodiment I-51, wherein the extracellular ligand binding domain of the activator receptor comprises an antibody fragment, a single chain Fv antibody fragment (scFv), a β chain variable domain (Vβ), or a TCR α chain variable domain and a TCR β chain variable domain.

Embodiment I-53 provides the immune cell of embodiments 51 or 52, wherein the extracellular binding domain of the activator receptor comprises a VH region comprising any one of the sequences of SEQ ID NOs: 16, 17, 20, 21, 23, 25, 27 or a sequence having at least 85%, at least 90%, at least 95%, at least 97%, at least 99% identity, or 100% identical thereto; and a VL region comprising any one of the sequences of SEQ ID NOs: 18, 19, 22, 24, 26, 28, or a sequence having at least 85%, at least 90%, at least 95%, at least 97%, at least 99% identity, or 100% identical thereto.

Embodiment I-54 provides the immune cell of Embodiment I-53, wherein the VH region comprises one or more CDR sequences selected from the group consisting of RYWMS (SEQ ID NO: 10), EINPTSSTINFTPSLKD (SEQ ID NO: 11), and GNYYRYGDAMDY (SEQ ID NO: 12); and the VL region comprises one or more CDR sequences selected from the group consisting of RASKSVSTSGYSYLH (SEQ ID NO: 13), LASNLES (SEQ ID NO: 14), and QHSRELPFTFGSGT (SEQ ID NO: 15).

Embodiment I-55 provides the immune cell of Embodiment I-52, wherein the scFv of the activator receptor comprises a sequence having at least 85%, at least 90%, at least 95%, at least 97%, at least 99% identity, or 100% identical to a sequence selected from the group consisting of SEQ ID NOS: 29-32.

Embodiment I-56 provides the immune cell of any one of embodiments 42-55, wherein the activator receptor comprises a sequence having at least 85%, at least 90%, at least 95%, at least 97%, at least 99% identity, or 100% identical to any one of SEQ ID NOs: 133-140, 142, 144, 146, 148, 151, 153, 155-168, or 328-330.

Embodiment I-57 provides the immune cell of any one of embodiments 42-56, wherein the inhibitor receptor is a TCR or a CAR.

Embodiment I-58 provides the immune cell of Embodiment I-57, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-59 provides the immune cell of any one of embodiments 42-58, wherein the inhibitor receptor comprises LILRB1 hinge and transmembrane domains, or functional variants thereof.

Embodiment I-60 provides a pharmaceutical composition, comprising an effective amount of the immune cells of any one of embodiments 42-59.

Embodiment I-61 provides the pharmaceutical composition of Embodiment I-60, for use as a medicament in the treatment of cancer.

Embodiment I-62 provides a polynucleotide system, comprising one or more polynucleotides comprising polynucleotide sequences encoding:
a) an activator receptor comprising an extracellular ligand binding domain specific to a CD45 antigen; and
b) an inhibitor receptor comprising an extracellular ligand binding domain specific to a PECAM-1 antigen.

Embodiment I-63 provides the immune cell of any one of embodiments 1-5, wherein the first activator antigen is CD11a or a peptide antigen of CD11a.

Embodiment I-64 provides the immune cell of Embodiment I-63, wherein the first inhibitor antigen is PECAM-1 or a peptide antigen of PECAM-1.

Embodiment I-65 provides the immune cell of Embodiment I-63, wherein the first activator antigen is CD11a and the first inhibitor antigen is PECAM-1.

Embodiment I-66 provides the immune cell of Embodiment I-63-65, wherein the cancer cell expresses CD11a.

Embodiment I-67 provides the immune cell of any one of embodiments 63-66, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-68 provides the immune cell of any one of embodiments 63-67, wherein the blood cancer cell is a leukemia cell or a lymphoma cell.

Embodiment I-69 provides the immune cell of any one of embodiments 63-68, wherein the PECAM-1 antigen is expressed by non-cancerous blood cells of a subject.

Embodiment I-70 provides the immune cell of any one of embodiments 63-69, wherein the immune cell is a T cell.

Embodiment I-71 provides the immune cell of any one of embodiments 63-70, wherein the CD11a antigen comprises a sequence or subsequence at least 95% identical to a sequence or subsequence of any one of SEQ ID NOs: 169-171.

Embodiment I-72 provides the immune cell of any one of embodiments 63-71, wherein the activator receptor is a TCR or a CAR.

Embodiment I-73 provides the immune cell of Embodiment I-72, wherein the extracellular ligand binding domain of the activator receptor comprises an antibody fragment, a single chain Fv antibody fragment (scFv), a β chain variable domain (Vβ), or a TCR α chain variable domain and a TCR β chain variable domain.

Embodiment I-74 provides the immune cell of Embodiment I-72 or 73, wherein the extracellular binding domain of the activator receptor comprises a VH region comprising any one of the sequences of SEQ ID NOs: 179, 247, or a sequence having at least 85%, at least 90%, at least 95%, at least 97%, at least 99% identity, or 100% identical thereto; and a VL region comprising any one of the sequences of SEQ ID NOs: 180, 248, or a sequence having at least 85%, at least 90%, at least 95%, at least 97%, at least 99% identity, or 100% identical thereto.

Embodiment I-75 provides the immune cell of Embodiment I-73, wherein the scFv of the activator receptor comprises a sequence having at least 85%, at least 90%, at least 95%, at least 97%, at least 99% identity, or 100% identical to a sequence selected from the group consisting of SEQ ID NOS: 304-312.

Embodiment I-76 provides the immune cell of any one of embodiments 63-75, wherein the activator receptor comprises a sequence having at least 85%, at least 90%, at least 95%, at least 97%, at least 99% identity, or 100% identical to any one of SEQ ID NOs: 313-318.

Embodiment I-77 provides the immune cell of any one of embodiments 63-76, wherein the inhibitor receptor is a TCR or a CAR.

Embodiment I-78 provides the immune cell of Embodiment I-77, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-79 provides the immune cell of any one of embodiments 63-78, wherein the inhibitor receptor comprises LILRB1 hinge and transmembrane domains, or functional variants thereof.

Embodiment I-80 provides a pharmaceutical composition, comprising an effective amount of the immune cells of any one of embodiments 63-79.

Embodiment I-81 provides the pharmaceutical composition of Embodiment I-80, for use as a medicament in the treatment of cancer.

Embodiment I-82 provides a polynucleotide system, comprising one or more polynucleotides comprising polynucleotide sequences encoding:
a) an activator receptor comprising an extracellular ligand binding domain specific to a CD11a antigen; and
b) an inhibitor receptor comprising an extracellular ligand binding domain specific to a PECAM-1 antigen.

Embodiment I-83 provides the immune cell of any one of embodiments 1-5, wherein the first activator antigen is SPN or a peptide antigen of SPN.

Embodiment I-84 provides the immune cell of Embodiment I-83, comprising a second extracellular ligand binding domain specific to a second activator antigen expressed by blood cancer cells, wherein the second activator antigen is CD33 or a peptide antigen of CD33.

Embodiment I-85 provides the immune cell of Embodiment I-84, wherein the first inhibitor antigen is PECAM-1 or a peptide antigen of PECAM-1.

Embodiment I-86 provides the immune cell of Embodiment I-83, wherein the first activator antigen is SPN, the second activator antigen is CD33, and the first inhibitor antigen is PECAM-1.

Embodiment I-87 provides the immune cell of embodiments 83-86, wherein the cancer cell expresses SPN or CD33.

Embodiment I-88 provides the immune cell of any one of embodiments 83-87, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-89 provides the immune cell of any one of embodiments 83-88, wherein the blood cancer cell is a leukemia cell or a lymphoma cell.

Embodiment I-90 provides the immune cell of any one of embodiments 83-89, wherein the PECAM-1 antigen is expressed by non-cancerous blood cells of a subject.

Embodiment I-91 provides the immune cell of any one of embodiments 83-90, wherein the immune cell is a T cell.

Embodiment I-92 provides the immune cell of any one of embodiments 83-91, wherein the SPN antigen comprises a sequence or subsequence at least 95% identical to a sequence or subsequence of any one of SEQ ID NOs: 172-173 and/or wherein the CD33 antigen comprises a sequence or subsequence at least 95% identical to a sequence or subsequence of any one of SEQ ID NOs: 174-176.

Embodiment I-93 provides the immune cell of any one of embodiments 83-92, wherein the first and second activator receptors are a TCR or a CAR.

Embodiment I-94 provides the immune cell of Embodiment I-93, wherein the extracellular ligand binding domain of the first activator receptor comprises an antibody fragment, a single chain Fv antibody fragment (scFv), a β chain variable domain (Vβ), or a TCR α chain variable domain and a TCR β chain variable domain.

Embodiment I-95 provides the immune cell of Embodiment I-94, wherein the extracellular binding domain of the first activator receptor comprises a VH region comprising any one of the sequences of SEQ ID NOs: 249-252, 257, 259, 261-280, or a sequence having at least 85%, at least 90%, at least 95%, at least 97%, at least 99% identity, or 100% identical thereto; and a VL region comprising any one of the sequences of SEQ ID NOs: 253-256, 258, 260, 281-303, or a sequence having at least 85%, at least 90%, at least 95%, at least 97%, at least 99% identity, or 100% identical thereto.

Embodiment I-96 provides the immune cell of Embodiment I-93, wherein the extracellular ligand binding domain of the second activator receptor comprises an antibody fragment, a single chain Fv antibody fragment (scFv), a β chain variable domain (Vβ), or a TCR α chain variable domain and a TCR β chain variable domain.

Embodiment I-97 provides the immune cell of Embodiment I-94, wherein the extracellular binding domain of the second activator receptor comprises a VH region comprising any one of the sequences of SEQ ID NOs: 181, 331, 333, 336, 337, 339, 341, or a sequence having at least 85%, at least 90%, at least 95%, at least 97%, at least 99% identity, or 100% identical thereto; and a VL region comprising any one of the sequences of SEQ ID NOs: 182, 332, 334, 335, 338, 340, 342, or a sequence having at least 85%, at least 90%, at least 95%, at least 97%, at least 99% identity, or 100% identical thereto.

Embodiment I-98 provides the immune cell of any one of embodiments 83-97, wherein the inhibitor receptor is a TCR or a CAR.

Embodiment I-99 provides the immune cell of Embodiment I-98, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-100 provides the immune cell of any one of embodiments 63-99, wherein the inhibitor receptor comprises LILRB1 hinge and transmembrane domains, or functional variants thereof.

Embodiment I-101 provides a pharmaceutical composition, comprising an effective amount of the immune cells of any one of embodiments 83-100.

Embodiment I-102 provides the pharmaceutical composition of Embodiment I-101, for use as a medicament in the treatment of cancer.

Embodiment I-103 provides a polynucleotide system, comprising one or more polynucleotides comprising polynucleotide sequences encoding:
a) A first activator receptor comprising an extracellular ligand binding domain specific to a SPN antigen;
b) a second activator receptor comprising an extracellular ligand binding domain specific to a CD33 antigen; and
c) a first inhibitor receptor comprising an extracellular ligand binding domain specific to a PECAM-1 antigen.

Embodiment I-104 provides the immune cell of any one of embodiments 1-5, wherein the first activator antigen is SPN or a peptide antigen of SPN.

Embodiment I-105 provides the immune cell of Embodiment I-104, comprising a second extracellular ligand binding domain specific to a second activator antigen expressed by blood cancer cells, wherein the second activator antigen is CD45 or a peptide antigen of CD45.

Embodiment I-106 provides the immune cell of Embodiment I-105, wherein the first inhibitor antigen is PECAM-1 or a peptide antigen of PECAM-1.

Embodiment I-107 provides the immune cell of any one of embodiments 104-106, wherein the first activator antigen is SPN, the second activator antigen is CD45, and the first inhibitor antigen is PECAM-1.

Embodiment I-108 provides the immune cell of any one of embodiments 104-107, wherein the cancer cell expresses SPN or CD45.

Embodiment I-109 provides the immune cell of any one of embodiments 104-108, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-110 provides the immune cell of any one of embodiments 104-109, wherein the blood cancer cell is a leukemia cell or a lymphoma cell.

Embodiment I-111 provides the immune cell of any one of embodiments 104-110, wherein the PECAM-1 antigen is expressed by non-cancerous blood cells of a subject.

Embodiment I-112 provides the immune cell of any one of embodiments 104-111, wherein the immune cell is a T cell.

Embodiment I-113 provides the immune cell of any one of embodiments 83-91, wherein the SPN antigen comprises a sequence or subsequence at least 95% identical to a sequence or subsequence of any one of SEQ ID NOs: 172-173 and/or wherein the CD45 antigen comprises a sequence or subsequence at least 95% identical to a sequence or subsequence of any one of SEQ ID NOs: 1-9 or 327.

Embodiment I-114 provides the immune cell of any one of embodiments 104-113, wherein the first and second activator receptors are a TCR or a CAR.

Embodiment I-115 provides the immune cell of Embodiment I-114, wherein the extracellular ligand binding domain of the first activator receptor comprises an antibody fragment, a single chain Fv antibody fragment (scFv), a β chain variable domain (Vβ), or a TCR α chain variable domain and a TCR β chain variable domain.

Embodiment I-116 provides the immune cell of Embodiment I-115, wherein the extracellular binding domain of the first activator receptor comprises a VH region comprising any one of the sequences of SEQ ID NOs: 249-252, 257, 259, 261-280, or a sequence having at least 85%, at least 90%, at least 95%, at least 97%, at least 99% identity, or 100% identical thereto; and a VL region comprising any one of the sequences of SEQ ID NOs: 253-256, 258, 260, 281-303, or a sequence having at least 85%, at least 90%, at least 95%, at least 97%, at least 99% identity, or 100% identical thereto.

Embodiment I-117 provides the immune cell of Embodiment I-114, wherein the extracellular ligand binding domain of the second activator receptor comprises an antibody fragment, a single chain Fv antibody fragment (scFv), a β chain variable domain (Vβ), or a TCR α chain variable domain and a TCR β chain variable domain.

Embodiment I-118 provides the immune cell of Embodiment I-117, wherein the extracellular binding domain of the second activator receptor comprises a VH region comprising any one of the sequences of SEQ ID NOs: 16, 17, 20, 21, 23, 25, 27 or a sequence having at least 85%, at least 90%, at least 95%, at least 97%, at least 99% identity, or 100% identical thereto; and a VL region comprising any one of the sequences of SEQ ID NOs: 18, 19, 22, 24, 26, 28, or a sequence having at least 85%, at least 90%, at least 95%, at least 97%, at least 99% identity, or 100% identical thereto.

Embodiment I-119 provides the immune cell of Embodiment I-118, wherein the VH region comprises one or more CDR sequences selected from the group consisting of RYWMS (SEQ ID NO: 10), EINPTSSTINFTPSLKD (SEQ ID NO: 11), and GNYYRYGDAMDY (SEQ ID NO: 12); and the VL region comprises one or more CDR sequences selected from the group consisting of RASKSVSTSGYSYLH (SEQ ID NO: 13), LASNLES (SEQ ID NO: 14), and QHSRELPFTFGSGT (SEQ ID NO: 15).

Embodiment I-120 provides the immune cell of Embodiment I-117, wherein the scFv of the second activator receptor comprises a sequence having at least 85%, at least 90%, at least 95%, at least 97%, at least 99% identity, or 100% identical to a sequence selected from the group consisting of SEQ ID NOS: 29-32.

Embodiment I-121 provides the immune cell of any one of embodiments 104-120, wherein the second activator receptor comprises a sequence having at least 85%, at least 90%, at least 95%, at least 97%, at least 99% identity, or 100% identical to any one of SEQ ID NOs: 133-140, 142, 144, 146, 148, 151, 153, 155-168, or 328-330.

Embodiment I-122 provides the immune cell of any one of embodiments 104-121, wherein the inhibitor receptor is a TCR or a CAR.

Embodiment I-123 provides the immune cell of Embodiment I-122, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-124 provides the immune cell of any one of embodiments 104-123, wherein the inhibitor receptor comprises LILRB1 hinge and transmembrane domains, or functional variants thereof.

Embodiment I-125 provides a pharmaceutical composition, comprising an effective amount of the immune cells of any one of embodiments 104-124.

Embodiment I-126 provides the pharmaceutical composition of Embodiment I-125, for use as a medicament in the treatment of cancer.

Embodiment I-127 provides a polynucleotide system, comprising one or more polynucleotides comprising polynucleotide sequences encoding:
a) a first activator receptor comprising an extracellular ligand binding domain specific to a SPN antigen;
b) a second activator receptor comprising an extracellular ligand binding domain specific to a CD45 antigen; and
c) a first inhibitor receptor comprising an extracellular ligand binding domain specific to a PECAM-1 antigen.

Embodiment I-128 provides the immune cell of any one of embodiments 1-5, wherein the first activator antigen is SPN or a peptide antigen of SPN.

Embodiment I-129 provides the immune cell of Embodiment I-128, comprising a second extracellular ligand binding domain specific to a second activator antigen expressed by blood cancer cells, wherein the second activator antigen is FLT3 or a peptide antigen of FLT3.

Embodiment I-130 provides the immune cell of Embodiment I-129, wherein the first inhibitor antigen is PECAM-1 or a peptide antigen of PECAM-1.

Embodiment I-131 provides the immune cell of Embodiment I-128-130, wherein the first activator antigen is SPN, the second activator antigen is FLT3, and the first inhibitor antigen is PECAM-1.

Embodiment I-132 provides the immune cell of embodiments 128-131, wherein the cancer cell expresses SPN or FLT3.

Embodiment I-133 provides the immune cell of any one of embodiments 128-132, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-134 provides the immune cell of any one of embodiments 128-133, wherein the blood cancer cell is a leukemia cell or a lymphoma cell.

Embodiment I-135 provides the immune cell of any one of embodiments 128-134, wherein the PECAM-1 antigen is expressed by non-cancerous blood cells of a subject.

Embodiment I-136 provides the immune cell of any one of embodiments 128-135, wherein the immune cell is a T cell.

Embodiment I-137 provides the immune cell of any one of embodiments 128-136, wherein the SPN antigen comprises a sequence or subsequence at least 95% identical to a sequence or subsequence of any one of SEQ ID NOs: 172-173 and/or wherein the FLT3 antigen comprises a sequence or subsequence at least 95% identical to a sequence or subsequence of any one of SEQ ID NOs: 177-178.

Embodiment I-138 provides the immune cell of any one of embodiments 128-137, wherein the first and second activator receptors are a TCR or a CAR.

Embodiment I-139 provides the immune cell of Embodiment I-138, wherein the extracellular ligand binding domain of the first activator receptor comprises an antibody fragment, a single chain Fv antibody fragment (scFv), a β chain variable domain (Vβ), or a TCR α chain variable domain and a TCR β chain variable domain.

Embodiment I-140 provides the immune cell of Embodiment I-139, wherein the extracellular binding domain of the first activator receptor comprises a VH region comprising any one of the sequences of SEQ ID NOs: 249-252, 257, 259, 261-280, or a sequence having at least 85%, at least 90%, at least 95%, at least 97%, at least 99% identity, or 100% identical thereto; and a VL region comprising any one of the sequences of SEQ ID NOs: 253-256, 258, 260, 281-303, or a sequence having at least 85%, at least 90%, at least 95%, at least 97%, at least 99% identity, or 100% identical thereto.

Embodiment I-141 provides the immune cell of Embodiment I-138, wherein the extracellular ligand binding domain of the second activator receptor comprises an antibody fragment, a single chain Fv antibody fragment (scFv), a β chain variable domain (Vβ), or a TCR α chain variable domain and a TCR β chain variable domain.

Embodiment I-142 provides the immune cell of Embodiment I-141, wherein the extracellular binding domain of the second activator receptor comprises a VH region comprising any one of the sequences of SEQ ID NOs: 319, 321, 344, 346, 348, 350, 352, 353, 355, 357, 359, 361, 363, 365, 367, 369, 371, 373, 375, 377, or a sequence having at least 85%, at least 90%, at least 95%, at least 97%, at least 99% identity, or 100% identical thereto; and a VL region comprising any one of the sequences of SEQ ID NOs: 320, 322, 343, 345, 347, 349, 351, 354, 356, 358, 360, 362, 364, 366, 368, 370, 372, 374, 376, 378, or a sequence having at least 85%, at least 90%, at least 95%, at least 97%, at least 99% identity, or 100% identical thereto.

Embodiment I-143 provides the immune cell of any one of embodiments 128-142, wherein the inhibitor receptor is a TCR or a CAR.

Embodiment I-144 provides the immune cell of Embodiment I-143, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-145 provides the immune cell of any one of embodiments 128-144, wherein the inhibitor receptor comprises LILRB1 hinge and transmembrane domains, or functional variants thereof.

Embodiment I-146 provides a pharmaceutical composition, comprising an effective amount of the immune cells of any one of embodiments 128-145.

Embodiment I-147 provides the pharmaceutical composition of claim 146, for use as a medicament in the treatment of cancer.

Embodiment I-148 provides a polynucleotide system, comprising one or more polynucleotides comprising polynucleotide sequences encoding:
a) a first activator receptor comprising an extracellular ligand binding domain specific to a SPN antigen;
b) a second activator receptor comprising an extracellular ligand binding domain specific to a FLT3 antigen; and
c) a first inhibitor receptor comprising an extracellular ligand binding domain specific to a PECAM-1 antigen.

Embodiment I-149 provides the immune cell of any one of embodiments 1-5, wherein the first activator antigen is CD33 or a peptide antigen of CD33.

Embodiment I-150 provides the immune cell of Embodiment I-149, comprising a second extracellular ligand binding domain specific to a second activator antigen expressed by blood cancer cells, wherein the second activator antigen is FLT3 or a peptide antigen of FLT3.

Embodiment I-151 provides the immune cell of Embodiment I-150, wherein the first inhibitor antigen is PECAM-1 or a peptide antigen of PECAM-1.

Embodiment I-152 provides the immune cell of Embodiment I-149-151, wherein the first activator antigen is CD33, the second activator antigen is FLT3, and the first inhibitor antigen is PECAM-1.

Embodiment I-153 provides the immune cell of Embodiment I-149-152, wherein the cancer cell expresses CD33 or FLT3.

Embodiment I-154 provides the immune cell of any one of embodiments 149-153, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-155 provides the immune cell of any one of embodiments 149-154, wherein the blood cancer cell is a leukemia cell or a lymphoma cell.

Embodiment I-156 provides the immune cell of any one of embodiments 149-155, wherein the PECAM-1 antigen is expressed by non-cancerous blood cells of a subject.

Embodiment I-157 provides the immune cell of any one of embodiments 149-156, wherein the immune cell is a T cell.

Embodiment I-158 provides the immune cell of any one of embodiments 149-157, wherein the CD33 antigen comprises a sequence or subsequence at least 95% identical to a sequence or subsequence of any one of SEQ ID NOs: 174-176 and/or wherein the FLT3 antigen comprises a sequence or subsequence at least 95% identical to a sequence or subsequence of any one of SEQ ID NOs: 177-178.

Embodiment I-159 provides the immune cell of any one of embodiments 149-158, wherein the first and second activator receptors are a TCR or a CAR.

Embodiment I-160 provides the immune cell of Embodiment I-159, wherein the extracellular ligand binding domain of the first activator receptor comprises an antibody fragment, a single chain Fv antibody fragment (scFv), a β chain variable domain (Vβ), or a TCR α chain variable domain and a TCR β chain variable domain.

Embodiment I-161 provides the immune cell of Embodiment I-160, wherein the extracellular binding domain of the first activator receptor comprises a VH region comprising any one of the sequences of SEQ ID NOs: 181, 331, 333, 336, 337, 339, 341, or a sequence having at least 85%, at least 90%, at least 95%, at least 97%, at least 99% identity, or 100% identical thereto; and a VL region comprising any one of the sequences of SEQ ID NOs: 182, 332, 334, 335, 338, 340, 342, or a sequence having at least 85%, at least 90%, at least 95%, at least 97%, at least 99% identity, or 100% identical thereto.

Embodiment I-162 provides the immune cell of Embodiment I-159, wherein the extracellular ligand binding domain of the second activator receptor comprises an antibody fragment, a single chain Fv antibody fragment (scFv), a β chain variable domain (Vβ), or a TCR α chain variable domain and a TCR β chain variable domain.

Embodiment I-163 provides the immune cell of Embodiment I-162, wherein the extracellular binding domain of the second activator receptor comprises a VH region comprising any one of the sequences of SEQ ID NOs: 319, 321, 344, 346, 348, 350, 352, 353, 355, 357, 359, 361, 363, 365, 367, 369, 371, 373, 375, 377, or a sequence having at least 85%, at least 90%, at least 95%, at least 97%, at least 99% identity, or 100% identical thereto; and a VL region comprising any one of the sequences of SEQ ID NOs: 320, 322, 343, 345, 347, 349, 351, 354, 356, 358, 360, 362, 364, 366, 368, 370, 372, 374, 376, 378, or a sequence having at least 85%, at least 90%, at least 95%, at least 97%, at least 99% identity, or 100% identical thereto.

Embodiment I-164 provides the immune cell of any one of embodiments 149-163, wherein the inhibitor receptor is a TCR or a CAR.

Embodiment I-165 provides the immune cell of Embodiment I-164, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-166 provides the immune cell of any one of embodiments 149-165, wherein the inhibitor receptor comprises LILRB1 hinge and transmembrane domains, or functional variants thereof.

Embodiment I-167 provides a pharmaceutical composition, comprising an effective amount of the immune cells of any one of embodiments 149-166.

Embodiment I-168 provides the pharmaceutical composition of embodiment 167, for use as a medicament in the treatment of cancer.

Embodiment I-169 provides a polynucleotide system, comprising one or more polynucleotides comprising polynucleotide sequences encoding:
a) a first activator receptor comprising an extracellular ligand binding domain specific to a CD33 antigen;
b) a second activator receptor comprising an extracellular ligand binding domain specific to a FLT3 antigen; and
c) a first inhibitor receptor comprising an extracellular ligand binding domain specific to a PECAM-1 antigen.

Embodiment I-170 provides the immune cell of any one of embodiments 1-5, wherein the first activator antigen is SPN or a peptide antigen of SPN.

Embodiment I-171 provides the immune cell of Embodiment I-170, wherein the first inhibitor antigen is MME or a peptide antigen of MME.

Embodiment I-172 provides the immune cell of Embodiment I-170, wherein the first activator antigen is SPN and the first inhibitor antigen is MME.

Embodiment I-173 provides the immune cell of Embodiment I-170, wherein the cancer cell expresses SPN.

Embodiment I-174 provides the immune cell of any one of embodiments 170-173, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment I-175 provides the immune cell of any one of embodiments 170-174, wherein the blood cancer cell is a leukemia cell or a lymphoma cell.

Embodiment I-176 provides the immune cell of any one of embodiments 171-175, wherein the MME antigen is expressed by non-cancerous blood cells of a subject.

Embodiment I-177 provides the immune cell of any one of embodiments 170-176, wherein non-cancerous blood cells of the subject express both the first activator antigen and the first inhibitor antigen.

Embodiment I-178 provides the immune cell of any one of embodiments 170-177, wherein the activator receptor and the inhibitor receptor specifically activate the immune cell when in contact with the blood cancer cell.

Embodiment I-179 provides the immune cell of any one of embodiments 170-178, wherein the activator receptor specifically activates the immune cell when in contact with the blood cancer cell.

Embodiment I-180 provides the immune cell of any one of embodiments 170-179, wherein the inhibitor receptor specifically inhibits the immune cell when in contact with the non-cancerous blood cell.

Embodiment I-181 provides the immune cell of any one of embodiments 170-180, wherein the immune cell is a T cell.

Embodiment I-182 provides the immune cell of Embodiment I-181, wherein the T cell is a CD8+ CD4− T cell.

Embodiment I-183 provides the immune cell of any one of embodiments 170-182, wherein the SPN antigen comprises a sequence or subsequence at least 95% identical to a sequence or subsequence of any one of SEQ ID NOs: 172-173.

Embodiment I-184 provides the immune cell of any one of embodiments 170-183, wherein the activator receptor is a T cell receptor (TCR) or a chimeric antigen receptor (CAR).

Embodiment I-185 provides the immune cell of Embodiment I-184, wherein the extracellular ligand binding domain of the activator receptor comprises an antibody fragment, a single chain Fv antibody fragment (scFv), a β chain variable domain (Vβ), or a TCR α chain variable domain and a TCR β chain variable domain.

Embodiment I-186 provides the immune cell of Embodiment I-185, wherein the extracellular ligand binding domain of the activator receptor comprises a heavy chain variable (VH) region and a light chain variable (VL) region.

Embodiment I-187 provides the immune cell of Embodiment I-186, wherein the VH region comprises the sequence of any one of SEQ ID NOs: 249-252, 257, 259, or 261-280; and the VL region comprises the sequence of any one of SEQ ID NOs: 253-256, 258, 260, or 281-303.

Embodiment I-188 provides the immune cell of any one of embodiments 184-187, wherein the CAR comprises a hinge sequence isolated or derived from CD8, CD28, IgG1, or IgG4, or a synthetic hinge.

Embodiment I-189 provides the immune cell of any one of embodiments 184-187, wherein the CAR comprises a transmembrane domain isolated or derived from CD8 or CD28.

Embodiment I-190 provides the immune cell of any one of embodiments 184-189, wherein the CAR comprises an intracellular domain isolated or derived from CD28, 4-1BB or CD3z, or a combination thereof.

Embodiment I-191 provides the immune cell of any one of embodiments 170-190, wherein the inhibitor receptor is a TCR or a CAR.

Embodiment I-192 provides the immune cell of Embodiment I-191, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment I-193 provides the immune cell of Embodiment I-192, wherein the extracellular ligand binding domain of the inhibitor receptor comprises a heavy chain variable (VH) region and a light chain variable (VL) region.

Embodiment I-194 provides the immune cell of Embodiment I-193, wherein the VH region comprises the sequence of any one of SEQ ID NOs: 394, 397, 400-402, 404, or 406; and the VL region comprises the sequence of any one of SEQ ID NOs: 395, 396, 398, 399, 403, 405, or 407.

Embodiment I-195 provides the immune cell of any one of embodiments 170-193, wherein the inhibitor receptor comprises a LILRB1 intracellular domain or a functional variant thereof.

Embodiment I-196 provides the immune cell of any one of embodiments 170-194, wherein the inhibitor receptor comprises LILRB1 hinge and transmembrane domains, or functional variants thereof.

Embodiment I-197 provides a pharmaceutical composition, comprising an effective amount of the immune cells of any one of embodiments 170-196.

Embodiment I-198 provides the pharmaceutical composition of Embodiment I-197, further comprising a pharmaceutically acceptable carrier, diluent or excipient.

Embodiment I-199 provides the pharmaceutical composition of Embodiment I-197 or 198, for use as a medicament in the treatment of cancer.

Embodiment I-200 provides a polynucleotide system, comprising one or more polynucleotides comprising polynucleotide sequences encoding:
a) an activator receptor comprising an extracellular ligand binding domain specific to a SPN antigen; and
b) an inhibitor receptor comprising an extracellular ligand binding domain specific to an MME antigen.

Embodiment I-201 provides a method of making an immune cell therapy, comprising transforming immune cells with the polynucleotide system of Embodiment I-200.

Embodiment I-202 provides a kit comprising the immune cell of any one of embodiments 170-196 or the pharmaceutical composition of any one of claims 197-199.

Embodiment I-203 provides an immune cell comprising:
a) an activator receptor comprising an extracellular ligand binding domain specific to a SPN antigen, wherein the extracellular ligand binding domain of the activator receptor is an scFv; and
b) an inhibitor receptor comprising an extracellular ligand binding domain specific to an MME antigen that is not expressed by a blood cancer cell, wherein the extracellular ligand binding domain of the inhibitor receptor is an scFv.

Embodiment I-204 provides a method of treating cancer in a subject, comprising administering to the subject an immune cell comprising
a) an activator receptor comprising an extracellular ligand binding domain specific to a SPN antigen, wherein the extracellular ligand binding domain of the activator receptor is an scFv; and
b) an inhibitor receptor comprising an extracellular ligand binding domain specific to an MME antigen that is not expressed by a blood cancer cell, wherein the extracellular ligand binding domain of the inhibitory receptor is an scFv.

### Enumerated Embodiments Set II

Embodiment II-1 provides a nanocarrier comprising one or more polynucleotides, wherein the one or more polynucleotides encode:
a. an activator receptor comprising an extracellular ligand binding domain specific to a first activator antigen expressed by blood cancer cells; and
b. an inhibitor receptor comprising an extracellular ligand binding domain specific to a first inhibitor antigen expressed by non-cancerous blood cells.

Embodiment II-2 provides the nanocarrier of Embodiment II-1, wherein the nanocarrier is capable of delivering the one or more polynucleotides to an immune cell in vivo or ex vivo.

Embodiment II-3 provides the nanocarrier of any one of the preceding embodiments, wherein the nanocarrier is a lipid nanoparticle (LNP).

Embodiment II-4 provides the nanocarrier of any one of the preceding embodiments, wherein the one or more polynucleotides are one or more messenger ribonucleic acids (mRNAs) or modified mRNAs (mmRNAs).

Embodiment II-5 provides the nanocarrier of any one of the preceding embodiments, wherein the first activator antigen is expressed by hematopoietic cells.

Embodiment II-6 provides the nanocarrier of any one of the preceding embodiments, wherein the first inhibitor antigen is expressed by myeloid cells.

Embodiment II-7 provides the nanocarrier of any one of the preceding embodiments, wherein the myeloid cells are differentiated.

Embodiment II-8 provides the nanocarrier of any one of the preceding embodiments, wherein the first inhibitor antigen is expressed by hematopoietic stem cells.

Embodiment II-9 provides the nanocarrier of any one of the preceding embodiments, wherein the first activator antigen is SPN or a peptide antigen of SPN.

Embodiment II-10 provides the nanocarrier of any one of the preceding embodiments, wherein the first inhibitor antigen is MME or a peptide antigen of MME.

Embodiment II-11 provides the nanocarrier of any one of the preceding embodiments, wherein the first activator antigen is SPN and the first inhibitor antigen is MME.

Embodiment II-12 provides the nanocarrier of any one of the preceding embodiments, wherein the cancer cell expresses SPN.

Embodiment II-13 provides the nanocarrier of any one of the preceding embodiments, wherein the blood cancer cell is a lymphoma cell, a leukemia cell, a myeloma cell, a Reed-Sternberg cell, a myeloproliferative neoplasm cell, or a Waldenstrom macroglobulinemia cell.

Embodiment II-14 provides the nanocarrier of any one of the preceding embodiments, wherein the blood cancer cell is a leukemia cell or a lymphoma cell.

Embodiment II-15 provides the nanocarrier of any one of the preceding embodiments, wherein the MME antigen is expressed by non-cancerous blood cells of a subject.

Embodiment II-16 provides the nanocarrier of any one of the preceding embodiments, wherein non-cancerous blood cells of the subject express both the first activator antigen and the first inhibitor antigen.

Embodiment II-17 provides the nanocarrier of any one of the preceding embodiments, wherein the activator receptor and the inhibitor receptor specifically activate the immune cell when in contact with the blood cancer cell.

Embodiment II-18 provides the nanocarrier of any one of the preceding embodiments, wherein the activator receptor specifically activates the immune cell when in contact with the blood cancer cell.

Embodiment II-19 provides the nanocarrier of any one of the preceding embodiments, wherein the inhibitor receptor specifically inhibits the immune cell when in contact with the non-cancerous blood cell.

Embodiment II-20 provides the nanocarrier of any one of the preceding embodiments, wherein the immune cell is a T cell.

Embodiment II-21 provides the nanocarrier of any one of the preceding embodiments, wherein the T cell is a CD8+ CD4− T cell.

Embodiment II-22 provides the nanocarrier of any one of the preceding embodiments, wherein the SPN antigen comprises a sequence or subsequence at least 95% identical to a sequence or subsequence of any one of SEQ ID NOs: 172-173.

Embodiment II-23 provides the nanocarrier of any one of the preceding embodiments, wherein the activator receptor is a T cell receptor (TCR) or a chimeric antigen receptor (CAR).

Embodiment II-24 provides the nanocarrier of any one of the preceding embodiments, wherein the extracellular ligand binding domain of the activator receptor comprises an antibody fragment, a single chain Fv antibody fragment (scFv), a β chain variable domain (Vβ), or a TCR α chain variable domain and a TCR β chain variable domain.

Embodiment II-25 provides the nanocarrier of any one of the preceding embodiments, wherein the extracellular ligand binding domain of the activator receptor comprises a heavy chain variable (VH) region and a light chain variable (VL) region.

Embodiment II-26 provides the nanocarrier of any one of the preceding embodiments, wherein the VH region comprises the sequence of any one of SEQ ID NOs: 249-252, 257, 259, or 261-280; and the VL region comprises the sequence of any one of SEQ ID NOs: 253-256, 258, 260, or 281-303.

Embodiment II-27 provides the nanocarrier of any one of the preceding embodiments, wherein the CAR comprises a hinge sequence isolated or derived from CD8, CD28, IgG1, or IgG4, or a synthetic hinge.

Embodiment II-28 provides the nanocarrier of any one of the preceding embodiments, wherein the CAR comprises a transmembrane domain isolated or derived from CD8 or CD28.

Embodiment II-29 provides the nanocarrier of any one of the preceding embodiments, wherein the CAR comprises an intracellular domain isolated or derived from CD28, 4-1BB or CD3z, or a combination thereof.

Embodiment II-30 provides the nanocarrier of any one of the preceding embodiments, wherein the inhibitor receptor is a TCR or a CAR.

Embodiment II-31 provides the nanocarrier of any one of the preceding embodiments, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

Embodiment II-32 provides the nanocarrier of any one of the preceding embodiments, wherein the extracellular ligand binding domain of the inhibitor receptor comprises a heavy chain variable (VH) region and a light chain variable (VL) region.

Embodiment II-33 provides the nanocarrier of any one of the preceding embodiments, wherein the VH region comprises the sequence of any one of SEQ ID NOs: 394, 397, 400-402, 404, or 406; and the VL region comprises the sequence of any one of SEQ ID NOs: 395, 396, 398, 399, 403, 405, or 407.

Embodiment II-34 provides the nanocarrier of any one of the preceding embodiments, wherein the inhibitor receptor comprises a LILRB1 intracellular domain or a functional variant thereof.

Embodiment II-35 provides the nanocarrier of any one of the preceding embodiments, wherein the inhibitor receptor comprises LILRB1 hinge and transmembrane domains, or functional variants thereof.

Embodiment II-36 provides a pharmaceutical composition, comprising an effective amount of the nanocarriers of any one of the preceding embodiments.

Embodiment II-37 provides a pharmaceutical composition of Embodiment II-36, further comprising a pharmaceutically acceptable carrier, diluent or excipient.

Embodiment II-38 provides a pharmaceutical composition of any one of Embodiments 36-37, for use as a medicament in the treatment of cancer.

Embodiment II-39 provides a method of generating engineered immune cells in a subject in need thereof, comprising administering to the subject a nanocarrier according to any one of the preceding embodiments.

Embodiment II-40 provides a method of killing tumor cells in a subject in need thereof, comprising administering to the subject a nanocarrier according to any one of the preceding embodiments.

Embodiment II-41 provides a method of treating cancer in a subject in need thereof, comprising administering to the subject a nanocarrier according to any one of the preceding embodiments.

Embodiment II-42 provides the method of any one of the preceding embodiments, wherein the nanocarrier or pharmaceutical composition is administered in an amount effective to specifically kill blood cancer cells in the subject.

Embodiment II-43 provides the method of any one of the preceding embodiments, wherein the nanocarrier is administered by intravenous injection.

Embodiment II-44 provides the method of any one of the preceding embodiments, wherein the nanocarrier is a lipid nanoparticle (LNP).

Embodiment II-45 provides the method of any one of the preceding embodiments, wherein the one or more polynucleotides are delivered ex vivo.

Embodiment II-46 provides a kit comprising the nanocarrier of any one of nanocarrier or pharmaceutical composition according to any one of the preceding embodiments.

Embodiment II-47 provides a method of treating cancer in a subject, comprising administering to the subject a nanocarrier comprising one or more polynucleotides encoding
(a) an activator receptor comprising an extracellular ligand binding domain specific to a SPN antigen, wherein the extracellular ligand binding domain of the activator receptor comprises
(i) a VH region comprising sequences of any one of SEQ ID NOs: 249-252, 257, 259, or 261-280; and (ii) a VL region comprising sequences of any one of SEQ ID NOs: 253-256, 258, 260, or 281-303; and
(b) an inhibitor receptor comprising an extracellular ligand binding domain specific to an MME antigen that is not expressed by a blood cancer cell, wherein the extracellular ligand binding domain of the inhibitor receptor comprises
(i) a VH region comprising sequences of any one of SEQ ID NOs: 394, 397, 400-402, 404, or 406; and
(ii) a VL region comprising sequences of any one of SEQ ID NOs: 395, 396, 398, 399, 403, 405, or 407.

Embodiment II-48 provides the method of any one of the preceding embodiments, wherein the nanocarrier is administered by intravenous injection.

Embodiment II-49 provides a polynucleotide encoding:
a. an activator receptor comprising an extracellular ligand binding domain specific to a first activator antigen expressed by blood cancer cells; and
b. an inhibitor receptor comprising an extracellular ligand binding domain specific to a first inhibitor antigen expressed by non-cancerous blood cells;
wherein the polynucleotide is an mRNA; and wherein the polynucleotide is delivered to an immune cell without any carrier molecules.

### Enumerated Embodiments Set III

Embodiment III-1 provides an immune cell, comprising
a. an activator receptor comprising an extracellular ligand-binding domain specific to an activator antigen expressed by blood cells; and
b. an inhibitor receptor comprising an extracellular ligand-binding domain specific to an inhibitor antigen,

wherein the inhibitor antigen is an allelic variant of a Human Leukocyte Antigen (HLA).

Embodiment III-2 provides the immune cell of Embodiment III-1, wherein the activator antigen is expressed by hematopoietic cells, myeloid cells, or hematopoietic stem cells, or a combination thereof.

Embodiment III-3 provides the immune cell of any one of the preceding embodiments, wherein the activator antigen is SPN, CD45, ITGAL, CD33, or FLT3.

Embodiment III-4 provides the immune cell of any one of the preceding embodiments, wherein the activator antigen is a peptide antigen of SPN, CD45, ITGAL, CD33, or FLT3 in complex with a Major Histocompatibility Complex (MHC).

Embodiment III-5 provides the immune cell of any one of the preceding embodiments, wherein the inhibitor antigen is an allelic variant of a major HLA.

Embodiment III-6 provides the immune cell of any one of the preceding embodiments, wherein the inhibitor antigen is an allelic variant of HLA-A, HLA-B, or HLA-C.

Embodiment III-7 provides the immune cell of any one of the preceding embodiments, wherein the inhibitor antigen is HLA-A*02, HLA-B*07, HLA-C*07, or HLA-A*69.

Embodiment III-8 provides the immune cell of any one of the preceding embodiments, wherein the inhibitor antigen is an allelic variant of HLA-DRB1, HLA-DQB1, or HLA-DPB1.

Embodiment III-9 provides the immune cell of any one of the preceding embodiments, wherein the inhibitor antigen is an allelic variant of a minor HLA.

Embodiment III-10 provides the immune cell of any one of the preceding embodiments, wherein the immune cell is a T cell.

Embodiment III-11 provides the immune cell of any one of the preceding embodiments, wherein the extracellular-ligand binding domains of the activator receptor and/or of the inhibitor receptor each individually comprises a single chain Fv antibody fragment (scFv).

Embodiment III-12 provides the immune cell of any one of the preceding embodiments, wherein the activator receptor is a chimeric antigen receptor comprising the extracellular ligand-binding domain; a transmembrane domain; and a CD28 intracellular domain, a 4-1BB intracellular domain, or a CD3z intracellular domain.

Embodiment III-13 provides the immune cell of any one of the preceding embodiments, wherein the inhibitor receptor comprises the extracellular ligand-binding domain; a transmembrane domain; and an LILRB1 intracellular domain.

Embodiment III-14 provides the immune cell of Embodiment III-13, wherein the inhibitor receptor comprises an LILRB1 hinge and/or an LILRB1 transmembrane domains.

Embodiment III-15 provides a pharmaceutical composition, comprising the immune cell of any one the preceding embodiments and one or more pharmaceutically acceptable excipients or diluents.

Embodiment III-16 provides a kit comprising the pharmaceutical composition of Embodiment III-15, and instructions for use.

Embodiment III-17 provides a polynucleotide system, encoding the activator receptor and/or the inhibitor receptor of the immune cell of any one of embodiments 1-14.

Embodiment III-18 provides a method of killing blood cells, comprising contacting blood cells and allogeneic stem cells with the immune cell of any one of embodiments 1-14, wherein the inhibitor receptor of the immune cell is specific to an inhibitor antigen expressed by the allogenic stem cells, such that the allogenic stem cells are spared from killing by the immune cells, and wherein the blood cells lack the inhibitor antigen, such that the immune cell kills the blood cells.

Embodiment III-19 provides a method of treating or preventing relapse of blood cancer in a subject treated for blood cancer with allogeneic stem cell transplant, comprising administering to the subject the immune cell of any one of embodiments 1-14 or the pharmaceutical composition of Embodiment III-15.

Embodiment III-20 provides a method of conditioning a subject for allogeneic stem cell transplant, comprising administering to the subject the immune cell of any one of embodiments 1-14 or the pharmaceutical composition of Embodiment III-15.

Embodiment III-21 provides the method of Embodiment III-20, wherein the immune cell or the pharmaceutical composition is administered in an amount effective to condition the subject for stem cell transplant without another conditioning therapy.

Embodiment III-22 provides the method of Embodiment III-20 or 21, wherein the immune cell or the pharmaceutical composition is administered in an amount effective to treat blood cancer in the subject without another conditioning therapy.

Embodiment III-23 provides the method of Embodiment III-20, wherein the method comprises administering a second conditioning therapy.

Embodiment III-24 provides the method of Embodiment III-23, wherein the second conditioning therapy is chemotherapy.

Embodiment III-25 provides the method of Embodiment III-23, wherein the second conditioning therapy is radiation therapy.

Embodiment III-26 provides the method of any one of embodiments 23-25, wherein the second conditioning therapy is administered in amount less than an amount effective to condition the subject for stem cell transplant without the immune cell or pharmaceutical composition.

Embodiment III-27 provides the method of any one of embodiments 23-26, wherein the second conditioning therapy is administered in amount less than an amount effective to treat blood cancer in the subject without the immune cell or pharmaceutical composition.

Embodiment III-28 provides a method of allogeneic stem cell transplant in a subject in need thereof, comprising:
a. administering to the subject the immune cell of any one of embodiments 1-14 or the pharmaceutical composition of Embodiment III-15; and
b. administering to the subject an allogeneic stem cell transplant,

wherein inhibitor receptor of the immune cell is specific to an inhibitor antigen expressed by the cells of the stem cell transplant, such that the immune cells spare the stem cell transplant, and

wherein the cells of the subject lack the inhibitor antigen, such that the immune cell or pharmaceutical composition kills blood cells in the subject to condition the subject for the stem cell transplant.

Embodiment III-29 provides a method of treating blood cancer in a subject in need thereof, comprising:
a. administering to the subject the immune cell of any one of embodiments 1-14 or the pharmaceutical composition of Embodiment III-15; and
b. administering to the subject an allogeneic stem cell transplant,

wherein inhibitor receptor of the immune cell is specific to an inhibitor antigen expressed by the cells of the stem cell transplant, such that the immune cells spare the stem cell transplant, and

wherein the cells of the blood cancer lack the inhibitor antigen, such that the immune cell or pharmaceutical composition kills blood cancer cells in the subject.

Embodiment III-30 provides the method of Embodiment III-29, wherein the blood cancer is leukemia.

Embodiment III-31 provides the method of Embodiment III-30, wherein the blood cancer acute myeloid leukemia (AML).

Embodiment III-32 provides the method of Embodiment III-29, wherein the blood cancer is a lymphoma.

Embodiment III-33 provides the method of any one of embodiments 28-32, wherein the stem cell transplant comprises hematopeotic stem cells.

Embodiment III-34 provides the method of Embodiment III-33, wherein the hematopeotic stem cells are CD34+ positively selected hematopeotic stem cells.

Embodiment III-35 provides the method of any one of embodiments 33 or 34, wherein the hematopeotic stem cells are CD3-negatively selected hematopeotic stem cells.

Embodiment III-36 provides the method of any one of embodiments 33-35, wherein the hematopeotic stem cells are CD19-negatively selected hematopeotic stem cells.

Embodiment III-37 provides the method of any one of embodiments 19-36, wherein the immune cell and allogeneic stem cell transplant are selected such that:
a. if the subject is homozygous null HLA-A*02−/−, the inhibitor antigen is HLA-A*02 and the allogeneic stem cell transplant is HLA-A*02+;
b. if the subject is homozygous null HLA-B*07−/−, the inhibitor antigen is HLA-B*07 and the allogeneic stem cell transplant is HLA-B*07+;
c. if the subject is homozygous null HLA-C*07−/−, the inhibitor antigen is HLA-C*07 and the allogeneic stem cell transplant is HLA-C*07+; or
d. if the subject is homozygous null HLA-A*69−/−, the inhibitor antigen is HLA-A*69 and the allogeneic stem cell transplant is HLA-A*69+.

Embodiment III-38 provides a biobank, comprising a collection of immune cells according to any one of embodiments 1-14 and a collection of allogeneic stem cell transplants, wherein each allogeneic stem cell transplant is positive for the allelic variant of an HLA and each immune cell comprises an inhibitor receptor specific to one of the alleleic variants.

Embodiment III-39 provides the biobank of Embodiment III-38, wherein the biobank comprises
a. an immune cell whose inhibitor antigen is HLA-A*02 and the allogeneic stem cell transplant that are HLA-A*02+;
b. an immune cell whose inhibitor antigen is HLA-B*07−/−, the inhibitor antigen is HLA-B*07 and the allogeneic stem cell transplant are HLA-B*07+;
c. an immune cell whose inhibitor antigen is HLA-C*07−/−, the inhibitor antigen is HLA-C*07 and the allogeneic stem cell transplant are HLA-C*07+; and/or
d. an immune cell whose inhibitor antigen is HLA-A*69−/−, the inhibitor antigen is HLA-A*69 and the allogeneic stem cell transplant are HLA-A*69+.

Embodiment III-40 provides a method of allogeneic stem cell transplant, comprising
a. identifying a subject as homozygous null for an allelic variant of an HLA; and
b. matching the subject to an immune cells comprising an inhibitor receptor specific to the allelic variant and an allogeneic stem cell positive for the allelic variant.

Embodiment III-41 provides the method of any one of embodiments 20-36, wherein the method comprises identifying the subject as homozygous null for the allelic variant of the HLA to which the inhibitor receptor of the immune cell is specific.

Embodiment III-42 provides the method of Embodiment III-41, wherein the method comprises selecting the allogeneic stem cell transplant as positive for the allelic variant.

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Illustrative Algorithm Process

To identify B antigens for use in an A NOT B logic gate, a bioinformatics screen was performed and a list of antigens was filtered by antigens rarely expressed in blood tumor specimens, and then filtered by antigens rarely expressed in cell lines derived from blood tumors. FIG. 5 is a schematic of an illustrative algorithm.

The following filters are applied to the starting list of antigens:
i) Antigens which are present on blood tissues;
ii) Antigens which are present on non-blood tissues;
iii) Antigens which are rarely expressed in bulk blood tumor specimens Bulk blood tumor specimens include Acute Myeloid Leukemia (AML), Acute Lymphoblastic Leukemia (ALL), Chronic Lymphocytic Leukemia (CLL), Multiple Myeloma (MM), or non-Hodgkin Lymphoma (NHL). Bulk tumors refer to primary tumors removed from a patient;
iv) Antigens which are rarely expressed in cell lines derived from blood tumors (AML, ALL, CLL, NHL, MM);
following the analysis of the bioinformatics screen, potential B antigen candidates were curated through additional analysis; and
v) Antigens which are expressed above the median RNA expression level.

Predicted membrane proteins were analyzed using The Human Protein Atlas. Healthy adult tissue expression was analyzed using The Genotype-Tissue Expression (GTEx) database. Expression in cell lines was analyzed using The Cancer Dependency Map (DepMap). Tumor (AML) expression was analyzed using The Cancer Genome Atlas (TCGA).

All candidate antigens were then subjected to manual curation where several further characteristics were examined. Subcellular localization to plasma membrane was analyzed using GeneCards. Expression in human HSCs and other blood cell subtypes was analyzed using Haemosphere.org. In some cases, numbers were rounded and approximated from graphs during manual confirmation process.

To identify A antigens for use in an A NOT B logic gate, a list of putative antigens was filtered by antigens expressed in bulk tumor specimens of blood tumors, and then filtered by antigens expressed in cell lines derived from blood tumors. FIG. 5 is a schematic of an illustrative algorithm.

The following filters are applied to the starting list of antigens:
i) Antigens which are present on blood tissues;
ii) Antigens which are present on non-blood tissues (excluding spleen, lung, intestine);
iii) Antigens which are expressed in bulk tumor specimens of blood tumors. Bulk tumors refer to primary tumors removed from a patient;
iv) Antigens which are expressed in cell lines derived from blood tumors (AML, CLL, NHL, MM);
v) Antigens which are expressed above the median RNA expression level (soft filter used for ranking).

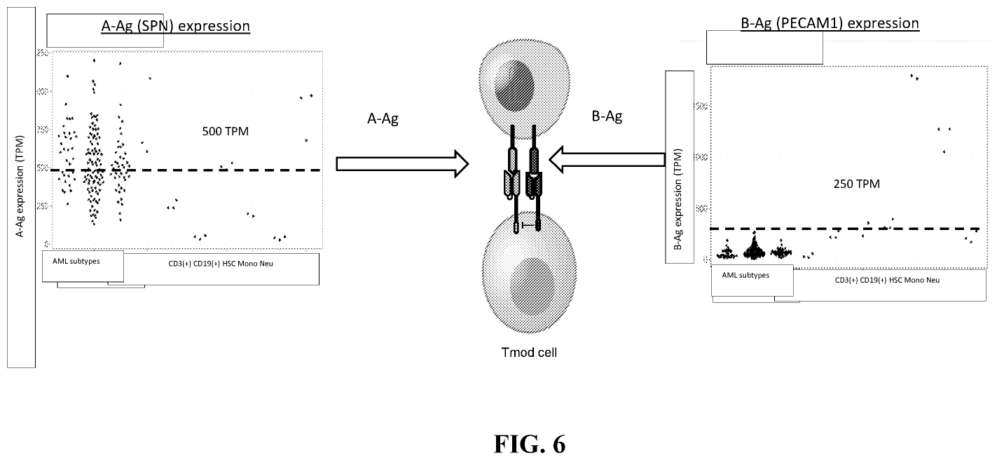

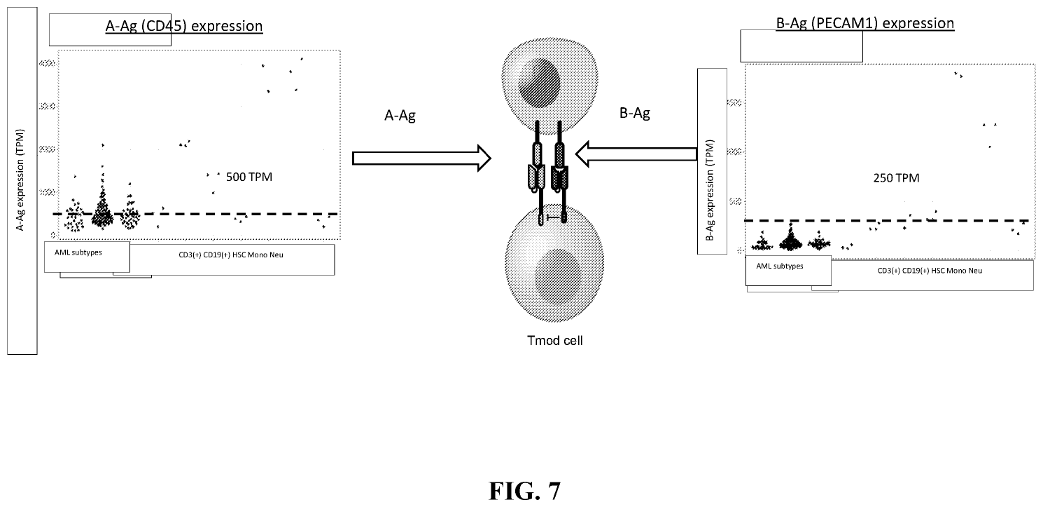

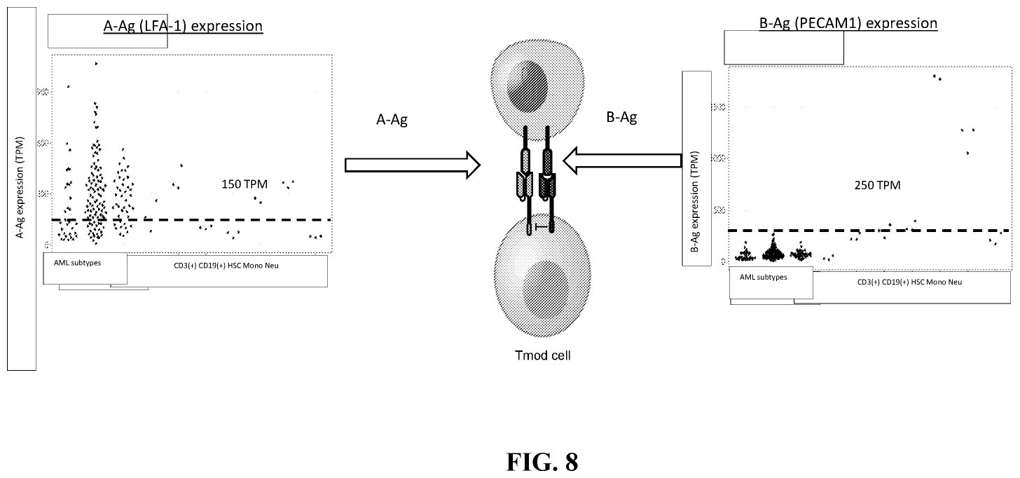

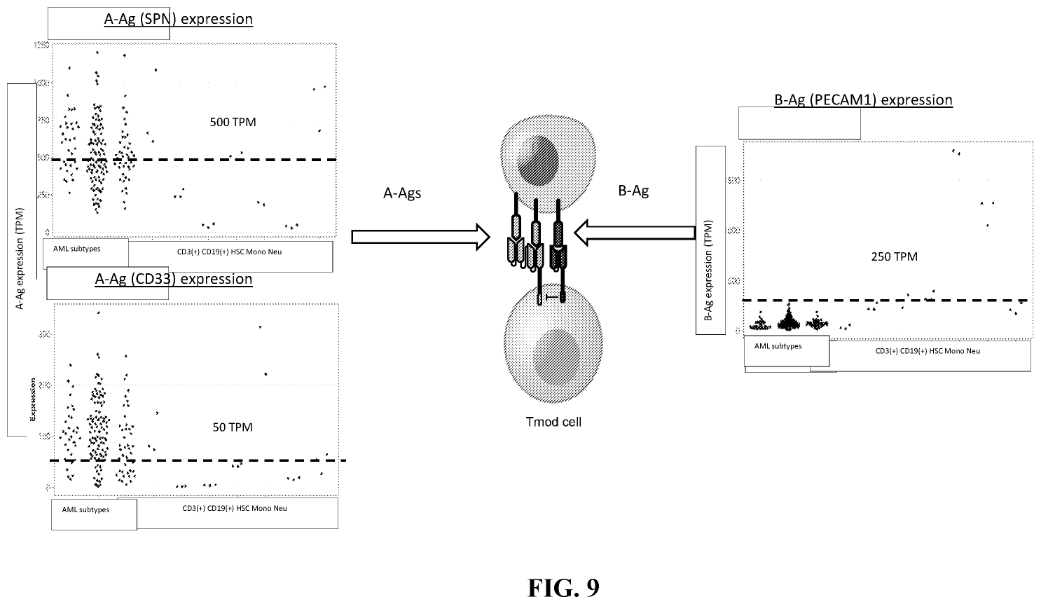

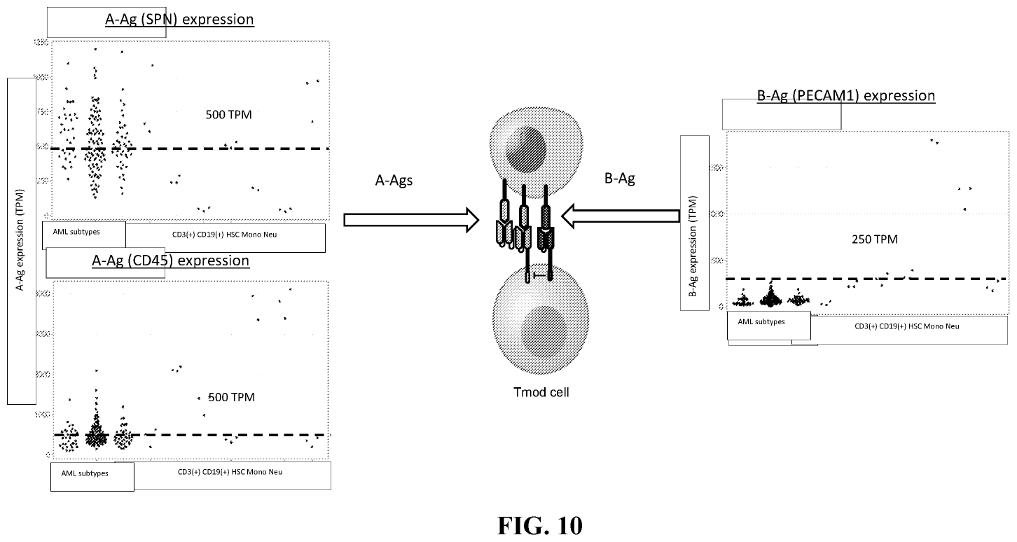

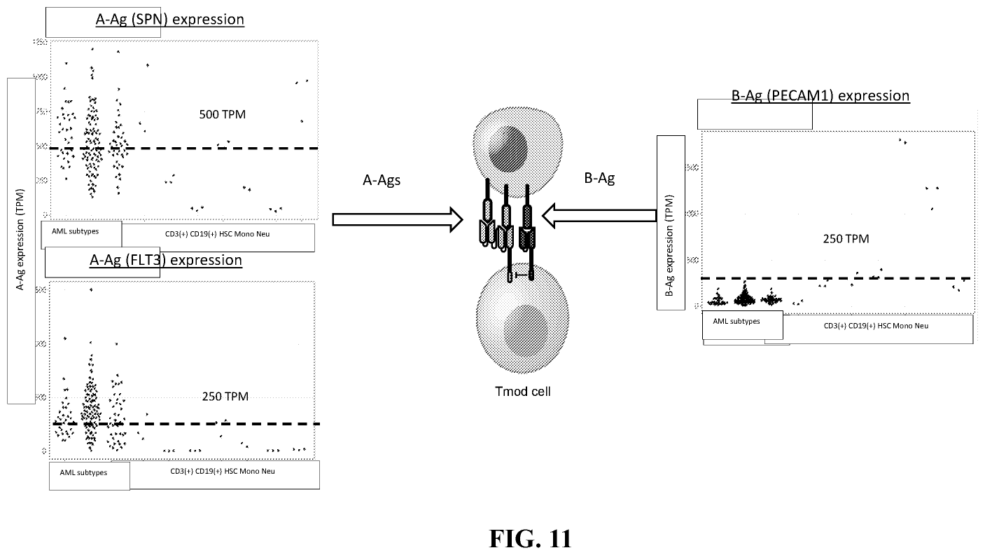

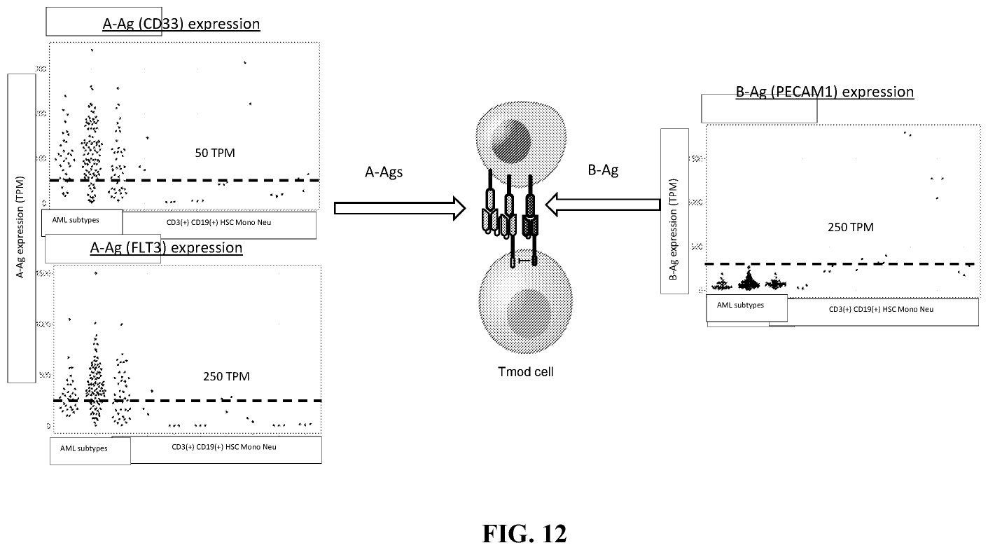

The A antigens SPN, CD45, ITGAL, CD33, and FLT3 and the B antigens PECAM-1, HLA-G, ABCG2, SIGLEC5, and PDZK1IP1 were selected for further testing. Expression data for illustrative combinations of A and B antigens is shown in FIGS. 6-12.

### Example 2: Activity of Anti-CD19 and/or Anti-CD20 CAR and HLA-A*02 Inhibitor Receptor Expressed in Jurkat Cells

Three candidates of receptor pairs were tested for sensitivity: anti-CD19 scFv CAR, anti-CD20 scFv CAR, or tandem anti-CD20 scFv/anti-CD19 scFv CAR were paired with a HLA-A*02 inhibitor receptor.

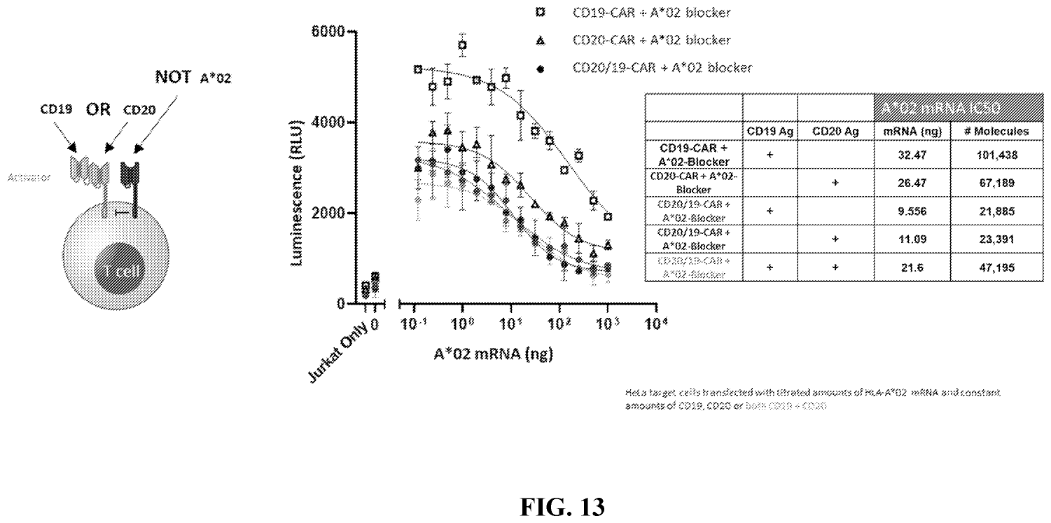

Inhibitor sensitivity was measured using the mRNA titration assay. HLA-A*02 mRNA was titrated and used to transfect HeLa target cells also transfected with constant amounts of CD 19 and/or CD20 mRNA, which were then co-cultured with Jurkat cells expressing the appropriate anti-CD19 and/or anti-CD20 constructs and NFAT luciferase. After 6 hours of co-culture, Jurkat cell activation was measured by luminescence intensity. Results are shown in FIG. 13. HLA-A*02 inhibitor receptor inhibits a tandem anti-CD20/anti-CD19-CAR similarly to single-scFv-CARs in the presence of either CD19 or CD20 or both CD19 and CD20 activator antigens.

### Example 3: Activity of Tandem Anti-CD19 and Anti-CD20 CAR and HLA-A*02 Inhibitor Receptor

A tandem anti-CD20 scFv/anti-CD19 scFv CAR paired with a HLA-A*02 inhibitor receptor was tested in three cell lines: K562 cells (CD19+), Raji cells (CD20+ and CD19 KO), and Raji cells (CD20+ and CD19+).

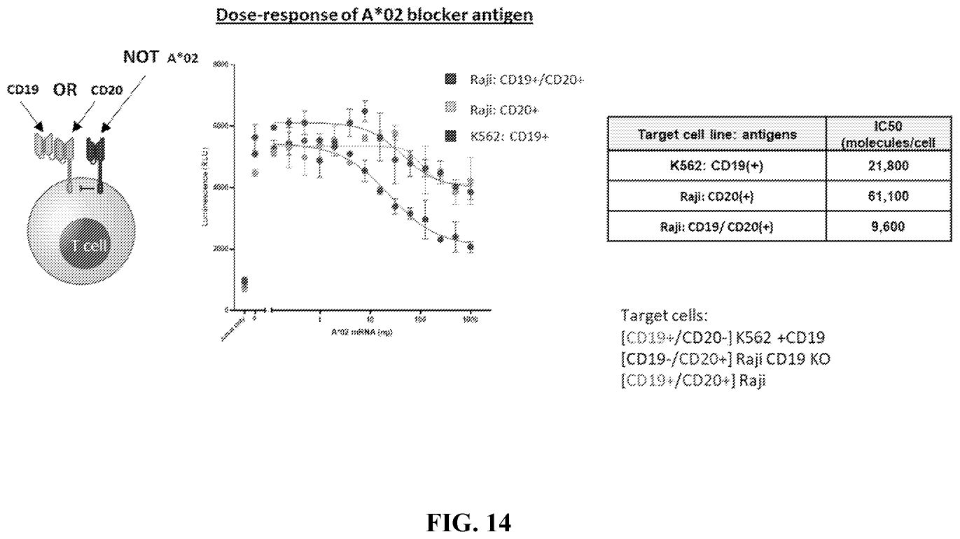

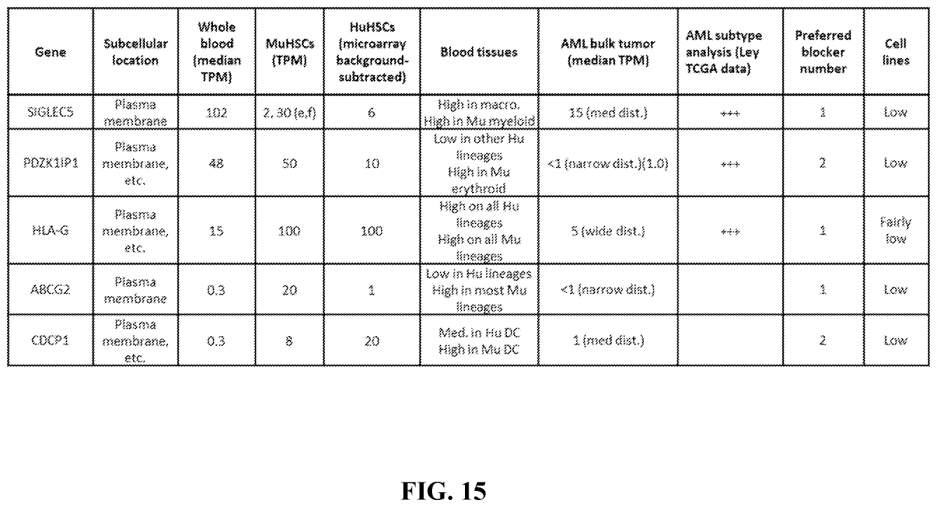

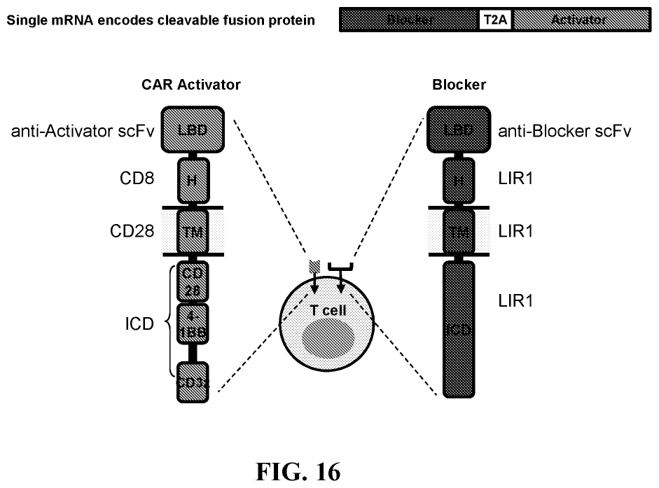

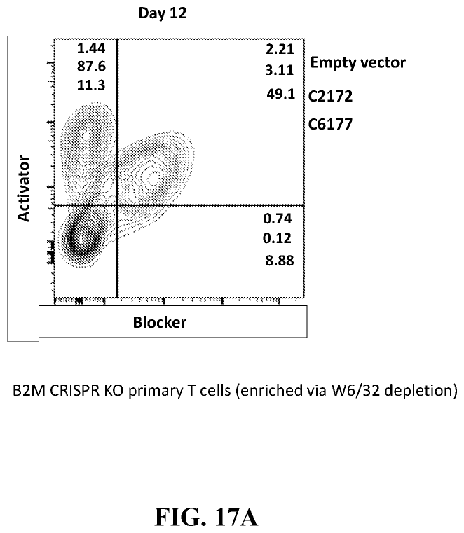

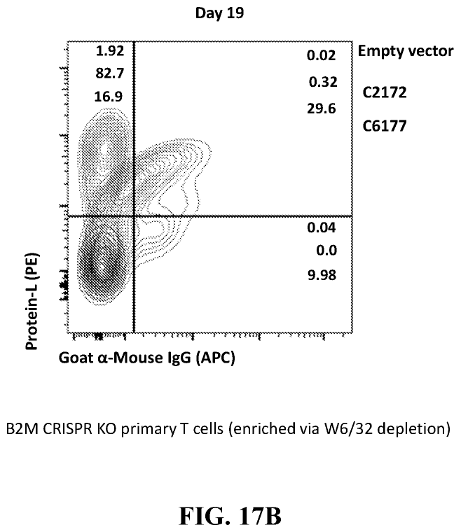

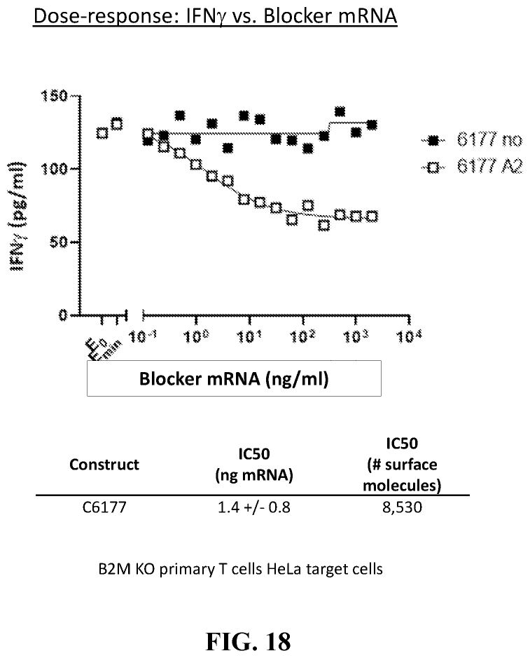

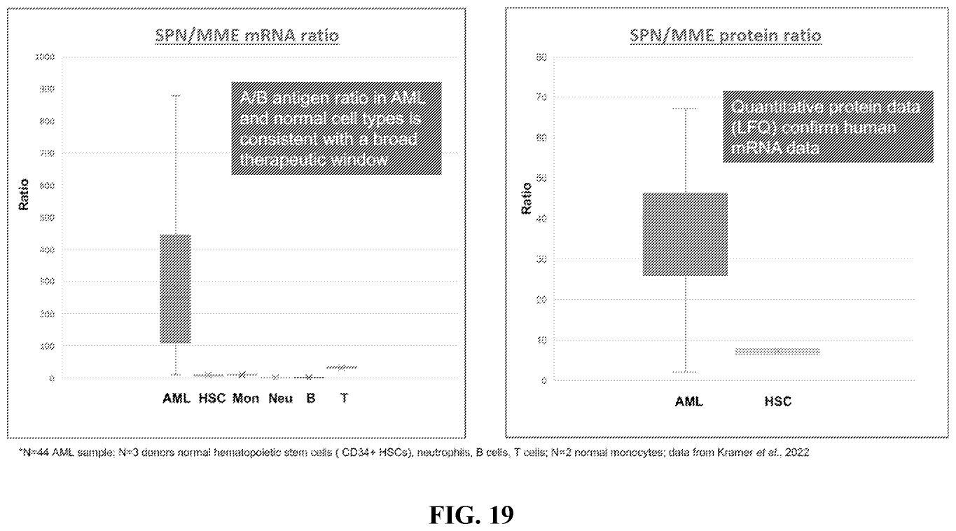

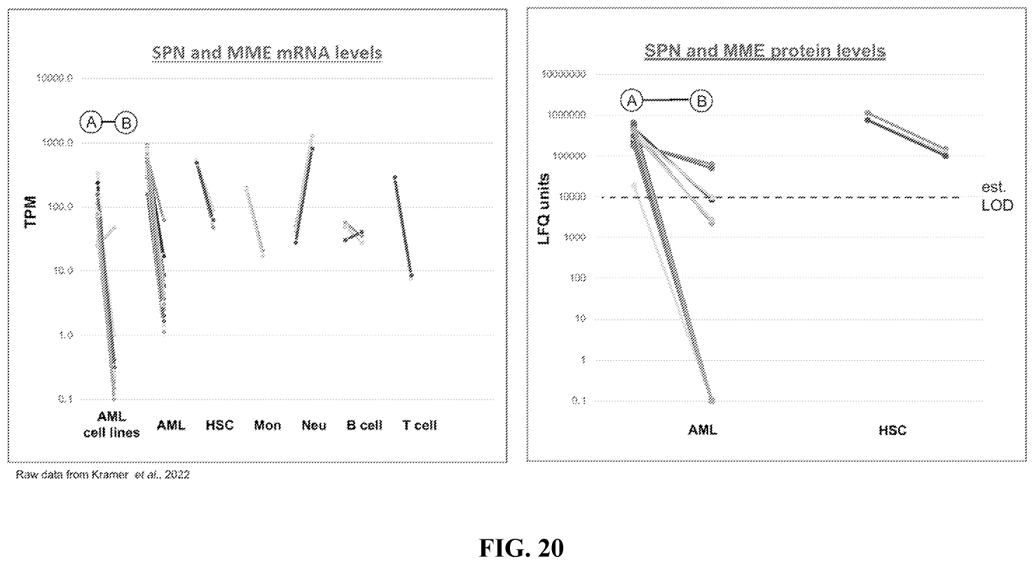

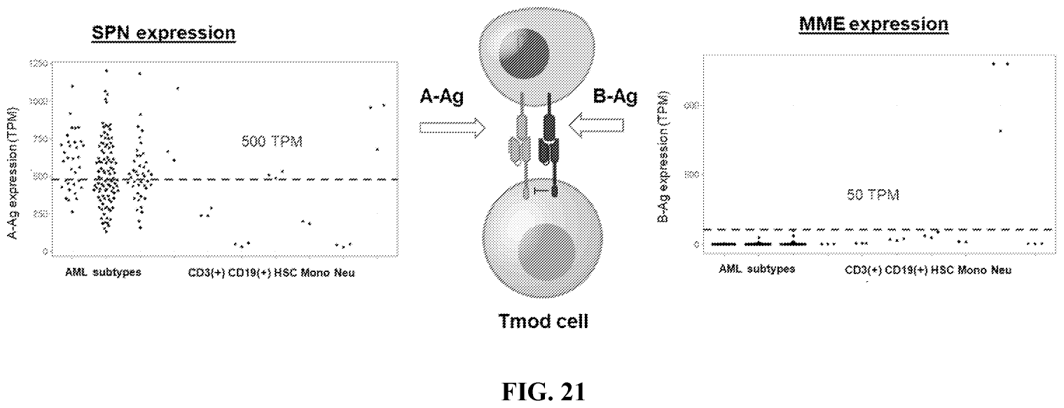

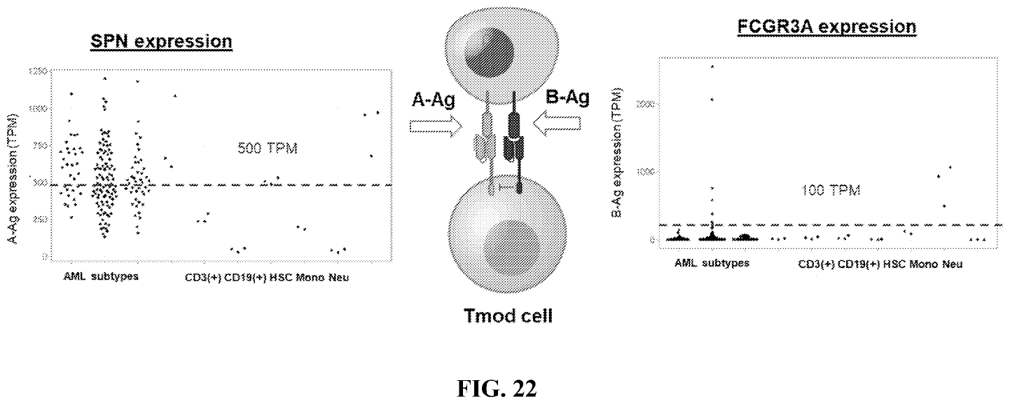

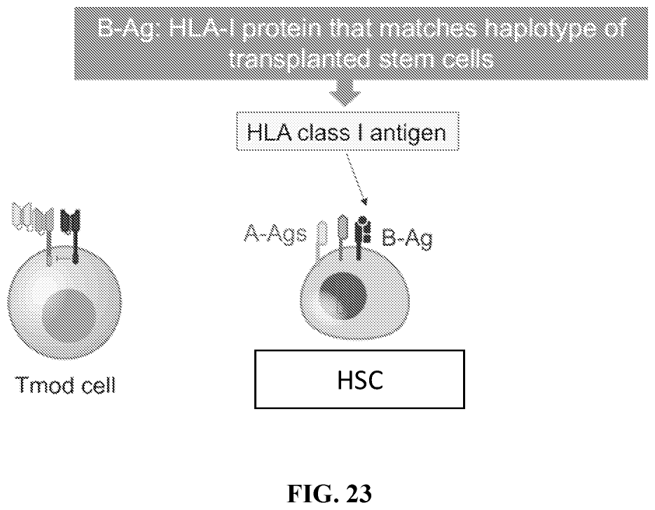

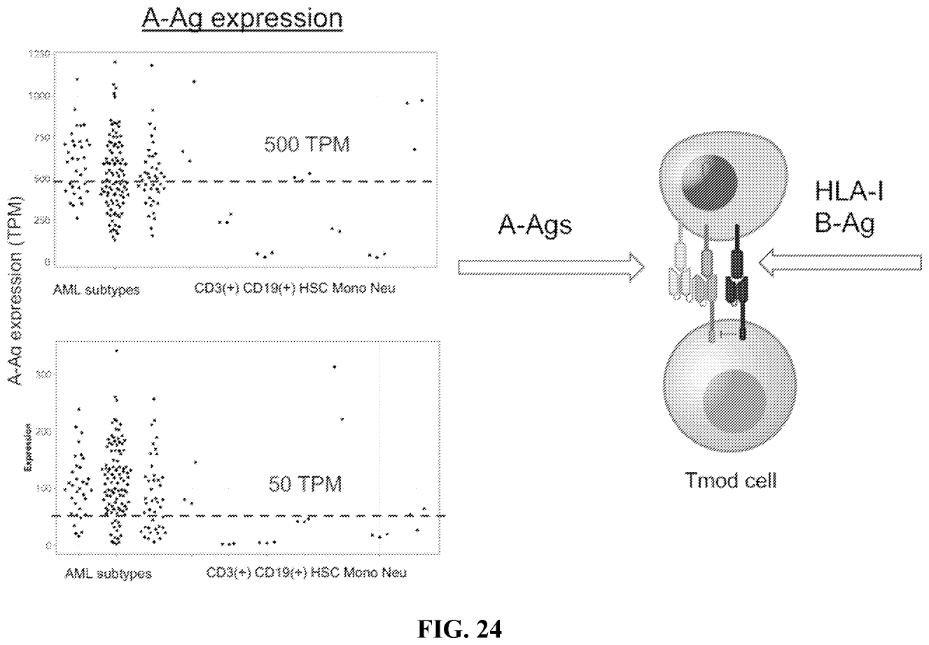

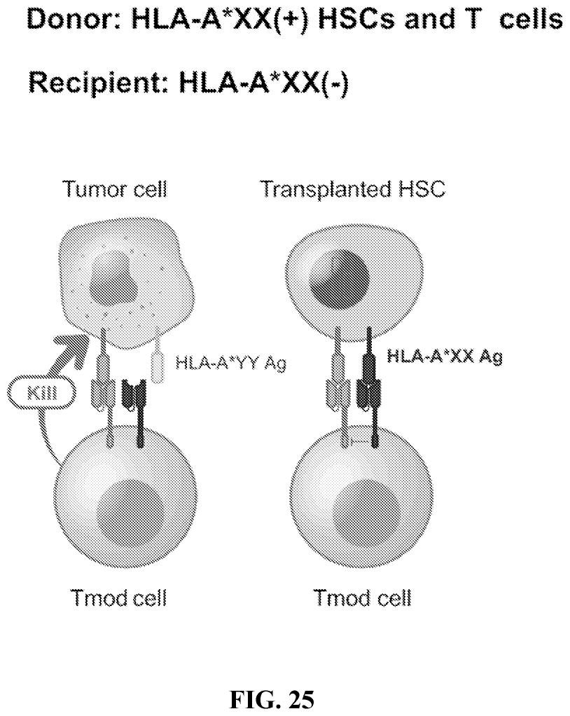

The dose response of HLA-A*02 inhibitor antigen was measured using the mRNA titration assay. HLA-A*02 mRNA was titrated and used to transfect target cells described above, which were then co-cultured with Jurkat cells expressing the tandem anti-CD20 scFv/anti-CD19 scFv CAR constructs and NFAT luciferase. After 6 hours of co-culture, Jurkat cell activation was measured by luminescence intensity. Results are shown in FIG. 14. HLA-A*02 inhibitor receptor inhibits a tandem anti-CD20/anti-CD19-CAR in cell lines that express the CD 19 and or CD20 activator antigens endogenously.

### Example 4: Methods of Assaying CAR Activity in Jurkat Cells

### Cell Culture

Jurkat cells encoding an NFAT luciferase reporter are obtained from BPS Bioscience. In culture, Jurkat cells are maintained in RPMI media supplemented with 10% FBS, 1% Pen/Strep and 0.4 mg/mL G418/Geneticin. HCT.116 and HeLa cells are maintained as suggested by ATCC.

### Jurkat Cell Transfection

Jurkat cells are transiently transfected via 100 μL or 20 μl format 4D Nucleofactor (Lonza®) according to manufacturer's protocol. Cotransfection is performed with 1-3 μg of activator receptor construct and 1-3 μg of inhibitor receptor constructs or empty vector per 1e6 cells and recovered in RPMI media supplemented with 20% heat-inactivated FBS and 0.1% Pen/Strep. To confirm inhibitor receptor surface expression, Jurkat cells are stained 18-24 hours post-transfection with 10 μg/mL streptavidin-PE-HLA-A*02-pMHC tetramer for 60 minutes at 4° C. in PBS with 1% BSA and characterized by flow cytometry (BD FACSCanto II™).

### Jurkat-NFAT-Luciferase Activation Studies

Briefly, Jurkat NFAT-Firefly-Luciferase cells are transfected with activator and inhibitor (blocker) receptor constructs using Lonza 4D Nucleofector™ (AAF-1002B) or Neon™ transfection systems (ThermoFisher®, MPK5000). mRNA transfected target cells (10,000 cells/well), activator only, or activator/blocker expressing target cells at different densities are added to transfected Jurkat-NFAT-Firefly-Luciferase cells (12,000 cells/well) to a final volume of 20 mL in 384-well plates (Corning® 3570). After a 6-hour incubation at 37° C., the One-Step™ Luciferase firefly assay system (BPS Bioscience™, 60690) is used to determine luminescence intensity on a Tecan Infinite® M1000.

### Solid Substrate Antigen Titration

Biotinylated pMHCs are added to streptavidin-coated plates with and without biotinylated recombinant CD28 agonist, biotinylated recombinant PD-L1, or both at concentrations equimolar to the highest pMHC molarity. Mouse IgG1 is used as a control to ensure the binding capacity of the well is not exceeded. The unbound molecules are washed off and transduced T-cells are added. Half of the T-cells are recovered and stained with anti-CD69 at 24 hours and the rest are stained with anti-CD25 at 48 hours.

Activator mRNA Titration

CD19, CD20, HLA-A*02, or HLA-A*03 mRNA are synthesized as described below and are diluted two fold 12 times. Each dilution of mRNA is mixed with two million HeLa cells and electroporated using 4D Nucleofactor™. mRNA transfected target cells are then co-cultured with Jurkat cells transfected with activator receptor CAR or inhibitor receptor CAR for 6 hours before the degree of activation is measured as described above.

In Vitro Transcription of mRNA

CD19, CD20, HLA-A*02, or HLA-A*03 sequences are synthesized by Genscript® with T7 promoter in the 5′ end together with 5′ and 3′ synthetic UTR sequences. In vitro transcription is done in 1×IVT buffer and (40 mM Tris, 10 mM DTT, 2 mM Spermidine, 0.002% Triton X-100, and 27 mM MgCl₂) with T7 RNA polymerase (NEB®, M0251S), inorganic pyrophosphatase (NEB®, M2403S), and murine RNAse inhibitor (NEB®, M0314S). In addition, 500 ng PCR purified template, 5 mM CleanCap™ Cap1 AG trimer, 5 mM each of ATP, CTP, GTP and pseudo-uridine triphosphate (pseudo-UTP) are added, and the transcription reaction is incubated at 37° C. for 2 hours, followed by addition of DNase I (NEB®, M0303S) for additional 15 minutes incubation at 37° C.

In vitro transcription reaction is finalized by the addition of poly A tailing with poly A enzyme (NEB®, M0276S) and incubation at 37° C. for 30 minutes. The mRNA was then cleaned using the NEBR Monarch kit. The purified mRNA was treated with Antarctic phosphatase (NEB®, M0289S) for 1 hour at 37° C. in 1× Antarctic phosphatase buffer (NEB®). mRNA is then purified again using the NEB® Monarch kit. mRNA concentrations are measured by Nanodrop®. The quality of mRNA is accessed by gel electrophoresis. The final in vitro transcribed mRNA is aliquoted and stored at −80° C. until shortly before use.

### Statistical Analysis

Statistical analyses is performed using GraphPad® Prism software. All peptide and cell titration studies are shown as mean±standard deviation (SD). Peptide and cell titration curves are fit using a four-parameter non-linear regression analysis. EC50 values are calculated directly from the curves.

### Example 5: Characterization of the SPN and PECAM-1 Receptor Combination

This prophetic Example describes testing of A antigen SPN and B antigen PECAM-1 in a Tmod™ system.

Experience with the Tmod™ system shows achieving selective activation when in contact with target cells is unpredictable. Various representative species of activator receptors and inhibitory receptors are generated. Subsequently, various combinations of activator receptors and inhibitory receptors are tested as described below.

Selective cancer cell cytotoxicity will be assessed by co-culturing cells expressing SPN activator receptor and PECAM-1 inhibitor receptor with tumor and normal HeLa target cells mixed at a 1:1 ratio for three 48-hour rounds. Tumor (cancerous) and normal (non-cancerous) HeLa target cells will be prepared in a mixed culture and co-cultured with untransduced T cells.

To study the effects of the SPN and PECAM-1 receptor combination in vivo, NSG mice will be injected intravenously via tail vein with approximately 1E6 normal (non-cancerous) and tumor cells modified with firefly luciferase (fLuc) or renilla luciferase (rLuc) on day 0. On day 7, untransduced (“UTD”) cells and cells expressing SPN activator receptor and PECAM-1 inhibitor receptor will be injected at 10-20 million cells per mouse. Tumor growth will be monitored by bioluminescence post T cell injection.

### Cell Culture

Jurkat cells encoding an NFAT luciferase reporter are obtained from BPS Bioscience. In culture, Jurkat cells are maintained in RPMI media supplemented with 10% FBS, 1% Pen/Strep and 0.4 mg/mL G418/Geneticin. HCT.116 and HeLa cells are maintained as suggested by ATCC.

### Jurkat Cell Transfection

Jurkat cells are transiently transfected via 100 μL or 20 μl format 4D Nucleofactor (Lonza®) according to manufacturer's protocol. Cotransfection is performed with 1-3 μg of activator receptor construct and 1-3 μg of inhibitor receptor constructs or empty vector per 1e6 cells and recovered in RPMI media supplemented with 20% heat-inactivated FBS and 0.1% Pen/Strep. To confirm inhibitor receptor surface expression, Jurkat cells are stained 18-24 hours post-transfection with 10 μg/mL streptavidin-PE-PECAM-1-pMHC tetramer for 60 minutes at 4° C. in PBS with 1% BSA and characterized by flow cytometry (BD FACSCanto II™).

### Jurkat-NFAT-Luciferase Activation Studies

Briefly, Jurkat NFAT-Firefly-Luciferase cells are transfected with activator and inhibitor (blocker) receptor constructs using Lonza 4D Nucleofector™ (AAF-1002B) or Neon™ transfection systems (ThermoFisher®, MPK5000). mRNA transfected target cells (10,000 cells/well), activator only, or activator/blocker expressing target cells at different densities are added to transfected Jurkat-NFAT-Firefly-Luciferase cells (12,000 cells/well) to a final volume of 20 mL in 384-well plates (Corning® 3570). After a 6-hour incubation at 37° C., the One-Step™ Luciferase firefly assay system (BPS Bioscience™, 60690) is used to determine luminescence intensity on a Tecan Infinite® M1000.

### Solid Substrate Antigen Titration

Biotinylated pMHCs are added to streptavidin-coated plates with and without biotinylated recombinant CD28 agonist, biotinylated recombinant PD-L1, or both at concentrations equimolar to the highest pMHC molarity. Mouse IgG1 is used as a control to ensure the binding capacity of the well is not exceeded. The unbound molecules are washed off and transduced T-cells are added. Half of the T-cells are recovered and stained with anti-CD69 at 24 hours and the rest are stained with anti-CD25 at 48 hours.

Activator mRNA Titration

SPN or PECAM-1 mRNA are synthesized as described below and are diluted two fold 12 times. Each dilution of mRNA is mixed with two million HeLa cells and electroporated using 4D Nucleofactor™. mRNA transfected target cells are then co-cultured with Jurkat cells transfected with activator receptor CAR or inhibitor receptor CAR for 6 hours before the degree of activation is measured as described above.

In Vitro Transcription of mRNA

SPN or PECAM-1 sequences are synthesized by Genscript® with T7 promoter in the 5′ end together with 5′ and 3′ synthetic UTR sequences. In vitro transcription is done in 1× IVT buffer and (40 mM Tris, 10 mM DTT, 2 mM Spermidine, 0.002% Triton X-100, and 27 mM MgCl₂) with T7 RNA polymerase (NEB®, M0251S), inorganic pyrophosphatase (NEB®, M2403S), and murine RNAse inhibitor (NEB®, M0314S). In addition, 500 ng PCR purified template, 5 mM CleanCap™ Cap1 AG trimer, 5 mM each of ATP, CTP, GTP and pseudo-uridine triphosphate (pseudo-UTP) are added, and the transcription reaction is incubated at 37° C. for 2 hours, followed by addition of DNase I (NEB®, M0303S) for additional 15 minutes incubation at 37° C.

In vitro transcription reaction is finalized by the addition of poly A tailing with poly A enzyme (NEB®, M0276S) and incubation at 37° C. for 30 minutes. The mRNA was then cleaned using the NEB® Monarch kit. The purified mRNA was treated with Antarctic phosphatase (NEB®, M0289S) for 1 hour at 37° C. in 1× Antarctic phosphatase buffer (NEB®). mRNA is then purified again using the NEB® Monarch kit. mRNA concentrations are measured by Nanodrop®. The quality of mRNA is accessed by gel electrophoresis. The final in vitro transcribed mRNA is aliquoted and stored at −80° C. until shortly before use.

### Statistical Analysis

Statistical analyses is performed using GraphPad® Prism software. All peptide and cell titration studies are shown as mean±standard deviation (SD). Peptide and cell titration curves are fit using a four-parameter non-linear regression analysis. EC50 values are calculated directly from the curves.

### Example 6: Relapse-after Stem Cell Transplant

Relapse: A patient having cancer has been treated with a conditioning therapy and stem cell transplant. The patient experiences relapse. The patient is treated with the transplant-sparing immune cells of the present disclosure as a rescue therapy. The immune cells kill the patient cancer cells responsible for relapse, but spare the graft cells. Therefore, the graft cells survive the rescue therapy. Variations include:
Insufficient engraftment of donor cells is observed. The patient is then treated with the transplant-sparing immune cells to suppress the host versus graft response, improving engraftment. (If targets T cells or hematopoietic cell broadly—so CD45, SPN, but not CD33)

### Example 7: Liquid Tumor (Leukemia or Lymphoma) or Other Blood Cell Conditions-Before Stem Cell Transplant

Primary treatment for cancer: A patient receives transplant-sparing immune cells of the present disclosure before a stem cell transplant. By eliminating host effectors, myeloablative conditioning becomes unnecessary, or can be administered as reduced intensity; rather, the transplant-sparing immune cells serve to facilitate conditioning, prevent graft rejection, and also kill cancer cells. The stem cell transplant may be performed with reduced conditioning or no conditioning (i.e., as non-myeloblative transplant). Because the transplant-sparing immune cells are engineered to spare the transplant cells, transplant-sparing immune cells facilitate engraftment of the donor cells and killing of cancer cells. Indications include AML, MDS, ALL, MPNs, lymphoma, and myeloma. Variations include:
Myeloablative conditioning
Reduced intensity conditioning
Non-myeloablative therapy (i.e., limited or no prior conditioning)
Stem cell transplant with CD34 positively selected or CD3/CD19 negatively selected cells. The stem cells may be hematopoietic stem cells. (Gottlieb et al, 2022). This further reduces the risk of graft vs. host disease. The increased risk of relapse associated with a purified stem cell transplant (due to reduced graft vs. tumor effects) is mitigated by the transplant-sparing immune cells.

### Example 8: Gene Therapy—Conditioning Patient for Receipt of Genetically Engineered Cells

A patient receives transplant-sparing immune cells of the present disclosure before a stem cell transplant. By eliminating host effectors, myeloablative conditioning becomes unnecessary, or can be administered as reduced intensity; rather, the transplant-sparing immune cells of the present disclosure serve to facilitate conditioning, prevent graft rejection, and also kill cancer cells. The stem cell transplant may be performed with reduced conditioning or no conditioning (i.e., as non-myeloblative transplant). Because the transplant-sparing immune cells are engineered to spare the transplant cells, transplant-sparing immune cells of the present disclosure facilitate engraftment of the donor cells. The stem cell transplant contains engineered stem cells. Ex vivo genetic engineering of stem cells for gene therapy implements conditions that best preserve the biological properties of the stem cells and applies transduction-enhancing compounds to support ex vivo stem cell expansion and subsequent allogeneic stem cell transplantation.

### Example 9: Conditioning for Non-Malignant Conditions

A patient is treated with transplant-sparing immune cells of the present disclosure, which deplete the patient's own stem and immune cells. The patient is then provided a stem cell transplant to restore blood-forming cells. Because the transplant-sparing immune cells are engineered to spare the transplant cells, transplant results in engraftment of the donor cells (similar to myeloablative therapy followed by autologous transplant). Indications include aplastic anemia (acquired or inherited), hemoglobinopathies, inborn errors of metabolism, and severe immunodeficiencies.

### Example 10: Characterization of the CD33 and CD16 Receptor Combination

This Example describes testing of A antigen CD33 and B antigen CD16 in a Tmod™ system.

Experience with the Tmod™ system shows achieving selective activation when in contact with target cells is unpredictable. Various representative species of activator receptors and inhibitory receptors are generated. Subsequently, various combinations of activator receptors and inhibitory receptors are tested as described below.

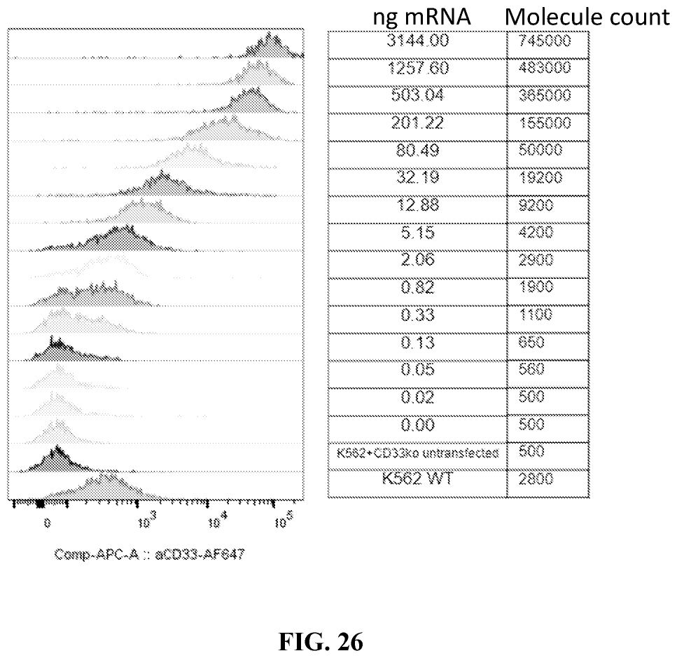

Flow cytometry analysis of CD33 expression levels in K562 target cells in which CD33 was knocked out and then CD33 mRNA titration was transfected (FIG. 26). CD33 antibody clone WM53 used for staining. CD33 molecule count (molecules/cell) was interpolated using the QIFIKIT™ for quantitative analysis.

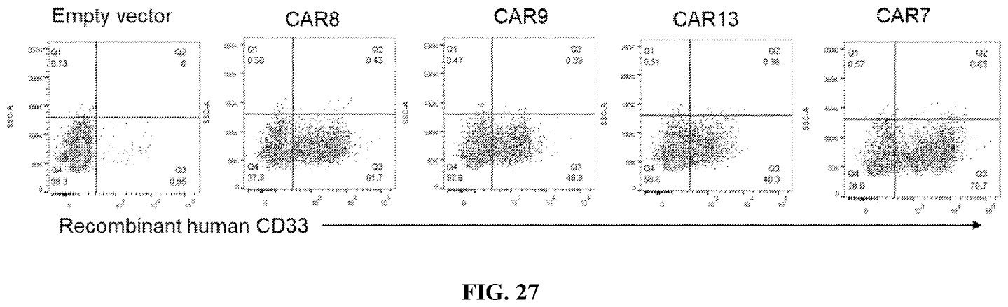

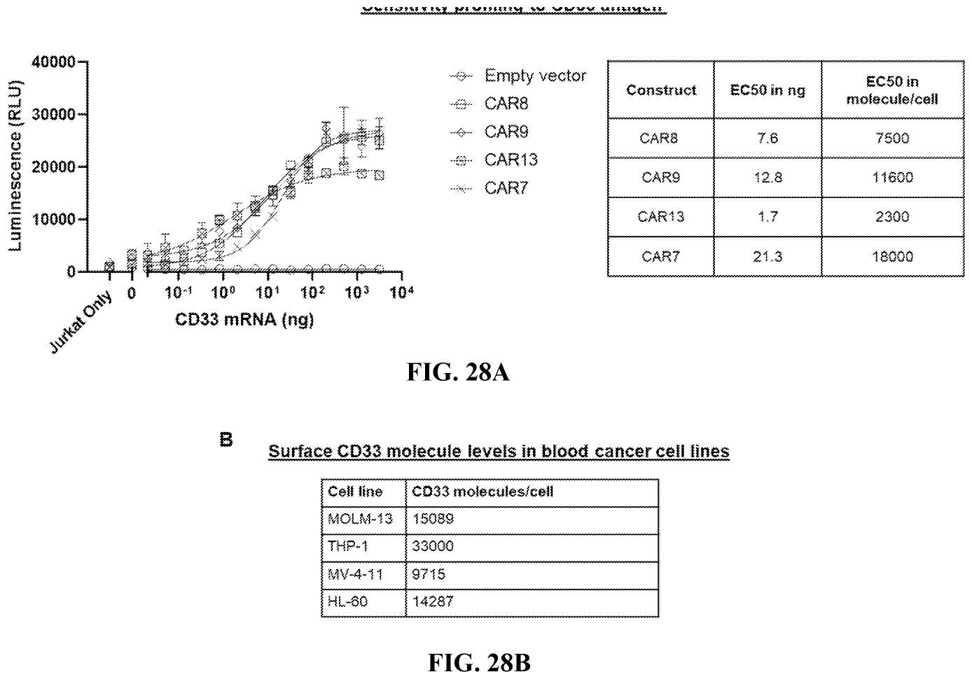

CD33 reactive CAR expression was observed in Jurkat reporter cells and detected by staining with recombinant CD33 protein (FIG. 27; CAR-8: VH SEQ ID NO: 341 and VL SEQ ID NO: 342; CAR-9: Light Chain SEQ ID NO: 682 and Heavy Chain SEQ ID NO: 683; CAR-13: VH SEQ ID NO: 684 and VL SEQ ID NO: 685; CAR-7: VH SEQ ID NO: 337 and VL SEQ ID NO: 338). The Jurkat reporter cells expressing CD33 reactive CARs were then co-cultured with target K562 cells that were transfected with a titration of CD33 mRNA. Six hours post co-culture start time, cells were lysed and luciferase signal was measured (FIG. 28A). CD33 molecules/cell was interpolated using the QIFIKIT™ for quantitative analysis. Surface CD33 molecule levels in molecules/cell were also analyzed in blood cancer cell lines (FIG. 28B).

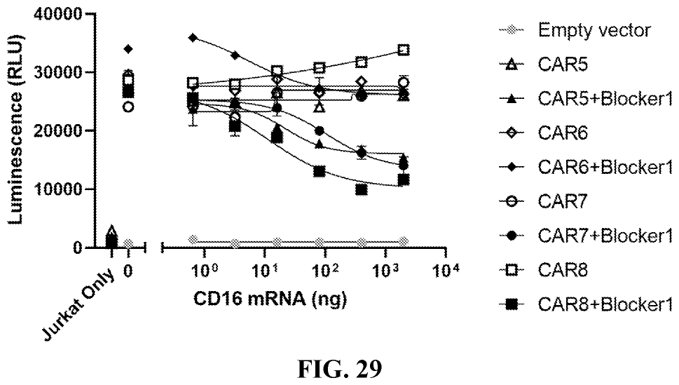

Jurkat reporter cells were transfected with anti-CD33 Activator CAR plasmid and an anti-CD16 Blocker 1 plasmid and co-cultured with K562 target cells that overexpressed CD33 and were transfected with a titration of CD16b mRNA. After six hours of coculture, cells were lysed, and luciferase signal was measured (FIG. 29; CAR-5: Heavy Chain SEQ ID NO: 333 and Light Chain SEQ ID NO: 334; CAR-6: Heavy Chain SEQ ID NO: 331 and Light Chain SEQ ID NO: 332; CAR-7: VH SEQ ID NO: 337 and VL SEQ ID NO: 338; CAR-8: VH SEQ ID NO: 341 and VL SEQ ID NO: 342; anti-CD16 Blocker 1: VH SEQ ID NO: 686 and VL SEQ ID NO: 687). VH SEQ ID NO: 686 is the amino acid translation of SEQ ID NO: 245 and VL SEQ ID NO: 687 is the amino acid translation of SEQ ID NO: 246.

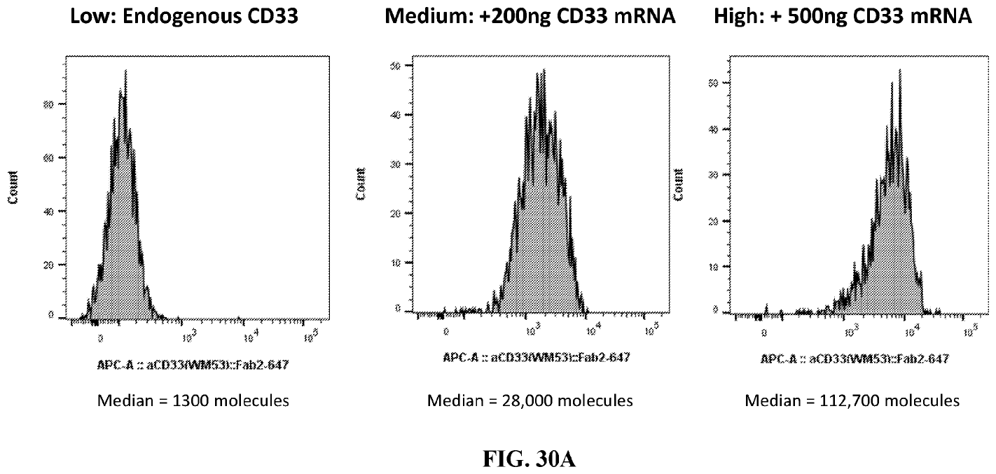

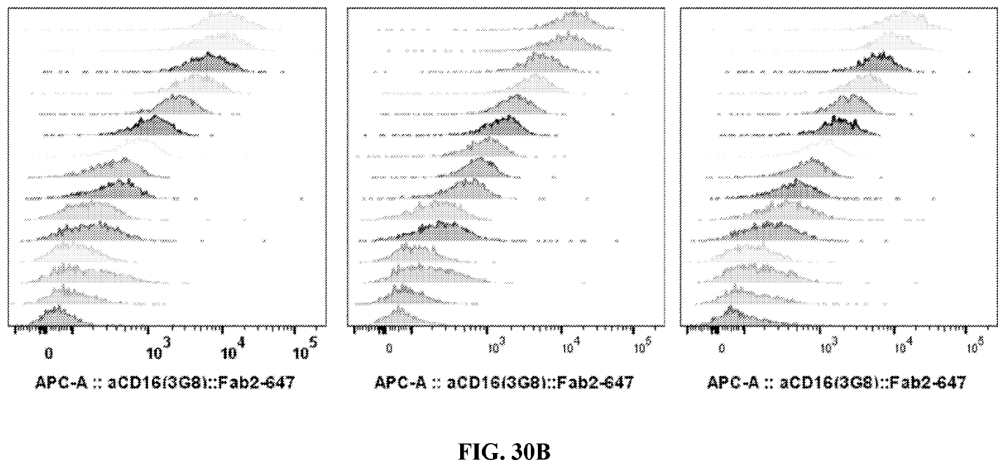

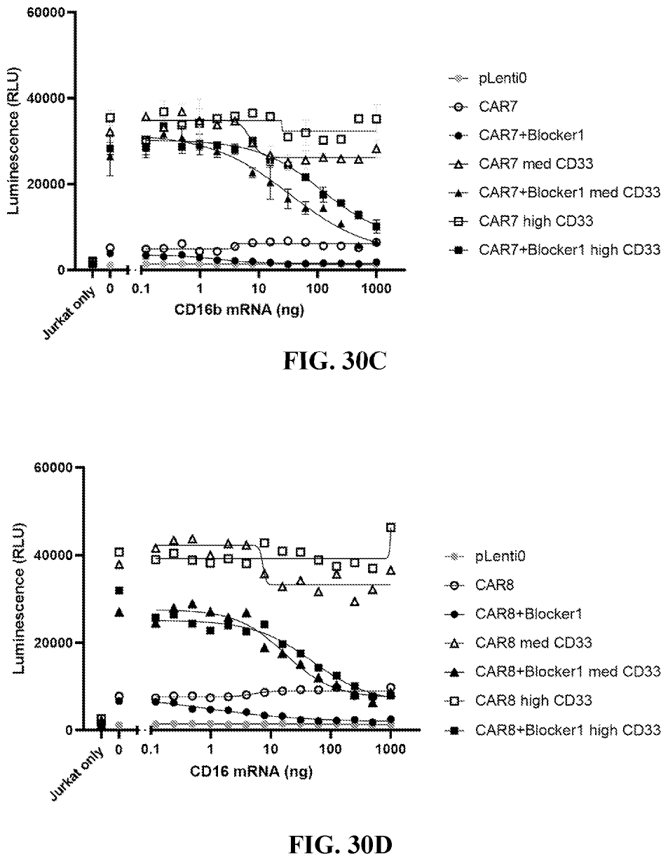

CD16b mRNA was transfected into either wild type K562 cells (FIG. 30A “Low Endogenous CD33”), K562 cells transfected with 200 ng CD33 mRNA (FIG. 30A “Medium CD33”), or K562 cells transfected with 500 ng CD33 mRNA (FIG. 30A “High CD33”). The “medium” level of CD33 expression is representative of CD33 expression in AML cell lines. FIG. 30B depicts the titration of CD16b mRNA at different levels of CD33 expression.

Jurkat reporter cells were transfected with anti-CD33 Activator CAR and anti-CD16 Blocker 1 plasmids and co-cultured with prepared K562 target cells that overexpressed CD33 and were transfected with a titration of CD16b mRNA. After six hours of coculture, cells were lysed, and luciferase signal was measured (FIG. 30C; CAR-7: VH SEQ ID NO: 337 and VL SEQ ID NO: 338; Blocker 1: VH SEQ ID NO: 686 and VL SEQ ID NO: 687; and FIG. 30D; CAR-8: VH SEQ ID NO: 341 and VL SEQ ID NO: 342; anti-CD16 Blocker 1: VH SEQ ID NO: 686 and VL SEQ ID NO: 687).

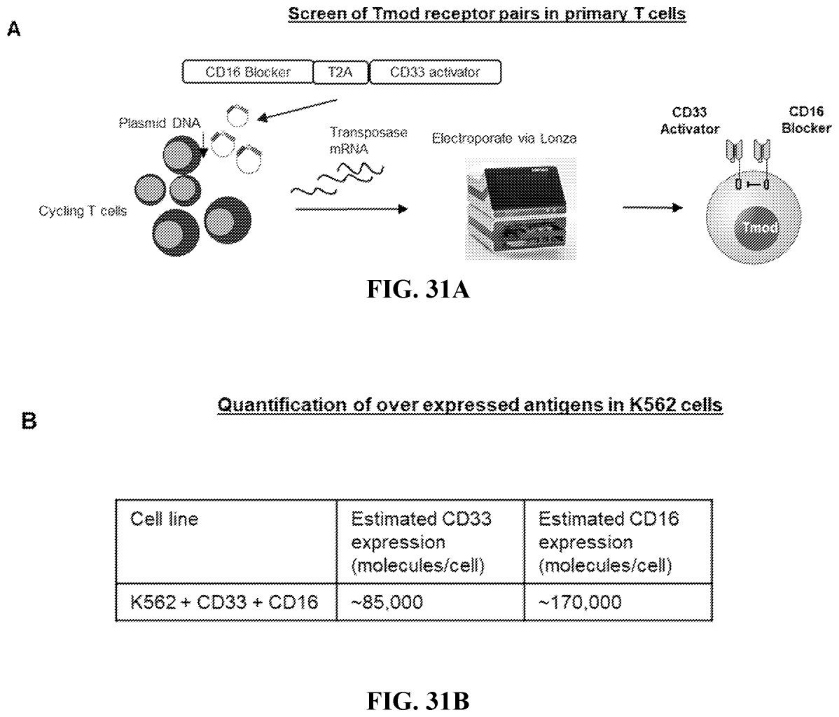

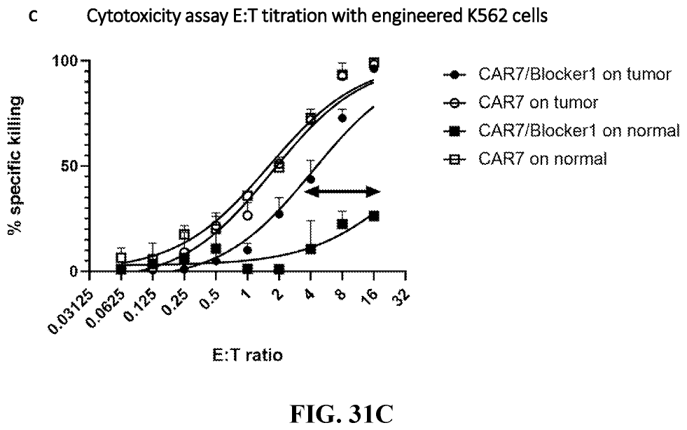

Primary T cells were engineered to stably express Tmod anti-CD16 Blocker 1/anti-CD33 Activator constructs using the piggyBAC™ system (FIG. 31A). The K562 cell line was engineered to model “normal” tissue by overexpressing CD33 and CD16. K562 cells that only overexpressed CD33 were engineered to model the “tumor” tissue. The number of CD33 and CD16 molecules on the surface of the K562 cells was analyzed using QIFIKIT™ (FIG. 31B). Tmod cells expressing anti-CD16 Blocker 1/anti-CD33 Activator constructs “Effector cells” and engineered K562 “Target cells” were co-cultured with a titration of E: T ratios (FIG. 31C; CAR-7: VH SEQ ID NO: 337 and VL SEQ ID NO: 338; anti-CD16 Blocker 1: VH SEQ ID NO: 686 and VL SEQ ID NO: 687). The percent (%) specific killing was calculated at 48 hours of co-culture.

### Example 11: Characterization of the SPN and CD16 Receptor Combination

This Example describes testing of A antigen SPN and B antigen CD16 in a Tmod™ system.

Experience with the Tmod™ system shows achieving selective activation when in contact with target cells is unpredictable. Various representative species of activator receptors and inhibitory receptors are generated. Subsequently, various combinations of activator receptors and inhibitory receptors are tested as described below.

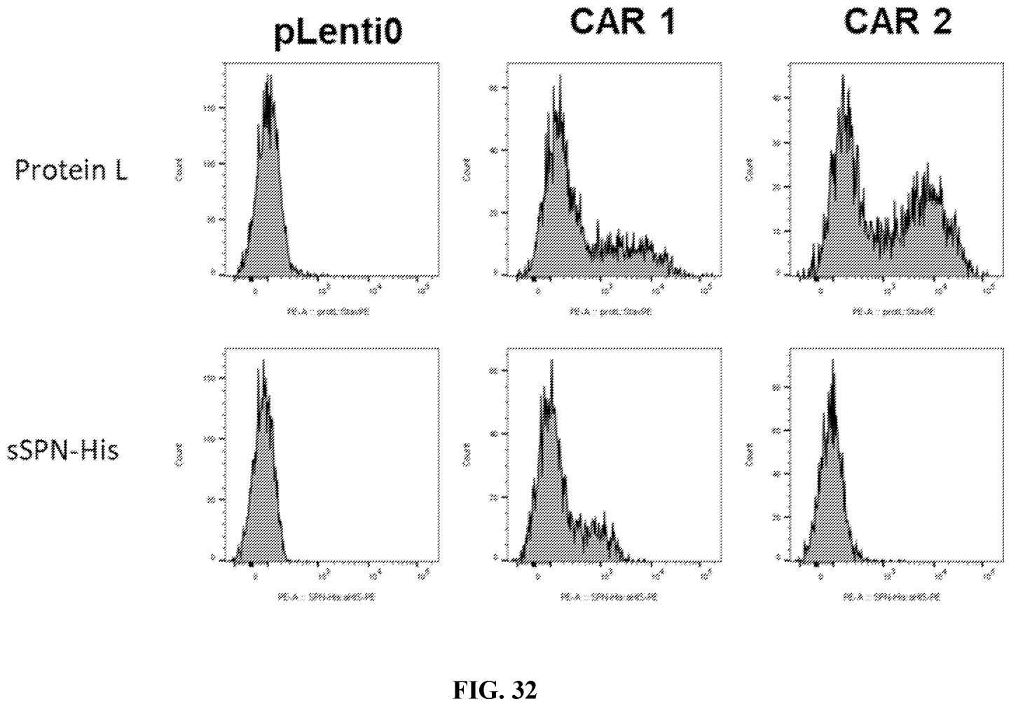

SPN reactive CAR expression was observed in Jurkat reporter cells. Protein L or soluble recombinant human His-tagged SPN protein (sSPN-His) was used to stain the CARs followed by analysis by flow cytometry (FIG. 32; CAR-1: VH SEQ ID NO: 269 and VL SEQ ID NO: 289; CAR-2: Light Chain SEQ ID NO: 260 and Heavy Chain SEQ ID NO: 259).

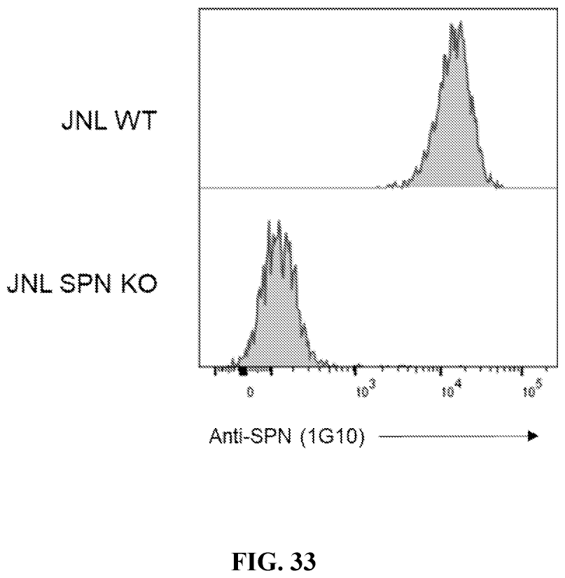

For an SPN depleted cell line, Jurkat nuclear factor of activated T cells (NFAT) luciferase (JNL) SPN knock out (KO) cells were produced using CRISPR and a guide RNA targeting the SPN gene followed by cell sorting to enrich the SPN KO population. Cells were stained using anti-SPN antibody clone 1G10 followed by analysis by flow cytometry. The histograms in FIG. 33 show SPN staining of JNL SPN KO and JNL wild type (WT) cells.

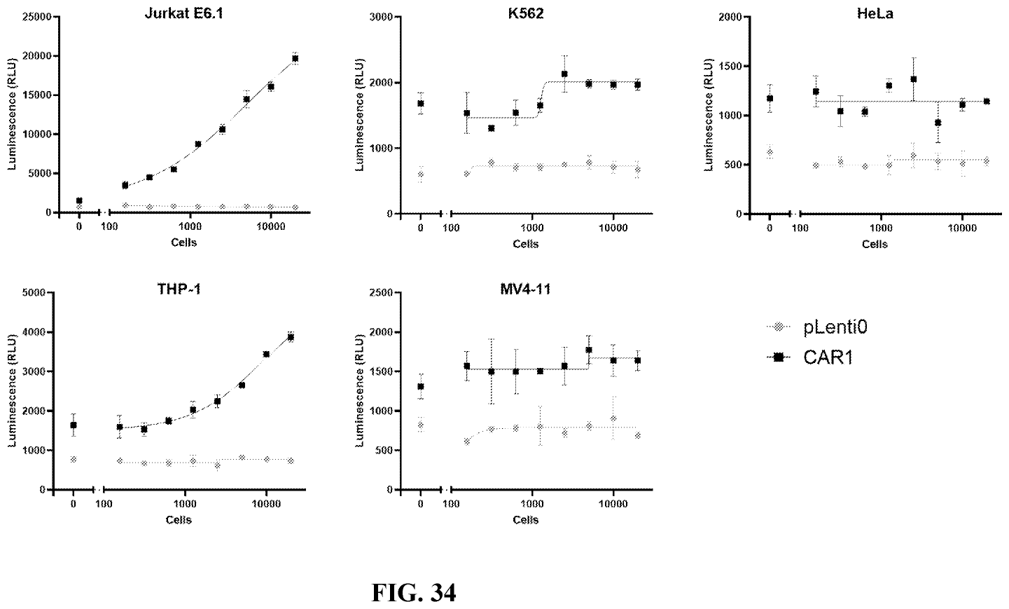

To analyze anti-SPN CAR expression, JNL cells were transfected with 2 μg of activator anti-SPN CAR DNA per 1E6 cells using the Neon™ electroporation system. 18-24 hours post-transfection, 10,000 Jurkat cells were co-cultured with serially titrated Target cells (Jurkat E6.1, K562, HeLa, THP-1, and MV4-11) for 6 hours. The functional response (RLU) was assessed using ONE™ step luciferase assay system. Anti-SPN CARI showed a robust dose-dependent response to Jurkat and THP-1 cells and low response to MV4-11, HeLa, and K562 cells (FIG. 34; CAR-1: VH SEQ ID NO: 269 and VL SEQ ID NO: 289).

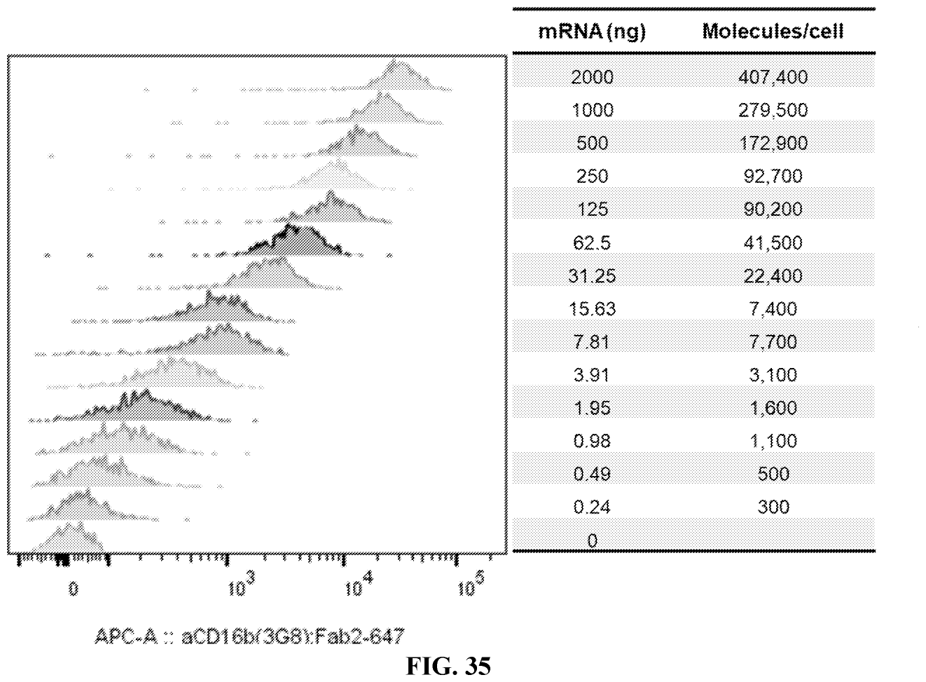

Flow cytometry histogram analysis of Flag-CD16b mRNA titrated in Jurkat WT cells was performed (FIG. 35). CD16 expression was detected with anti-CD16 antibody 3G8 and quantified by converting flow-cytometry mean fluorescence intensity (MFI) signal to molecules of CD16 per cell using QIFIKIT™ quantitative analysis kit.

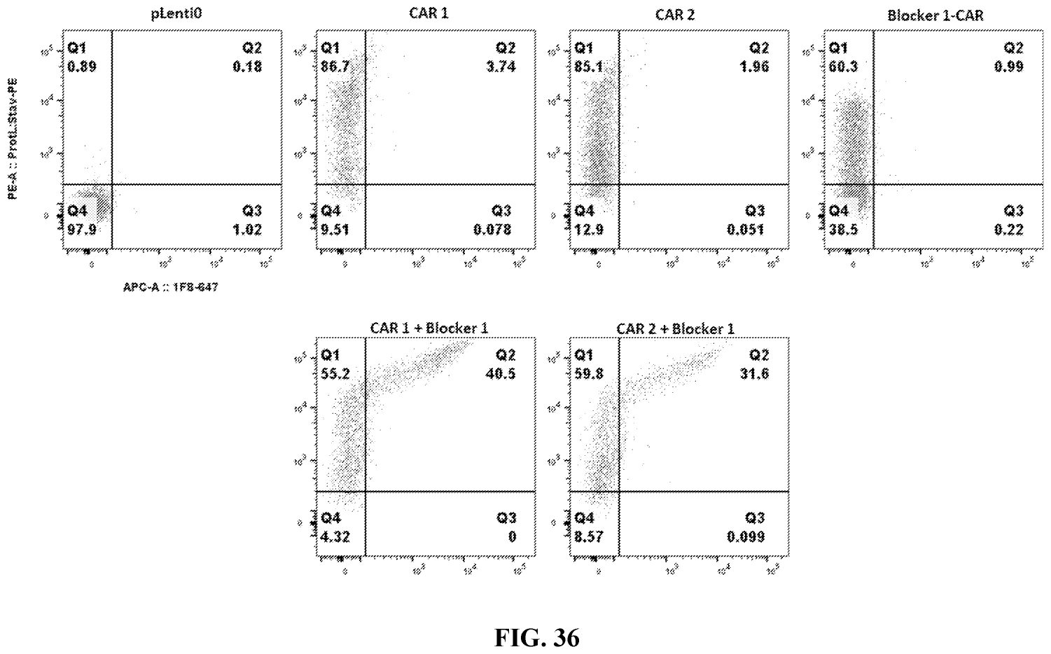

FIG. 36 shows flow cytometry plots of anti-SPN CAR expression “CAR 1” and “CAR 2” with or without anti-CD16 Blocker 1 in JNL SPN KO cells (CAR-1: VH SEQ ID NO: 269 and VL SEQ ID NO: 289; CAR-2: Light Chain SEQ ID NO: 260 and Heavy Chain SEQ ID NO: 259; anti-CD16 Blocker 1: VH SEQ ID NO: 686 and VL SEQ ID NO: 687). VH SEQ ID NO: 686 is the amino acid translation of SEQ ID NO: 245 and VL SEQ ID NO: 687 is the amino acid translation of SEQ ID NO: 246. anti-CD16 Blocker 1 control was generated through fusing the anti-CD16 Blocker 1 scFv to CAR domains. Anti-SPN CAR staining was performed using Protein L and anti-CD16 Blocker 1 staining was performed using an anti-CD16 Blocker 1 hinge-specific antibody. Percentages for gated populations shown on plots.

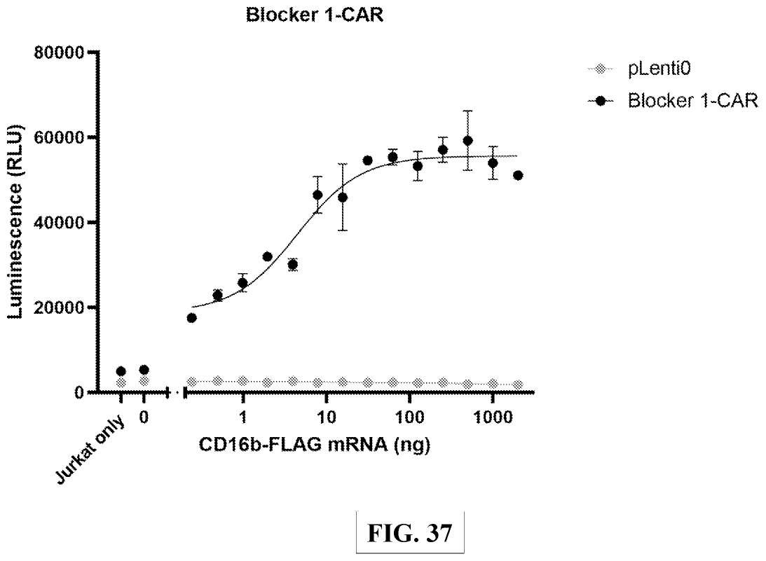

Jurkat NFAT luciferase (JNL) cells were transfected with an anti-CD16 Blocker 1 scFv fused to CAR domains in an mRNA titration assay. Jurkat WT target cells were transfected with serially diluted Flag-CD16b mRNA and JNL SPN KO cells were transfected with 2 μg of activator anti-SPN CAR DNA per 1E6 cells using the Neon™ electroporation system. 18-24 hours post-transfection, 10,000 JNL anti-SPN CAR cells were co-cultured with 5000 transfected Jurkat WT target cells for 6 hours. The functional response (RLU) was assessed using ONE™ step luciferase assay system. The CAR containing anti-CD16 Blocker 1 scFv showed a robust dose-dependent response (FIG. 37; CAR-1: VH SEQ ID NO: 269 and VL SEQ ID NO: 289; anti-CD16 Blocker 1: VH SEQ ID NO: 686 and VL SEQ ID NO: 687).

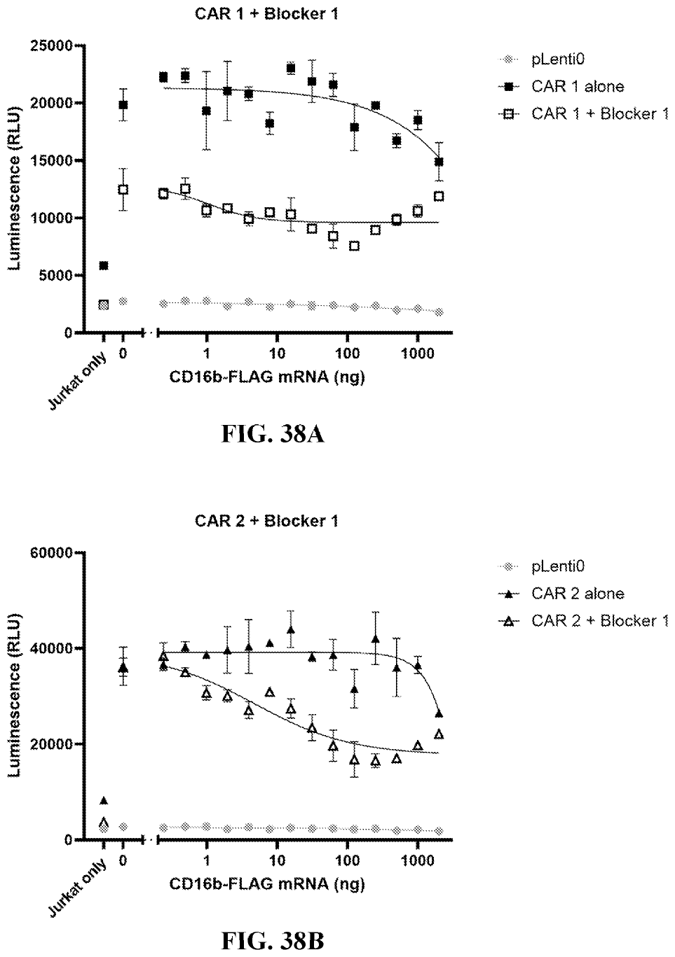

Jurkat NFAT luciferase (JNL) cells transfected with an anti-CD16 Blocker 1 displayed blocking of anti-SPN CAR 1 (FIG. 38A) or CAR 2 (FIG. 38B) activator CARs (FIG. 38; CAR-1: VH SEQ ID NO: 269 and VL SEQ ID NO: 289; CAR-2: Light Chain SEQ ID NO: 260 and Heavy Chain SEQ ID NO: 259; anti-CD16 Blocker 1: VH SEQ ID NO: 686 and VL SEQ ID NO: 687). Jurkat WT target cells were transfected with serially diluted FLAG-CD16b mRNA and JNL SPN KO cells were transfected with 1 μg of activator anti-SPN CAR DNA and 2 μg of anti-CD16 Blocker 1 DNA per 1E6 cells using the Neon™ electroporation system. 18-24 hours post-transfection, 10,000 JNL cells were co-cultured with 5000 transfected Jurkat cells for 6 hours. The activation of JNL cells was assessed by measuring the functional response (RLU) using the ONE™ step luciferase assay system.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. An immune cell for treatment of blood cancer, comprising
a. an activator receptor comprising an extracellular ligand binding domain specific to a first activator antigen expressed by blood cancer cells; and
b. an inhibitor receptor comprising an extracellular ligand binding domain specific to a first inhibitor antigen expressed by non-cancerous blood cells.

2. The immune cell of claim 1, wherein the first activator antigen is expressed by hematopoietic cells.

3. The immune cell of claim 2, wherein the first inhibitor antigen is expressed by myeloid cells.

4. The immune cell of claim 2, wherein the first inhibitor antigen is expressed by hematopoietic stem cells.

5. The immune cell of claim 1, wherein
a. the first activator antigen is SPN and the first inhibitor antigen is PECAM-1;
b. the first activator antigen is CD45 and the first inhibitor antigen is PECAM-1;
c. the first activator antigen is CD11a and the first inhibitor antigen is PECAM-1;
d. the first activator antigen is SPN, the second activator antigen is CD33, and the first inhibitor antigen is PECAM-1;
e. the first activator antigen is SPN, the second activator antigen is CD45, and the first inhibitor antigen is PECAM-1;
f. the first activator antigen is SPN, the second activator antigen is FLT3, and the first inhibitor antigen is PECAM-1;
g. the first activator antigen is CD33, the second activator antigen is FLT3, and the first inhibitor antigen is PECAM-1.

6. The immune cell of any one of claims 1-5, wherein the activator receptor is a T cell receptor (TCR) or a chimeric antigen receptor (CAR).

7. The immune cell of claim 6, wherein the extracellular ligand binding domain of the activator receptor comprises an antibody fragment, a single chain Fv antibody fragment (scFv), a β chain variable domain (Vβ), or a TCR α chain variable domain and a TCR β chain variable domain.

8. The immune cell of claim 6 or 7, wherein the extracellular ligand binding domain of the activator receptor comprises a heavy chain variable (VH) region and a light chain variable (VL) region.

9. The immune cell of claim 8, wherein the VH region comprises the sequence of any one of SEQ ID NOs: 249-252, 257, 259, 261-280, or a sequence having at least 85%, at least 90%, at least 95%, at least 97%, at least 99% identity, or 100% identical thereto; and the VL region comprises the sequence of any one of SEQ ID NOs: 253-256, 258, 260, 281-303, or a sequence having at least 85%, at least 90%, at least 95%, at least 97%, at least 99% identity, or 100% identical thereto.

10. The immune cell of any one of claims 6-9, wherein the CAR comprises a hinge sequence isolated or derived from CD8, CD28, IgG1, or IgG4, or a synthetic hinge.

11. The immune cell of any one of claims 6-10, wherein the CAR comprises a transmembrane domain isolated or derived from CD8 or CD28.

12. The immune cell of any one of claims 6-11, wherein the CAR comprises an intracellular domain isolated or derived from CD28, 4-1BB or CD3z, or a combination thereof.

13. The immune cell of any one of claims 1-12, wherein the inhibitor receptor is a TCR or a CAR.

14. The immune cell of claim 13, wherein the extracellular ligand binding domain of the inhibitor receptor comprises an antibody fragment, a single chain Fv antibody fragment (scFv), a β chain variable domain (Vβ), or a TCR α chain variable domain and a TCR β chain variable domain.

15. The immune cell of claim 13 or 14, wherein the extracellular ligand binding domain of the inhibitor receptor comprises a heavy chain variable (VH) region and a light chain variable (VL) region.

16. The immune cell of any one of claims 1-15, wherein the inhibitor receptor comprises a LILRB1 intracellular domain or a functional variant thereof.

17. The immune cell of any one of claims 1-16, wherein the inhibitor receptor comprises LILRB1 hinge and transmembrane domains, or functional variants thereof.

18. A pharmaceutical composition, comprising an effective amount of the immune cells of any one of claims 1-17.

19. A polynucleotide system, comprising one or more polynucleotides comprising polynucleotide sequences encoding:
a. an activator receptor comprising an extracellular ligand binding domain specific to a SPN antigen; and
b. an inhibitor receptor comprising an extracellular ligand binding domain specific to a PECAM-1 antigen.

20. A method of making an immune cell therapy, comprising transforming immune cells with the polynucleotide system of claim 19.

21. A kit comprising the immune cell of any one of claims 1-17 or the pharmaceutical composition of claim 18.

22. A method of treating cancer in a subject, comprising administering to the subject an immune cell comprising
(a) an activator receptor comprising an extracellular ligand binding domain specific to a SPN antigen, wherein the extracellular ligand binding domain of the activator receptor is an scFv; and
(b) an inhibitor receptor comprising an extracellular ligand binding domain specific to a PECAM-1 antigen that is not expressed by a blood cancer cell, wherein the extracellular ligand binding domain of the inhibitory receptor is an scFv.

23. A method of identifying antigens for use in treating blood cancers with chimeric antigen receptor immune cell therapy, comprising:
a. selecting an antigen expressed on blood cancer cells; and
b. selecting an antigen expressed on non-cancerous blood cells.

24. The method of claim 23, comprising generating an immune cell comprising two or more receptors comprising antigen-binding domains, wherein one receptor binds to a first antigen expressed on blood cancer cells and one receptor binds to the second antigen expressed on non-cancerous blood cells.

25. The immune cell of claim 8, wherein the extracellular binding domain of the activator receptor comprises a VH region comprising any one of the sequences of SEQ ID NOs: 16, 17, 20, 21, 23, 25, 27 or a sequence having at least 85%, at least 90%, at least 95%, at least 97%, at least 99% identity, or 100% identical thereto; and a VL region comprising any one of the sequences of SEQ ID NOs: 18, 19, 22, 24, 26, 28, or a sequence having at least 85%, at least 90%, at least 95%, at least 97%, at least 99% identity, or 100% identical thereto.

26. The immune cell of claim 25, wherein the VH region comprises one or more CDR sequences selected from the group consisting of RYWMS (SEQ ID NO: 10), EINPTSSTINFTPSLKD (SEQ ID NO: 11), and GNYYRYGDAMDY (SEQ ID NO: 12); and the VL region comprises one or more CDR sequences selected from the group consisting of RASKSVSTSGYSYLH (SEQ ID NO: 13), LASNLES (SEQ ID NO: 14), and QHSRELPFTFGSGT (SEQ ID NO: 15).

27. The immune cell of claim 14, wherein the scFv of the activator receptor comprises a sequence having at least 85%, at least 90%, at least 95%, at least 97%, at least 99% identity, or 100% identical to a sequence selected from the group consisting of SEQ ID NOS: 29-32.

28. The immune cell of any one of claims 25-27 wherein the activator receptor comprises a sequence having at least 85%, at least 90%, at least 95%, at least 97%, at least 99% identity, or 100% identical to any one of SEQ ID NOs: 133-140, 142, 144, 146, 148, 151, 153, 155-168, or 328-330.

29. The immune cell of claim 8, wherein the extracellular binding domain of the activator receptor comprises a VH region comprising any one of the sequences of SEQ ID NOs: 179, 247, or a sequence having at least 85%, at least 90%, at least 95%, at least 97%, at least 99% identity, or 100% identical thereto; and a VL region comprising any one of the sequences of SEQ ID NOs: 180, 248, or a sequence having at least 85%, at least 90%, at least 95%, at least 97%, at least 99% identity, or 100% identical thereto.

30. The immune cell of claim 14, wherein the scFv of the activator receptor comprises a sequence having at least 85%, at least 90%, at least 95%, at least 97%, at least 99% identity, or 100% identical to a sequence selected from the group consisting of SEQ ID NOS: 304-312.

31. The immune cell of any one of claims 29-30, wherein the activator receptor comprises a sequence having at least 85%, at least 90%, at least 95%, at least 97%, at least 99% identity, or 100% identical to any one of SEQ ID NOs: 313-318.

32. The immune cell of claim 1, comprising a second extracellular ligand binding domain specific to a second activator antigen expressed by blood cancer cells.

33. The immune cell of claim 8, wherein the extracellular binding domain of the second activator receptor comprises a VH region comprising any one of the sequences of SEQ ID NOs: 181, 331, 333, 336, 337, 339, 341, or a sequence having at least 85%, at least 90%, at least 95%, at least 97%, at least 99% identity, or 100% identical thereto; and a VL region comprising any one of the sequences of SEQ ID NOs: 182, 332, 334, 335, 338, 340, 342, or a sequence having at least 85%, at least 90%, at least 95%, at least 97%, at least 99% identity, or 100% identical thereto.

34. The immune cell of claim 8, wherein the extracellular binding domain of the second activator receptor comprises a VH region comprising any one of the sequences of SEQ ID NOs: 16, 17, 20, 21, 23, 25, 27 or a sequence having at least 85%, at least 90%, at least 95%, at least 97%, at least 99% identity, or 100% identical thereto; and a VL region comprising any one of the sequences of SEQ ID NOs: 18, 19, 22, 24, 26, 28, or a sequence having at least 85%, at least 90%, at least 95%, at least 97%, at least 99% identity, or 100% identical thereto.

35. The immune cell of claim 34, wherein the VH region comprises one or more CDR sequences selected from the group consisting of RYWMS (SEQ ID NO: 10), EINPTSSTINFTPSLKD (SEQ ID NO: 11), and GNYYRYGDAMDY (SEQ ID NO: 12); and the VL region comprises one or more CDR sequences selected from the group consisting of RASKSVSTSGYSYLH (SEQ ID NO: 13), LASNLES (SEQ ID NO: 14), and QHSRELPFTFGSGT (SEQ ID NO: 15).

36. The immune cell of claim 33, wherein the scFv of the second activator receptor comprises a sequence having at least 85%, at least 90%, at least 95%, at least 97%, at least 99% identity, or 100% identical to a sequence selected from the group consisting of SEQ ID NOS: 29-32.

37. The immune cell of any one of claims 34-36, wherein the second activator receptor comprises a sequence having at least 85%, at least 90%, at least 95%, at least 97%, at least 99% identity, or 100% identical to any one of SEQ ID NOs: 133-140, 142, 144, 146, 148, 151, 153, 155-168, or 328-330.

38. The immune cell of claim 8, wherein the extracellular binding domain of the second activator receptor comprises a VH region comprising any one of the sequences of SEQ ID NOs: 319, 321, 344, 346, 348, 350, 352, 353, 355, 357, 359, 361, 363, 365, 367, 369, 371, 373, 375, 377, or a sequence having at least 85%, at least 90%, at least 95%, at least 97%, at least 99% identity, or 100% identical thereto; and a VL region comprising any one of the sequences of SEQ ID NOs: 320, 322, 343, 345, 347, 349, 351, 354, 356, 358, 360, 362, 364, 366, 368, 370, 372, 374, 376, 378, or a sequence having at least 85%, at least 90%, at least 95%, at least 97%, at least 99% identity, or 100% identical thereto.

39. The immune cell of claim 8, wherein the extracellular binding domain of the first activator receptor comprises a VH region comprising any one of the sequences of SEQ ID NOs: 181, 331, 333, 336, 337, 339, 341, or a sequence having at least 85%, at least 90%, at least 95%, at least 97%, at least 99% identity, or 100% identical thereto; and a VL region comprising any one of the sequences of SEQ ID NOs: 182, 332, 334, 335, 338, 340, 342, or a sequence having at least 85%, at least 90%, at least 95%, at least 97%, at least 99% identity, or 100% identical thereto.

40. The immune cell of claim 8, wherein the extracellular binding domain of the second activator receptor comprises a VH region comprising any one of the sequences of SEQ ID NOs: 319, 321, 344, 346, 348, 350, 352, 353, 355, 357, 359, 361, 363, 365, 367, 369, 371, 373, 375, 377, or a sequence having at least 85%, at least 90%, at least 95%, at least 97%, at least 99% identity, or 100% identical thereto; and a VL region comprising any one of the sequences of SEQ ID NOs: 320, 322, 343, 345, 347, 349, 351, 354, 356, 358, 360, 362, 364, 366, 368, 370, 372, 374, 376, 378, or a sequence having at least 85%, at least 90%, at least 95%, at least 97%, at least 99% identity, or 100% identical thereto.

41. The immune cell of any one of the preceding claims, wherein the blood cancer cell is a lymphoma cell, a leukemia cell, a myeloma cell, a Reed-Sternberg cell, a myeloproliferative neoplasm cell, or a Waldenstrom macroglobulinemia cell.

42. The immune cell of any one of the preceding claims, wherein the blood cancer cell is a leukemia cell or a lymphoma cell.

43. The immune cell of any one of the preceding claims, wherein the immune cell is a T cell.

44. The immune cell of claim 43, wherein the T cell is a CD8+ CD4− T cell.

45. A nanocarrier comprising one or more polynucleotides, wherein the one or more polynucleotides encode:
a. an activator receptor comprising an extracellular ligand binding domain specific to a first activator antigen expressed by blood cancer cells; and
b. an inhibitor receptor comprising an extracellular ligand binding domain specific to a first inhibitor antigen expressed by non-cancerous blood cells.

46. The nanocarrier or claim 45, wherein the nanocarrier is capable of delivering the one or more polynucleotides to an immune cell in vivo or ex vivo.

47. The nanocarrier of claim 45 or claim 46, wherein the nanocarrier is a lipid nanoparticle (LNP).

48. The nanocarrier of any one of claims 45-47, wherein the one or more polynucleotides are one or more messenger ribonucleic acids (mRNAs) or modified mRNAs (mmRNAs).

49. The nanocarrier of any one of claims 45-48, wherein
a. the first activator antigen is SPN and the first inhibitor antigen is PECAM-1;
b. the first activator antigen is CD45 and the first inhibitor antigen is PECAM-1;
c. the first activator antigen is CD11a and the first inhibitor antigen is PECAM-1;
d. the first activator antigen is SPN, the second activator antigen is CD33, and the first inhibitor antigen is PECAM-1;
e. the first activator antigen is SPN, the second activator antigen is CD45, and the first inhibitor antigen is PECAM-1;
f. the first activator antigen is SPN, the second activator antigen is FLT3, and the first inhibitor antigen is PECAM-1;
g. the first activator antigen is CD33, the second activator antigen is FLT3, and the first inhibitor antigen is PECAM-1;

50. An immune cell, comprising
a. an activator receptor comprising an extracellular ligand-binding domain specific to an activator antigen expressed by blood cells; and
b. an inhibitor receptor comprising an extracellular ligand-binding domain specific to an inhibitor antigen,
wherein the inhibitor antigen is an allelic variant of a Human Leukocyte Antigen (HLA).

51. The immune cell of claim 50, wherein the activator antigen is expressed by hematopoietic cells, myeloid cells, or hematopoietic stem cells, or a combination thereof.

52. The immune cell of any one of the preceding claims, wherein the activator antigen is SPN, CD45, ITGAL, CD33, or FLT3.

53. The immune cell of any one of the preceding claims, wherein the activator antigen is a peptide antigen of SPN, CD45, ITGAL, CD33, or FLT3 in complex with a Major Histocompatibility Complex (MHC).

54. The immune cell of any one of the preceding claims, wherein the inhibitor antigen is an allelic variant of a major HLA.

55. The immune cell of any one of the preceding claims, wherein the inhibitor antigen is HLA-A*02, HLA-B*07, HLA-C*07, or HLA-A*69.

56. The immune cell of any one of the preceding claims, wherein the immune cell is a T cell.

57. The immune cell of any one of the preceding claims, wherein the extracellular-ligand binding domains of the activator receptor and/or of the inhibitor receptor each individually comprises a single chain Fv antibody fragment (scFv).

58. The immune cell of any one of the preceding claims, wherein the activator receptor is a chimeric antigen receptor comprising the extracellular ligand-binding domain; a transmembrane domain; and a CD28 intracellular domain, a 4-1BB intracellular domain, or a CD3z intracellular domain.

59. The immune cell of any one of the preceding claims, wherein the inhibitor receptor comprises the extracellular ligand-binding domain; a transmembrane domain; and an LILRB1 intracellular domain.

60. The immune cell of claim 59, wherein the inhibitor receptor comprises an LILRB1 hinge and/or an LILRB1 transmembrane domains.

61. A pharmaceutical composition, comprising the immune cells of any one of claims 50-60 and one or more pharmaceutically acceptable excipients or diluents.

62. A kit comprising the pharmaceutical composition of claim 61, and instructions for use.

63. A polynucleotide system, encoding the activator receptor and/or the inhibitor receptor of the immune cell of any one of claims 50-60.

64. A method of killing blood cells, comprising contacting blood cells and allogeneic stem cells with the immune cell of any one of claims 50-60,
wherein the inhibitor receptor of the immune cell is specific to an inhibitor antigen expressed by the allogenic stem cells, such that the allogenic stem cells are spared from killing by the immune cells, and
wherein the blood cells lack the inhibitor antigen, such that the immune cell kills the blood cells.

65. A method of treating or preventing relapse of blood cancer in a subject treated for blood cancer with allogeneic stem cell transplant, comprising administering to the subject the immune cell of any one of claim 50-60 or the pharmaceutical composition of claim 61.

66. A method of conditioning a subject for allogeneic stem cell transplant, comprising administering to the subject the immune cell of any one of claim 50-60 or the pharmaceutical composition of claim 61.

67. The method of claim 66, wherein the immune cell or the pharmaceutical composition is administered in an amount effective to condition the subject for stem cell transplant without another conditioning therapy.

68. The method of claim 66 or claim 67, wherein the immune cell or the pharmaceutical composition is administered in an amount effective to treat blood cancer in the subject without another conditioning therapy.

69. The method of claim 66, wherein the method comprises administering a second conditioning therapy.

70. The method of claim 69, wherein the second conditioning therapy is chemotherapy.

71. The method of claim 69, wherein the second conditioning therapy is radiation therapy.

72. The method of claim one of claims 69-71, wherein the second conditioning therapy is administered in amount less than an amount effective to condition the subject for stem cell transplant without the immune cell or pharmaceutical composition.

73. The method of claim one of claims 69-72, wherein the second conditioning therapy is administered in amount less than an amount effective to treat blood cancer in the subject without the immune cell or pharmaceutical composition.

74. A method of allogeneic stem cell transplant in a subject in need thereof, comprising:
a. administering to the subject the immune cell of any one of claim 50-60 or the pharmaceutical composition of claim 61; and
b. administering to the subject an allogeneic stem cell transplant,
wherein inhibitor receptor of the immune cell is specific to an inhibitor antigen expressed by the cells of the stem cell transplant, such that the immune cells spare the stem cell transplant, and
wherein the cells of the subject lack the inhibitor antigen, such that the immune cell or pharmaceutical composition kills blood cells in the subject to condition the subject for the stem cell transplant.

75. A method of treating blood cancer in a subject in need thereof, comprising:
a. administering to the subject the immune cell of any one of claim 50-60 or the pharmaceutical composition of claim 61; and
b. administering to the subject an allogeneic stem cell transplant,
wherein inhibitor receptor of the immune cell is specific to an inhibitor antigen expressed by the cells of the stem cell transplant, such that the immune cells spare the stem cell transplant, and
wherein the cells of the blood cancer lack the inhibitor antigen, such that the immune cell or pharmaceutical composition kills blood cancer cells in the subject.

76. A biobank, comprising a collection of immune cells according to any one of claim 1 14 and a collection of allogeneic stem cell transplant, wherein each allogeneic stem cell transplant is positive for the allelic variant of an HLA and each immune cell comprises an inhibitor receptor specific to one of the alleleic variants.

77. A method of allogeneic stem cell transplant, comprising
a. identifying a subject as homozygous null for an allelic variant of an HLA; and
b. matching the subject to an immune cells comprising an inhibitor receptor specific to the allelic variant and an allogeneic stem cell positive for the allelic variant.


SEQUENCE LISTING (687 sequences)

SEQ ID NO: 1 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 2 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 3 - protein, 1258 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 4 - protein, 1259 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 5 - protein, 1240 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 6 - protein, 1211 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 7 - protein, 1192 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 8 - protein, 1193 aa, Homo sapiens
MTMYLWLKLLAFGFAFLDTEVFVTGQSPTPSPTDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 9 - protein, 480 aa, synthetic construct
QSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKC

SEQ ID NO: 10 - protein, 5 aa, synthetic construct
RYWMS

SEQ ID NO: 11 - protein, 17 aa, synthetic construct
EINPTSSTINFTPSLKD

SEQ ID NO: 12 - protein, 12 aa, synthetic construct
GNYYRYGDAMDY

SEQ ID NO: 13 - protein, 15 aa, synthetic construct
RASKSVSTSGYSYLH

SEQ ID NO: 14 - protein, 7 aa, synthetic construct
LASNLES

SEQ ID NO: 15 - protein, 14 aa, synthetic construct
QHSRELPFTFGSGT

SEQ ID NO: 16 - protein, 123 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKIS

SEQ ID NO: 17 - protein, 121 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSK

SEQ ID NO: 18 - protein, 111 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 19 - protein, 218 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 121 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSS

SEQ ID NO: 21 - protein, 121 aa, synthetic construct
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGNYYRYGDAMDYWGQGTLVTVSS

SEQ ID NO: 22 - protein, 111 aa, synthetic construct
EIVLTQSPATLSLSLGERATISCRASKSVSTSGYSYLHWYQQKPGQAPKLLIYLASNRATGVPARFSGSGPGTDFTLTISSLEPEDFATYYCQHSRELPFTFGQGTKLEIK

SEQ ID NO: 23 - protein, 121 aa, synthetic construct; identical to SEQ ID NO: 20

SEQ ID NO: 24 - protein, 111 aa, synthetic construct; identical to SEQ ID NO: 18

SEQ ID NO: 25 - protein, 117 aa, synthetic construct
QVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSS

SEQ ID NO: 26 - protein, 112 aa, synthetic construct
DIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIK

SEQ ID NO: 27 - protein, 117 aa, synthetic construct
QVQLQQSGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSS

SEQ ID NO: 28 - protein, 112 aa, synthetic construct
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVK

SEQ ID NO: 29 - protein, 260 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 30 - protein, 246 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVS

SEQ ID NO: 31 - DNA, 738 bp, synthetic construct
  source 1..738: mol_type = other DNA
gacatcgtcctgacccagtccccagccagcctggcggtgtccctgggtcaacgagctaccatttcctgcagagcctctaagtccgtatccacttccggctatagttacctgcattggtaccaacagaaaccagggcagccacccaagctgctcatctatctggcctcaaacctggagtccggcgtgcccgccagattctccggaagtgggagcggcaccgactttacattgaacatccacccagtcgaagaggaagatgctgccacttattactgtcagcactccagggagctgccatttacatttggctctggcacaaaactggagatcaaaggcggaggcggaagtggaggcggaggatctggcggcggaggctctcaggttcagctggtcgagtccgggggaggtctggtacagccgggtggcagcctcaagctgtcatgtgccgcatctggttttgactttagcaggtattggatgagctgggtccgccaggccccgggcaagggacttgagtggattggagaaataaaccctacttccagcacaatcaacttcacaccttctttgaaagataaggtttttatctctcgggataacgctaaaaacacactgtacttgcagatgtctaaggtgcgaagcgaagacaccgccctctattattgcgcccggggtaattactaccgctatggggatgcgatggactattgggggcaaggtacctccgtcacggtctca

SEQ ID NO: 32 - protein, 249 aa, synthetic construct
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSGGGGSGGGGSGGGGSGGGGSDIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIK

SEQ ID NO: 33 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 35 - protein, 45 aa, synthetic construct
TTTPAPRPPTPAPTIASQPLSLRPEACRPAAGGAVHTRGLDFACD

SEQ ID NO: 36 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 37 - protein, 41 aa, synthetic construct
CTIEVMYPPPYLDNEKSNGTIIHVKGKHLCPSPLFPGPSKP

SEQ ID NO: 38 - DNA, 123 bp, synthetic construct
  source 1..123: mol_type = other DNA
tgtaccattgaagttatgtatcctcctccttacctagacaatgagaagagcaatggaaccattatccatgtgaaagggaaacacctttgtccaagtcccctatttcccggaccttctaagccc

SEQ ID NO: 39 - protein, 55 aa, synthetic construct
FVPVFLPAKPTTTPAPRPPTPAPTIASQPLSLRPEACRPAAGGAVHTRGLDFACD

SEQ ID NO: 40 - DNA, 165 bp, synthetic construct
  source 1..165: mol_type = other DNA
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 41 - protein, 39 aa, synthetic construct
IEVMYPPPYLDNEKSNGTIIHVKGKHLCPSPLFPGPSKP

SEQ ID NO: 42 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 43 - protein, 10 aa, synthetic construct
GGGSSGGGSG

SEQ ID NO: 44 - DNA, 30 bp, synthetic construct
  source 1..30: mol_type = other DNA
ggtggcggttcttccggcggtggctctggt

SEQ ID NO: 45 - protein, 229 aa, synthetic construct
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - DNA, 687 bp, synthetic construct
  source 1..687: mol_type = other DNA
gagtccaaatatggtcccccatgcccatcatgcccagcacctgagttcgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttccagagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 47 - protein, 20 aa, synthetic construct
AEPKSPDKTHTCPPCPKDPK

SEQ ID NO: 48 - DNA, 60 bp, synthetic construct
  source 1..60: mol_type = other DNA
gccgagcccaagtcccctgataaaactcacacctgcccaccctgtcctaaggacccgaag

SEQ ID NO: 49 - protein, 27 aa, synthetic construct
FWVLVVVGGVLACYSLLVTVAFIIFWV

SEQ ID NO: 50 - DNA, 81 bp, synthetic construct
  source 1..81: mol_type = other DNA
ttctgggtgctggtcgttgtgggcggcgtgctggcctgctacagcctgctggtgacagtggccttcatcatcttttgggtg

SEQ ID NO: 51 - protein, 25 aa, synthetic construct
IPWLGHLLVGLSGAFGFIILVYLLI

SEQ ID NO: 52 - DNA, 75 bp, synthetic construct
  source 1..75: mol_type = other DNA
attccgtggctcggccacctcctcgtgggcctcagcggggcttttggcttcatcatcttagtgtacttgctgatc

SEQ ID NO: 53 - protein, 21 aa, synthetic construct
IYIWAPLAGTCGVLLLSLVIT

SEQ ID NO: 54 - DNA, 63 bp, synthetic construct
  source 1..63: mol_type = other DNA
atctacatctgggcgcccttggccgggacttgtggggtccttctcctgtcactggttatcacc

SEQ ID NO: 55 - protein, 28 aa, synthetic construct
IYIWAPLAGTCGVLLLSLVITLYCNHRN

SEQ ID NO: 56 - DNA, 84 bp, synthetic construct
  source 1..84: mol_type = other DNA
atctacatctgggcgcccttggccgggacttgtggggtccttctcctgtcactggttatcaccctttactgcaaccacaggaac

SEQ ID NO: 57 - skipped

SEQ ID NO: 58 - protein, 43 aa, synthetic construct
RVKFSRSADAPAYQQGQNQLYNELNLGRREEYDVLHMQALPPR

SEQ ID NO: 59 - DNA, 129 bp, synthetic construct
  source 1..129: mol_type = other DNA
agagtgaagttcagcaggagcgcagacgcccccgcgtaccagcagggccagaaccagctctataacgagctcaatctaggacgaagagaggagtacgatgttttgcacatgcaggccctgccccctcgc

SEQ ID NO: 60 - protein, 41 aa, synthetic construct
RSKRSRLLHSDYMNMTPRRPGPTRKHYQPYAPPRDFAAYRS

SEQ ID NO: 61 - DNA, 123 bp, synthetic construct
  source 1..123: mol_type = other DNA
aggagcaagcggagcagactgctgcacagcgactacatgaacatgaccccccggaggcctggccccacccggaagcactaccagccctacgcccctcccagggatttcgccgcctaccggagc

SEQ ID NO: 62 - protein, 94 aa, synthetic construct
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 63 - DNA, 282 bp, synthetic construct
  source 1..282: mol_type = other DNA
aactgcaggaacaccgggccatggctgaagaaggtcctgaagtgtaacaccccagacccctcgaagttcttttcccagctgagctcagagcatggaggcgacgtccagaagtggctctcttcgcccttcccctcatcgtccttcagccctggcggcctggcacctgagatctcgccactagaagtgctggagagggacaaggtgacgcagctgctccccctgaacactgatgcctacttgtctctccaagaactccagggtcaggacccaactcacttggtg

SEQ ID NO: 64 - protein, 4 aa, synthetic construct
YLSL

SEQ ID NO: 65 - protein, 200 aa, synthetic construct
RSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 603 bp, synthetic construct
  source 1..603: mol_type = other DNA
aggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 67 - protein, 153 aa, synthetic construct
RSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 462 bp, synthetic construct
  source 1..462: mol_type = other DNA
aggagcaagcggagcagactgctgcacagcgactacatgaacatgaccccccggaggcctggccccacccggaagcactaccagccctacgcccctcccagggatttcgccgcctaccggagcagagtgaagttcagcaggagcgcagacgcccccgcgtacaagcagggccagaaccagctctataacgagctcaatctaggacgaagagaggagtacgatgttttggacaagcgtagaggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgctag

SEQ ID NO: 69 - protein, 157 aa, synthetic construct
KRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - DNA, 474 bp, synthetic construct
  source 1..474: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactgggcggtggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 71 - protein, 23 aa, synthetic construct
VIGFRILLLKVAGFNLLMTLRLW

SEQ ID NO: 72 - DNA, 69 bp, synthetic construct
  source 1..69: mol_type = other DNA
gtgattgggttccgaatcctcctcctgaaagtggccgggtttaatctgctcatgacgctgcggctgtgg

SEQ ID NO: 73 - protein, 23 aa, synthetic construct
TILYEILLGKATLYAVLVSALVL

SEQ ID NO: 74 - DNA, 69 bp, synthetic construct
  source 1..69: mol_type = other DNA
accatcctctatgagatcttgctagggaaggccaccttgtatgccgtgctggtcagtgccctcgtgctg

SEQ ID NO: 75 - skipped

SEQ ID NO: 76 - protein, 10 aa, synthetic construct
MAMVKRKDSR

SEQ ID NO: 77 - DNA, 30 bp, synthetic construct
  source 1..30: mol_type = other DNA
atggccatggtcaagagaaaggattccaga

SEQ ID NO: 78 - protein, 118 aa, synthetic construct
EVQLQQFGAELVKPGASVKISCKASGYSFSDFNIEWLKQSHGKSLEWIGDINPNYDSTNYNQKFKGRATLTVDKSSSTAYMEVRSLTSEDTAVYYCARLGYGYAMDYWGQGTSVTVSS

SEQ ID NO: 79 - protein, 116 aa, synthetic construct
NIVMTQSPQSMSMSVGERVTLSCKASENVGAYVSWFQQKPDQSPKLLILAASNRYTGVPARFIGSGSATDFTLTISSVQAEDLADYHCGQSYTYPYTFGGGTKLEIKRADAAPTVS

SEQ ID NO: 80 - protein, 448 aa, synthetic construct
EVQLQQFGAELVKPGASVKISCKASGYSFSDFNIEWLKQSHGKSLEWIGDINPNYDSTNYNQKFKGRATLTVDKSSSTAYMEVRSLTSEDTAVYYCARLGYGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 213 aa, synthetic construct
IVMTQSPQSMSMSVGERVTLSCKASENVGAYVSWFQQKPDQSPKLLILAASNRYTGVPARFIGSGSATDFTLTISSVQAEDLADYHCGQSYTYPYTFGGGTKLEIKRADAAPTVSIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 8 aa, synthetic construct
GYSFSDFN

SEQ ID NO: 83 - protein, 8 aa, synthetic construct
INPNYDST

SEQ ID NO: 84 - protein, 11 aa, synthetic construct
ARLGYGYAMDY

SEQ ID NO: 85 - protein, 6 aa, synthetic construct
ENVGAY

SEQ ID NO: 86 - skipped

SEQ ID NO: 87 - protein, 9 aa, synthetic construct
GQSYTYPYT

SEQ ID NO: 88 - protein, 11 aa, synthetic construct
  VAR_SEQ 5
  VAR_SEQ 7..8
SIVLXYXXIVL

SEQ ID NO: 89 - protein, 6 aa, synthetic construct
NLYAAV

SEQ ID NO: 90 - protein, 6 aa, synthetic construct
VTYAEV

SEQ ID NO: 91 - protein, 6 aa, synthetic construct
VTYAQL

SEQ ID NO: 92 - protein, 6 aa, synthetic construct
SIYATL

SEQ ID NO: 93 - protein, 35 aa, synthetic construct
NLYAAVKHTQPEDGVEMDTRSPHDEDPQAVTYAEV

SEQ ID NO: 94 - protein, 36 aa, synthetic construct
VTYAQLHSLTLRREATEPPPSQEGPSPAVPSIYATL

SEQ ID NO: 95 - protein, 87 aa, synthetic construct
NLYAAVKHTQPEDGVEMDTRSPHDEDPQAVTYAEVKHSRPRREMASPPSPLSGEFLDTKDRQAEEDRQMDTEAAASEAPQDVTYAQL

SEQ ID NO: 96 - protein, 88 aa, synthetic construct
VTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 97 - protein, 117 aa, synthetic construct
NLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATL

SEQ ID NO: 98 - protein, 167 aa, synthetic construct
RHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 99 - protein, 20 aa, synthetic construct
YGSQSSKPYLLTHPSDPLEL

SEQ ID NO: 100 - protein, 650 aa, synthetic construct
MTPILTVLICLGLSLGPRTHVQAGHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 101 - DNA, 3229 bp, synthetic construct
  source 1..3229: mol_type = other DNA
aaatgagttttaaaaaggcttgtccaggaagcacatatgggagctggtcactctgcattttgggccctcctggaggtgtttagaccttccgagagagaaactgagacacatgagagggaagaaatgactcagtggtgagaccctgtggagtcccacccacaaccagcacactgtgacccactgcacaaacctctagcccacagctcacttcctcctttaagaagagaagagaaaagaggagaggagaggaggaacagaaaagaaaagaaaagaaaaagtgggaaacaaataatctaagaatgaggagaaagcaagaagagtgacccccttgtgggcactccattggttttatggcgcctctactttctggagtttgtgtaaaacaaaaatattatggtctttgtgcacatttacatcaagctcagcctgggcggcacagccagatgcgagatgcgtctctgctgatctgagtctgcctgcagcatggacctgggtcttccctgaagcatctccagggctggagggacgactgccatgcaccgagggctcatccatccacagagcagggcagtgggaggagacgccatgacccccatcctcacggtcctgatctgtctcgggctgagtctgggcccccggacccacgtgcaggcagggcacctccccaagcccaccctctgggctgaaccaggctctgtgatcacccaggggagtcctgtgaccctcaggtgtcaggggggccaggagacccaggagtaccgtctatatagagaaaagaaaacagcaccctggattacacggatcccacaggagcttgtgaagaagggccagttccccatcccatccatcacctgggaacacacagggcggtatcgctgttactatggtagcgacactgcaggccgctcagagagcagtgaccccctggagctggtggtgacaggagcctacatcaaacccaccctctcagcccagcccagccccgtggtgaactcaggagggaatgtaaccctccagtgtgactcacaggtggcatttgatggcttcattctgtgtaaggaaggagaagatgaacacccacaatgcctgaactcccagccccatgcccgtgggtcgtcccgcgccatcttctccgtgggccccgtgagcccgagtcgcaggtggtggtacaggtgctatgcttatgactcgaactctccctatgagtggtctctacccagtgatctcctggagctcctggtcctaggtgtttctaagaagccatcactctcagtgcagccaggtcctatcgtggcccctgaggagaccctgactctgcagtgtggctctgatgctggctacaacagatttgttctgtataaggacggggaacgtgacttccttcagctcgctggcgcacagccccaggctgggctctcccaggccaacttcaccctgggccctgtgagccgctcctacgggggccagtacagatgctacggtgcacacaacctctcctccgagtggtcggcccccagcgaccccctggacatcctgatcgcaggacagttctatgacagagtctccctctcggtgcagccgggccccacggtggcctcaggagagaacgtgaccctgctgtgtcagtcacagggatggatgcaaactttccttctgaccaaggagggggcagctgatgacccatggcgtctaagatcaacgtaccaatctcaaaaataccaggctgaattccccatgggtcctgtgacctcagcccatgcggggacctacaggtgctacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactagcccagggggggacgcagaccccacactccatggagtctggaatgcatgggagctgcccccccagtggacaccattggaccccacccagcctggatctaccccaggagactctgggaacttttaggggtcactcaattctgcagtataaataactaatgtctctacaattttgaaataaagcaacagacttctcaataatcaatgaagtagctgagaaaactaagtcagaaagtgcattaaactgaatcacaatgtaaatattacacatcaagcgatgaaactggaaaactacaagccacgaatgaatgaattaggaaagaaaaaaagtaggaaatgaatgatcttggctttcctataagaaatttagggcagggcacggtggctcacgcctgtaattccagcactttgggaggccgaggcgggcagatcacgagttcaggagatcgagaccatcttggccaacatggtgaaaccctgtctctcctaaaaatacaaaaattagctggatgtggtggcagtgcctgtaatcccagctatttgggaggctgaggcaggagaatcgcttgaaccagggagtcagaggtttcagtgagccaagatcgcaccactgctctccagcctggcgacagagggagactccatctcaaattaaaaaaaaaaaaaaaaaagaaagaaaaaaaaaaaaaaaaaa

SEQ ID NO: 102 - protein, 23 aa, synthetic construct
VVIGILVAVILLLLLLLLLFLIL

SEQ ID NO: 103 - protein, 63 aa, synthetic construct
YGSQSSKPYLLTHPSDPLELVVSGPSGGPSSPTTGPTSTSGPEDQPLTPTGSDPQSGLGRHLG

SEQ ID NO: 104 - protein, 43 aa, synthetic construct
VVSGPSGGPSSPTTGPTSTSGPEDQPLTPTGSDPQSGLGRHLG

SEQ ID NO: 105 - protein, 86 aa, synthetic construct
YGSQSSKPYLLTHPSDPLELVVSGPSGGPSSPTTGPTSTSGPEDQPLTPTGSDPQSGLGRHLGVVIGILVAVILLLLLLLLLFLIL

SEQ ID NO: 106 - protein, 190 aa, synthetic construct
VVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 107 - protein, 233 aa, synthetic construct
VVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 108 - protein, 253 aa, synthetic construct
YGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 109 - DNA, 762 bp, synthetic construct
  source 1..762: mol_type = other DNA
tacggctcacagagctccaaaccctacctgctgactcaccccagtgaccccctggagctcgtggtctcaggaccgtctgggggccccagctccccgacaacaggccccacctccacatctggccctgaggaccagcccctcacccccaccgggtcggatccccagagtggtctgggaaggcacctgggggttgtgatcggcatcttggtggccgtcatcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgatgcccaggaagaaaacctctatgctgccgtgaagcacacacagcctgaggatggggtggagatggacactcggagcccacacgatgaagacccccaggcagtgacgtatgccgaggtgaaacactccagacctaggagagaaatggcctctcctccttccccactgtctggggaattcctggacacaaaggacagacaggcggaagaggacaggcagatggacactgaggctgctgcatctgaagccccccaggatgtgacctacgcccagctgcacagcttgaccctcagacgggaggcaactgagcctcctccatcccaggaagggccctctccagctgtgcccagcatctacgccactctggccatccactag

SEQ ID NO: 110 - protein, 650 aa, synthetic construct; identical to SEQ ID NO: 100

SEQ ID NO: 111 - skipped

SEQ ID NO: 112 - protein, 233 aa, synthetic construct; identical to SEQ ID NO: 107

SEQ ID NO: 113 - protein, 167 aa, synthetic construct; identical to SEQ ID NO: 98

SEQ ID NO: 114 - protein, 58 aa, synthetic construct
VTYAEVKHSRPRREMASPPSPLSGEFLDTKDRQAEEDRQMDTEAAASEAPQDVTYAQL

SEQ ID NO: 115 - protein, 64 aa, synthetic construct
YGSQSSKPYLLTHPSDPLELVVSGPSGGPSSPTTGPTSTSGPEDQPLTPTGSDPQSGLGRHLGV

SEQ ID NO: 116 - protein, 44 aa, synthetic construct
VVSGPSGGPSSPTTGPTSTSGPEDQPLTPTGSDPQSGLGRHLGV

SEQ ID NO: 117 - protein, 67 aa, synthetic construct
AGSGGSGGSGGSPVPSTPPTPSPSTPPTPSPSGGSGNSSGSGGSPVPSTPPTPSPSTPPTPSPSASV

SEQ ID NO: 118 - protein, 75 aa, synthetic construct
AGSGGSGGSGGSPVPSTPPTNSSSTPPTPSPSPVPSTPPTNSSSTPPTPSPSPVPSTPPTNSSSTPPTPSPSASV

SEQ ID NO: 119 - protein, 85 aa, synthetic construct
VVSGPSGGPSSPTTGPTSTSGPEDQPLTPTGSDPQSGLGRHVVSGPSGGPSSPTTGPTSTSGPEDQPLTPTGSDPQSGLGRHLGV

SEQ ID NO: 120 - protein, 32 aa, synthetic construct
TTGPTSTSGPEDQPLTPTGSDPQSGLGRHLGV

SEQ ID NO: 121 - protein, 86 aa, synthetic construct; identical to SEQ ID NO: 105

SEQ ID NO: 122 - protein, 190 aa, synthetic construct; identical to SEQ ID NO: 106

SEQ ID NO: 123 - protein, 18 aa, synthetic construct
EGRGSLLTCGDVEENPGP

SEQ ID NO: 124 - protein, 19 aa, synthetic construct
ATNFSLLKQAGDVEENPGP

SEQ ID NO: 125 - protein, 20 aa, synthetic construct
QCTNYALLKLAGDVESNPGP

SEQ ID NO: 126 - protein, 22 aa, synthetic construct
VKQTLNFDLLKLAGDVESNPGP

SEQ ID NO: 127 - protein, 21 aa, synthetic construct
GSGEGRGSLLTCGDVEENPGP

SEQ ID NO: 128 - DNA, 63 bp, synthetic construct
  source 1..63: mol_type = other DNA
ggatccggagagggcagaggcagcctgctgacatgtggcgacgtggaagagaaccctggcccc

SEQ ID NO: 129 - DNA, 255 bp, synthetic construct
  source 1..255: mol_type = other DNA
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaagga

SEQ ID NO: 130 - DNA, 223 bp, synthetic construct
  source 1..223: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttccc

SEQ ID NO: 131 - DNA, 354 bp, synthetic construct
  source 1..354: mol_type = other DNA
gaggtccagctgcaacagtttggagctgagttggtgaagcctggggcttcagtgaagatatcctgcaaggcttctggctactcattcagtgacttcaacatagaatggctgaagcagagccatggaaagagccttgagtggattggagatattaatcctaactatgatagtactaactacaaccagaagttcaagggaagggccacattgactgtagacaagtcctccagcacagcctacatggaggtccgcagcctgacatctgaggacactgcagtctattactgtgcaagattggggtacggctatgctatggactactggggtcaaggaacctcagtcaccgtctcctca

SEQ ID NO: 132 - DNA, 348 bp, synthetic construct
  source 1..348: mol_type = other DNA
aacattgtgatgacccaatctccccaatccatgtccatgtcagtaggagagagggtcaccttgagctgcaaggccagtgagaatgtgggtgcttatgtatcctggtttcaacagaaaccagaccagtctcctaaattgctgatattagcggcatccaaccggtacactggggtccccgctcgtttcataggcagtggatctgcaacagacttcactctgaccatcagcagtgtgcaggctgaagaccttgcagattatcactgtggacagagttatacctatccgtacacgttcggaggggggaccaaactggaaataaaacgggctgatgctgcaccaactgtatcc

SEQ ID NO: 133 - protein, 673 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 573 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 745 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 645 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 558 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDPFFFCCFIAVAMGIRFIIMVTWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 480 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 568 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 668 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSKISGGGGSGGGGSGGGGSGGGGSGGGGSSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - DNA, 2560 bp, synthetic construct
  source 1..2560: mol_type = other DNA
taatacgactcactatagggaaagctcgagtatgttgctgctggttacatctctgctgctgtgcgagctgccccatcctgcctttctgctgattcctcaggtgcagctggtggaatctggcggaggacttgttcagcctggcggctctctgaagctgtcttgtgccgccagcggcttcgacttcagcagatactggatgagctgggtccgacaggcccctggcaaaggactggaatggatcggcgagatcaaccccacctccagcaccatcaacttcacccctagcctgaaggacaaggtgttcatcagccgggacaacgccaagaacaccctgtacctgcagatgagcaaagtgcggagcgaggacacagccctgtactactgtgccagaggcaactactacagatacggcgacgccatggactactggggccagggaacaagcgtgaccgtgtctaagatttctggcggcggaggaagcggaggcggaggatccggcggtggtggtagtggtggtggcggttcaggtggcggtggaagctctgatatcgtgctgacacagagccccgcttctctggccgtttctctgggacagagagccaccatcagctgcagagccagcaagagcgtgtccacaagcggctacagctacctgcactggtatcagcaaaagcccggccagcctcctaagctgctgatctacctggccagcaacctggaaagcggagtgcctgccagattttctggcagcggctctggcaccgacttcaccctgaatatccatcctgtggaagaagaggacgccgccacttactactgccagcacagcagagaactgcccttcacattcggcagcggcaccaagctggaaatcaaagagtctaagtacggccctccttgtcctccatgtcctgctccagagtttgaaggcggcccttccgtgttcctgtttcctccaaagcctaaggacaccctgatgatcagcaggacccctgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgcggagcaagcggagcagaggcggccacagcgactacatgaacatgacccccagacggcctggccccacccggaagcactaccagccctacgccccacccagggactttgccgcctaccggtccggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgacagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcctcgacgaattcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 142 - protein, 546 aa, synthetic construct
MALPVTALLLPLALLLHAARPSDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKKISGGGGSGGGGSGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 143 - DNA, 1657 bp, synthetic construct
  source 1..1657: mol_type = other DNA
gcgatcgcatggccttaccagtgaccgccttgctcctgccgctggccttgctgctccacgccgccaggccgagcgacatcgtgctgacccagagccccgccagcctggccgtgagcctgggccagagagccaccatcagctgcagagccagcaagagcgtgagcaccagcggctacagctacctgcactggtaccagcagaagcccggccagccccccaagctgctgatctacctggccagcaacctggagagcggcgtgcccgccagattcagcggcagcggcagcggcaccgacttcaccctgaacatccaccccgtggaggaggaggacgccgccacctactactgccagcacagcagagagctgcccttcaccttcggcagcggcaccaagctggagatcaagaagatcagcggcggcggcggcagcggcggcggcggcagcggcggcggcggcagcggcggcggcggcagcggcggcggcggcagccaggtgcagctggtggagagcggcggcggcctggtgcagcccggcggcagcctgaagctgagctgcgccgccagcggcttcgacttcagcagatactggatgagctgggtgagacaggcccccggcaagggcctggagtggatcggcgagatcaaccccaccagcagcaccatcaacttcacccccagcctgaaggacaaggtgttcatcagcagagacaacgccaagaacaccctgtacctgcagatgagcaaggtgagaagcgaggacaccgccctgtactactgcgccagaggcaactactacagatacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 144 - protein, 490 aa, synthetic construct
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 145 - DNA, 1489 bp, synthetic construct
  source 1..1489: mol_type = other DNA
gcgatcgcatggccttaccagtgaccgccttgctcctgccgctggccttgctgctccacgccgccaggccggacatcgtgctgacccagagccccgccagcctggccgtgagcctgggccagagggccaccatcagctgcagggccagcaagagcgtgagcaccagcggctacagctacctgcactggtaccagcagaagcccggccagccccccaagctgctgatctacctggccagcaacctggagagcggcgtgcccgccaggttcagcggcagcggcagcggcaccgacttcaccctgaacatccaccccgtggaggaggaggacgccgccacctactactgccagcacagcagggagctgcccttcaccttcggcagcggcaccaagctggagatcaagggaggggggggatccgggggaggaggctccggcggaggcggaagccaggtgcagctggtggagagcggcggcggcctggtgcagcccggcggcagcctgaagctgagctgcgccgccagcggcttcgacttcagcaggtactggatgagctgggtgaggcaggcccccggcaagggcctggagtggatcggcgagatcaaccccaccagcagcaccatcaacttcacccccagcctgaaggacaaggtgttcatcagcagggacaacgccaagaacaccctgtacctgcagatgagcaaggtgaggagcgaggacaccgccctgtactactgcgccaggggcaactactacaggtacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 146 - protein, 491 aa, synthetic construct
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 147 - DNA, 1492 bp, synthetic construct
  source 1..1492: mol_type = other DNA
gcgatcgcatggccttaccagtgaccgccttgctcctgccgctggccttgctgctccacgccgccaggccggacatcgtgctgacccagagccccgccagcctggccgtgagcctgggccagagggccaccatcagctgcagggccagcaagagcgtgagcaccagcggctacagctacctgcactggtaccagcagaagcccggccagccccccaagctgctgatctacctggccagcaacctggagagcggcgtgcccgccaggttcagcggcagcggcagcggcaccgacttcaccctgaacatccaccccgtggaggaggaggacgccgccacctactactgccagcacagcagggagctgcccttcaccttcggcagcggcaccaagctggagatcaagggaggggggggatccgggggaggaggctccggcggaggcggaagccaggtgcagctggtggagagcggcggcggcctggtgcagcccggcggcagcctgaagctgagctgcgccgccagcggcttcgacttcagcaggtactggatgagctgggtgaggcaggcccccggcaagggcctggagtggatcggcgagatcaaccccaccagcagcaccatcaacttcacccccagcctgaaggacaaggtgttcatcagcagggacaacgccaagaacaccctgtacctgcagatgagcaaggtgaggagcgaggacaccgccctgtactactgcgccaggggcaactactacaggtacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagtttaaac

SEQ ID NO: 148 - protein, 765 aa, synthetic construct
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 149 - DNA, 2314 bp, synthetic construct
  source 1..2314: mol_type = other DNA
gcgatcgcatggccttaccagtgaccgccttgctcctgccgctggccttgctgctccacgccgccaggccggacatcgtgctgacccagagccccgccagcctggccgtgagcctgggccagagggccaccatcagctgcagggccagcaagagcgtgagcaccagcggctacagctacctgcactggtaccagcagaagcccggccagccccccaagctgctgatctacctggccagcaacctggagagcggcgtgcccgccaggttcagcggcagcggcagcggcaccgacttcaccctgaacatccaccccgtggaggaggaggacgccgccacctactactgccagcacagcagggagctgcccttcaccttcggcagcggcaccaagctggagatcaagggaggggggggatccgggggaggaggctccggcggaggcggaagccaggtgcagctggtggagagcggcggcggcctggtgcagcccggcggcagcctgaagctgagctgcgccgccagcggcttcgacttcagcaggtactggatgagctgggtgaggcaggcccccggcaagggcctggagtggatcggcgagatcaaccccaccagcagcaccatcaacttcacccccagcctgaaggacaaggtgttcatcagcagggacaacgccaagaacaccctgtacctgcagatgagcaaggtgaggagcgaggacaccgccctgtactactgcgccaggggcaactactacaggtacggcgacgccatggactactggggccagggcaccagcgtgaccgtgagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagcggcgaaggccgcggcagcctgctgacctgcggcgatgtggaagaaaacccgggccccatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataagtttaaac

SEQ ID NO: 150 - DNA, 2757 bp, synthetic construct
  source 1..2757: mol_type = other DNA
atggccttaccagtgaccgccttgctcctgccgctggccttgctgctccacgccgccaggccggacatcgtcctgacccagtccccagccagcctggcggtgtccctgggtcaacgagctaccatttcctgcagagcctctaagtccgtatccacttccggctatagttacctgcattggtaccaacagaaaccagggcagccacccaagctgctcatctatctggcctcaaacctggagtccggcgtgcccgccagattctccggaagtgggagcggcaccgactttacattgaacatccacccagtcgaagaggaagatgctgccacttattactgtcagcactccagggagctgccatttacatttggctctggcacaaaactggagatcaaaggcggaggcggaagtggaggcggaggatctggcggcggaggctctcaggttcagctggtcgagtccgggggaggtctggtacagccgggtggcagcctcaagctgtcatgtgccgcatctggttttgactttagcaggtattggatgagctgggtccgccaggccccgggcaagggacttgagtggattggagaaataaaccctacttccagcacaatcaacttcacaccttctttgaaagataaggtttttatctctcgggataacgctaaaaacacactgtacttgcagatgtctaaggtgcgaagcgaagacaccgccctctattattgcgcccggggtaattactaccgctatggggatgcgatggactattgggggcaaggtacctccgtcacggtctcaacgcgtgagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcgggcgcgccagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagaggatctggagcaacaaacttctcactactcaaacaagcaggtgacgtggaggagaatcccgggcctgggatccacatggccacttacgaggttctctgtgaggtggcgcggaaactgggcacggatgacagggaagtggtattgttcctcctaaacgtgttcatacctcaacccacactggcccaattaattggagctcttagagctttaaaggaggagggcaggttaacgtttcccctgttagcggaatgtctgtttcgtgcaggtcgcagagacctcttgcgcgacctgcttcacttagacccgcgttttttagagcgccacctagcgggcacaatgagttatttcagcccttatcagctcactgttctccacgtagacggggagctgtgtgcgagggatattaggtctttgatatttttaagcaaggacactatagggtctcgcagcacaccacagacattcttacactgggtgtactgtatggaaaacttagacctactgggtcccactgacgtggatgccctaatgtcaatgcttagatctttgtcaagagtagacctacagcgccaagtgcaaaccctaatgggccttcacctttccggcccatcacactcccaacactatcgccatacaccactcgagaccgatttctacgactacaaggacgacgatgacaagactagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 151 - protein, 918 aa, synthetic construct
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTREQKLISEEDLAKPTTTPAPRPPTPAPTIASQPLSLRPEACRPAAGGAVHTRGLDFACDIYIWAPLAGTCGVLLLSLVITLYCGRARVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSRGSGATNFSLLKQAGDVEENPGPGIHMATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQTLMGLHLSGPSHSQHYRHTPLETDFYDYKDDDDKTSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 152 - DNA, 2874 bp, synthetic construct
  source 1..2874: mol_type = other DNA
atggccttaccagtgaccgccttgctcctgccgctggccttgctgctccacgccgccaggccggacatcgtcctgacccagtccccagccagcctggcggtgtccctgggtcaacgagctaccatttcctgcagagcctctaagtccgtatccacttccggctatagttacctgcattggtaccaacagaaaccagggcagccacccaagctgctcatctatctggcctcaaacctggagtccggcgtgcccgccagattctccggaagtgggagcggcaccgactttacattgaacatccacccagtcgaagaggaagatgctgccacttattactgtcagcactccagggagctgccatttacatttggctctggcacaaaactggagatcaaaggcggaggcggaagtggaggcggaggatctggcggcggaggctctcaggttcagctggtcgagtccgggggaggtctggtacagccgggtggcagcctcaagctgtcatgtgccgcatctggttttgactttagcaggtattggatgagctgggtccgccaggccccgggcaagggacttgagtggattggagaaataaaccctacttccagcacaatcaacttcacaccttctttgaaagataaggtttttatctctcgggataacgctaaaaacacactgtacttgcagatgtctaaggtgcgaagcgaagacaccgccctctattattgcgcccggggtaattactaccgctatggggatgcgatggactattgggggcaaggtacctccgtcacggtctcaacgcgtgagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagaggatctggagcaacaaacttctcactactcaaacaagcaggtgacgtggaggagaatcccgggcctgggatccacatggccacttacgaggttctctgtgaggtggcgcggaaactgggcacggatgacagggaagtggtattgttcctcctaaacgtgttcatacctcaacccacactggcccaattaattggagctcttagagctttaaaggaggagggcaggttaacgtttcccctgttagcggaatgtctgtttcgtgcaggtcgcagagacctcttgcgcgacctgcttcacttagacccgcgttttttagagcgccacctagcgggcacaatgagttatttcagcccttatcagctcactgttctccacgtagacggggagctgtgtgcgagggatattaggtctttgatatttttaagcaaggacactatagggtctcgcagcacaccacagacattcttacactgggtgtactgtatggaaaacttagacctactgggtcccactgacgtggatgccctaatgtcaatgcttagatctttgtcaagagtagacctacagcgccaagtgcaaaccctaatgggccttcacctttccggcccatcacactcccaacactatcgccatacaccactcgagaccgatttctacgactacaaggacgacgatgacaagactagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 153 - protein, 957 aa, synthetic construct
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTREQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSRGSGATNFSLLKQAGDVEENPGPGIHMATYEVLCEVARKLGTDDREVVLFLLNVFIPQPTLAQLIGALRALKEEGRLTFPLLAECLFRAGRRDLLRDLLHLDPRFLERHLAGTMSYFSPYQLTVLHVDGELCARDIRSLIFLSKDTIGSRSTPQTFLHWVYCMENLDLLGPTDVDALMSMLRSLSRVDLQRQVQTLMGLHLSGPSHSQHYRHTPLETDFYDYKDDDDKTSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 154 - DNA, 2187 bp, synthetic construct
  source 1..2187: mol_type = other DNA
atggccttaccagtgaccgccttgctcctgccgctggccttgctgctccacgccgccaggccggacatcgtcctgacccagtccccagccagcctggcggtgtccctgggtcaacgagctaccatttcctgcagagcctctaagtccgtatccacttccggctatagttacctgcattggtaccaacagaaaccagggcagccacccaagctgctcatctatctggcctcaaacctggagtccggcgtgcccgccagattctccggaagtgggagcggcaccgactttacattgaacatccacccagtcgaagaggaagatgctgccacttattactgtcagcactccagggagctgccatttacatttggctctggcacaaaactggagatcaaaggcggaggcggaagtggaggcggaggatctggcggcggaggctctcaggttcagctggtcgagtccgggggaggtctggtacagccgggtggcagcctcaagctgtcatgtgccgcatctggttttgactttagcaggtattggatgagctgggtccgccaggccccgggcaagggacttgagtggattggagaaataaaccctacttccagcacaatcaacttcacaccttctttgaaagataaggtttttatctctcgggataacgctaaaaacacactgtacttgcagatgtctaaggtgcgaagcgaagacaccgccctctattattgcgcccggggtaattactaccgctatggggatgcgatggactattgggggcaaggtacctccgtcacggtctcaacgcgtgagcagaaactgatctcggaagaggatctggcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctgaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagtggctccggcgagggcagaggaagtctactaacctgcggagatgtggaagaaaatcctggcccacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 155 - protein, 728 aa, synthetic construct
MALPVTALLLPLALLLHAARPDIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKGGGGSGGGGSGGGGSQVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSTREQKLISEEDLAKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSGSGEGRGSLLTCGDVEENPGPHMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 156 - protein, 298 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSITCPPPMSVEHADIWVKSYSLYSRERYICNSGFKRKAGTSSLTECVLNKATNVAHWTTPSLKCIR

SEQ ID NO: 157 - protein, 224 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 158 - protein, 448 aa, synthetic construct
QVQLVESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 220 aa, synthetic construct
QVQLQQLGAELARPGASVKMSCKASGYTFTSYSIQWVKQRPGQGLEWIGYINPSSGYIKYNQHFRDRATLTADRSSSTAYMQLSSLTSEDSAVYYCARGNSGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 160 - protein, 348 aa, synthetic construct
DIVMTQAAPSVPVTPGESLSISCRSSKSLLHSSGITYLYWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 161 - protein, 220 aa, synthetic construct
QVQLQQSGAELARPGASVKMSCKASGYTFTSYTMQWVKQRPGQGLEWIGYINPSSGYIKYNQKFKDKVTLTADKSSTTAYMQLSRLTSEDSAVYYCARRGSYFFDFWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 162 - protein, 348 aa, synthetic construct
DIVITQDELSNPVTSGESVSISCRSSKSLLYKDGKTYLNWFLQRPGQSPQLLIYLMSTRASGVSDRFSGSGSGTDFTLEISRVKAEDVGVYYCQQLVEYPFTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 163 - protein, 224 aa, synthetic construct
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGNYYRYGDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 164 - protein, 347 aa, synthetic construct
EIVLTQSPATLSLSLGERATISCRASKSVSTSGYSYLHWYQQKPGQAPKLLIYLASNRATGVPARFSGSGPGTDFTLTISSLEPEDFATYYCQHSRELPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 165 - protein, 224 aa, synthetic construct
EVQLVESGGGLVQPGGSLRLSCAASGFDFSRYWMSWVRQAPGKGLEWIGEINPTSSTINFADSVKGRFTISRDNAKNSLYLQMNSVRDEDTAVYYCARGSGGGSGDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 166 - protein, 340 aa, synthetic construct
DIVLTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 167 - protein, 987 aa, synthetic construct
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSGGGGSGGGGSGGGGSGGGGSDIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIKGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 168 - protein, 602 aa, synthetic construct
EVQLVQSGAEVKKPGASVKVSCKASGYTFTSYSIQWVRQAPGQRLEWIGYINPSSGYIKYNQHFRGRATLTADRSASTAYMELSSLRSEDTAVYYCARGNSGSFDYWGQGTLVTVSSGGGGSGGGGSGGGGSGGGGSDIVMTQSPLSLPVTPGEPASISCRSSQSLLHSSGITYLYWFLQKPGQSPQLLIYRMSNLASGVPDRFSGSGSGTDFTLKISRVEAEDVGVYYCMQHLEYPFTFGQGTKLEIKGGGGSGGGGSGGGGSGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 169 - protein, 1170 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 170 - protein, 1223 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQGVHGLVEMQTSKQILCRPAGDAEHTVGAQEGELPCPWGVSEAFRDNIRAGPCRVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 171 - protein, 1086 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 172 - protein, 400 aa, Homo sapiens
MATLLLLLGVLVVSPDALGSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRGMLPVAVLVALLAVIVLVALLLLWRRRQKRRTGALVLSRGGKRNGVVDAWAGPAQVPEEGAVTVTVGGSGGDKGSGFPDGEGSSRRPTLTTFFGRRKSRQGSLAMEELKSGSGPSLKGEEEPLVASEDGAVDAPAPDEPEGGDGAAP

SEQ ID NO: 173 - protein, 355 aa, Homo sapiens
MATLLLLLGVLVVSPDALGSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRGMLPVAVLVALLAVIVLVALLLLWRRRQKRRTGALVLSRGGKRNGVVDAWAGPAQVPEEGAVTVTVGGSGGDKGSGFPDGEGSSRRPTLTTFFGRRKSRQGS

SEQ ID NO: 174 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 175 - protein, 310 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPVR

SEQ ID NO: 176 - protein, 237 aa, Homo sapiens
MPLLLLLPLLWADLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 177 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 178 - protein, 952 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 179 - protein, 125 aa, synthetic construct
EVQLVESGGGLVQPGGSLRLSCAASGYSFTGHWMNWVRQAPGKGLEWVGIMIHPSDSETRYNQKFKDIRFTISVDKSKNTLYLQMNSLRAEDTAVYYCARIGIYFYGTTYFDYIWGQGTLVTVSS

SEQ ID NO: 180 - protein, 108 aa, synthetic construct
DIQMTQSPSSLSASVGDRVTITCRASKTISKYLAWXQQKPGKAPKLLIYSGSTLQSGVPSRFSGSGSGTDFTLTISSLQPEDFATYYCQQHNEYPLTFGQGTKVEIKR

SEQ ID NO: 181 - protein, 443 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 182 - protein, 218 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 120 aa, synthetic construct
EVMLVESGGALVKPGGSLKLSCAASGFTFSNNAMSWVRQTPETRLEWVATITGGGSYTYYPDSVKGRFTISRDNARNTLYLQMSSLRSEDTATYYCASPDGNYEGVLAYWGQGTLVTVSA

SEQ ID NO: 184 - protein, 5 aa, synthetic construct
NNAMS

SEQ ID NO: 185 - protein, 17 aa, synthetic construct
TITGGGSYTYYPDSVKG

SEQ ID NO: 186 - protein, 11 aa, synthetic construct
PDGNYEGVLAY

SEQ ID NO: 187 - protein, 118 aa, synthetic construct
QVQLQQSGADLVRPGASVKLSCTASGFNIKDDYVHWVKQRPEQGLEWIGRIDPANGNTRYAPKFRGKATMTADTSSNTAYLQLSSLTSADTAVYYCSPPLWVGGFAYWGQGTLVTVSS

SEQ ID NO: 188 - protein, 118 aa, synthetic construct
EVQLVQSGAEVKKPGASVKVSCKASGFNIKDDYVHWVRQAPGQGLEWIGRIDPANGNTRYAPKFRGRATMTADTSISTAYMELSRLRSDDTAVYYCSPPLWVGGFAYWGQGTLVTVSS

SEQ ID NO: 189 - protein, 118 aa, synthetic construct
EVQLVQSGAEVKKPGASVKVSCKASGFNIKDDYVHWVRQAPGQGLEWIGRIDPAQGNTRYAPKFRGRATMTADTSISTAYMELSRLRSDDTAVYYCSPPLWVGGFAYWGQGTLVTVSS

SEQ ID NO: 190 - protein, 118 aa, synthetic construct
EVQLVQSGAEVKKPGASVKVSCKASGFNIKDDYVHWVRQAPGQGLEWIGRIDPASGNTRYAPKFRGRATMTADTSISTAYMELSRLRSDDTAVYYCSPPLWVGGFAYWGQGTLVTVSS

SEQ ID NO: 191 - protein, 5 aa, synthetic construct
DDYVH

SEQ ID NO: 192 - protein, 17 aa, synthetic construct
RIDPANGNTRYAPKFRG

SEQ ID NO: 193 - protein, 9 aa, synthetic construct
PLWVGGFAY

SEQ ID NO: 194 - protein, 112 aa, synthetic construct
DVVMTQTPLSLPVSLGDQASISCRSSQSLVHSDVNTYLHWYLQRPGQSPKLLIYKVSNRFSGVPDRFSGSGSGTDFTLKISRVESEDLGIYFCSQTTHVPYTFGGGTKLEIK

SEQ ID NO: 195 - protein, 112 aa, synthetic construct
DVVMTQSPLSLPVTLGQPASISCRSSQSLVHSDVNTYLHWYQQRPGQSPRLLIYKVSNRFSGVPDRFSGSGSGTDFTLKISRVEAEDVGVYFCSQTTHVPYTFGGGTKLEIK

SEQ ID NO: 196 - protein, 16 aa, synthetic construct
RSSQSLVHSDVNTYLH

SEQ ID NO: 197 - protein, 7 aa, synthetic construct
KVSNRFS

SEQ ID NO: 198 - protein, 9 aa, synthetic construct
SQTTHVPYT

SEQ ID NO: 199 - protein, 117 aa, synthetic construct
QGQMHQSGAELVKPGASVKLSCKTSGFTFNSGYISWLKQKPRQSLEWIAWIYAGTGISNFNQKFTGKAQLTVDTSSSTAYMQLSSLTSADSAIYFCASGARKTLDFWGQGTSVTVSS

SEQ ID NO: 200 - protein, 5 aa, synthetic construct
SGYIS

SEQ ID NO: 201 - protein, 17 aa, synthetic construct
WIYAGTGISNFNQKFTG

SEQ ID NO: 202 - protein, 8 aa, synthetic construct
GARKTLDF

SEQ ID NO: 203 - protein, 107 aa, synthetic construct
DIQMTQSSSYLSVSVGGRVTITCKASDQINYWLAWYQQKPGNAPRLLISGATSLETGVPSRFSGSGSGKDYTLSITSFQTEDVATYYCQQYWTTPYTFGGGTKVEIK

SEQ ID NO: 204 - protein, 11 aa, synthetic construct
KASDQINYWLA

SEQ ID NO: 205 - protein, 7 aa, synthetic construct
GATSLET

SEQ ID NO: 206 - protein, 9 aa, synthetic construct
QQYWTTPYT

SEQ ID NO: 207 - protein, 192 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 208 - protein, 192 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 209 - protein, 5 aa, synthetic construct
SYDMH

SEQ ID NO: 210 - protein, 17 aa, synthetic construct
WIYPGNGNTKYNQKFNG

SEQ ID NO: 211 - protein, 12 aa, synthetic construct
PTVATRYNWFAY

SEQ ID NO: 212 - protein, 10 aa, synthetic construct
SASSSVNYMH

SEQ ID NO: 213 - protein, 7 aa, synthetic construct
LTSNLAS

SEQ ID NO: 214 - protein, 10 aa, synthetic construct
QQWSSNLPIT

SEQ ID NO: 215 - protein, 5 aa, synthetic construct
TYSVS

SEQ ID NO: 216 - protein, 16 aa, synthetic construct
AIWSGGSTDYNSALKS

SEQ ID NO: 217 - protein, 12 aa, synthetic construct
MNNKFGYWYFDL

SEQ ID NO: 218 - protein, 11 aa, synthetic construct
RASETVSTMLH

SEQ ID NO: 219 - protein, 7 aa, synthetic construct
LASHLES

SEQ ID NO: 220 - protein, 9 aa, synthetic construct
QQTWNDPLT

SEQ ID NO: 221 - protein, 121 aa, synthetic construct
QVQLQQSGAELVKPGSSVKISCKASGYSFPSYDMHWIKQQPGNGLEWIGWIYPGNGNTKYNQKFNGKASLTADKSSSTAYMQLSSLTSEDSAVYFCAKPTVATRYNWFAYWGQGTLVTVSS

SEQ ID NO: 222 - protein, 107 aa, synthetic construct
QIVLTQSPALMSASPGEKVTMTCSASSSVNYMHWYQQKPRSSPKPWIYLTSNLASGVPARFSGSGSGTSYSLTISTMEAEDAATYYCQQWSSNLPITFGSGTKLEIK

SEQ ID NO: 223 - protein, 120 aa, synthetic construct
QVQLKESGPGLVQPSQTLSLTCSVSGFSLTTYSVSWVRQPPGKGLEWIGAIWSGGSTDYNSALKSRLRISRDTSKSQVLLKMNSLQTEDRAMYFCVRMNNKFGYWYFDLWGPGTMVTVSS

SEQ ID NO: 224 - protein, 106 aa, synthetic construct
DTVLTQSPTLAVSPGEKITISCRASETVSTMLHWYQQKPGQQPKLLISLASHLESGVPARFTGSGSGTDFTLTIDPVEADDTATYYCQQTWNDPLTFGSGTKLEIK

SEQ ID NO: 225 - protein, 8 aa, synthetic construct
GYTFTDYW

SEQ ID NO: 226 - protein, 8 aa, synthetic construct
IYPSDSST

SEQ ID NO: 227 - protein, 13 aa, synthetic construct
AREGLAGVFYFDY

SEQ ID NO: 228 - protein, 11 aa, synthetic construct
QSIVHRSGNTY

SEQ ID NO: 229 - skipped

SEQ ID NO: 230 - protein, 9 aa, synthetic construct
FQGSHLPPT

SEQ ID NO: 231 - protein, 120 aa, synthetic construct
QVQLQQPGAELVRPGSSVKLSCKASGYTFTDYWMDWVKQRPGQGLEWIGTIYPSDSSTHYNQEFKGKATMTVDKSSSTAYMHLSSLTSEDSAVYYCAREGLAGVFYFDYWGQGTTLTVSS

SEQ ID NO: 232 - protein, 111 aa, synthetic construct
DVLMTQIPFSLPVSLGDQASISCRSSQSIVHRSGNTYLEWYLQKPGQSPKLLIYKVSNRFSGVPDRFSGSGSGTDFTLKISRVEAEDLGVYYCFQGSHLPPTFGGTTLEIK

SEQ ID NO: 233 - protein, 221 aa, synthetic construct
QVQLQESGPGLVKPSQSLSLTCTVTGFSITSDYAWNWIRQFPGKKLEWMGYINFDGGTTYNPSLRGRISITRDTSKNQFFLQLRSVTPEDTATYYCATFYGAKGTLDYWGQGTSVTVSSAKTTPPSVYPLAPVCGDTSGSSVTLGCLVKGYFPEPVTLTWNSGSLSSGVHTFPAVLQSDLYTLSSSVTVTSSTWPSQSITCNVAHPASSTKVDKKIEPRGP

SEQ ID NO: 234 - protein, 212 aa, synthetic construct
IVLTQSPSSFSVSLGDRVTISCKASGYILNRLAWYQQKPGNAPRLLISGATSLETGFPSRFSGTGSGKDYTLSISSLQTEDVGTYYCQQYWSTPWTFGGGTKLEIRRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNE

SEQ ID NO: 235 - protein, 120 aa, synthetic construct
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 236 - protein, 106 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 237 - protein, 121 aa, synthetic construct
EEVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 238 - protein, 106 aa, synthetic construct
SYELMQPPSVSVSSGQTASIPCSGDKLEEKYVSWYQQRPGQSPVLVIYQDNKRPSGIPERFSGSNSGNTATLTISGTQAMDEADYYCQVWDNYSVLFGGGTKLTVL

SEQ ID NO: 239 - protein, 106 aa, synthetic construct
SYELTQPLSESVAQGQTARITCGGNNIESRNVHWYQQKPGQAPVLVIYRDNNRPSGIPERFSGSNSGNMATLTISRAQAGDAADYYCQVWDNYTVLFGGGTKLTVL

SEQ ID NO: 240 - protein, 106 aa, synthetic construct
SYELTQPPSVAVAPGKTARITCGGNNIGSKNVHWYQQKPGQAPVLVIYRDSNRPSGIPERFSGSNSGNTATLTISRAQAGDEADFYCQVWDNYIVLFGGGTKLTVL

SEQ ID NO: 241 - protein, 106 aa, synthetic construct
QAVLTQPPSVSVAPGQTARIPCEGNNIGSKNVHWYRQKPGQVPVLVMYDDSDRPSGIPERFSGSNSGNTATLTISGTQAMDEADYYCQVWDNYSVLFGGGTKLTVL

SEQ ID NO: 242 - protein, 106 aa, synthetic construct
QPVLTQPLSVSVAPGQTARITCGGNNIGSKNVHWYQQKPGQAPVLVIYRDSSRPSGIPERLSGSNSGDTATLTISRAQAGDEADYYCQVWDDYIVVFGGGTKLTVL

SEQ ID NO: 243 - protein, 106 aa, synthetic construct
SYELTQPPSVSVTPGQTATITCGANDIGKRNVHWYQQRPGQSPVLVIYQDNKRPSGIPERFSGSNSGNTATLTISGTQAMDEADYYCQVWDNYSVLFGGGTKLTVL

SEQ ID NO: 244 - protein, 106 aa, synthetic construct
QPVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 245 - DNA, 354 bp, synthetic construct
  source 1..354: mol_type = other DNA
caggttactctgaaagagtctggccctgggatattgcagccctcccagaccctcagtctgacttgttctttctctgggttttcactgaggacttctggtatgggtgtaggctggattcgtcagccttcagggaagggtctagagtggctggcacacatttggtgggatgatgacaagcgctataatccagccctgaagagccgactgacaatctccaaggatacctccagcaaccaggtattcctcaaaatcgccagtgtggacactgcagatactgccacatactactgtgctcaaataaaccccgcctggtttgcttactggggccaagggactctggtcactgtctctgca

SEQ ID NO: 246 - DNA, 333 bp, synthetic construct
  source 1..333: mol_type = other DNA
gacactgtgctgacccaatctccagcttctttggctgtgtctctagggcagagggccaccatctcctgcaaggccagccaaagtgttgattttgatggtgatagttttatgaactggtaccaacagaaaccaggacagccacccaaactcctcatctatactacatccaatctagaatctgggatcccagccaggtttagtgccagtgggtctgggacagacttcaccctcaacatccatcctgtggaggaggaggatactgcaacctattactgtcagcaaagtaatgaggatccgtacacgttcggaggggggaccaagctggaaataaaa

SEQ ID NO: 247 - protein, 121 aa, synthetic construct [sequence omitted]

SEQ ID NO: 248 - protein, 108 aa, synthetic construct
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKR

SEQ ID NO: 249 - protein, 122 aa, synthetic construct
EVQLVESGGGLVQPGGSLRLSCAASGFTFSSFGMHWVRQAPGKGLEWVSYISSGSGNFYYVDTVKGRFTISRDNAKNSLYLQMNSLRAEDTAVYYCARSTYYHGSRGAMDYWGQGTLVTVSS

SEQ ID NO: 250 - protein, 122 aa, synthetic construct
EVQLVESGGGLVQPGGSLRLSCVASGFTFSSFGMHWVRQAPGKGLEWVSYISSGSGNFYYVDTVKGRFTISRDNAKNSLYLQMNSLRAEDTAVYYCARSTYYHGSRGAMDYWGQGTLVTVSS

SEQ ID NO: 251 - protein, 122 aa, synthetic construct
EVQLVESGGGLVQPGGSLRLSCVASGFTFSSFGMHWVRQAPGKGLEWVAYISSGSGNFYYVDTVKGRFTISRDNAKNSLYLQMNSLRAEDTAVYYCARSTYYHGSRGAMDYWGQGTLVTVSS

SEQ ID NO: 252 - protein, 122 aa, synthetic construct
QVQLVESGGGVVQPGGSLRLSCVASGFTFSSFGMHWVRQAPGKGLEWVAYISSGSGNFYYVDTVKGRFTISRDNSKNTLYLQMNSLRAEDTAVYYCARSTYYHGSRGAMDYWGQGTLVTVSS

SEQ ID NO: 253 - protein, 107 aa, synthetic construct
EIVLTQSPATLSLSPGERATLSCSASSSVSSMYWYQQKPGLAPRLLIYDTSKMASGIPDRFSGSGSGTDFTLTISRLEPEDFAVYYCQQWSSYPPITFGQGTRLEIK

SEQ ID NO: 254 - protein, 107 aa, synthetic construct
EIALTQSPATLSLSPGERATLSCSASSSVSSMYWYQLKPGLAPRLLIYDTSKMASGIPIRFSGSGSGTDFTLTVSRVEPEDFAVYYCQQWSSYPPITFGQGTRLEIK

SEQ ID NO: 255 - protein, 107 aa, synthetic construct
QVVMTQSPAFLSVTPGEKVTITCSASSSVSSMYWYQQKPDQAPKLLIYDTSKMASGVPSRFSGSGSGTDFTFTISSLEAEDAATYYCQQWSSYPPITFGGGTKVEIK

SEQ ID NO: 256 - protein, 107 aa, synthetic construct
QVVMTQSPAFLSVTPGEKVTITCSASSSVSSMYWYQLKPDQAPKLLIYDTSKMASGVPIRFSGSGSGTDFTFTVSSVEAEDAATYYCQQWSSYPPITFGGGTKVEIK

SEQ ID NO: 257 - protein, 122 aa, synthetic construct
DVQLVESGGGLVQPGGSRKLSCVASGFTFSSFGMHWVRQAPEKGLEWVAYISSGSGNFYYVDTVKGRFTISRDNPKNTLFLQMTSLRSEDTAMYYCARSTYYHGSRGAMDYWGQGTSVTVSS

SEQ ID NO: 258 - protein, 107 aa, synthetic construct
QIALTQSPAIMSASPGEKVTMTCSASSSVSSMYWYQLKPGSSPRLLIYDTSKMASGVPIRFSGSGSGTSFSLTVSRVEAEDAATYYCQQWSSYPPITFGAGSKLELK

SEQ ID NO: 259 - protein, 452 aa, synthetic construct
EVQLQQSGPELVKPGASVKISCKASGYSIGGYFMNWVKQSHGKSPEWIGRINPNNGDSFYNQKFKGTATLTVDRSSDTVHMEVLSLTSEDSAVYYCGREGYYGGRGYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 214 aa, synthetic construct
DIQMTQTTSSLSASLGDRVTISCRTSQDISNYLNWYQQKPDGTVKLLIYNTSRLHSGVPSRFSGSGSGTDYSLTIRNLEQKDIATYFCQQSNMFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 122 aa, synthetic construct
QVQLVQSGAEVKKPGASVKVSCKASGYSIGGYYMNWVRQAPGQGLEWMGRINPNSGDSFYNQKFKGRVTTTRDTSTSTMYMELSSLRSEDTAVYYCAREGYYGGRGYALDYWGQGTLVTVSS

SEQ ID NO: 262 - protein, 122 aa, synthetic construct
QVQLVQSGAEVKKPGASVKVSCKASGYTFNGYFMNWVRQAPGQGLERMGRINPNNGDSFYNQKFQGRVTMTRDTSTSTVYMELPSLRSEDTAVYYCAREGYYGGRGYALDYWGQGTLVTVSS

SEQ ID NO: 263 - protein, 122 aa, synthetic construct; identical to SEQ ID NO: 262

SEQ ID NO: 264 - protein, 122 aa, synthetic construct
QVQLVQSGAEVKKPGASVKVSCKASGYSIGGYYMNWVRQAPGQGLEWMGRINPNSGDSFYNQKFKGRVTMTRDTSTSTVYMELSSLRSEDTAVYYCAREGYYGGRGYALDYWGQGTLVTVSS

SEQ ID NO: 265 - protein, 122 aa, synthetic construct
QVQLVQSGAEVKKPGASVKVSCKASGYSIGGYFMNWVRQAPGQGLEWMGRINPNNGDSFYNQKFKGRVTMTRDTSTSTVYMELSSLRSEDTAVYYCAREGYYGGRGYALDYWGQGTLVTVSS

SEQ ID NO: 266 - protein, 122 aa, synthetic construct; identical to SEQ ID NO: 265

SEQ ID NO: 267 - protein, 122 aa, synthetic construct
QVQLVQSGAEVKKPGASVKVSCKASGYSIGGYFMNWVRQAPGQGLEWMGRINPNNGDSFYNQKFKGRVTMTRDTSTSTMYMELSSLRSEDTAVYYCAREGYYGGRGYALDYWGQGTLVTVSS

SEQ ID NO: 268 - protein, 122 aa, synthetic construct
QVQLVQSGAEVKKPGASVKVSCKASGYSIGGYFMNWVRQAPGQGLEWMGRINPNNGDSFYNQKFKGRVTTTRDTSTSTVYMELSSLRSEDTAVYYCAREGYYGGRGYALDYWGQGTLVTVSS

SEQ ID NO: 269 - protein, 122 aa, synthetic construct
QVQLVQSGAEVKKPGASVKVSCKASGYTFNGYFMNWVRQAPGQGLEWMGRINPNNGDSFYNQKFQGRVTMTRDTSTSTVYMELSSLRSEDTAVYYCAREGYYGGRGYALDYWGQGTLVTVSS

SEQ ID NO: 270 - protein, 122 aa, synthetic construct
QVQLVQSGAEVKKPGASVKVSCKASGYSIGGYFMNWVRQAPGQGLEWMGRINPNNGDSFYNQKFKGRVTMTRDRSTSTVYMELSSLRSEDTAVYYCAREGYYGGRGYALDYWGQGTLVTVSS

SEQ ID NO: 271 - protein, 122 aa, synthetic construct; identical to SEQ ID NO: 265

SEQ ID NO: 272 - protein, 122 aa, synthetic construct
QVQLVQSGAEVKKPGASVKVSCKASGYSIGGYYMNWVRQAPGQGLEWMGRINPNSGDSFYNQKFKGRVTMTRDRSTSTVYMELSSLRSEDTAVYYCAREGYYGGRGYALDYWGQGTLVTVSS

SEQ ID NO: 273 - protein, 122 aa, synthetic construct; identical to SEQ ID NO: 265

SEQ ID NO: 274 - protein, 122 aa, synthetic construct; identical to SEQ ID NO: 265

SEQ ID NO: 275 - protein, 122 aa, synthetic construct; identical to SEQ ID NO: 267

SEQ ID NO: 276 - protein, 122 aa, synthetic construct; identical to SEQ ID NO: 268

SEQ ID NO: 277 - protein, 122 aa, synthetic construct
QVQLVQSGAEVKKPGASVKVSCKASGYSIGGYYMNWVRQAPGQGLEWMGRINPNSGDSFYNQKFKGRVTTTRDRSTSTMYMELSSLRSEDTAVYYCAREGYYGGRGYALDYWGQGTLVTVSS

SEQ ID NO: 278 - protein, 122 aa, synthetic construct
QVQLVQSGAEVKKPGASVKVSCKASGYTFNGYFMNWVRQAPGQGLERMGRINPNNGDSFYNQKFQGRVTMTRDTSTSTVYMELSSLRSEDTAVYYCAREGYYGGRGYALDYWGQGTLVTVSS

SEQ ID NO: 279 - protein, 122 aa, synthetic construct
QVQLVQSGAEVKKPGASVKVSCKASGYTFNGYFMNWVRQAPGQGLRWMGRINPNNGDSFYNQKFQGRVTMTRDTSTSTVYMEPSSLRSEDTAVYYCAREGYYGGRGYALDYWGQGTLVTVSS

SEQ ID NO: 280 - protein, 122 aa, synthetic construct; identical to SEQ ID NO: 262

SEQ ID NO: 281 - protein, 108 aa, synthetic construct
DIQMTRSPSSVSASVGGRVPITCRTSQDISNYLNWYQQKPDGTVKLLIYNTSRLHSGVPSRFSGSGSGTDFTLTISSLQPEDFATYYCQQSNMFPYTFGQGIKLEIKR

SEQ ID NO: 282 - protein, 108 aa, synthetic construct
DTQMTQSPSSVSASVGDRVTITCRTSQDISNYLNWYQQKPGKAPKLLIYNTSRLHSGVPSRFSGSGSGTDFTLTISSLQPEDFATYYCQQSNMFPYTFGQGTKLEIKR

SEQ ID NO: 283 - protein, 108 aa, synthetic construct
DTQMTQSPSSVSASVGDRVTITCRTSQDISNYLNWYQQEPGKAPKLLIYNTSRLHSGVPSRFSGSGSGTDFTLTISSLQPEDFATYYCQQSNMFPYTFGQGTKLEIKR

SEQ ID NO: 284 - protein, 108 aa, synthetic construct
DIQMTRSPSSVSASVGDRVSITCRTSQDISNYLNWYQQKPDGTVKLLIYNTSRLHSGVPSRFSGSGSGTDFTLTISSLQPEDFATYYCQQSNMFPYTFGQGIKLEIKR

SEQ ID NO: 285 - protein, 108 aa, synthetic construct
DIQMTRSPSSVSASVGGRVSITCRTSQDISNYLNWYQQKPDGTVKLLIYNTSRLHSGVPSRFSGSGSGTDFTLTISSLQPEDFATYYCQQSNMFPYTFGQGIKLEIKR

SEQ ID NO: 286 - protein, 108 aa, synthetic construct; identical to SEQ ID NO: 285

SEQ ID NO: 287 - protein, 108 aa, synthetic construct; identical to SEQ ID NO: 284

SEQ ID NO: 288 - protein, 108 aa, synthetic construct; identical to SEQ ID NO: 284

SEQ ID NO: 289 - protein, 108 aa, synthetic construct
DIQMTQSPSSVSASVGDRVTITCRTSQDISNYLNWYQQKPGKAPKLLIYNTSRLHSGVPSRFSGSGSGTDFTLTISSLQPEDFATYYCQQSNMFPYTFGQGTKLEIKR

SEQ ID NO: 290 - protein, 108 aa, synthetic construct
DIQMTRSPSSVSASVGDRVTITCRTSQDISNYLNWYQQKPDGTVKLLIYNTSRLHSGVPSRFSGSGSGTDFTLTISSLQPEDFATYYCQQSNMFPYTFGQGTKLEIKR

SEQ ID NO: 291 - protein, 108 aa, synthetic construct; identical to SEQ ID NO: 284

SEQ ID NO: 292 - protein, 108 aa, synthetic construct; identical to SEQ ID NO: 284

SEQ ID NO: 293 - protein, 108 aa, synthetic construct; identical to SEQ ID NO: 285

SEQ ID NO: 294 - protein, 108 aa, synthetic construct
DIQMTRSPSSVSASVGDRVPITCRTSQDISNYLNWYQQKPDGTVKLLIYNTSRLHSGVPSRFSGSGSGTDFTLTISSLQPEDFATYYCQQSNMFPYTFGQGIKLEIKR

SEQ ID NO: 295 - protein, 108 aa, synthetic construct; identical to SEQ ID NO: 284

SEQ ID NO: 296 - protein, 108 aa, synthetic construct; identical to SEQ ID NO: 284

SEQ ID NO: 297 - protein, 108 aa, synthetic construct; identical to SEQ ID NO: 281

SEQ ID NO: 298 - protein, 108 aa, synthetic construct; identical to SEQ ID NO: 282

SEQ ID NO: 299 - protein, 108 aa, synthetic construct; identical to SEQ ID NO: 282

SEQ ID NO: 300 - protein, 108 aa, synthetic construct; identical to SEQ ID NO: 283

SEQ ID NO: 301 - protein, 107 aa, synthetic construct
DTQMTQSPSSVSASVGDRVTITCRTSQDISNYLNWYQQKPGKAPKLLIYNTARLHSGVPSRFSGSGSGTDFTLTISSLQPEDFATYYCQQSNMFPYTFGQGTKLEIK

SEQ ID NO: 302 - protein, 107 aa, synthetic construct
DTQMTQSPSSVSASVGDRVTITCRTSQDISNYLNWYQQKPGKAPKLLIYQTSRLHSGVPSRFSGSGSGTDFTLTISSLQPEDFATYYCQQSNMFPYTFGQGTKLEIK

SEQ ID NO: 303 - protein, 107 aa, synthetic construct
DTQMTQSPSSVSASVGDRVTITCRTSQDISNYLNWYQQKPGKAPKLLIYATSRLHSGVPSRFSGSGSGTDFTLTISSLQPEDFATYYCQQSNMFPYTFGQGTKLEIK

SEQ ID NO: 304 - protein, 250 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPDYGMDVWGQGTMVTVSSASGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLITFGQGTKVDIK

SEQ ID NO: 305 - protein, 251 aa, synthetic construct
QVQLVQSGAEVKKPGASVRVSCKASGYIFTNYYVHWVRQAPGQGLEWMGIISPSGGSPTYAQRLQGRVTMTRDLSTSTVYMELSSLTSEDTAVYFCARESRLRGNRLGLQSSIFDHWGQGTLVTVSSASGGGGSGGGGSGGGGSDIRMTQSPPSLSASVGDRVTIPCQASQDINNHLNWYQQKPGKAPQLLIYDTSNLEIGVPSRFSGSGSGTDFTLTISSLQPEDIATYYCQQYENLPLTFGGGTKVEIK

SEQ ID NO: 306 - protein, 250 aa, synthetic construct
QVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 307 - protein, 240 aa, synthetic construct
QVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 308 - protein, 248 aa, synthetic construct
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 309 - protein, 245 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSSASGGGGSGGGGSGGGGSDIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 310 - protein, 244 aa, synthetic construct
QVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSSASGGGGSGGGGSGGGGSDIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 311 - protein, 269 aa, synthetic construct
MALPVTALLLPLALLLHAARPGSNIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKRGGGGSGGGGSSGGGSQVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 312 - protein, 246 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGGGGSGGGGSSGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 313 - protein, 491 aa, synthetic construct
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 314 - protein, 486 aa, synthetic construct
MALPVTALLLPLALLLHAARPEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 315 - protein, 511 aa, synthetic construct
MALPVTALLLPLALLLHAARPMALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIKTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 316 - protein, 510 aa, synthetic construct
MALPVTALLLPLALLLHAARPMALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLNISSLQPDDFATYYCQQSKEVPWTFGQGTKVEIKTSSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 317 - protein, 488 aa, synthetic construct
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 318 - protein, 487 aa, synthetic construct
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 319 - protein, 118 aa, synthetic construct
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 320 - protein, 108 aa, synthetic construct
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 321 - protein, 123 aa, synthetic construct
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSS

SEQ ID NO: 322 - protein, 114 aa, synthetic construct
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 323 - protein, 122 aa, synthetic construct
QIQLVQSGPELKKPGETVKISCKASGYTFTTYGMSWVKQAPGKGLKWMGWIYTYSGVPTYADDLEGRFAFSLETSASTAYLQINNLKNEDTATYFCARVRDGYYRYAMDYWGQGTSVTVSSC

SEQ ID NO: 324 - protein, 106 aa, synthetic construct
DIHMTQSPSSLSASLGGKVTITCKASQDINRYIAWYQHKPGKGPRLLIHFTSTLQSGIPSRFSGSGSGRDYSFSISNLEPEDIATYYCLQYDDLRTFGGGTKLEIK

SEQ ID NO: 325 - protein, 120 aa, synthetic construct; identical to SEQ ID NO: 231

SEQ ID NO: 326 - protein, 113 aa, synthetic construct
DDVLMTQIPFSLPVSLGDQASISCRSSQSIVHRSGNTYLEWYLQKPGQSPKLLIYKVSNRFSGVPDRFSGSGSGTDFTLKISRVEAEDLGVYYCFQGSHLPPTFGGGTTLEIK

SEQ ID NO: 327 - protein, 480 aa, synthetic construct; identical to SEQ ID NO: 9

SEQ ID NO: 328 - protein, 546 aa, synthetic construct; identical to SEQ ID NO: 142

SEQ ID NO: 329 - protein, 490 aa, synthetic construct; identical to SEQ ID NO: 144

SEQ ID NO: 330 - protein, 491 aa, synthetic construct; identical to SEQ ID NO: 146

SEQ ID NO: 331 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 332 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 333 - protein, 116 aa, synthetic construct [sequence omitted]

SEQ ID NO: 334 - protein, 112 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 335 - protein, 111 aa, synthetic construct; identical to SEQ ID NO: 332

SEQ ID NO: 336 - protein, 116 aa, synthetic construct; identical to SEQ ID NO: 331

SEQ ID NO: 337 - protein, 117 aa, synthetic construct
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSS

SEQ ID NO: 338 - protein, 108 aa, synthetic construct
DIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKR

SEQ ID NO: 339 - protein, 120 aa, synthetic construct
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSS

SEQ ID NO: 340 - protein, 115 aa, synthetic construct
DIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVS

SEQ ID NO: 341 - protein, 118 aa, synthetic construct
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 342 - protein, 113 aa, synthetic construct
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 343 - protein, 107 aa, synthetic construct
EIVLTQSPGTLSLSPGERATLSCRASQYVSASLLAWYQQKPGQAPRLLIYGASTRATGIPDRFSGSGSGTDFTLTISRLEPEDFAVYYCQQYARSSTFGQGTKVEIK

SEQ ID NO: 344 - protein, 120 aa, synthetic construct
QVQLVQSGAEVKKPGSSVKVSCKASGGTFSSYTISWVRQAPGQGLEWMGGIIPAFGIANYAQKFQGRVTITADKSTSTAYMELSSLRSEDTAVYYCAKGGSYSLDYFDIWGQGTLVTVSS

SEQ ID NO: 345 - protein, 110 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVL

SEQ ID NO: 346 - protein, 123 aa, synthetic construct
EVQLLESGGGLVQPGGSLRLSCAASGFIFASYAMSWVRQAPGKGLEWVSEISSSGGSTTYADSVKGRFTISRDNSKNTLYLQMNSLRAEDTAVYYCARDRVMAGLGYDPFDYWGQGTLVTVSS

SEQ ID NO: 347 - protein, 107 aa, synthetic construct
QSVLTQPPSVSVAPGKTARITCGGNNIGSKSVHWYQQKPGQAPVLVIYYDSDRPSGIPERFSGSNSGNTATLTISRVEAGDEADYYCQVWDSSTAWVFGGGTKLTVL

SEQ ID NO: 348 - protein, 121 aa, synthetic construct
EVQLLESGGGLVQPGGSLRLSCAASGFTFSSYAMHWVRQAPGKGLEWVSAISGSGGSTYYADSVKGRFTISRDNSKNTLYLQMNSLRAEDTAVYYCARGTRWWWGDAFDHWGQGTLVTVSS

SEQ ID NO: 349 - protein, 107 aa, synthetic construct [sequence omitted]

SEQ ID NO: 350 - protein, 119 aa, synthetic construct
QVQLVQSGAEVKKPGSSVKVSCKASGGTFSSYDISWVRQAPGQGLEWMGGIIPVSGRANYAQKFQGRVTITTDKSTSTAYMELSSLRSEDTAVYYCARVRPTYWPLDYWGQGTLVTVSS

SEQ ID NO: 351 - protein, 108 aa, synthetic construct
EIVLTQSPGTLSLSPGERATLSCRASQSVPSSQLAWYQQKPGQAPRLLIYDASSRATGIPDRFSGSGSGTDFTLTISRLEPEDFAVYYCQQYGSSPLTFGQGTKVEIK

SEQ ID NO: 352 - protein, 117 aa, synthetic construct
QVQLVQSGAEVKKPGSSVKVSCKASGGTFSSYAIQWVRQAPGQGLEWMGGIVGSWGLANYAQKFQGRVTITTDKSTSTAYMELSSLRSEDTAVYYCATSAFGELASWGQGTLVTVSS

SEQ ID NO: 353 - protein, 126 aa, synthetic construct
QVTLKESGPVLVKPTETLTLTCTVSGFSLINARMGVSWIRQPPGKALEWLAHIFSNAEKSYRTSLKSRLTISKDTSKSQVVLTMTNMDPVTATYYCARIPGYGGNGDYHYYGMDVWGQGTTVTVSS

SEQ ID NO: 354 - protein, 108 aa, synthetic construct
DIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIKR

SEQ ID NO: 355 - protein, 125 aa, synthetic construct
QVTLKESGPVLVKPTETLTLTCTVSGFSLRNARMGVSWIRQPPGKALEWLAHIFSNDEKTYSTSLKSRLTISRDTSKGQVVLTMTKMDPVDTATYYCARIPYYGSGSHNYGMDVWGQGTTVTVSS

SEQ ID NO: 356 - protein, 108 aa, synthetic construct
DIQMTQSPSSLSASVGDRVTITCRASQDIRNDFGWYQQKPGKAPQRLLYAASTLQSGVPSRFSGSGSGTEFTLTISSLQPEDFATYYCLQYNTYPWTFGQGTKVEIKR

SEQ ID NO: 357 - protein, 120 aa, synthetic construct
QIQLVESGGGVVQPGRSLRLSCVASGFTFKNYGMHWVRQAPGKGLEWVAVIWYDGSNEYYGDPVKGRFTISRDNSKNMLYLQMNSLRADDTAVYYCARSGIAVAGAFDYWGQGTLVTVSS

SEQ ID NO: 358 - protein, 109 aa, synthetic construct
EIVLTQSPDTLSLSPGEKATLSCRASQSVSSSFLAWYQQKPGQAPSLLIYVASRRAAGIPDRFSGSGSGTDFTLTISRLEPEDFGMFYCQHYGRTPFTFGPGTKVDIKR

SEQ ID NO: 359 - protein, 119 aa, synthetic construct
QVQLVQSGAEVKKPGASVKVSCKASGYTFTGYYIHWVRQAPEQGLEWMGWINPNSGGTNYAQKFQGRVTMARDTSISTVYMDLSRLRSDDTAVYYCARIRGGNSVFDYWGQGTLVTVSS

SEQ ID NO: 360 - protein, 114 aa, synthetic construct
DIVMTQSPDSLAVSLGERATINCKSTQSILYTSNNKNFLAWYQQKPGQPPKLLISWASIRESGVPDRFSGSGSGTDFALTISSLQAEDVAVYYCQQYFSTMFSFGQGTKLEIKR

SEQ ID NO: 361 - protein, 120 aa, synthetic construct
QVQLQESGPGLVKPSQTLSLTCTVSGGSISSGAYYWTWIRQHPGKGLEWIGYIHYSGSTYSNPSLKSRITISLDTSKNQFSLKLNSVTAADTAVYYCARQEDYGGLFDYWGQGTLVTVSS

SEQ ID NO: 362 - protein, 108 aa, synthetic construct
EIVMTQSPATLSVSPGERITLSCRASQSVTTDLAWYQQMPGQAPRLLIYDASTRATGFPARFSGSGSGTDFTLTISSLQAEDFAVYYCQHYKTWPLTFGGGTKVEIKR

SEQ ID NO: 363 - protein, 118 aa, synthetic construct
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 364 - protein, 85 aa, synthetic construct
SLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 365 - protein, 122 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 366 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 367 - protein, 119 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 368 - protein, 112 aa, synthetic construct
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 369 - protein, 118 aa, synthetic construct; identical to SEQ ID NO: 319

SEQ ID NO: 370 - protein, 108 aa, synthetic construct; identical to SEQ ID NO: 320

SEQ ID NO: 371 - protein, 124 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 372 - protein, 107 aa, synthetic construct
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 373 - protein, 124 aa, synthetic construct
QVTLKESGPVLVKPTETLTLTCTVSGFSLRNARMAVSWIRQPPGKTLEWLAHIFSNDEKSYSTSLKSRLTISKDTSKSQVVLTMTNMDPVDTATYYCARIVGYGSGWYGYFDYWGQGTLVTVSS

SEQ ID NO: 374 - protein, 107 aa, synthetic construct
DIQMTQSPSSVSASVGDRVTITCRASQDIRYDLAWYQQKPGKAPKRLIYAASSLQSGVPSRFSGSGSGTEFTLTISSLQPEDFATYYCLQHNFYPLTFGGGTKVEIK

SEQ ID NO: 375 - protein, 116 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCANLAPWAAYWGQGTLVTVSS

SEQ ID NO: 376 - protein, 112 aa, synthetic construct
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 377 - protein, 123 aa, synthetic construct; identical to SEQ ID NO: 321

SEQ ID NO: 378 - protein, 114 aa, synthetic construct; identical to SEQ ID NO: 322

SEQ ID NO: 379 - protein, 120 aa, synthetic construct; identical to SEQ ID NO: 231

SEQ ID NO: 380 - protein, 111 aa, synthetic construct; identical to SEQ ID NO: 232

SEQ ID NO: 381 - protein, 122 aa, synthetic construct; identical to SEQ ID NO: 323

SEQ ID NO: 382 - protein, 106 aa, synthetic construct; identical to SEQ ID NO: 324

SEQ ID NO: 383 - protein, 122 aa, synthetic construct
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSS

SEQ ID NO: 384 - protein, 108 aa, synthetic construct
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIK

SEQ ID NO: 385 - protein, 452 aa, synthetic construct
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 386 - protein, 215 aa, synthetic construct
EIVLTQSPGTLSLSPGERATLSCRASNAVSSSYLAWYQQKPGQAPRLLIYGASYRATGIPDRFSGSGSGTDFTLTISRLEPEDFAVYYCQQHSLYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 387 - protein, 449 aa, synthetic construct
QVQLQESGPGLVKPSETLSLTCAVSGYSILSGYYWFWIRQPPGKGLEWIGGIYHSGSTAYNPSLKSRVTISVDTSKNQFSLKLSSVTAADTAVYYCARGGEVTYSRGPLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 388 - protein, 122 aa, synthetic construct; identical to SEQ ID NO: 323

SEQ ID NO: 389 - protein, 106 aa, synthetic construct; identical to SEQ ID NO: 324

SEQ ID NO: 390 - protein, 242 aa, synthetic construct
DIHMTQSPSSLSASLGGKVTITCKASQDINRYIAWYQHKPGKGPRLLIHFTSTLQSGIPSRFSGSGSGRDYSFSISNLEPEDIATYYCLQYDDLRTFGGGTKLEIKGGGGSGGGGSGGGGSQIQLVQSGPELKKPGETVKISCKASGYTFTTYGMSWVKQAPGKGLKWMGWIYTYSGVPTYADDLEGRFAFSLETSASTAYLQINNLKNEDTATYFCARVRDGYYRYAMDYWGQGTSVTVSS

SEQ ID NO: 391 - protein, 1046 aa, synthetic construct
MKLPVRLLVLMFWIPASSSDIHMTQSPSSLSASLGGKVTITCKASQDINRYIAWYQHKPGKGPRLLIHFTSTLQSGIPSRFSGSGSGRDYSFSISNLEPEDIATYYCLQYDDLRTFGGGTKLEIKGGGGSGGGGSGGGGSQIQLVQSGPELKKPGETVKISCKASGYTFTTYGMSWVKQAPGKGLKWMGWIYTYSGVPTYADDLEGRFAFSLETSASTAYLQINNLKNEDTATYFCARVRDGYYRYAMDYWGQGTSVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 392 - protein, 937 aa, synthetic construct
MKLPVRLLVLMFWIPASSSDIHMTQSPSSLSASLGGKVTITCKASQDINRYIAWYQHKPGKGPRLLIHFTSTLQSGIPSRFSGSGSGRDYSFSISNLEPEDIATYYCLQYDDLRTFGGGTKLEIKGGGGSGGGGSGGGGSQIQLVQSGPELKKPGETVKISCKASGYTFTTYGMSWVKQAPGKGLKWMGWIYTYSGVPTYADDLEGRFAFSLETSASTAYLQINNLKNEDTATYFCARVRDGYYRYAMDYWGQGTSVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 393 - protein, 830 aa, synthetic construct
MKLPVRLLVLMFWIPASSSDIHMTQSPSSLSASLGGKVTITCKASQDINRYIAWYQHKPGKGPRLLIHFTSTLQSGIPSRFSGSGSGRDYSFSISNLEPEDIATYYCLQYDDLRTFGGGTKLEIKGGGGSGGGGSGGGGSQIQLVQSGPELKKPGETVKISCKASGYTFTTYGMSWVKQAPGKGLKWMGWIYTYSGVPTYADDLEGRFAFSLETSASTAYLQINNLKNEDTATYFCARVRDGYYRYAMDYWGQGTSVTVSSESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 394 - protein, 119 aa, synthetic construct
EVQLVESGGGVVQPGRSLRLSCAASGFTFSSFGMHWVRQAPGKGLEWVAYISGGSYTIYYADTVKGRFTISRDNSKNTLYLQMNSLRAEDTAVYYCARSYGNFWYFDVWGQGTTVTVSS

SEQ ID NO: 395 - protein, 109 aa, synthetic construct
DIVMTQSPDSLAVSLGERATINCSVSSSISSSNLHWYQQKPGQPPKLLIYGTSNLASGVPDRFSGSGSGTDFTLTISSLQAEDVAVYYCQQWSSYPLTFGQGTKVEIKR

SEQ ID NO: 396 - protein, 109 aa, synthetic construct
DIVMTQSPDSLAVSLGERATIACSVSSSISSSNLHWYQQKPGQSPKPWIYGTSNLASGVPVRFSGSGSGTSYFLTISSLQAEDVATYYCQQWSSYPLTFGQGTKVEIKR

SEQ ID NO: 397 - protein, 119 aa, synthetic construct
DVQLVESGGGLXQPGGSRKLSCAASGFTFSSFGMHWVRQAPEKGLEWVAYISGGSYTIYYADTVKGRFTISRDNPKNTLFLQMTSLRSEDTAMYYCASSYGNFWYFDVWGAGTTVTVSS

SEQ ID NO: 398 - protein, 108 aa, synthetic construct
EIVLTQSPALMAASPGEKVTIACSVSSSISSSNLHWYQQKSETSPKPWIYGTSNLASGVPVRFSGSGSGTSYFLTISSMEAEDAATYYCQQWSSYPLTFGGGTKLEIK

SEQ ID NO: 399 - protein, 109 aa, synthetic construct
DIVMTQSPALMAVSLGERATIACSVSSSISSSNLHWYQQKPETPPKPWIYGTSNLASGVPDRFSGSGSGTSYFLTISSMEAEDVAVYYCQQWSSYPLTFGQGTKVEIKR

SEQ ID NO: 400 - protein, 119 aa, synthetic construct
DVQLVESGGGVVQPGGSRRLSCAASGFTFSSFGMHWVRQAPGKGLEWVAYISGGSYTIYYADTVKGRFTISRDNSKNTLFLQMTSLRAEDTAVYYCASSYGNFWYFDVWGQGTTVTVSS

SEQ ID NO: 401 - protein, 119 aa, synthetic construct
EVQLVESGGGVVQPGRSLRLSCAASGFTFSSFGMHWVRQAPEKGLEWVAYISGGSYTIYYADTVKGRFTISRDNSKNTLYLQMNSLRAEDTAVYYCASSYGNFWYFDVWGQGTTVTVSS

SEQ ID NO: 402 - protein, 138 aa, synthetic construct
MDSRLNLVFLXLILKGVQCDVQLVESGGGLXQPGGSRKLSCAASGFTFSSFGMHWVRQAPEKGLEWVAYISGGSYTIYYADTVKGRFTISRDNPKNTLFLQMTSLRSEDTAMYYCASSYGNFWYFDVWGAGTTVTVSS

SEQ ID NO: 403 - protein, 130 aa, synthetic construct
MDFHVQIFSFMLISVTVILSSGEIVLTQSPALMAASPGEKVTIACSVSSSISSSNLHWYQQKSETSPKPWIYGTSNLASGVPVRFSGSGSGTSYFLTISSMEAEDAATYYCQQWSSYPLTFGGGTKLEIK

SEQ ID NO: 404 - protein, 117 aa, synthetic construct
EVQLQQSGPDLVKPGASVKMSCKASGYTFTDYFMKWVKQSHGKSLEWIGDINPNNGDTFYNQKFKGKATLTVDKSSSTAYMQLSSLTSEDSAVYYCAKGGFNPGDYWGQGTTLTVSS

SEQ ID NO: 405 - protein, 112 aa, synthetic construct
DAVLTQAPLSLPVSLGDQASISCRSSQSLVHRNGNTYLHWFLQRPGQSPKLLIDKVSNRFSGVPDRFSGSGSGTDFTLKISRVEAEDLGVYFCSQSTHVPLTFGAGTKLELK

SEQ ID NO: 406 - protein, 321 aa, synthetic construct
MGEPQGSRRILVTGGSGLVGRAIQKVVADGAGLPGEEWVFVSSKDADLTDAAQTQALFQKVQPTHVIHLAAMVGGLFRNIKYNLDFWRKNVHINDNVLHSAFEVGTRKVVSCLSTCIFPDKTTYPIDETMIHNGPPHSSNFGYSYAKRMIDVQNRAYFQQHGCTFTAVIPTNVFGPHDNFNIEDGHVLPGLIHKVHLAKSNGSALTVWGTGKPRRQFIYSLDLARLFIWVLREYNEVEPIILSVGEEDEVSIKEAAEAVVEAMDFCGEVTFDSTKSDGQYKKTASNGKLRAYLPDFRFTPFKQAVKETCAWFTDNYEQARK

SEQ ID NO: 407 - protein, 575 aa, synthetic construct
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 408 - protein, 141 aa, synthetic construct
MGWSWIFLFLVSGTGGVLSEVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAAS

SEQ ID NO: 409 - protein, 134 aa, synthetic construct
MESQTQVLMSLLFWVSGTCGDFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKR

SEQ ID NO: 410 - protein, 140 aa, synthetic construct
WTWRFLFVVAAATGVQSQVQLLQSGAEVKKPGSSVKVSCKASGGTFSTYAISWVRQAPGQGLEWMGGIIPIFGIVNYAQKFQGRVTITADESTSTAYMELSSLRSEDTAVYYCARGGGSGPDVLDIWGQGTMVTVSSAST

SEQ ID NO: 411 - protein, 132 aa, synthetic construct
MDMRVPAQLLGLLLLWLPGARCVIWMTQSPSLLSASTGDRVTISCRMSQGIRSYLAWYQQKPGKAPELLIYAASTLQSGVPSRFSGSGSGTDFTLTISSLQSEDFATYYCQQYYSFPYTFGQGTKLEIKRTV

SEQ ID NO: 412 - protein, 115 aa, synthetic construct
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 413 - protein, 107 aa, synthetic construct
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 414 - protein, 118 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 415 - protein, 111 aa, synthetic construct
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 416 - protein, 127 aa, synthetic construct
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 417 - protein, 113 aa, synthetic construct
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 418 - protein, 119 aa, synthetic construct
VQLQESGPGLVKPSQSLSLTCSVTDYSITSGYYWNWIRQFPGNKLEWMGYISYDGSNNYNPSLKNRISITRDTSKNQFFLKLSSVTTEDTATYYCSRGEGFYFDSWGQGTTLTVSSARS

SEQ ID NO: 419 - protein, 113 aa, synthetic construct
DIMMSQSPSSLAVSVGEKFTMTCKSSQSLFFGSTQKNYLAWYQQKPGQSPKLLIYWASTRESGVPDRFTGSGSGTDFTLAISSVMPEDLAVYYCQQYYNYPWTFGGGTKLEIK

SEQ ID NO: 420 - protein, 679 aa, synthetic construct
MLLLVTSLLLCELPHPAFLLIPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 421 - protein, 672 aa, synthetic construct
MLLLVTSLLLCELPHPAFLLIPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 422 - protein, 679 aa, synthetic construct
MLLLVTSLLLCELPHPAFLLIPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 423 - protein, 672 aa, synthetic construct
MLLLVTSLLLCELPHPAFLLIPQVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSSGGGGSGGGGSGGGGSDVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 424 - protein, 118 aa, synthetic construct
QVQLQESGPGLVQPSQTLSLTCTVSGFSLTTYDVHWVRQPPGKGLEWMGRIQNGGITDYNSALKSRLIISRDTSKSQVFLKMNSVQTEDTAMYFCAKTGSYFYAFDHWGQGTLVTVSS

SEQ ID NO: 425 - protein, 118 aa, synthetic construct
QVQLQESGPGLVQPSQTLSLTCTVSGFSLTTYDVHWVRQPPGKGLEWMGRIQNAGITDYNSALKSRLIISRDTSKSQVFLKMNSVQTEDTAMYFCAKTGSYFYAFDHWGQGTLVTVSS

SEQ ID NO: 426 - protein, 118 aa, synthetic construct
QVQLQESGPGLVQPSQTLSLTCTVSGFSLTTYDVHWVRQPPGKGLEWMGRIQDGGITDYNSALKSRLIISRDTSKSQVFLKMNSVQTEDTAMYFCAKTGSYFYAFDHWGQGTLVTVSS

SEQ ID NO: 427 - protein, 111 aa, synthetic construct
QFVLTQPNSVSTNLGSTVKLSCKRNTGNIGSNYVNWYQQHEGRSPTTMIYRDDKRPDGVPDRFSGSIDRSSNSALLTINNVQTEDEADYFCQSYSSGINIIFGGGTKLTVL

SEQ ID NO: 428 - protein, 119 aa, synthetic construct
QVQLQESGPTLVKPGDSVKMSCKAFGYTFTDHIIHWVKQSHGKSLEWIGYINPYSGGTNYNEKFKSKATLTVDKSSSTAYMEFSRLTSEDSAICYCALNYGSYYAMDAWGQGTSVTVSS

SEQ ID NO: 429 - protein, 107 aa, synthetic construct
DIQMTQSPASLSASLGQTVTIECRPSEDIYSNLAWFQQKPGSSPQLLIYDANNLADGVPSRFSGSGSGTQYSLKINSLQSEDVASYFCQQYNKYPYTFGTGTKLELK

SEQ ID NO: 430 - protein, 119 aa, synthetic construct
EVQLVESGGGLVQPGRSLKLSCAASGFTFSDYNMAWVRQAPKKGLEWVATILYDGGRTYYRGSVKGRFTISRDNAKSTLYLRMDSLRSEDTATYYCATHSRGTDYFDYWGQGVMVTVSS

SEQ ID NO: 431 - protein, 106 aa, synthetic construct
EIVLTQSPTSMTASPGEQVTITCRASSSINYMHWYQQKPGASPRPWIYETSKLASGVPDRFSGSASGTSYSLTINNMEAEDAATYYCQQWNYPSWTFGGGTKLELK

SEQ ID NO: 432 - protein, 123 aa, synthetic construct
QVQLKESGPGLVQPSQTLSLTCTVSGFSLTSYGVSWVRQPPGKGLEWIATISSAGSTYYDLVLKSRLSITRDTSKSQVFLKVHSLQTEDTAIYLCARDAPVFNYGSYNAMDSWGQGTSVTVSS

SEQ ID NO: 433 - protein, 106 aa, synthetic construct
DIQMTQSPSFLSATVGDRVTINCKASQNINKYLNWYQQKLGEAPKRLIYNTNSLQTGIPSRFSGSGSGTDYTLTISSLQPEDVATYFCLQHKSGLTFGSGTKLEIK

SEQ ID NO: 434 - protein, 125 aa, synthetic construct
QVQLQQSGPELRRPGSSVRLSCKASGYRIKDFLIHWIKNRPEHGLEWIGWIDPEDGETKYAQKFQTKATLTADTSSNTAYMQLSSLTSEDTATYFCARWGDVYYGLMRGHVMDAWGQGTSVTVSS

SEQ ID NO: 435 - protein, 113 aa, synthetic construct
DVLMTQTPVSLPVSLGGQVSISCRSSQSLVHSDGDTYLHWYLQKPGQSPQLLIYRVSNRFSGVPDRFSGSGSGTDFTLKISRVEPEDLGLYYCLQTTHFPPWTFGGGTKLEMK

SEQ ID NO: 436 - protein, 120 aa, synthetic construct
QIQIVQSGSDVTSTCNGCGTCYFSGFSLSTTGICVSWIRQPSGKGQEWLADFCWDDGKGYNPSLKNRLSISKDTSNNQVFLKITSVDTADTATYYCARRRVYYGIYFDYWGQGVMVTVSS

SEQ ID NO: 437 - protein, 106 aa, synthetic construct
DIVMTQSPALAVSPGERVSISCRASNSVSTRMHWYQQKPGQQPKLLIYGASNLESGVPARFSGSGSGTDFTLTIDPVEADDIATYFCQQSWNDPLTFGSGTKLEIK

SEQ ID NO: 438 - protein, 117 aa, synthetic construct
EVQLVESGGGLVQPGRSLKLSCAASGFTFSHYNMAWVRQAPKKGLEWVATITYDDHSTYYRDSVKGRFTISRDTAKSTLYLQMDSLRSEDTATYYCARLVNYAFAYWGQGTLVTVSS

SEQ ID NO: 439 - protein, 106 aa, synthetic construct
NIVMTQSPKSMSISVGDRVTMNCKASQTVGNNIAWYQQKPGLSPQLLIDYASNRYTGVPNRFTGGGYGTDFILTINSVQAEDAAFYYCQRMYNSPTFGGGTKLELK

SEQ ID NO: 440 - protein, 116 aa, synthetic construct
EVKLQESGPSLVQPSETLSLTCTVSGFSLTSYSVHWVRQHSGKSLEWMGRMWNDGDTSYNSAFTSRLSISRDTSKGQVFLKMNSLQTEDTGTYYCARGHRTPFDYWGQGVMVTVSS

SEQ ID NO: 441 - protein, 113 aa, synthetic construct
DIVMTQSPSSLAVSAGETVTINCKSSQSFLSSGDERNYVAWYQHKPGQSPKLLIYWASTRHSGVPDRFIGSGSGTDFTLTISSVQAEDLAIYYCQQYYDTPLTFGSGTKLEIK

SEQ ID NO: 442 - protein, 119 aa, synthetic construct
QVQLQESGPTLVKPGDSVKMSCKASAYTFTDNIIHWVKQSHGKSLDWIGYINPYSGGTNYNGWFRSKATLTVDKSSSTAYMEFSRLTSDDSAIYYCALNYGSYYAMDAWGQGTSVTVSS

SEQ ID NO: 443 - protein, 107 aa, synthetic construct
DIQMTQSPASLSASLGETVTIDCRPSEDIFNNLAWYQQKPGNSPQLLIYDANSLADGVPSRFSGSGSGTQYSLMIIRLQSEDVASYFCHQYNIYPYTFGAGTKLELK

SEQ ID NO: 444 - protein, 119 aa, synthetic construct
QVQLQESGPTLVNPGDSVKMSCKASGYTFTDHIIHWVKQSHGKSLEWIGYINPYSGGANYNGKFKSKATLTIDKSSSTAYMEFSRLTSGDSAIYYCALNYGSYYAMDAWGQGTSVTVSS

SEQ ID NO: 445 - protein, 107 aa, synthetic construct
DIQMTQSPASLSASLGETVTIECRTSKDIYSNLAWFQQEPGNSPQLLIYDASNLADGVPSRFSGSGSGTQYSLQINNLQSEDVASYFCHQYNNYPYTFGTGTKLELK

SEQ ID NO: 446 - protein, 121 aa, synthetic construct
EVKLQESGPSLVQSSQTLSLTCTVSGFSLTSYHIHWVRQPPGKGLEWMGVMWSDGDTSYSSALKSRLSISRDTSQSQVFLKMNSLQTEDTATYYCARGDYSSYIYLWFAYWGQGTLVTVSS

SEQ ID NO: 447 - protein, 113 aa, synthetic construct; identical to SEQ ID NO: 441

SEQ ID NO: 448 - protein, 121 aa, synthetic construct
EVKLQESGPSLVQSSQTLSLTCTVSGFSLTSYHIHWVRQTPGKGLEWMGVMWSDGDTSYNSALKSRLSISRDTSQSQVFLKMNSLQTEDTATYYCARGYYSSYLYLWFAYWGQGTLVTVSS

SEQ ID NO: 449 - protein, 113 aa, synthetic construct
DIVMTQSPSSLAVSEGETVTINCKSSQSFLSSGDGKNYVAWYQYKPGQSPKLLIYWASTRQSGVPDRFIGSGSGTDFTLTISTVQAEDLAIYYCQQYYDTPLTFGSGTKLEIK

SEQ ID NO: 450 - protein, 119 aa, synthetic construct
QVQLQESGPTLVKPGDSVKMSCKASGYTFTDYIIHWVKQSHGKSLEWIGYINPYSDGTNYNEKFKSKATLTVDKSTSTAYMEFSRLTSEDSAIYFCALNYGSYYAMDAWGQGTSVTVSS

SEQ ID NO: 451 - protein, 107 aa, synthetic construct
DIQMTQSPASLSASLGETVTIECRPSEDIHSNVAWYQQKPGNSPQLLIYDASNLADGVPSRFSGSGSGTQYSLKINSLQSEDVASYFCHQYNIYPYTFGSGTKLELK

SEQ ID NO: 452 - protein, 534 aa, synthetic construct
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQEEETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 453 - protein, 271 aa, synthetic construct
MPEAPPLLLAAVLLGLVLLVVLLLLLRHWGWGLCLIGWNEFILQPIHNLLMGDTKEQRILNHVLQHAEPGNAQSVLEAIDTYCEQKEWAMNVGDKKGKIVDAVIQEHQPSVLLELGAYCGYSAVRMARLLSPGARLITIEINPDCAAITQRMVDFAGVKDKVTLVVGASQDIIPQLKKKYDVDTLDMVFLDHWKDRYLPDTLLLEECGLLRKGTVLLADNVICPGAPDFLAHVRGSSCFECTHYQSFLEYREVVDGLEKAIYKGPGSEAGP

SEQ ID NO: 454 - protein, 273 aa, synthetic construct
MSGSQSEVAPSPQSPRSPEMGRDLRPGSRVLLLLLLLLLVYLTQPGNGNEGSVTGSCYCGKRISSDSPPSVQFMNRLRKHLRAYHRCLYYTRFQLLSWSVCGGNKDPWVQELMSCLDLKECGHAYSGIVAHQKHLLPTSPPISQASEGASSDIHTPAQMLLSTLQSTQRPTLPVGSLSSDKELTRPNETTIHTAGHSLAAGPEAGENQKQPEKNAGPTARTSATVPVLCLLAIIFILTAALSYVLCKRRRGQSPQSSPDLPVHYIPVAPDSNT

SEQ ID NO: 455 - protein, 247 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 456 - DNA, 741 bp, synthetic construct
  source 1..741: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttcctcggacgtccggtggaggcaccaagctggaaatcaaaggcggaggtggaagcggagggggaggatctggcggcggaggaagcggaggccaggtccagctgcagcagtctggacctgagctggtgaagcctggggcttcagtgaggatatcctgcaaggcttctggctacaccttcacaagttaccatatacattgggtgaagcagaggcctggacagggacttgagtggattggatggatttatcctggaaatgttaatactgagtacaatgagaagttcaagggcaaggccacactgactgcagacaaatcgtccagcacagcctacatgcacctcagcagcctgacctctgaggactctgcggtctatttctgtgccagagaggagattacctatgctatggactactggggtcaaggaacctcagtcaccgtgtcctca

SEQ ID NO: 457 - protein, 247 aa, synthetic construct
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 458 - DNA, 741 bp, synthetic construct
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatgggatggatctaccctggcaatgttaacacagaatataatgagaagttcaagggcaaagccaccattaccgcggacaaatccacgagcacagcctacatggagctgagcagcctgagatctgaagacacggctgtgtattactgtgcgagggaggaaattacctacgctatggactactggggccagggaaccacagtcaccgtgtcctcaggcggaggtggaagcggagggggaggatctggcggcggaggaagcggaggcgagattgtattgacccagagcccaggcaccctgagcctctctccaggagagcgggccaccctcagttgtagatccagtcagagtattgtacacagtaatgggaacacctatttggaatggtatcagcagaaaccaggtcaagccccaagattgctcatctacaaagtctctaacagatttagtggtattccagacaggttcagcggttccggaagtggtactgatttcaccctcacgatctccaggctcgagccagaagatttcgccgtttattactgttttcaaggttcacatgtgccgcgcacattcggtgggggtactaaagtagaaatcaaa

SEQ ID NO: 459 - protein, 247 aa, synthetic construct
QVQLVQSGAEVKKPGSSVKVSCKASGYTFTSYHIHWVRQAPGQGLEWMGWIYPGNVNTEYNEKFKGKATITADKSTSTAYMELSSLRSEDTAVYYCAREEITYAMDYWGQGTTVTVSSGGGGSGGGGSGGGGSGGDIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPRTFGGGTKVEIK

SEQ ID NO: 460 - DNA, 741 bp, synthetic construct
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatgggatggatctaccctggcaatgttaacacagaatataatgagaagttcaagggcaaagccaccattaccgcggacaaatccacgagcacagcctacatggagctgagcagcctgagatctgaagacacggctgtgtattactgtgcgagggaggaaattacctacgctatggactactggggccagggaaccacagtcaccgtgtcctcaggcggaggtggaagcggagggggaggatctggcggcggaggaagcggaggcgacattgtaatgacccagaccccactcagcctgcccgtcactccaggagagccggccagcatcagttgtagatccagtcagagtattgtacacagtaatgggaacacctatttggaatggtatctgcagaaaccaggtcaatccccacaattgctcatctacaaagtctctaacagatttagtggtgtaccagacaggttcagcggttccggaagtggtactgatttcaccctcaagatctccagggtcgaggcagaagatgtcggcgtttattactgttttcaaggttcacatgtgccgcgcacattcggtgggggtactaaagtagaaatcaaa

SEQ ID NO: 461 - protein, 247 aa, synthetic construct
EVQLVESGGGLVKPGGSLRLSCAASGYTFTSYHIHWVRQAPGKGLEWVGWIYPGNVNTEYNEKFKGRFTISRDDSKNTLYLQMNSLKTEDTAVYYCAREEITYAMDYWGQGTTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTLTISSLQPEDFATYYCFQGSHVPRTFGGGTKVEIK

SEQ ID NO: 462 - DNA, 741 bp, synthetic construct
  source 1..741: mol_type = other DNA
gaggtgcagctggtggagtctgggggtgggctggtgaagcctgggggctcactgaggctttcctgcgcggcttctggatacaccttcactagctatcatatacattgggtgcgccaggcccccggaaaagggcttgagtgggtgggatggatctaccctggcaatgttaacacagaatataatgagaagttcaagggcagattcaccattagcagggacgattccaagaacacactctacctgcagatgaacagcctgaaaactgaagacacggctgtgtattactgtgcgagggaggaaattacctacgctatggactactggggccagggaaccacagtcaccgtgtcctcaggcggaggtggaagcggagggggaggatctggcggcggaggaagcggaggcgacattcaaatgacccagagcccatccagcctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccaagcaggttcagcggttccggaagtggtactgatttcaccctcacgatctcctctctccagccagaagatttcgccacttattactgttttcaaggttcacatgtgccgcgcacattcggtgggggtactaaagtagaaatcaaa

SEQ ID NO: 463 - protein, 247 aa, synthetic construct
QVQLVQSGAEVKKPGSSVKVSCKASGYTFTSYHIHWVRQAPGQGLEWIGWIYPGNVNTEYNEKFKGKATITADESTNTAYMELSSLRSEDTAVYYCAREEITYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPARFSGSGSGTEFTLTISSLQPDDFATYYCFQGSHVPRTFGQGTKVEVK

SEQ ID NO: 464 - DNA, 741 bp, synthetic construct
  source 1..741: mol_type = other DNA
caggtgcagctggtgcagtctggggctgaggtgaagaagcctgggtcctcagtgaaggtttcctgcaaggcttctggatacaccttcactagctatcatatacattgggtgcgccaggcccccggacaagggcttgagtggatcggatggatctaccctggcaatgttaacacagaatataatgagaagttcaagggcaaagccaccattaccgcggacgaatccacgaacacagcctacatggagctgagcagcctgagatctgaagacacggctgtgtattactgtgcgagggaggaaattacctacgctatggactactggggccagggaaccctggtcaccgtgtcctcaggcggaggtggaagcggagggggaggatctggcggcggaggaagcggaggcgacattcaaatgacccagagcccatccaccctgagcgcatctgtaggtgaccgggtcaccatcacttgtagatccagtcagagtattgtacacagtaatgggaacacctatttggaatggtatcagcagaaaccaggtaaagccccaaaattgctcatctacaaagtctctaacagatttagtggtgtaccagccaggttcagcggttccggaagtggtactgaattcaccctcacgatctcctctctccagccagatgatttcgccacttattactgttttcaaggttcacatgtgccgcgcacattcggtcagggtactaaagtagaagtcaaa

SEQ ID NO: 465 - protein, 247 aa, synthetic construct
QVQLVQSGAEVKKPGSSVKVSCKASGYTFTSYHMHWVRQAPGQGLEWIGYIYPGNVNTEYNEKFKGKATLTADKSTNTAYMELSSLRSEDTAVYFCAREEITYAMDYWGQGTLVTVSSGGGGSGGGGSGGGGSGGDVQMTQSPSTLSASVGDRVTITCSSSQSIVHSNGNTYMEWYQQKPGKAPKLLIYKVSNRFSGVPDRFSGSGSGTEFTLTISSLQPDDFATYYCHQGSHVPRTFGQGTKVEVK

SEQ ID NO: 466 - DNA, 741 bp, synthetic construct
  source 1..741: mol_type = other DNA
caggtgcagctggtgcagtctggggctgaggtgaagaagcctgggtcctcagtgaaggtttcctgcaaggcttctggatacaccttcactagctatcatatgcattgggtgcgccaggcccccggacaagggcttgagtggatcggatacatctaccctggcaatgttaacacagaatataatgagaagttcaagggcaaagccacccttaccgcggacaaatccacgaacacagcctacatggagctgagcagcctgagatctgaagacacggctgtgtatttctgtgcgagggaggaaattacctacgctatggactactggggccagggaaccctggtcaccgtgtcctcaggcggaggtggaagcggagggggaggatctggcggcggaggaagcggaggcgacgttcaaatgacccagagcccatccaccctgagcgcatctgtaggtgaccgggtcaccatcacttgtagctccagtcagagtattgtacacagtaatgggaacacctatatggaatggtatcagcagaaaccaggtaaagccccaaaattgctcatctacaaagtctctaacagatttagtggtgtaccagacaggttcagcggttccggaagtggtactgaattcaccctcacgatctcctctctccagccagatgatttcgccacttattactgtcatcaaggttcacatgtgccgcgcacattcggtcagggtactaaagtagaagtcaaa

SEQ ID NO: 467 - protein, 247 aa, synthetic construct
QVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSSGGGGSGGGGSGGGGSGGDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 468 - DNA, 741 bp, synthetic construct
  source 1..741: mol_type = other DNA
caggtgcagctgcagcagtctgggcctgagctggtgaagcctggggcctcagtgaagatgtcctgcaaggcttctggatacaccttcactagctatcatatccagtgggtgaagcagaggcctggacaagggcttgagtggatcggatggatctaccctggcgatggtagtacacagtataatgagaagttcaagggcaaaaccacccttaccgcggacaaatcctccagcacagcctacatgttgctgagcagcctgacctctgaagactctgctatctatttctgtgcgagggaggggacctactacgctatggactactggggccagggaacctcagtcaccgtgtcctcaggcggaggtggaagcggagggggaggatctggcggcggaggaagcggaggcgatgttttgatgacccagactccactctccctgcctgtctctcttggagaccaagtctccatctcttgtagatccagtcagagtattgtacacagtaatgggaacacctatttagaatggtatctgcagaaaccaggtcagtctccaaagttgctcatctacaaagtctctaacagatttagtggtgtaccagacaggttcagcggttccggaagtggtactgatttcaccctcaagatctcgagagtggaggctgaggatctgggagtttattactgttttcaaggttcacatgtgccgcgcacattcggtggaggtactaaactggaaatcaaa

SEQ ID NO: 469 - protein, 247 aa, synthetic construct
QLQLQESGPGLVKPSETLSLTCTVSGYTFTSYHIQWIRQPPGKGLEWIGWIYPGDGSTQYNEKFKGRATISVDTSKNQFSLNLDSVSAADTAIYYCAREGTYYAMDYWGKGSTVTVSSGGGGSGGGGSGGGGSGGDIQMTQSPSSLSASVGDRVTITCRSSQSIVHSNGNTYLEWYQQKPGKAPKLLIYKVSNRFSGVPSRFSGSGSGTDFTFTISSLQPEDIATYYCFQGSHVPRTFGPGTKVDIK

SEQ ID NO: 470 - DNA, 741 bp, synthetic construct
  source 1..741: mol_type = other DNA
cagctgcagctgcaggagtctgggcccgggctggtgaagccttcggaaacgctgagcctcacctgcacggtttctggatacaccttcaccagctatcatatccagtggatccgacagccccctggaaaagggcttgagtggatcggatggatctaccctggcgatggttcaacacagtacaatgagaagttcaagggcagagccacgattagcgtggacacatccaagaaccaattctccctgaacctggacagcgtgagtgctgcggacacggccatttattactgtgcgagagagggaacttactacgctatggactactggggcaaagggagcacggtcaccgtgtcctcaggcggaggtggaagcggagggggaggatctggcggcggaggaagcggaggcgacatccagatgacccagagcccaagctccctgagtgcgtccgtgggcgaccgcgtgaccatcacttgcagatcctctcagtccatcgtgcactccaacggcaacacgtacctcgagtggtaccagcagaagcccgggaaggccccgaaactgctcatctacaaggtgagcaaccggttctccggcgtccccagccgcttctcagggtccggctcggggacggatttcaccttcacgattagcagcttgcagcccgaagacatcgccacgtactactgctttcagggaagtcacgtgccgcgtaccttcgggccgggcacgaaagtggatattaag

SEQ ID NO: 471 - protein, 247 aa, synthetic construct
EVQLVQSGAELKKPGSSVKVSCKASGYTFTSYHIQWVKQAPGQGLEWIGWIYPGDGSTQYNEKFKGKATLTVDKSTNTAYMELSSLRSEDTAVYYCAREGTYYAMDYWGQGTLVTVSSGGGGSGGGGSGGGGSGGDIQMTQSPSTLSASVGDRVTITCRSSQSIVHSNGNTYLEWYQQKPGKAPKLLIYKVSNRFSGVPSRFSGSGSGTDFTLTISSLQPDDFATYYCFQGSHVPRTFGQGTKVEVK

SEQ ID NO: 472 - DNA, 741 bp, synthetic construct
  source 1..741: mol_type = other DNA
gaggtgcagctggtgcagtctggggccgagctgaagaagcctgggtcctcggtgaaggtgtcctgcaaggcttctggatacaccttcaccagctatcatatccagtgggtaaaacaggcccctggacaagggcttgagtggatcggatggatctaccctggcgatggttcaacacagtacaatgagaagttcaagggcaaagccacgcttaccgtggacaaatccacgaacacagcctacatggagctgagcagcctgagatctgaggacacggccgtatattactgtgcgagagagggaacttactacgctatggactactggggccaagggaccctggtcaccgtgtcctcaggcggaggtggaagcggagggggaggatctggcggcggaggaagcggaggcgacatccagatgacccagagcccatccaccctgagtgcgtccgtgggcgaccgcgtgaccatcacttgcagatcctctcagtccatcgtgcactccaacggcaacacgtacctcgagtggtaccagcagaagcccgggaaggccccgaaactgctcatctacaaggtgagcaaccggttctccggcgtccccagccgcttctcagggtccggctcggggacggatttcaccctcacgattagcagcttgcagcccgatgacttcgccacgtactactgctttcagggaagtcacgtgccgcgtaccttcgggcagggcacgaaagtggaagttaag

SEQ ID NO: 473 - protein, 247 aa, synthetic construct
QVQLVQSGAEVKKPGSSVKVSCKASGYTFTSYHIQWVRQAPGQGLEWMGWIYPGDGSTQYNEKFKGRVTITADKSTSTAYMELSSLRSEDTAVYYCAREGTYYAMDYWGQGTTVTVSSGGGGSGGGGSGGGGSGGEIVLTQSPGTLSLSPGERATLSCRSSQSIVHSNGNTYLEWYQQKPGQAPRLLIYKVSNRFSGIPDRFSGSGSGTDFTLTISRLEPEDFAVYYCFQGSHVPRTFGGGTKVEIK

SEQ ID NO: 474 - DNA, 741 bp, synthetic construct
  source 1..741: mol_type = other DNA
caggtgcagctggtgcagtctggggccgaggtgaagaagcctgggtcctcggtgaaggtgtcctgcaaggcttctggatacaccttcaccagctatcatatccagtgggtacgacaggcccctggacaagggcttgagtggatgggatggatctaccctggcgatggttcaacacagtacaatgagaagttcaagggcagagtcacgattaccgcggacaaatccacgagcacagcctacatggagctgagcagcctgagatctgaggacacggccgtatattactgtgcgagagagggaacttactacgctatggactactggggccaagggaccacggtcaccgtgtcctcaggcggaggtggaagcggagggggaggatctggcggcggaggaagcggaggcgagatcgtcctgacccagagcccagggaccctgagtttgtccccgggcgagcgcgcgaccctcagttgcagatcctctcagtccatcgtgcactccaacggcaacacgtacctcgagtggtaccagcagaagcccgggcaggccccgcgactgctcatctacaaggtgagcaaccggttctccggcatccccgaccgcttctcagggtccggctcggggacggatttcaccctcacgattagccgcttggagcccgaagacttcgccgtgtactactgctttcagggaagtcacgtgccgcgtaccttcggggggggcacgaaagtggaaattaag

SEQ ID NO: 475 - protein, 247 aa, synthetic construct
QVTLKQSGAEVKKPGSSVKVSCTASGYTFTSYHVSWVRQAPGQGLEWLGRIYPGDGSTQYNEKFKGKVTITADKSMDTSFMELTSLTSEDTAVYYCAREGTYYAMDLWGQGTLVTVSSGGGGSGGGGSGGGGSGGEIVLTQSPGTLSLSPGERATLSCRSSQSIVHSNGNTYLAWYQQKPGQAPRLLISKVSNRFSGVPDRFSGSGSGTDFTLTISRLEPEDFAVYYCQQGSHVPRTFGGGTKVEIK

SEQ ID NO: 476 - DNA, 741 bp, synthetic construct
  source 1..741: mol_type = other DNA
caggtgaccctgaagcagtctggggccgaggtgaagaagcctgggtcctcggtgaaggtgtcctgcacggcttctggatacaccttcaccagctatcatgtcagctgggtacgacaggcccctggacaagggcttgagtggttgggaaggatctaccctggcgatggttcaacacagtacaatgagaagttcaagggcaaagtcacgattaccgcggacaaatccatggacacatccttcatggagctgaccagcctgacatctgaggacacggccgtatattactgtgcgagagagggaacttactacgctatggacctctggggccaagggaccctggtcaccgtgtcctcaggcggaggtggaagcggagggggaggatctggcggcggaggaagcggaggcgagatcgtcctgacccagagcccagggaccctgagtttgtccccgggcgagcgcgcgaccctcagttgcagatcctctcagtccatcgtgcactccaacggcaacacgtacctcgcgtggtaccagcagaagcccgggcaggccccgcgactgctcatctccaaggtgagcaaccggttctccggcgtccccgaccgcttctcagggtccggctcggggacggatttcaccctcacgattagccgcttggagcccgaagacttcgccgtgtactactgccaacagggaagtcacgtgccgcgtaccttcggggggggcacgaaagtggaaattaag

SEQ ID NO: 477 - protein, 247 aa, synthetic construct
QVQLVQSGAEVKKPGASVKVSCKASGYTFTSYHMHWVRQAPGQRLEWMGWIYPGDGSTQYNEKFKGKVTITRDTSASTAYMELSSLRSEDTAVYYCAREGTYYAMDYWGQGTLVTVSSGGGGSGGGGSGGGGSGGDIVMTQTPLSLPVTPGEPASISCRSSQSIVHSNGNTYLDWYLQKPGQSPQLLIYKVSNRFSGVPDRFSGSGSGTDFTLKISRVEAEDVGVYYCMQGSHVPRTFGGGTKVEIK

SEQ ID NO: 478 - DNA, 741 bp, synthetic construct
  source 1..741: mol_type = other DNA
caggtgcagctggtgcagtctggggccgaggtgaagaagcctggggcctcggtgaaggtgtcctgcaaggcttctggatacaccttcaccagctatcatatgcactgggtacgacaggcccctggacaaaggcttgagtggatgggatggatctaccctggcgatggttcaacacagtacaatgagaagttcaagggcaaagtcacgattacccgggacacatccgcgagcacagcctacatggagctgagcagcctgagatctgaggacacggccgtatattactgtgcgagagagggaacttactacgctatggactactggggccaagggaccctggtcaccgtgtcctcaggcggaggtggaagcggagggggaggatctggcggcggaggaagcggaggcgacatcgtcatgacccagaccccactgtccctgcctgtgaccccgggcgagcccgcgagcatcagttgcagatcctctcagtccatcgtgcactccaacggcaacacgtacctcgactggtacctgcagaagcccgggcagtccccgcaactgctcatctacaaggtgagcaaccggttctccggcgtccccgaccgcttctcagggtccggctcggggacggatttcaccctcaagattagccgcgtggaggccgaagacgtcggcgtgtactactgcatgcagggaagtcacgtgccgcgtaccttcggggggggcacgaaagtggaaattaag

SEQ ID NO: 479 - protein, 244 aa, synthetic construct
QVQLQESGPGLVKPSQTLSLTCTVSGYSITSGYSWHWIRQPPGKGLEWIGYIHFSGSTHYHPSLKSRVTISVDTSKNQFSLKLSSVTAADTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 480 - protein, 244 aa, synthetic construct
EVQLVESGGGLVKPGGSLRLSCAASGYSITSGYSWHWVRQAPGKGLEWVSYIHFSGSTHYHPSLKSRFTISRDNAKNSLYLQMNSLRAEDTAVYYCARGGVVSHYAMDCWGQGTTVTVSSGGGGSGGGGSGGGGSGGDIQMTQSPSSVSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 481 - protein, 244 aa, synthetic construct
EVQLVESGGGLVKPGGSLRLSCAASGYSITSGYSWHWVRQAPGKGLEWVGYIHFSGSTHYHPSLKSRFTISRDDSKNTLYLQMNSLKTEDTAVYYCARGGVVSHYAMDCWGQGTTVTVSSGGGGSGGGGSGGGGSGGEIVLTQSPATLSLSPGERATLSCRASENIYSNLAWYQQKPGQAPRLLIYAATYLPDGIPARFSGSGSGTDFTLTISSLEPEDFAVYYCQHFWVTPYTFGGGTKVEIK

SEQ ID NO: 482 - protein, 244 aa, synthetic construct
QVQLQQSGPGLVKPSQTLSLTCAISGYSITSGYSWHWIRQSPSRGLEWLGYIHFSGSTHYHPSLKSRITINPDTSKNQFSLQLNSVTPEDTAVYYCARGGVVSHYAMDCWGQGTTVTVSSGGGGSGGGGSGGGGSGGEIVLTQSPATLSLSPGERATLSCRASENIYSNLAWYQQKPGQAPRLLIYAATYLPDGIPARFSGSGSGTDFTLTISRLEPEDFAVYYCQHFWVTPYTFGGGTKVEIK

SEQ ID NO: 483 - protein, 244 aa, synthetic construct
EVQLVESGGGLVKPGGSLRLSCAASGYSITSGYSWHWVRQAPGKGLEWVGYIHFSGSTHYHPSLKSRFTISRDDSKNTLYLQMNSLKTEDTAVYYCARGGVVSHYAMDCWGQGTTVTVSSGGGGSGGGGSGGGGSGGDIQMTQSPSSVSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 484 - protein, 244 aa, synthetic construct
EVQLVESGGGLVQPGGSLRLSCAASGYSITSGYSWHWVRQAPGKGLEWVSYIHFSGSTHYHPSLKSRFTISRDNSKNTLYLQMNSLRAEDTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 485 - protein, 244 aa, synthetic construct
EVQLVESGGGLVKPGGSLRLSCAASGYSITSGYSWHWVRQAPGKGLEWVGYIHFSGSTHYHPSLKSRFTISRDDSKNTLYLQMNSLKTEDTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 486 - protein, 244 aa, synthetic construct
QVQLQQWGAGLLKPSETLSLTCAVYGYSITSGYSWHWIRQPPGKGLEWIGYIHFSGSTHYHPSLKSRVTISVDTSKNQFSLKLSSVTAADTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 487 - protein, 244 aa, synthetic construct
QVQLQESGPGLVKPSQTLSLTCTVSGYSITSGYSWHWIRQHPGKGLEWIGYIHFSGSTHYHPSLKSRVTISVDTSKNQFSLKLSSVTAADTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 488 - protein, 244 aa, synthetic construct
QVQLQQSGPGLVKPSQTLSLTCAISGYSITSGYSWHWIRQSPSRGLEWLGYIHFSGSTHYHPSLKSRITINPDTSKNQFSLQLNSVTPEDTAVYYCARGGVVSHYAMDCWGQGTTVTVSSGGGGSGGGGSGGGGSGGDIQMTQSPSSLSASVGDRVTITCRASENIYSNLAWYQQKPGKAPKLLIYAATYLPDGVPSRFSGSGSGTDFTLTISSLQPEDFATYYCQHFWVTPYTFGGGTKVEIK

SEQ ID NO: 489 - protein, 243 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 490 - DNA, 729 bp, synthetic construct
  source 1..729: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 491 - protein, 244 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 492 - DNA, 732 bp, synthetic construct
  source 1..732: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 493 - protein, 245 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 494 - DNA, 735 bp, synthetic construct
  source 1..735: mol_type = other DNA
caggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 495 - protein, 243 aa, synthetic construct
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 496 - protein, 729 aa, synthetic construct
GAAGTGCAGCTGGTGGAAAGCGGCGGAGGCCTGGTGCAGCCTGGCGGCAGCCTGAGACTGTCTTGCGCCGCCAGCGGCTTCACCTTCAGCAGCTACGACATGCACTGGGTCCGCCAGGCCACCGGCAAGGGACTGGAATGGGTGTCCGCCATCGGCACAGCCGGCGACACTTACTACCCCGGCAGCGTGAAGGGCCGGTTCACCATCAGCAGAGAGAACGCCAAGAACAGCCTGTACCTGCAGATGAACAGCCTTCGAGCCGGCGATACCGCCGTGTATTACTGTGCAAGAGATCTCCCTGGTAGCTACTGGTACTTCGATCTCTGGGGCCGTGGCACCCTGGTCACTGTGTCCTCAGGCGGAGGTGGAAGCGGAGGGGGAGGATCTGGCGGCGGAGGAAGCGGAGGCGACATCCAGATGACCCAGTCTCCATCCTCCCTGTCTGCATCTGTAGGAGACAGAGTCACCATCACTTGCCGGGCAAGTCAGAGCATTAGCAGCTATTTAAATTGGTATCAGCAGAAACCAGGGAAAGCCCCTAAGCTCCTGATCTATGCTGCATCCAGTTTGCAAAGTGGGGTCCCATCAAGGTTCAGTGGCAGTGGATCTGGGACAGATTTCACTCTCACCATCAGCAGTCTGCAACCTGAAGATTTTGCAACTTACTACTGTCAACAGAGTTACAGTACCCCTCTCACTTTCGGCGGCGGAACAAAGGTGGAGATCAAG

SEQ ID NO: 497 - protein, 243 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 498 - DNA, 729 bp, synthetic construct
  source 1..729: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 499 - protein, 243 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 500 - DNA, 729 bp, synthetic construct
  source 1..729: mol_type = other DNA
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 501 - protein, 242 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 502 - DNA, 726 bp, synthetic construct
  source 1..726: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 503 - protein, 243 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 504 - DNA, 729 bp, synthetic construct
  source 1..729: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 505 - protein, 243 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 506 - DNA, 729 bp, synthetic construct
  source 1..729: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctcaggcggaggtggaagcggagggggaggatctggcggcggaggaagcggaggcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 507 - protein, 244 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARERVSQRGAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 508 - protein, 243 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARGNPDKDPFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 509 - protein, 245 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARDFYCTNWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 510 - protein, 244 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARESSSGSYWYFDLWGRGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 511 - protein, 250 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDSGYKYNLYYYYYYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 512 - protein, 249 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGDLSHYYYYMDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 513 - protein, 246 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARENRRYNSCYYFDYWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 514 - protein, 249 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGDLSHYYYYLDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 515 - protein, 245 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARATLLSLSYDAFDIWGQGTMVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 516 - protein, 248 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGDLSHYYYMDVWGKGTTVTVSSGGGGSGGGGSGGGGSGGQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQWVFGGGTKLTVL

SEQ ID NO: 517 - protein, 241 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARERDRWFDPWGQGTLVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 518 - protein, 247 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARETPPSLGAFDIWGQGTMVTVSSGGGGSGGGGSGGGGSGGQSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNWVFGGGTKLTVL

SEQ ID NO: 519 - protein, 251 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAREAYCLSDSYWYFDLWGRGTLVTVSSGGGGSGGGGSGGGGSGGQSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 520 - protein, 248 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARESWKYFYPRGYMDVWGKGTTVTVSSGGGGSGGGGSGGGGSGGDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 521 - protein, 16 aa, synthetic construct
RSSQSIVHSNGNTYLE

SEQ ID NO: 522 - protein, 12 aa, synthetic construct
KVSNRFSGVPDR

SEQ ID NO: 523 - protein, 9 aa, synthetic construct
FQGSHVPRT

SEQ ID NO: 524 - protein, 12 aa, synthetic construct
ASGYTFTSYHIH

SEQ ID NO: 525 - protein, 18 aa, synthetic construct
WIYPGNVNTEYNEKFKGK

SEQ ID NO: 526 - protein, 9 aa, synthetic construct
EEITYAMDY

SEQ ID NO: 527 - protein, 16 aa, synthetic construct
RSSQSIVHSNGNTYLD

SEQ ID NO: 528 - protein, 12 aa, synthetic construct
KVSNRFSGVPDR

SEQ ID NO: 529 - protein, 9 aa, synthetic construct
MQGSHVPRT

SEQ ID NO: 530 - protein, 11 aa, synthetic construct
SGYTFTSYHMH

SEQ ID NO: 531 - protein, 17 aa, synthetic construct
WIYPGDGSTQYNEKFKG

SEQ ID NO: 532 - protein, 9 aa, synthetic construct
EGTYYAMDY

SEQ ID NO: 533 - protein, 11 aa, synthetic construct
RASQSISSYLN

SEQ ID NO: 534 - protein, 7 aa, synthetic construct
AASSLQS

SEQ ID NO: 535 - protein, 9 aa, synthetic construct
QQSYSTPLT

SEQ ID NO: 536 - protein, 5 aa, synthetic construct
SYGIS

SEQ ID NO: 537 - protein, 17 aa, synthetic construct
WISAYNGNTNYAQKLQG

SEQ ID NO: 538 - protein, 11 aa, synthetic construct
ERVSQRGAFDI

SEQ ID NO: 539-541 - skipped (3 entries)

SEQ ID NO: 542 - protein, 5 aa, synthetic construct
SYSMN

SEQ ID NO: 543 - protein, 17 aa, synthetic construct
YISSSSSTIYYADSVKG

SEQ ID NO: 544 - protein, 10 aa, synthetic construct
GNPDKDPFDY

SEQ ID NO: 545-547 - skipped (3 entries)

SEQ ID NO: 548 - protein, 7 aa, synthetic construct
SGSYYWS

SEQ ID NO: 549 - protein, 16 aa, synthetic construct
YIYYSGSTNYNPSLKS

SEQ ID NO: 550 - protein, 11 aa, synthetic construct
DFYCTNWYFDL

SEQ ID NO: 551-553 - skipped (3 entries)

SEQ ID NO: 554 - protein, 5 aa, synthetic construct
SYYWS

SEQ ID NO: 555 - protein, 16 aa, synthetic construct
YIYYSGSTNYNPSLKS

SEQ ID NO: 556 - protein, 12 aa, synthetic construct
ESSSGSYWYFDL

SEQ ID NO: 557-559 - skipped (3 entries)

SEQ ID NO: 560 - protein, 5 aa, synthetic construct
SYWIG

SEQ ID NO: 561 - protein, 17 aa, synthetic construct
IIYPGDSDTRYSPSFQG

SEQ ID NO: 562 - protein, 17 aa, synthetic construct
DSGYKYNLYYYYYYMDV

SEQ ID NO: 563 - protein, 14 aa, synthetic construct
ASSTGAVTSGYYPN

SEQ ID NO: 564 - protein, 7 aa, synthetic construct
STSNKHS

SEQ ID NO: 565 - protein, 10 aa, synthetic construct
LLYYGGAQWV

SEQ ID NO: 566 - skipped

SEQ ID NO: 567 - protein, 17 aa, synthetic construct
WISAYNGNTNYAQKLQG

SEQ ID NO: 568 - protein, 13 aa, synthetic construct
GGDLSHYYYYMDV

SEQ ID NO: 569-572 - skipped (4 entries)

SEQ ID NO: 573 - protein, 17 aa, synthetic construct
WISAYNGNTNYAQKLQG

SEQ ID NO: 574 - protein, 13 aa, synthetic construct
ENRRYNSCYYFDY

SEQ ID NO: 575 - protein, 14 aa, synthetic construct
ASSTGAVTSGYYPN

SEQ ID NO: 576 - protein, 7 aa, synthetic construct
STSNKHS

SEQ ID NO: 577 - protein, 10 aa, synthetic construct
LLYYGGAQWV

SEQ ID NO: 578 - skipped

SEQ ID NO: 579 - protein, 17 aa, synthetic construct
WISAYNGNTNYAQKLQG

SEQ ID NO: 580 - protein, 13 aa, synthetic construct
GGDLSHYYYYLDV

SEQ ID NO: 581-583 - skipped (3 entries)

SEQ ID NO: 584 - protein, 5 aa, synthetic construct
SNYMS

SEQ ID NO: 585 - protein, 16 aa, synthetic construct
VIYSGGSTYYADSVKG

SEQ ID NO: 586 - protein, 13 aa, synthetic construct
ATLLSLSYDAFDI

SEQ ID NO: 587 - protein, 14 aa, synthetic construct
ASSTGAVTSGYYPN

SEQ ID NO: 588 - protein, 7 aa, synthetic construct
STSNKHS

SEQ ID NO: 589 - protein, 7 aa, synthetic construct
LLYYGGA

SEQ ID NO: 590 - protein, 5 aa, synthetic construct
SYGIS

SEQ ID NO: 591 - protein, 17 aa, synthetic construct
WISAYNGNTNYAQKLQG

SEQ ID NO: 592 - protein, 12 aa, synthetic construct
GGDLSHYYYMDV

SEQ ID NO: 593 - protein, 11 aa, synthetic construct
RASQSISSYLN

SEQ ID NO: 594 - protein, 7 aa, synthetic construct
AASSLQS

SEQ ID NO: 595 - protein, 9 aa, synthetic construct
QQSYSTPLT

SEQ ID NO: 596 - protein, 5 aa, synthetic construct
SYWIG

SEQ ID NO: 597 - protein, 17 aa, synthetic construct
IIYPGDSDTRYSPSFQG

SEQ ID NO: 598 - protein, 8 aa, synthetic construct
ERDRWFDP

SEQ ID NO: 599 - protein, 14 aa, synthetic construct
TGTSSDVGGYNYVS

SEQ ID NO: 600 - protein, 7 aa, synthetic construct
EVSKRPS

SEQ ID NO: 601 - protein, 10 aa, synthetic construct
SSYAGSNNWV

SEQ ID NO: 602 - protein, 5 aa, synthetic construct
SYGIS

SEQ ID NO: 603 - protein, 17 aa, synthetic construct
WISAYNGNTNYAQKLQG

SEQ ID NO: 604 - protein, 11 aa, synthetic construct
ETPPSLGAFDI

SEQ ID NO: 605 - protein, 13 aa, synthetic construct
SGSSSNIGSNTVN

SEQ ID NO: 606 - protein, 7 aa, synthetic construct
SNNQRPS

SEQ ID NO: 607 - protein, 11 aa, synthetic construct
AAWDDSLNGWV

SEQ ID NO: 608 - protein, 7 aa, synthetic construct
SSSYYWG

SEQ ID NO: 609 - protein, 16 aa, synthetic construct
SIYYSGSTYYNPSLKS

SEQ ID NO: 610 - protein, 14 aa, synthetic construct
EAYCLSDSYWYFDL

SEQ ID NO: 611 - protein, 11 aa, synthetic construct
RASQSISSYLN

SEQ ID NO: 612 - protein, 7 aa, synthetic construct
AASSLQS

SEQ ID NO: 613 - protein, 9 aa, synthetic construct
QQSYSTPLT

SEQ ID NO: 614 - protein, 7 aa, synthetic construct
SGGYYWS

SEQ ID NO: 615 - protein, 16 aa, synthetic construct
YIYYSGSTYYNPSLKS

SEQ ID NO: 616 - protein, 14 aa, synthetic construct
ESWKYFYPRGYMDV

SEQ ID NO: 617 - protein, 11 aa, synthetic construct
RASENIYSNLA

SEQ ID NO: 618 - protein, 7 aa, synthetic construct
AATYLPD

SEQ ID NO: 619 - protein, 9 aa, synthetic construct
QHFWVTPYT

SEQ ID NO: 620 - protein, 6 aa, synthetic construct
SGYSWH

SEQ ID NO: 621 - protein, 16 aa, synthetic construct
YIHFSGSTHYHPSLKS

SEQ ID NO: 622 - protein, 11 aa, synthetic construct
GGVVSHYAMDC

SEQ ID NO: 623 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIK

SEQ ID NO: 624 - protein, 118 aa, synthetic construct
QVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSS

SEQ ID NO: 625 - protein, 17 aa, synthetic construct
GGGGSGGGGSGGGGSGG

SEQ ID NO: 626 - protein, 7 aa, synthetic construct
SGGYYWS

SEQ ID NO: 627 - protein, 16 aa, synthetic construct
YIYYSGSTYYNPSLKS

SEQ ID NO: 628 - protein, 9 aa, synthetic construct
HYYYYYMDV

SEQ ID NO: 629 - protein, 7 aa, synthetic construct
TSGVGVG

SEQ ID NO: 630 - protein, 16 aa, synthetic construct
LIYWNDDKRYSPSLKS

SEQ ID NO: 631 - protein, 9 aa, synthetic construct
KTTSFYFDY

SEQ ID NO: 632 - protein, 7 aa, synthetic construct
SGGYYWS

SEQ ID NO: 633 - protein, 16 aa, synthetic construct
YIYYSGSTYYNPSLKS

SEQ ID NO: 634 - protein, 8 aa, synthetic construct
HYYYYMDV

SEQ ID NO: 635 - protein, 5 aa, synthetic construct
SYWMH

SEQ ID NO: 636 - protein, 17 aa, synthetic construct
RINSDGSSTSYADSVKG

SEQ ID NO: 637 - protein, 10 aa, synthetic construct
GVLLYNWFDP

SEQ ID NO: 638 - protein, 7 aa, synthetic construct
SGGYYWS

SEQ ID NO: 639 - protein, 16 aa, synthetic construct
YIYYSGSTYYNPSLKS

SEQ ID NO: 640 - protein, 9 aa, synthetic construct
HYYYYYLDV

SEQ ID NO: 641 - protein, 5 aa, synthetic construct
SYDMH

SEQ ID NO: 642 - protein, 16 aa, synthetic construct
AIGTAGDTYYPGSVKG

SEQ ID NO: 643 - protein, 11 aa, synthetic construct
DLPGSYWYFDL

SEQ ID NO: 644 - protein, 5 aa, synthetic construct
SYAMH

SEQ ID NO: 645 - protein, 17 aa, synthetic construct
WINAGNGNTKYSQKFQG

SEQ ID NO: 646 - protein, 12 aa, synthetic construct
EGNGANPDAFDI

SEQ ID NO: 647 - protein, 7 aa, synthetic construct
TSGVGVG

SEQ ID NO: 648 - protein, 16 aa, synthetic construct
LIYWNDDKRYSPSLKS

SEQ ID NO: 649 - protein, 10 aa, synthetic construct
RHMRLSCFDY

SEQ ID NO: 650 - protein, 7 aa, synthetic construct
SGGYYWS

SEQ ID NO: 651 - protein, 16 aa, synthetic construct
YIYYSGSTYYNPSLKS

SEQ ID NO: 652 - protein, 9 aa, synthetic construct
HYYYYSMDV

SEQ ID NO: 653 - protein, 11 aa, synthetic construct
RASQSISSYLN

SEQ ID NO: 654 - protein, 7 aa, synthetic construct
AASSLQS

SEQ ID NO: 655 - protein, 9 aa, synthetic construct
QQSYSTPLT

SEQ ID NO: 656 - protein, 119 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYSMDVWGKGTTVTVSS

SEQ ID NO: 657 - DNA, 357 bp, synthetic construct
  source 1..357: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactccatggacgtctggggcaaagggaccacggtcaccgtgtcctca

SEQ ID NO: 658 - protein, 120 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHRHMRLSCFDYWGQGTLVTVSS

SEQ ID NO: 659 - DNA, 360 bp, synthetic construct
  source 1..360: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacagacacatgcgtttaagctgttttgactactggggccagggaaccctggtcaccgtgtcctca

SEQ ID NO: 660 - protein, 121 aa, synthetic construct
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAREGNGANPDAFDIWGQGTMVTVSS

SEQ ID NO: 661 - DNA, 363 bp, synthetic construct
  source 1..363: mol_type = other DNA
caggtgcagctggtgcagtctggcgccgaagtgaagaaacctggcgcctccgtgaaggtgtcctgcaaggccagcggctacaccttcaccagctacgccatgcactgggttcgacaggcccctggccagagactggaatggatgggctggatcaacgccggcaacggcaacaccaagtacagccagaaattccagggcagagtgaccatcacccgggacaccagcgccagcaccgcctacatggaactgagcagcctgcggagcgaggacaccgctgtgtattactgtgcgagagaaggaaatggtgccaaccctgatgcttttgatatctggggccaagggacaatggtcaccgtgtcctca

SEQ ID NO: 662 - protein, 119 aa, synthetic construct
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARDLPGSYWYFDLWGRGTLVTVSS

SEQ ID NO: 663 - DNA, 357 bp, synthetic construct
  source 1..357: mol_type = other DNA
gaagtgcagctggtggaaagcggcggaggcctggtgcagcctggcggcagcctgagactgtcttgcgccgccagcggcttcaccttcagcagctacgacatgcactgggtccgccaggccaccggcaagggactggaatgggtgtccgccatcggcacagccggcgacacttactaccccggcagcgtgaagggccggttcaccatcagcagagagaacgccaagaacagcctgtacctgcagatgaacagccttcgagccggcgataccgccgtgtattactgtgcaagagatctccctggtagctactggtacttcgatctctggggccgtggcaccctggtcactgtgtcctca

SEQ ID NO: 664 - protein, 119 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYLDVWGKGTTVTVSS

SEQ ID NO: 665 - DNA, 357 bp, synthetic construct
  source 1..357: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacctggacgtctggggcaaagggaccacggtcaccgtgtcctca

SEQ ID NO: 666 - protein, 119 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCCLGVLLYNWFDPWGQGTLVTVSS

SEQ ID NO: 667 - DNA, 357 bp, synthetic construct
  source 1..357: mol_type = other DNA
gaagtgcagctggtggaaagcggcggaggcctggtgcagcctggcggcagcctgagactgtcttgcgccgccagcggcttcaccttcagcagctactggatgcactgggtccgccaggcccctggcaagggactggtctgggtgtctcgaatcaacagcgacggcagcagcaccagctacgccgacagcgtgaagggccggttcaccatcagccgggacaacgccaagaacaccctgtacctgcagatgaacagcctgcgggccgaggacaccgccgtgtattactgttgtttgggtgttttattatacaactggttcgacccctggggccagggaaccctggtcaccgtgtcctca

SEQ ID NO: 668 - protein, 118 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYMDVWGKGTTVTVSS

SEQ ID NO: 669 - DNA, 354 bp, synthetic construct
  source 1..354: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactacatggacgtctggggcaaagggaccacggtcaccgtgtcctca

SEQ ID NO: 670 - protein, 119 aa, synthetic construct
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHKTTSFYFDYWGQGTLVTVSS

SEQ ID NO: 671 - DNA, 357 bp, synthetic construct
  source 1..357: mol_type = other DNA
cagatcaccctgaaagagtccggccccaccctggtgaaacccacccagaccctgaccctgacatgcaccttcagcggcttcagcctgagcacctctggcgtgggcgtgggctggatcagacagcctcccggcaaggccctggaatggctggccctgatctactggaacgacgacaagcggtacagccccagcctgaagtcccggctgaccatcaccaaggacacctcgaagaaccaggtggtgctgaccatgacaaacatggaccccgtggacaccgccacatattactgtgcacacaaaacgacgtcgttttactttgactactggggccagggaaccctggtcaccgtgtcctca

SEQ ID NO: 672 - protein, 119 aa, synthetic construct
QVQLQESGPGLVKPSQTLSLTCTVSGGSISSGGYYWSWIRQPPGKGLEWIGYIYYSGSTYYNPSLKSRVTISVDTSKNQFSLKLSSVTAADTAVYYCARHYYYYYMDVWGKGTTVTVSS

SEQ ID NO: 673 - DNA, 357 bp, synthetic construct
  source 1..357: mol_type = other DNA
caggtgcagctgcaggaaagcggccctggcctggtgaaacccagccagaccctgagcctgacctgcacagtgtccggcggctcgatcagcagcggcggctactactggtcctggatcagacagccccctggcaagggcctggaatggatcggctacatctactacagcggcagcacctactacaaccccagcctgaagtccagagtgaccatcagcgtggacaccagcaagaaccagttcagcctgaagctgagcagcgtgacagccgccgacaccgctgtgtattactgtgcgagacactactactactactacatggacgtctggggcaaagggaccacggtcaccgtgtcctca

SEQ ID NO: 674 - protein, 107 aa, synthetic construct [sequence omitted]

SEQ ID NO: 675 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctcactttcggcggcggaacaaaggtggagatcaag

SEQ ID NO: 676 - skipped

SEQ ID NO: 677 - protein, 500 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTSGGGTKLEIKGGGGSGGGGSGGGGSGGQVQLQQSGPELVKPGASVRISCKASGYTFTSYHIHWVKQRPGQGLEWIGWIYPGNVNTEYNEKFKGKATLTADKSSSTAYMHLSSLTSEDSAVYFCAREEITYAMDYWGQGTSVTVSSYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 678 - RNA, 943 nt, Homo sp.
  source 1..943: mol_type = other RNA
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 679 - DNA, 6654 bp, Homo sp.
  source 1..6654: mol_type = other DNA
aagtggaggcgtcgcgctggcgggcattcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccattcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 680 - DNA, 1698 bp, Homo sp.
  source 1..1698: mol_type = other DNA
cagaagcagaggggtcagggcgaagtcccagggccccaggcgtggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaa

SEQ ID NO: 681 - DNA, 3217 bp, Homo sp.
  source 1..3217: mol_type = other DNA
atggccgtcatggcgccccgaaccctcgtcctgctactctcgggggctctggccctgacccagacctgggcgggtgagtgcggggtcgggagggaaacggcctctgtggggagaagcaacgggcccgcctggcgggggcgcaggacccgggaagccgcgccgggaggagggtcgggcgggtctcagccactcctcgtccccaggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctctttgaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaattttccacggaataggagattatcccaggtgcctgtgtccaggctggtgtctgggttctgtgctcccttccccatcccaggtgtcctgtccattctcaagatagccacatgtgtgctggaggagtgtcccatgacagatgcaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcctttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaa

SEQ ID NO: 682 - protein, 107 aa, synthetic construct
DIQMTQTTSSLSASLGDRVTISCRASQDINYYLNWYQQKPDGTVKLLIYYSSRLHSGVPSRFSGSGSGTDFSLTISNLEQEDIATYFCQQDDALPYTFGGGTKLEIK

SEQ ID NO: 683 - protein, 117 aa, synthetic construct
QVQLQQSGAELVKPGASVKISCKASGYAFSNYWMNWVKQRPGKGLEWIGQINPGDGDTNYNGKFKGKATLTADKSSSTAYMQLSSLTSEDSAVYFCAREDRDYFDYWGQGTTLTVSS

SEQ ID NO: 684 - protein, 240 aa, synthetic construct
QVQLVESGGGVVQPGRSLRLSCAASGFTFSSYDIHWVRQAPGKGLEWVAVIWYDGSHNYYSDSVKGRFTISRDNSKNTLYLQMNSLRAEDTAVYYCARDYSGSYYDYWGQGTLVTVSSGGGGSGGGGSGGGGSAIQMTQSPSSLSASVGDRVTITCRASQGIRNDLGWYQQKPGKAPKLLIYAASNLQSGVPSRFSGSGSGTDFTLTISSLQPEDFATYYCLQDYSYPRTFGQGTKVEIK

SEQ ID NO: 685 - protein, 107 aa, synthetic construct
AIQMTQSPSSLSASVGDRVTITCRASQGIRNDLGWYQQKPGKAPKLLIYAASNLQSGVPSRFSGSGSGTDFTLTISSLQPEDFATYYCLQDYSYPRTFGQGTKVEIK

SEQ ID NO: 686 - protein, 118 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 687 - protein, 111 aa, synthetic construct
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK